(19) United States
(12) Patent Application Publication
(10) Pub. No.: US 2009/0163434 A1
(43) Pub. Date: Jun. 25, 2009

(54) miR-20 Regulated Genes and Pathways as Targets for Therapeutic Intervention

(76) Inventors: Andreas G. BADER, Austin, TX (US); Mike BYROM, Austin, TX (US); Charles D. JOHNSON, Austin, TX (US); David BROWN, Austin, TX (US)

Correspondence Address: Fullbright & Jaworski L.L.P., 600 Congress Avenue, Suite 2400, Austin, TX, 78701, US

(21) Appl. No.: 12/112,291

(22) Filed: Apr. 30, 2008

Related U.S. Application Data
(60) Provisional application No. 60/915,026, filed on Apr. 30, 2007.

(51) Int. Cl. A61K 48/00; C12Q 1/68; A61P 43/00

(52) U.S. Cl. 514/44; 435/6; 977/773

(57) ABSTRACT

The present invention concerns methods and compositions for identifying genes or genetic pathways modulated by miR-20a, using miR-20a to modulate a gene or gene pathway, using this profile in assessing the condition of a patient and/or treating the patient with an appropriate miRNA.

This application claims priority to U.S. Provisional Patent application Ser. No. 60/915,026 filed Apr. 30, 2007, which is incorporated herein by reference in its entirety.

### BACKGROUND OF THE INVENTION

I. Field of the Invention

The present invention relates to the fields of molecular biology and medicine. More specifically, the invention relates to methods and compositions for the treatment of diseases or conditions that are affected by miR-20 microRNAs, microRNA expression, and genes and cellular pathways directly and indirectly modulated by such.

II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundred miRNAs have been identified in plants and animals—including humans—that do not appear to have endogenous siRNAs. Thus, while similar to siRNAs, miRNAs are distinct.

miRNAs thus far observed have been approximately 21-22 nucleotides in length, and they arise from longer precursors transcribed from non-protein-encoding genes. See review of Carrington et al. (2003). The precursors form structures that fold back on themselves in self-complementary regions; they are then processed by the nuclease Dicer (in animals) or DCL1 (in plants) to generate the short double-stranded miRNA. One of the miRNA strands is incorporated into a complex of proteins and miRNA called the RNA-induced silencing complex (RISC). The miRNA guides the RISC complex to a target mRNA, which is then cleaved or translationally silenced, depending on the degree of sequence complementarity of the miRNA to its target mRNA. Currently, it is believed that perfect or nearly perfect complementarity leads to mRNA degradation, as is most commonly observed in plants. In contrast, imperfect base pairing, as is primarily found in animals, leads to translational silencing. However, recent data suggest additional complexity (Bagga et al., 2005; Lim et al., 2005), and mechanisms of gene silencing by miRNAs remain under intense study.

Many miRNAs are conserved among diverse organisms, and this has led to the suggestion that miRNAs are involved in essential biological processes throughout the life span of an organism (Esquela-Kerscher and Slack, 2006). In particular, miRNAs have been implicated in regulating cell growth and cell and tissue differentiation—cellular processes that are associated with the development of cancer. For instance, lin-4 and let-7 both regulate passage from one larval state to another during C. elegans development (Ambros, 2001). mir-14 and bantam are Drosophila miRNAs that regulate cell death, apparently by regulating the expression of genes involved in apoptosis (Brennecke et al., 2003, Xu et al., 2003).

Research on microRNAs is increasing as scientists are beginning to appreciate the broad role that these molecules play in the regulation of eukaryotic gene expression. In particular, several recent studies have shown that expression levels of numerous miRNAs are associated with various cancers (reviewed in Esquela-Kerscher and Slack, 2006; Calin and Croce, 2006). Differential expression of almost all miRNAs across numerous cancer types has been observed (Lu et al., 2005). Most such studies link miRNAs to cancer only by indirect evidence. However, He et al. (2005a) has provided more direct evidence that miRNAs may contribute directly to causing cancer, by forcing the over-expression of six miRNAs in mice, including miR-20a, that resulted in a significant increase in B cell lymphomas.

The inventors previously demonstrated that hsa-miR-20a is involved with the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005, both of which are incorporated by reference). Over-expression of miR-20a significantly reduced viability of Jurkat cells, a human T-cell line derived from leukemic peripheral blood, while significantly increasing the viability and proliferation of primary normal human T-cells. Cell regulators that enhance viability of normal cells while decreasing viability of cancerous cells represent useful therapeutic treatments for cancer. Hsa-miR-20a increased apoptosis (induced death of cells with oncogenic potential) in A549 lung cancer cells and increased the percentage of BJ cells (human foreskin primary cells) in the S phase of the cell cycle while reducing the percentage of those cells in the G1 phase of the cell cycle. The inventors observed that expression of hsa-miR-20a is higher in white blood cells from patients with chronic lymphocytic leukemia than in the same cells from normal patients. Others have shown that hsa-miR-20a regulates the translational yield of the transcription factor, E2F1 (O'Donnell et al., 2005) and appears to be over-expressed in colon, pancreas, and prostate tumors while being down-regulated in breast cancer tumors (Volinia et al., 2006).

Bioinformatics analyses suggest that any given miRNA may bind to and alter the expression of up to several hundred different genes. In addition, a single gene may be regulated by several miRNAs. Thus, each miRNA may regulate a complex interaction among genes, gene pathways, and gene networks. Mis-regulation or alteration of these regulatory pathways and networks, involving miRNAs, are likely to contribute to the development of disorders and diseases such as cancer. Although bioinformatics tools are helpful in predicting miRNA binding targets, all have limitations. Because of the imperfect complementarity with their target binding sites, it is difficult to accurately predict the mRNA targets of miRNAs with bioinformatics tools alone. Furthermore, the complicated interactive regulatory networks among miRNAs and target genes make it difficult to accurately predict which genes will actually be mis-regulated in response to a given miRNA.

Correcting gene expression errors or modulating gene expression by manipulating miRNA expression or by repairing miRNA mis-regulation represent promising methods to repair genetic disorders and cure diseases like cancer. A current, disabling limitation of this approach is that, as mentioned above, the details of the regulatory pathways and networks that are affected by any given miRNA remain generally unidentified. Besides E2F1, the genes, gene pathways, and gene networks that are regulated by miR-20 in cancerous cells remain largely unknown. Currently, this represents a significant limitation for treatment of cancers in which miR-20 may play a role. A need exists to identify the genes, genetic pathways, and genetic networks that are regulated by or that may regulate hsa-miR-20 expression.

### SUMMARY OF THE INVENTION

The present invention provides additional compositions and methods by identifying genes that are direct targets for miR-20 regulation or that are indirect or downstream targets of regulation following the miR-20-mediated modification of another gene(s) expression. Furthermore, the invention describes gene, disease, and/or physiologic pathways and networks that are influenced by miR-20 and its family members. In certain aspects, compositions of the invention are administered to a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

In particular aspects, a subject or patient may be selected for treatment based on expression and/or aberrant expression of one or more miRNA or mRNA. In a further aspect, a subject or patient may be selected for treatment based on aberrations in one or more biologic or physiologic pathway(s), including aberrant expression of one or more gene associated with a pathway, or the aberrant expression of one or more protein encoded by one or more gene associated with a pathway. In still a further aspect, a subject or patient may be selected based on aberrations in both miRNA expression, or biologic or physiologic pathway(s). A subject may be assessed for sensitivity, resistance, and/or efficacy of a therapy or treatment regime based on the evaluation and/or analysis of miRNA or mRNA expression or lack thereof. A subject may be evaluated for amenability to certain therapy prior to, during, or after administration of one or therapy to a subject or patient. Typically, evaluation or assessment may be done by analysis of miRNA and/or mRNA, as well as combination of other assessment methods that include but are not limited to histology, immunohistochemistry, blood work, etc.

In some embodiments, an infectious disease or condition includes a bacterial, viral, parasite, or fungal infection. Many of these genes and pathways are associated with various cancers and other diseases. Cancerous conditions include, but are not limited to astrocytoma, acute myelogenous leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, neuroblastoma, non-Hodgkin lymphoma, lung carcinoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, urothelial carcinoma wherein the modulation of one or more gene is sufficient for a therapeutic response. Typically a cancerous condition is an aberrant hyperproliferative condition associated with the uncontrolled growth or inability to undergo cell death, including apoptosis.

The altered expression or function of miR-20 in cells would lead to changes in the expression of these key genes and contribute to the development of disease or other conditions. Introducing miR-20 (for diseases where the miRNA is down-regulated) or a miR-20 inhibitor (for diseases where the miRNA is up-regulated) into disease cells or tissues or subjects would result in a therapeutic response. The identities of key genes that are regulated directly or indirectly by miR-20 and the disease with which they are associated are provided herein. In certain aspects a cell may be an epithelial, stromal, or mucosal cell. The cell can be, but is not limited to brain, a neuronal, a blood, an esophageal, a lung, a cardiovascular, a liver, a breast, a bone, a thyroid, a glandular, an adrenal, a pancreatic, a stomach, a intestinal, a kidney, a bladder, a prostate, a uterus, an ovarian, a testicular, a splenic, a skin, a smooth muscle, a cardiac muscle, or a striated muscle cell. In certain aspects, the cell, tissue, or target may not be defective in miRNA expression yet may still respond therapeutically to expression or over expression of an miRNA. miR-20 could be used as a therapeutic target for any of these diseases.

In certain aspects, the cell, tissue, or target may not be defective in miRNA expression yet may still respond therapeutically to expression or over expression of a miRNA. miR-20 could be used as a therapeutic target for any of these diseases or conditions. In certain embodiments miR-20 or its compliment can be used to modulate the activity of miR-20 or a miR-20 regulated gene in a subject, organ, tissue, or cell.

A cell, tissue, or subject may be a cancer cell, a cancerous tissue, harbor cancerous tissue, or be a subject or patient diagnosed or at risk of developing a disease or condition. In certain aspects a cancer cell is a neuronal, glial, lung, liver, brain, breast, bladder, blood, leukemic, colon, endometrial, stomach, skin, ovarian, fat, bone, cervical, esophageal, pancreatic, prostate, kidney, or thyroid cell. In still a further aspect cancer includes, but is not limited to astrocytoma, acute myelogenous leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, neuroblastoma, non-Hodgkin lymphoma, lung carcinoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, urothelial carcinoma.

Embodiments of the invention include methods of modulating gene expression, or biologic or physiologic pathways in a cell, a tissue, or a subject comprising administering to the cell, tissue, or subject an amount of an isolated nucleic acid or mimetic thereof comprising a miR-20 nucleic acid sequence in an amount sufficient to modulate the expression of a gene or genes modulated by a miR-20 miRNA. A “miR-20 nucleic acid sequence” includes the full length precursor or processed (i.e., mature) sequence of miR-20 and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of the precursor miRNA or its processed sequence, including all ranges and integers there between. In certain embodiments, the miR-20 nucleic acid sequence contains the full-length processed miRNA sequence and is referred to as a “miR-20 full-length processed nucleic acid sequence.” In still further aspects, the miR-20 nucleic acid comprises at least a 5, 6, 7, 8, 9, 10, 11, 12, 13, 14, 15, 16, 17, 18, 19, 20, 21, 22, 23, 24, 25, or 50 nucleotide (including all ranges and integers there between) segment of miR-20 that is at least 75, 80, 85, 90, 95, 98, 99 or 100% identical to SEQ ID NO:1 to SEQ ID NO:269. In certain aspects, a subset of these miRNAs will be used that include some but not all of the listed miR-20 family members. It is contemplated that one or more miR-20 family members or miR-20 miRNAs may be specifically excluded from certain embodiments of the invention. For instance, in one embodiment only sequences comprising the consensus sequence of SEQ ID NO:269 will be included with all other miRNAs excluded. The general term miR-20 includes all members of the miR-20 family. The mature sequences of miR-20 family includes hsa-miR-20a (MIMAT0000075, SEQ ID NO:1); hsa-miR-20b (MIMAT0001413, SEQ ID NO:2); age-miR-20 (MIMAT0002676, SEQ ID NO:3); bta-miR-20a (MIMAT0003527, SEQ ID NO:4); bta-miR-20b (MIMAT0003796, SEQ ID NO:5); dre-miR-20a (MIMAT0001786, SEQ ID NO:6); dre-miR-20a* (MIMAT0003400, SEQ ID NO:7); dre-miR-20b (MIMAT0001778, SEQ ID NO:8); fru-miR-20 (MIMAT0003083, SEQ ID NO:9); gga-miR-20a (MIMAT0001111, SEQ ID NO:10); gga-miR-20b (MIMAT0001411, SEQ ID NO:11); ggo-miR-20 (MIMAT0002662, SEQ ID NO:12); lca-miR-20 (MIMAT0002669, SEQ ID NO:13); lla-miR-20 (MIMAT0002718, SEQ ID NO:14); mdo-miR-20 (MIMAT0004169, SEQ ID NO:15); mml-miR-20 (MIMAT0002704, SEQ ID NO:16); mmu-miR-20a (MIMAT0000529, SEQ ID NO:17); mmu-miR-20b (MIMAT0003187, SEQ ID NO:18); mne-miR-20 (MIMAT0002725, SEQ ID NO:19); ppa-miR-20 (MIMAT0002683, SEQ ID NO:20); ppy-miR-20 (MIMAT0002690, SEQ ID NO:21); ptr-miR-20 (MIMAT0002697, SEQ ID NO:22); rno-miR-20a (MIMAT0000602, SEQ ID NO:23); rno-miR-20a* (MIMAT0000603, SEQ ID NO:24); rno-miR-20b (MIMAT0003211, SEQ ID NO:25); rno-miR-20b* (MIMAT0003212, SEQ ID NO:26); sla-miR-20 (MIMAT0002711, SEQ ID NO:27); ssc-miR-20 (MIMAT0002129, SEQ ID NO:28); tni-miR-20 (MIMAT0003084, SEQ ID NO:29); xla-miR-20 (MIMAT0001348, SEQ ID NO:30); xtr-miR-20a (MIMAT0003669, SEQ ID NO:31); xtr-miR-20a* (MIMAT0003670, SEQ ID NO:32); and/or xtr-miR-20b (MIMAT0003707, SEQ ID NO:33).

Other members of the miR-20 family, as designated by the Sanger database, include age-miR-106a (MIMAT0002796, SEQ ID NO:63); age-miR-106b (MIMAT0002761 SEQ ID NO:64); age-miR-17-3p (MIMAT0002673 SEQ ID NO:65); age-miR-17-5p (MIMAT0002672 SEQ ID NO:66); age-miR-18 (MIMAT0002674 SEQ ID NO:67); age-miR-93 (MIMAT0002762 SEQ ID NO:68); bta-miR-106 (MIMAT0003784 SEQ ID NO:69); bta-miR-17-3p (MIMAT0003816 SEQ ID NO:70); bta-miR-17-5p (MIMAT0003815 SEQ ID NO:71); bta-miR-18a (MIMAT0003526 SEQ ID NO:72); bta-miR-18b (MIMAT0003517 SEQ ID NO:73); bta-miR-93 (MIMAT0003837 SEQ ID NO:74); dre-miR-17a (MIMAT0001777 SEQ ID NO:75); dre-miR-17a* (MIMAT0003396 SEQ ID NO:76); dre-miR-18a (MIMAT0001779 SEQ ID NO:77); dre-miR-18b (MIMAT0001780 SEQ ID NO:78); dre-miR-18b* (MIMAT0003397 SEQ ID NO:79); dre-miR-18c (MIMAT0001781 SEQ ID NO:80); dre-miR-93 (MIMAT0001810 SEQ ID NO:81); fru-miR-17 (MIMAT0002916 SEQ ID NO:82); fru-miR-18 (MIMAT0002918 SEQ ID NO:83); gga-miR-106 (MIMAT0001142 SEQ ID NO:84); gga-miR-17-3p (MIMAT0001115 SEQ ID NO:85); gga-miR-17-5p (MIMAT0001114 SEQ ID NO:86); gga-miR-18a (MIMAT0001113 SEQ ID NO:87); gga-miR-18b (MIMAT0001141 SEQ ID NO:88); ggo-miR-106a (MIMAT0002795 SEQ ID NO:89); ggo-miR-106b (MIMAT0002758 SEQ ID NO:90); ggo-miR-17-3p (MIMAT0002659 SEQ ID NO:91); ggo-miR-17-5p (MIMAT0002658 SEQ ID NO:92); ggo-miR-18 (MIMAT0002660 SEQ ID NO:93); ggo-miR-93 (MIMAT0002759 SEQ ID NO:94); hsa-miR-106a (MIMAT0000103 SEQ ID NO:95); hsa-miR-106b (MIMAT0000680 SEQ ID NO:96); hsa-miR-17-3p (MIMAT0000071 SEQ ID NO:97); hsa-miR-17-5p (MIMAT0000070 SEQ ID NO:98); hsa-miR-18a (MIMAT0000072 SEQ ID NO:99); hsa-miR-18a* (MIMAT0002891 SEQ ID NO:100); hsa-miR-18b (MIMAT0001412 SEQ ID NO:101); hsa-miR-93 (MIMAT0000093 SEQ ID NO:102); lca-miR-17-3p (MIMAT0002666 SEQ ID NO:103); lca-miR-17-5p (MIMAT0002665 SEQ ID NO:104); lca-miR-18 (MIMAT0002667 SEQ ID NO:105); lla-miR-106b (MIMAT0002777 SEQ ID NO:106); lla-miR-17-3p (MIMAT0002715 SEQ ID NO:107); lla-miR-17-5p (MIMAT0002714 SEQ ID NO:108); lla-miR-18 (MIMAT0002716 SEQ ID NO:109); lla-miR-93 (MIMAT0002778 SEQ ID NO:110); mdo-miR-17-3p (MIMAT0004166 SEQ ID NO:111); mdo-miR-17-5p (MIMAT0004165 SEQ ID NO:112); mdo-miR-18 (MIMAT0004167 SEQ ID NO:113); mdo-miR-93 (MIMAT0004178 SEQ ID NO:114); mml-miR-106a (MIMAT0002798 SEQ ID NO:115); mml-miR-106b (MIMAT0002772 SEQ ID NO:116); mml-miR-17-3p (MIMAT0002701 SEQ ID NO:117); mml-miR-17-5p (MIMAT0002700 SEQ ID NO:118); mml-miR-18 (MIMAT0002702 SEQ ID NO:119); mml-miR-93 (MIMAT0002773 SEQ ID NO:120); mmu-miR-106a (MIMAT0000385 SEQ ID NO:121); mmu-miR-106b (MIMAT0000386 SEQ ID NO:122); mmu-miR-17-3p (MIMAT0000650 SEQ ID NO:123); mmu-miR-17-5p (MIMAT0000649 SEQ ID NO:124); mmu-miR-18 (MIMAT0000528 SEQ ID NO:125); mmu-miR-93 (MIMAT0000540 SEQ ID NO:126); mne-miR-106a (MIMAT0002802 SEQ ID NO:127); mne-miR-106b (MIMAT0002780 SEQ ID NO:128); mne-miR-17-3p (MIMAT0002722 SEQ ID NO:129); mne-miR-17-5p (MIMAT0002721 SEQ ID NO:130); mne-miR-18 (MIMAT0002723 SEQ ID NO:131); mne-miR-93 (MIMAT0002781 SEQ ID NO:132); ppa-miR-106a (MIMAT0002797 SEQ ID NO:133); ppa-miR-106b (MIMAT0002763 SEQ ID NO:134); ppa-miR-17-3p (MIMAT0002680 SEQ ID NO:135); ppa-miR-17-5p (MIMAT0002679 SEQ ID NO:136); ppa-miR-18 (MIMAT0002681 SEQ ID NO:137); ppa-miR-93 (MIMAT0002764 SEQ ID NO:138); ppy-miR-106a (MIMAT0002799 SEQ ID NO:139); ppy-miR-106b (MIMAT0002766 SEQ ID NO:140); ppy-miR-17-3p (MIMAT0002687 SEQ ID NO:141); ppy-miR-17-5p (MIMAT0002686 SEQ ID NO:142); ppy-miR-18 (MIMAT0002688 SEQ ID NO:143); ppy-miR-93 (MIMAT0002767 SEQ ID NO:144); ptr-miR-106a (MIMAT0002800 SEQ ID NO:145); ptr-miR-106b (MIMAT0002769 SEQ ID NO:146); ptr-miR-17-3p (MIMAT0002694 SEQ ID NO:147); ptr-miR-17-5p (MIMAT0002693 SEQ ID NO:148); ptr-miR-18 (MIMAT0002695 SEQ ID NO:149); ptr-miR-93 (MIMAT0002770 SEQ ID NO:150); rno-miR-106b (MIMAT0000825 SEQ ID NO:151); rno-miR-17 (MIMAT0000786 SEQ ID NO:152); rno-miR-18 (MIMAT0000787 SEQ ID NO:153); rno-miR-93 (MIMAT0000817 SEQ ID NO:154); sla-miR-106a (MIMAT0002801 SEQ ID NO:155); sla-miR-106b (MIMAT0002775 SEQ ID NO:156); sla-miR-17-3p (MIMAT0002708 SEQ ID NO:157); sla-miR-17-5p (MIMAT0002707 SEQ ID NO:158); sla-miR-18 (MIMAT0002709 SEQ ID NO:159); sla-miR-93 (MIMAT0002776 SEQ ID NO:160); ssc-miR-106a (MIMAT0002118 SEQ ID NO:161); ssc-miR-18 (MIMAT0002161 SEQ ID NO:162); tni-miR-17 (MIMAT0002917 SEQ ID NO:163); tni-miR-18 (MIMAT0002919 SEQ ID NO:164); xla-miR-18 (MIMAT0001349 SEQ ID NO:165); xla-miR-20 (MIMAT0001348 SEQ ID NO:166); xtr-miR-106 (MIMAT0003583 SEQ ID NO:167); xtr-miR-17-3p (MIMAT0003565 SEQ ID NO:168); xtr-miR-17-5p (MIMAT0003564 SEQ ID NO:169); xtr-miR-18a (MIMAT0003652 SEQ ID NO:170); xtr-miR-18b (MIMAT0003706 SEQ ID NO:171); xtr-miR-93a (MIMAT0003659 SEQ ID NO:172); xtr-miR-93b (MIMAT0003660 SEQ ID NO:173).

Stem-loop sequences of miR-20 family members include hsa-mir-20a (MI0000076, SEQ ID NO:34); hsa-mir-20b (MI0001519, SEQ ID NO:35); age-mir-20, (MI0002980 SEQ ID NO:36); bta-mir-20a (MI0004741 SEQ ID NO:37); bta-mir-20b, (MI0005015 SEQ ID NO:38); dre-mir-20a (MI0001907 SEQ ID NO:39); dre-mir-20b (MI0001899 SEQ ID NO:40); fru-mir-20 (MI0003443 SEQ ID NO:41); gga-mir-20a (MI0001181 SEQ ID NO:42); gga-mir-20b (MI0001517 SEQ ID NO:43); ggo-mir-20 (MI0002968 SEQ ID NO:44); lca-mir-20 (MI0002974 SEQ ID NO:45); lla-mir-20 (MI0003016 SEQ ID NO:46); mdo-mir-20 (MI0005357 SEQ ID NO:47); mml-mir-20 (MI0003004 SEQ ID NO:48); mmu-mir-20a (MI0000568 SEQ ID NO:49); mmu-mir-20b (MI0003536 SEQ ID NO:50); mne-mir-20 (MI0003022 SEQ ID NO:51); ppa-mir-20 (MI0002986 SEQ ID NO:52); ppy-mir-20 (MI0002992 SEQ ID NO:53); ptr-mir-20 (MI0002998 SEQ ID NO:54); rno-mir-20a (MI0000638 SEQ ID NO:55); rno-mir-20b (MI0003554 SEQ ID NO:56); sla-mir-20 (MI0003010 SEQ ID NO:57); ssc-mir-20 (MI0002423 SEQ ID NO:58); tni-mir-20 (MI0003444 SEQ ID NO:59); xla-mir-20 (MI0001453 SEQ ID NO:60); xtr-mir-20a (MI0004911 SEQ ID NO:61); and xtr-mir-20b (MI0004961 SEQ ID NO:62).

In other aspects, the miR-20 family includes stem-loop sequences designated age-mir-106a (MI0003099 SEQ ID NO:174); age-mir-106b (MI0003062 SEQ ID NO:175); age-mir-17 (MI0002977 SEQ ID NO:176); age-mir-18 (MI0002978 SEQ ID NO:177); age-mir-93 (MI0003063 SEQ ID NO:178); bta-mir-106 (MI0005005 SEQ ID NO:179); bta-mir-17 (MI0005031 SEQ ID NO:180); bta-mir-18a (MI0004740 SEQ ID NO:181); bta-mir-18b (MI0004732 SEQ ID NO:182); bta-mir-93 (MI0005050 SEQ ID NO:183); dre-mir-17a-1 (MI0001897 SEQ ID NO:184); dre-mir-17a-2 (MI0001898 SEQ ID NO:185); dre-mir-18a (MI0001900 SEQ ID NO:186); dre-mir-18b (MI0001901 SEQ ID NO:187); dre-mir-18c (MI0001902 SEQ ID NO:188); dre-mir-93 (MI0001954 SEQ ID NO:189); fru-mir-17-1 (MI0003231 SEQ ID NO:190); fru-mir-17-2 (MI0003441 SEQ ID NO:191); fru-mir-18 (MI0003233 SEQ ID NO:192); gga-mir-106 (MI0001210 SEQ ID NO:193); gga-mir-17 (MI0001184 SEQ ID NO:194); gga-mir-18a (MI0001183 SEQ ID NO:195); gga-mir-18b (MI0001209 SEQ ID NO:196); ggo-mir-106a (MI0003096 SEQ ID NO:197); ggo-mir-106b (MI0003059 SEQ ID NO:198); ggo-mir-17 (MI0002965 SEQ ID NO:199); ggo-mir-18 (MI0002966 SEQ ID NO:200); ggo-mir-93 (MI0003060 SEQ ID NO:201); hsa-mir-106a (MI0000113 SEQ ID NO:202); hsa-mir-106b (MI0000734 SEQ ID NO:203); hsa-mir-17 (MI0000071 SEQ ID NO:204); hsa-mir-18a (MI0000072 SEQ ID NO:205); hsa-mir-18b (MI0001518 SEQ ID NO:206); hsa-mir-93 (MI0000095 SEQ ID NO:207); lca-mir-17 (MI0002971 SEQ ID NO:208); lca-mir-18 (MI0002972 SEQ ID NO:209); lla-mir-106b (MI0003078 SEQ ID NO:210); lla-mir-17 (MI0003013 SEQ ID NO:211); lla-mir-18 (MI0003014 SEQ ID NO:212); lla-mir-93 (MI0003079 SEQ ID NO:213); mdo-mir-17 (MI0005354 SEQ ID NO:214); mdo-mir-18 (MI0005355 SEQ ID NO:215); mdo-mir-93 (MI0005369 SEQ ID NO:216); mml-mir-106a (MI0003107 SEQ ID NO:217); mml-mir-106b (MI0003073 SEQ ID NO:218); mml-mir-17 (MI0003001 SEQ ID NO:219); mml-mir-18 (MI0003002 SEQ ID NO:220); mml-mir-93 (MI0003074 SEQ ID NO:221); mmu-mir-106a (MI0000406 SEQ ID NO:222); mmu-mir-106b (MI0000407 SEQ ID NO:223); mmu-mir-17 (MI0000687 SEQ ID NO:224); mmu-mir-18 (MI0000567 SEQ ID NO:225); mmu-mir-93 (MI0000581 SEQ ID NO:226); mne-mir-106a (MI0003120 SEQ ID NO:227); mne-mir-106b (MI0003081 SEQ ID NO:228); mne-mir-17 (MI0003019 SEQ ID NO:229); mne-mir-18 (MI0003020 SEQ ID NO:230); mne-mir-93 (MI0003082 SEQ ID NO:231); ppa-mir-106a (MI0003102 SEQ ID NO:232); ppa-mir-106b (MI0003064 SEQ ID NO:233); ppa-mir-17 (MI0002983 SEQ ID NO:234); ppa-mir-18 (MI0002984 SEQ ID NO:235); ppa-mir-93 (MI0003065 SEQ ID NO:236); ppy-mir-106a (MI0003109 SEQ ID NO:237); ppy-mir-106b (MI0003067 SEQ ID NO:238); ppy-mir-17 (MI0002989 SEQ ID NO:239); ppy-mir-18 (MI0002990 SEQ ID NO:240); ppy-mir-93 (MI0003068 SEQ ID NO:241); ptr-mir-106a (MI0003112 SEQ ID NO:242); ptr-mir-106b (MI0003070 SEQ ID NO:243); ptr-mir-17 (MI0002995 SEQ ID NO:244); ptr-mir-18 (MI0002996 SEQ ID NO:245); ptr-mir-93 (MI0003071 SEQ ID NO:246); rno-mir-106b (MI0000889 SEQ ID NO:247); rno-mir-17 (MI0000845 SEQ ID NO:248); rno-mir-18 (MI0000846 SEQ ID NO:249); rno-mir-93 (MI0000880 SEQ ID NO:250); sla-mir-106a (MI0003115 SEQ ID NO:251); sla-mir-106b (MI0003076 SEQ ID NO:252); sla-mir-17 (MI0003007 SEQ ID NO:253); sla-mir-18 (MI0003008 SEQ ID NO:254); sla-mir-93 (MI0003077 SEQ ID NO:255); ssc-mir-106a (MI0002412 SEQ ID NO:256); ssc-mir-18 (MI0002455 SEQ ID NO:257); tni-mir-17-1 (MI0003232 SEQ ID NO:258); tni-mir-17-2 (MI0003442 SEQ ID NO:259); tni-mir-18 (MI0003234 SEQ ID NO:260); xla-mir-18 (MI0001454 SEQ ID NO:261); xtr-mir-106 (MI0004822 SEQ ID NO:262); xtr-mir-17 (MI0004803 SEQ ID NO:263); xtr-mir-18a (MI0004893 SEQ ID NO:264); xtr-mir-18b (MI0004959 SEQ ID NO:265); xtr-mir-93a (MI0004900 SEQ ID NO:266); and xtr-mir-93b (MI0004901 SEQ ID NO:267). Generally the miR-20 family has a consensus sequence (as depicted using WIPO standard designations for nucleotides) of SUGCWNHNNRKGYASNU SEQ ID NO:268 in particular the miR-20 family members designated as miR-20s comprises a consensus of YAAAGUGCUYAYAGUGCAGGU SEQ ID NO:269.

In specific embodiments, a miR-20 containing nucleic acid or a miR-20 nucleic acid is hsa-miR-20a and/or hsa-miR-20b, or a variations thereof. In certain aspects miR-20 is miR-20a or miR-20b. miR-20 can be hsa-mir-20, including hsa-miR-20a or hsa-miR20b. In a further aspect, a miR-20 nucleic acid can be administered with 1, 2, 3, 4, 5, 6, 7, 8, 9, 10 or more miRNAs. miRNA can be administer concurrently, in sequence or in an ordered progression. In certain aspects miR-20 can be administered in combination with one or more of let-7, miR-15a, miR-16, miR-21, miR-26a, miR-31, miR-34a, miR-126, miR-143, miR-145, miR-147, miR-188, miR-200b, miR-200c, miR-215, miR-216, miR-292-3p, and/or miR-331. All or combinations of miRNAs may be administered in a single formulation. Administration may be before, during or after a second therapy.

miR-20 nucleic acids may also include various heterologous nucleic acid sequences, i.e., those sequences not typically found operatively coupled with miR-20 in nature, such as promoters, enhancers, and the like. The miR-20 nucleic acid can be a recombinant nucleic acid, and can be a ribonucleic acid or a deoxyribonucleic acid. The recombinant nucleic acid may comprise a miR-20 expression cassette, i.e., a nucleic acid segment that expresses a nucleic acid when introduce into an environment containing components for nucleic acid synthesis. In a further aspect, the expression cassette is comprised in a viral vector, or plasmid DNA vector or other therapeutic nucleic acid vector or delivery vehicle, including liposomes and the like. In certain aspects, viral vectors can be administered at 1×10², 1×10³, 1×10⁴ 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp).

In a particular aspect, the miR-20 nucleic acid is a synthetic nucleic acid. Moreover, nucleic acids of the invention may be fully or partially synthetic. In still further aspects, a nucleic acid of the invention or a DNA encoding such can be administered at 0.001, 0.01, 0.1, 1, 10, 20, 30, 40, 50, 100, 200, 400, 600, 800, 1000, 2000, to 4000 μg or mg, including all values and ranges there between. In yet a further aspect, nucleic acids of the invention, including synthetic nucleic acid, can be administered at 0.001, 0.01, 0.1, 1, 10, 20, 30, 40, 50, 100, to 200 μg or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

In certain aspects, the gene or genes modulated comprises 1, 2, 3, 4, 5, 6, 7, 8, 9, 10, 11, 12, 13, 14, 15, 20, 25, 30, 35, 40, 45, 50, 100, 150, 200 or more genes or combinations of genes identified in Tables 1, 3, 4, and 5. In still further aspects, the gene or genes modulated may exclude 1, 2, 3, 4, 5, 6, 7, 8, 9, 10, 11, 12, 13, 14, 15, 20, 25, 30, 35, 40, 45, 50, 100, 150, 175 or more genes or combinations of genes identified in Tables 1, 3, 4, and 5. Modulation includes modulating transcription, mRNA levels, mRNA translation, and/or protein levels in a cell, tissue, or organ. In certain aspects the expression of a gene or level of a gene product, such as mRNA, is down-regulated or up-regulated. In a particular aspect the gene modulated comprises or is selected from (and may even exclude) 1, 2, 3, 4, 5, 6, 7, 8, 9, 10, 11, 12, 13, 14, 15, 16, 17, 18, 19, 20, 21, 22, 23, 24, 25, 26. 27, 28, or all of the genes identified in Tables 1, 3, 4, and 5, or any combinations thereof. In certain embodiments a gene modulated or selected to be modulated is from Table 1. In further embodiments a gene modulated or selected to be modulated is from Table 3. In still further embodiments a gene modulated or selected to be modulated is from Table 4. In yet further embodiments a gene modulated or selected to be modulated is from Table 5. Embodiments of the invention may also include obtaining or assessing a gene expression profile or miRNA profile of a target cell prior to selecting the mode of treatment, e.g., administration of a miR-20 nucleic acid or mimetic. The database content related to nucleic acids and genes designated by an accession number or a database submission are incorporated herein by reference as of the filing date of this application. In certain aspects of the invention one or more miRNA may modulate a single gene. In a further aspect, one or more genes in one or more genetic, cellular, or physiologic pathways can be modulated by one or more miRNAs, including miR-20 nucleic acids in combination with other miRNAs.

TABLE 1
Genes with increased (positive values) or decreased (negative values)
expression following transfection of human cancer cells with
pre-miR hsa-miR-20a.

| Gene Symbol | Ref Seq Transcript ID (Pruitt et al., 2005) | Δ log₂ |
|---|---|---|
| ABCA1 | NM_005502 | −1.01473 |
| ALDH6A1 | NM_005589 | 1.04418 |
| ANG /// | NM_001145 /// NM_002937 /// | 0.831501 |
| RLNASE4 | NM_194430 /// NM_194431 | |
| ANK3 | NM_001149 /// NM_020987 | 1.16621 |
| ANKRD46 | NM_198401 | 0.746793 |
| ANTXR1 | NM_018153 /// NM_032208 /// | −1.13558 |
| | NM_053034 | |
| APOH | NM_000042 | 1.21612 |
| AQP3 | NM_004925 | 1.23947 |
| ARG2 | NM_001172 | 2.10966 |
| ARID5B | NM_032199 | 1.35503 |
| ARL7 | NM_005737 | −1.06672 |
| ARTS-1 | NM_016442 | −1.08712 |
| ATP6V0E | NM_003945 | −1.0247 |
| ATP9A | NM_006045 | 1.01985 |
| AXL | NM_001699 /// NM_021913 | 0.763332 |
| BCL2A1 | NM_004049 | −1.77411 |
| BEAN | XM_375359 | −0.714992 |
| BICD2 | NM_001003800 /// NM_015250 | −0.781188 |
| BTG3 | NM_006806 | −1.19255 |
| BTN3A2 | NM_007047 | −0.765137 |
| C19orf2 | NM_003796 /// NM_134447 | −0.755164 |
| C21orf25 | NM_199050 | −0.791738 |
| C2orf17 | NM_024293 | −0.945852 |
| C2orf31 | — | 0.942376 |
| C5orf13 | NM_004772 | 0.909743 |
| C6orf120 | NM_001029863 | −0.719609 |
| C6orf216 | NM_206908 /// NM_206910 /// | 0.743816 |
| | NM_206911 /// NM_206912 /// | |
| | XR_000259 | |
| CA12 | NM_001218 /// NM_206925 | −0.885975 |
| CCL2 | NM_002982 | −1.20227 |
| CCND1 | NM_053056 | −1.21374 |
| CCNG1 | NM_004060 /// NM_199246 | 0.901161 |
| CDC37L1 | NM_017913 | −0.940979 |
| CDH17 | NM_004063 | 0.855968 |
| CDH4 | NM_001794 | −0.99035 |
| CEBPD | NM_005195 | 0.826406 |
| CFH /// CFHL1 | NM_000186 /// NM_001014975 /// | 0.762913 |
| | NM_002113 | |
| CGI-38 | NM_015964 /// NM_016140 | 0.794501 |
| CLIC4 | NM_013943 | 0.705933 |
| COBLL1 | NM_014900 | 1.27699 |
| COL3A1 | NM_000090 | 0.878014 |
| COL4A1 | NM_001845 | −1.05154 |
| COL4A2 | NM_001846 | −1.19339 |
| COQ2 | NM_015697 | −0.707833 |
| CPM | NM_001005502 /// NM_001874 /// | −1.05328 |
| | NM_198320 | |
| CRIPT | NM_014171 | −0.903098 |
| CSPG2 | NM_004385 | −1.17186 |
| CTDSP2 | NM_005730 | 1.22904 |
| CTH | NM_001902 /// NM_153742 | 1.52696 |
| CXCL5 | NM_002994 | 0.702306 |
| DAZAP2 /// | NM_014764 | −1.12846 |
| DAZAP2 /// | NM_014764 /// XM_376165 | −0.826976 |
| LOC401029 | | |
| DCBLD2 | NM_080927 | −0.838774 |
| DCP2 | NM_152624 | 1.28955 |
| DDAH1 | NM_012137 | 1.25935 |
| DHCR24 | NM_014762 | 1.10459 |
| DKFZP586A0522 | NM_014033 | 0.837826 |
| DNAJB6 | NM_005494 /// NM_058246 | −0.983039 |
| DNAJC15 | NM_013238 | 0.799928 |
| DOCK4 | NM_014705 | −0.755571 |
| DPYSL4 | NM_006426 | 0.996621 |
| DSC2 | NM_004949 /// NM_024422 | 1.18113 |
| DST | NM_001723 /// NM_015548 /// | 1.31681 |
| | NM_020388 /// NM_183380 | |
| DSU | NM_018000 | 0.714098 |
| DUSP1 | NM_004417 | −0.823862 |
| DUSP5 | NM_004419 | 0.708305 |
| EHF | NM_012153 | 0.884735 |
| EIF2C1 | NM_012199 | −0.938174 |
| EIF2S1 | NM_004094 | −1.20235 |
| EPHB2 | NM_004442 /// NM_017449 | −1.25564 |
| EREG | NM_001432 | −1.14689 |
| ETS2 | NM_005239 | −0.702474 |
| F2RL1 | NM_005242 | −0.7278 |
| FAM18B | NM_016078 | −0.75677 |
| FAM45B /// | NM_018472 /// NM_207009 | −0.764547 |
| FAM45A | | |
| FAM46A | NM_017633 | 1.30368 |
| FGB | NM_005141 | 1.17875 |
| FGFR3 | NM_000142 /// NM_022965 | 1.01201 |
| FGFR4 | NM_002011 /// NM_022963 /// | 1.01795 |
| | NM_213647 | |
| FGG | NM_000509 /// NM_021870 | 1.22961 |
| FGL1 | NM_004467 /// NM_147203 /// | 1.0979 |
| | NM_201552 /// NM_201553 | |
| FJX1 | NM_014344 | −1.51629 |
| FLJ13910 | NM_022780 | 1.01348 |
| FLJ31568 | NM_152509 | 0.866822 |
| FLRT3 | NM_013281 /// NM_198391 | 1.05708 |
| FTS | NM_001012398 /// NM_022476 | −0.892226 |
| FYCO1 | NM_024513 | −1.48134 |
| FZD7 | NM_003507 | 0.83388 |
| GABRA5 | NM_000810 | −1.21465 |
| GATA6 | NM_005257 | 1.38308 |
| GFPT2 | NM_005110 | −0.719774 |
| GK | NM_000167 /// NM_203391 | 1.06082 |
| GLIPR1 | NM_006851 | −0.802136 |
| GLUL | NM_001033044 /// NM_001033056 /// | 1.16529 |
| | NM_002065 | |
| GNS | NM_002076 | −1.14826 |
| GOLPH2 | NM_016548 /// NM_177937 | −0.800666 |
| GYG2 | NM_003918 | 1.08933 |
| HAS2 | NM_005328 | −1.00653 |
| HCCS | NM_005333 | −1.01956 |
| HIC2 | NM_015094 | 1.19662 |
| HIPK3 | NM_005734 | 0.741004 |
| HMGA2 | NM_001015886 /// NM_003483 /// | 0.766307 |
| | NM_003484 | |
| HMGCS1 | NM_002130 | 0.829036 |
| HN1 | NM_001002032 /// NM_001002033 /// | −1.15736 |
| | NM_016185 | |
| ID4 | NM_001546 | 0.840565 |
| IGFBP1 | NM_000596 /// NM_001013029 | −1.31178 |
| IL11 | NM_000641 | −1.97819 |
| IL8 | NM_000584 | −1.61544 |
| IQGAP2 | NM_006633 | 1.09979 |
| ITGB4 | NM_000213 /// NM_001005619 /// | −1.03625 |
| | NM_001005731 | |
| JAK1 | NM_002227 | −0.988167 |
| JUN | NM_002228 | −0.905043 |
| KCNK5 | NM_003740 | 1.02097 |
| KCNMA1 | NM_001014797 /// NM_002247 | −1.19025 |
| KIAA0494 | NM_014774 | −1.27759 |
| KIAA0882 | NM_015130 | −1.01049 |
| KLF10 | NM_001032282 /// NM_005655 | −0.967187 |
| KRT20 | NM_019010 | 0.737754 |
| KRT4 | NM_002272 | 1.4643 |
| LEPROT | NM_017526 | −0.918245 |
| LHFP | NM_005780 | −0.788633 |
| LIMK1 | NM_002314 /// NM_016735 | −1.59588 |
| LOC257407 | — | 0.902938 |
| LRRC54 | NM_015516 | −0.738825 |
| M6PR | NM_002355 | −1.30233 |
| MAP3K1 | XM_042066 | 1.02679 |
| MAP3K2 | NM_006609 | −0.961694 |
| MARCH6 | NM_005885 | −1.04209 |
| MATN3 | NM_002381 | 0.899535 |
| MGAM | NM_004668 | 1.36376 |
| MGC11332 | NM_032718 | −0.904724 |
| MICA | NM_000247 | −1.15081 |
| MICAL2 | NM_014632 | −0.758803 |
| MICAL-L1 | NM_033386 | 0.719021 |
| MOBK1B | NM_018221 | −1.15411 |
| NAGK | NM_017567 | −1.08281 |
| NES | NM_006617 | 1.02351 |
| NID1 | NM_002508 | 0.856316 |
| NPAS2 | NM_002518 | −1.17566 |
| NPTX1 | NM_002522 | −1.44279 |
| NRP2 | NM_003872 /// NM_018534 /// | −0.811956 |
| | NM_201264 /// NM_201266 /// | |
| | NM_201267 /// NM_201279 | |
| NUPL1 | NM_001008564 /// NM_001008565 /// | −0.809253 |
| | NM_014089 | |
| OBSL1 | XM_051017 | 1.35426 |
| OLR1 | NM_002543 | 1.36616 |
| OSTM1 | NM_014028 | −1.05687 |
| OXTR | NM_000916 | −0.977849 |
| P8 | NM_012385 | 1.31518 |
| PDCD4 | NM_014456 /// NM_145341 | 0.823334 |
| PDGFRL | NM_006207 | 0.726654 |
| PDZK1 | NM_002614 | 1.23771 |
| PELI2 | NM_021255 | 1.00074 |
| PFKP | NM_002627 | −1.1192 |
| PGK1 | NM_000291 | 0.989946 |
| PKP2 | NM_001005242 /// NM_004572 | 1.03828 |
| PLAU | NM_002658 | −1.39659 |
| PLCB1 | NM_015192 /// NM_182734 | 0.891129 |
| POLR3G | NM_006467 | −1.6886 |
| PON2 | NM_000305 /// NM_001018161 | −0.827616 |
| PTHLH | NM_002820 /// NM_198964 /// | −0.902774 |
| | NM_198965 /// NM_198966 | |
| QKI | NM_006775 /// NM_206853 /// | 0.883687 |
| | NM_206854 /// NM_206855 | |
| RAB22A | NM_020673 | −1.26569 |
| RARRES1 | NM_002888 /// NM_206963 | 0.715317 |
| RBKS | NM_022128 | −0.842482 |
| RGC32 | NM_014059 | 0.866694 |
| RHOC | NM_175744 | −0.874504 |
| RNH1 | NM_002939 /// NM_203383 /// | −1.0531 |
| | NM_203384 /// NM_203385 /// | |
| | NM_203386 /// NM_203387 | |
| RRM2 | NM_001034 | −0.896356 |
| S100P | NM_005980 | 1.6654 |
| SERF1A /// | NM_021967 /// NM_022978 | −0.777057 |
| SERF1B | | |
| SERPINE1 | NM_000602 | −2.25784 |
| SESN1 | NM_014454 | 0.845489 |
| SGPL1 | NM_003901 | −1.01306 |
| SKP2 | NM_005983 /// NM_032637 | 0.744696 |
| SLC11A2 | NM_000617 | 0.845458 |
| SLC1A4 | NM_003038 | 0.721939 |
| SLC2A3 | NM_006931 | 0.879266 |
| SNAP23 | NM_003825 /// NM_130798 | 0.791062 |
| SPARC | NM_003118 | 1.39199 |
| SPFH2 | NM_001003790 /// NM_001003791 /// | 0.782553 |
| | NM_007175 | |
| SPOCK | NM_004598 | −1.19175 |
| SQLE | NM_003129 | 0.773943 |
| STC1 | NM_003155 | −1.38313 |
| STX3A | NM_004177 | 0.809319 |
| SYNE1 | NM_015293 /// NM_033071 /// | −0.721107 |
| | NM_133650 /// NM_182961 | |
| TBC1D2 | NM_018421 | −0.96565 |
| TGFBR2 | NM_001024847 /// NM_003242 | −0.924623 |
| TJP2 | NM_004817 /// NM_201629 | 1.19979 |
| TM4SF20 | NM_024795 | 1.0172 |
| TM4SF4 | NM_004617 | −0.700123 |
| TM7SF1 | NM_003272 | −1.8947 |
| TMEPAI | NM_020182 /// NM_199169 /// | −1.02732 |
| | NM_199170 /// NM_199171 | |
| TNFAIP6 | NM_007115 | −2.06788 |
| TNFRSF10B | NM_003842 /// NM_147187 | −0.725441 |
| TNRC9 | XM_049037 | 1.01681 |
| TSPAN8 | NM_004616 | 0.858077 |
| TXLNA | NM_175852 | −0.739199 |
| UEV3 | NM_018314 | −0.955638 |
| USP46 | NM_022832 | −1.54141 |
| VANGL1 | NM_138959 | −0.809203 |
| VLDLR | NM_001018056 /// NM_003383 | −0.99136 |
| VTN | NM_000638 | 1.29843 |
| WNT5A | NM_003392 | 1.06927 |
| ZBTB10 | NM_023929 | 0.763786 |
| ZNF331 | NM_018555 | 0.733817 |
| ZNF395 | NM_018660 | 0.710369 |
| ZNF467 | NM_207336 | 0.738748 |

A further embodiment of the invention is directed to methods of modulating a cellular pathway comprising administering to the cell an amount of an isolated nucleic acid comprising a miR-20 nucleic acid sequence in an amount sufficient to modulate the expression, function, status, or state of a cellular pathway, in particular those pathways described in Table 2 or the pathways known to include one or more genes from Table 1, 3, 4, and/or 5. Modulation of a cellular pathway includes, but is not limited to modulating the expression of one or more gene(s). Modulation of a gene can include inhibiting the function of an endogenous miRNA or providing a functional miRNA to a cell, tissue, or subject. Modulation refers to the expression levels or activities of a gene or its related gene product (e.g., mRNA) or protein, e.g., the mRNA levels may be modulated or the translation of an mRNA may be modulated. Modulation may increase or up regulate a gene or gene product or it may decrease or down regulate a gene or gene product (e.g., protein levels or activity).

Still a further embodiment includes methods of administering an miRNA or mimic thereof, and/or treating a subject or patient having, suspected of having, or at risk of developing a pathological condition comprising one or more of step (a) administering to a patient or subject an amount of an isolated nucleic acid comprising a miR-20 nucleic acid sequence in an amount sufficient to modulate expression of a cellular pathway; and (b) administering a second therapy, wherein the modulation of the cellular pathway sensitizes the patient or subject, or increases the efficacy of a second therapy. An increase in efficacy can include a reduction in toxicity, a reduced dosage or duration of the second therapy, or an additive or synergistic effect. A cellular pathway may include, but is not limited to one or more pathway described in Table 2 below or a pathway that is know to include one or more genes of Tables 1, 3, 4, and/or 5. The second therapy may be administered before, during, and/or after the isolated nucleic acid or miRNA is administered

A second therapy can include administration of a second miRNA or therapeutic nucleic acid such as a siRNA or antisense oligonucleotide, or may include various standard therapies, such as pharmaceuticals, chemotherapy, radiation therapy, drug therapy, immunotherapy, and the like. Embodiments of the invention may also include the determination or assessment of gene expression or gene expression profile for the selection of an appropriate therapy. In a particular aspect, a second therapy is a chemotherapy. A chemotherapy can include, but is not limited to paclitaxel, cisplatin, carboplatin, doxorubicin, oxaliplatin, larotaxel, taxol, lapatinib, docetaxel, methotrexate, capecitabine, vinorelbine, cyclophosphamide, gemcitabine, amrubicin, cytarabine, etoposide, camptothecin, dexamethasone, dasatinib, tipifarnib, bevacizumab, sirolimus, temsirolimus, everolimus, lonafarnib, cetuximab, erlotinib, gefitinib, imatinib mesylate, rituximab, trastuzumab, nocodazole, sorafenib, sunitinib, bortezomib, alemtuzumab, gemtuzumab, tositumomab or ibritumomab.

Embodiments of the invention include methods of treating a subject with a disease or condition comprising one or more of the steps of (a) determining an expression profile of one or more genes selected from Table 1, 3, 4, and/or 5; (b) assessing the sensitivity of the subject to therapy based on the expression profile; (c) selecting a therapy based on the assessed sensitivity; and (d) treating the subject using selected therapy. Typically, the disease or condition will have as a component, indicator, or result mis-regulation of one or more gene of Table 1, 3, 4, and/or 5.

In certain aspects, 2, 3, 4, 5, 6, 7, 8, 9, 10, or more miRNA may be used in sequence or in combination. For instance, any combination of miR-20 with another miRNA can be selected based on observing two given miRNAs share a set of target genes or pathways listed in Tables 1, 2, 4 and 5 that are altered in a particular disease or condition. These two miRNAs may result in an improved therapy (e.g., reduced toxicity, greater efficacy, prolong remission, or other improvements in a subjects condition), result in an increased efficacy, an additive efficacy, or a synergistic efficacy providing an additional or an improved therapeutic response. Without being bound by any particular theory, synergy of two miRNA can be a consequence of regulating the same genes or related genes (related by a common pathway or biologic end result) more effectively (e.g., due to distinct binding sites on the same target or related target(s)) and/or a consequence of regulating different genes, but all of which have been implicated in the same particular disease or condition.

In certain aspects, miR-20 and let-7 can be administered to patients with acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, myxofibrosarcoma, multiple myeloma, neuroblastoma, non-Hodgkin lymphoma, non-small cell lung carcinoma, ovarian carcinoma, esophageal carcinoma, pancreatic carcinoma, prostate carcinoma, squamous cell carcinoma of the head and neck, thyroid carcinoma, or urothelial carcinoma.

Further aspects include administering miR-20 and miR-15 to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, melanoma, mantle cell lymphoma, myxofibrosarcoma, multiple myeloma, neuroblastoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In still further aspects, miR-20 and miR-16 are administered to patients with astrocytoma, breast carcinoma, bladder carcinoma, colorectal carcinoma, endometrial carcinoma, glioblastoma, gastric carcinoma, hepatocellular carcinoma, Hodgkin lymphoma, melanoma, mantle cell lymphoma, myxofibrosarcoma, multiple myeloma, non-small cell lung carcinoma, ovarian carcinoma, esophageal carcinoma, pancreatic carcinoma, prostate carcinoma, squamous cell carcinoma of the head and neck, or thyroid carcinoma.

Aspects of the invention include methods where miR-20 and miR-21 are administered to patients with astrocytoma, acute myeloid leukemia, breast carcinoma, bladder carcinoma, colorectal carcinoma, endometrial carcinoma, glioma, glioblastoma, gastric carcinoma, hepatocellular carcinoma, melanoma, mantle cell lymphoma, neuroblastoma, non-small cell lung carcinoma, ovarian carcinoma, esophageal carcinoma, pancreatic carcinoma, prostate carcinoma, or squamous cell carcinoma of the head and neck.

In still further aspects, miR-20 and miR-26a are administered to patients with acute myeloid leukemia, breast carcinoma, bladder carcinoma, cervical carcinoma, colorectal carcinoma, glioma, glioblastoma, gastric carcinoma, hepatocellular carcinoma, leukemia, melanoma, multiple myeloma, neuroblastoma, non-Hodgkin lymphoma, non-small cell lung carcinoma, ovarian carcinoma, esophageal carcinoma, osteosarcoma, pancreatic carcinoma, or prostate carcinoma.

In yet further aspects, miR-20 and miR-34a are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, mantle cell lymphoma, multiple myeloma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, or urothelial carcinoma.

In certain aspects, miR-20 and miR-126 are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, mantle cell lymphoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In a further aspect, miR-20 and miR-143 are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, Hodgkin lymphoma, leukemia, melanoma, mantle cell lymphoma, multiple myeloma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In still a further aspect, miR-20 and miR-147 are administered to patients with astrocytoma, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

In yet another aspect, miR-20 and miR-188 are administered to patients with astrocytoma, acute myeloid leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, leukemia, melanoma, multiple myeloma, non-Hodgkin lymphoma, non-small cell lung carcinoma, ovarian carcinoma, esophageal carcinoma, pancreatic carcinoma, prostate carcinoma, squamous cell carcinoma of the head and neck, or thyroid carcinoma.

In other aspects, miR-20 and miR-215 are administered to patients with astrocytoma, acute myeloid leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, neuroblastoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, or urothelial carcinoma.

In certain aspects, miR-20 and miR-216 are administered to patients with astrocytoma, breast carcinoma, cervical carcinoma, colorectal carcinoma, endometrial carcinoma, glioma, glioblastoma, gastric carcinoma, hepatocellular carcinoma, Hodgkin lymphoma, leukemia, non-Hodgkin lymphoma, non-small cell lung carcinoma, ovarian carcinoma, esophageal carcinoma, osteosarcoma, prostate carcinoma, or squamous cell carcinoma of the head and neck.

In a further aspect, miR-20 and miR-292-3p are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, leukemia, lipoma, melanoma, myxofibrosarcoma, multiple myeloma, neuroblastoma, non-Hodgkin lymphoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, or urothelial carcinoma.

In still a further aspect, miR-20 and miR-331 are administered to patients with astrocytoma, acute myeloid leukemia, breast carcinoma, bladder carcinoma, cervical carcinoma, colorectal carcinoma, endometrial carcinoma, glioma, glioblastoma, gastric carcinoma, hepatocellular carcinoma, leukemia, melanoma, myxofibrosarcoma, multiple myeloma, neuroblastoma, non-Hodgkin lymphoma, ovarian carcinoma, esophageal carcinoma, osteosarcoma, pancreatic carcinoma, prostate carcinoma, squamous cell carcinoma of the head and neck, or thyroid carcinoma.

In yet a further aspect, miR-20 and miR-200b/c are administered to patients with breast carcinoma, cervical carcinoma, colorectal carcinoma, glioma, glioblastoma, gastric carcinoma, hepatocellular carcinoma, leukemia, lipoma, multiple myeloma, non-small cell lung carcinoma, ovarian carcinoma, esophageal carcinoma, osteosarcoma, pancreatic carcinoma, prostate carcinoma, squamous cell carcinoma of the head and neck, or thyroid carcinoma.

It is contemplated that when miR-20 is given in combination with one or more other miRNA molecules, the two different miRNAs may be given at the same time or sequentially. In some embodiments, therapy proceeds with one miRNA and that therapy is followed up with therapy with the other miRNA 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, or 1, 2, 3, 4, 5, 6, 7, 8, 9, 10, 11, or 12 months or any such combination later.

Further embodiments include the identification and assessment of an expression profile indicative of miR-20 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself or a mimetic thereof.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers or genes from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from a patient and a reference expression profile, such as an expression profile from a normal or non-pathologic sample, or a digitized reference, is indicative of a pathologic, disease, or cancerous condition. In certain aspects the expression profile is an indicator of a propensity to or probability of (i.e., risk factor for a disease or condition) develop such a condition. Such a risk or propensity may indicate a treatment, increased monitoring, prophylactic measures, and the like. A nucleic acid or probe set may comprise or identify a segment of a corresponding mRNA and may include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more segments, including any integer or range derivable there between, of a gene or genetic marker, or a nucleic acid, mRNA or a probe representative thereof that is listed in Tables 1, 3, 4, and/or 5 or identified by the methods described herein.

Certain embodiments of the invention are directed to compositions and methods for assessing, prognosing, or treating a pathological condition in a patient comprising measuring or determining an expression profile of one or more miRNA or marker(s) in a sample from the patient, wherein a difference in the expression profile in the sample from the patient and an expression profile of a normal sample or reference expression profile is indicative of pathological condition and particularly cancer (e.g., In certain aspects of the invention, the miRNAs, cellular pathway, gene, or genetic marker is or is representative of one or more pathway or marker described in Table 1, 2, 3, 4, and/or 5, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, 4, and/or 5, including any combination thereof.

TABLE 2
Significantly affected functional cellular pathways following
hsa-miR-20a over-expression in human cancer cells.

| Gene | |
|---|---|
| Number | Pathway Functions |
| 17 | Cellular Movement, Cellular Growth and Proliferation, |
| | Cardiovascular System Development and Function |
| 14 | Cell Morphology, Cardiovascular System Development and |
| | Function, Cell-To-Cell Signaling and Interaction |
| 13 | Endocrine System Disorders, Small Molecule Biochemistry, |
| | Immune Response |
| 13 | Cardiovascular System Development and Function, Tissue |
| | Morphology, Genetic Disorder |
| 12 | Lipid Metabolism, Molecular Transport, Small Molecule |
| | Biochemistry |
| 9 | Developmental Disorder, Tumor Morphology, Cancer |
| 1 | Cell Signaling, Molecular Transport, Neurological Disease |
| 1 | Cancer, Cell Cycle, Skeletal and Muscular Disorders |

TABLE 3
Predicted target genes of hsa-miR-20a.

| | Ref Seq | |
|---|---|---|
| Gene | Transcript ID | |
| Symbol | (Pruitt et al., 2005) | Description |
| 76P | NM_014444 | Gamma tubulin ring complex protein (76p gene) |
| A1BG | NM_130786 | alpha 1B-glycoprotein |
| A2ML1 | NM_144670 | alpha-2-macroglobulin-like 1 |
| AADAC | NM_001086 | arylacetamide deacetylase |
| AADACL1 | NM_020792 | arylacetamide deacetylase-like 1 |
| AADAT | NM_016228 | alpha-aminoadipate aminotransferase |
| AARSL | NM_020745 | Alanyl-tRNA synthetase like |
| ABAT | NM_000663 | 4-aminobutyrate aminotransferase precursor |
| ABCA1 | NM_005502 | ATP-binding cassette, sub-family A member 1 |
| ABCA10 | NM_080282 | ATP-binding cassette, sub-family A, member 10 |
| ABCB9 | NM_019624 | ATP-binding cassette, sub-family B (MDR/TAP), |
| ABCC13 | NM_172024 | ATP-binding cassette protein C13 isoform b |
| ABCC5 | NM_005688 | ATP-binding cassette, sub-family C, member 5 |
| ABCD2 | NM_005164 | ATP-binding cassette, sub-family D, member 2 |
| ABCE1 | NM_002940 | ATP-binding cassette, sub-family E, member 1 |
| ABCG2 | NM_004827 | ATP-binding cassette, sub-family G, member 2 |
| ABCG4 | NM_022169 | ATP-binding cassette, subfamily G, member 4 |
| ABHD11 | NM_031295 | Abhydrolase domain containing 11 isoform 4 |
| ABHD13 | NM_032859 | Hypothetical protein LOC84945 |
| ABHD2 | NM_007011 | alpha/beta hydrolase domain containing protein |
| ABHD4 | NM_022060 | Abhydrolase domain containing 4 |
| ABI1 | NM_001012750 | abl-interactor 1 isoform b |
| ABL1 | NM_005157 | v-abl Abelson murine leukemia viral oncogene |
| ABLIM1 | NM_001003407 | actin-binding LIM protein 1 isoform b |
| ABR | NM_001092 | Active breakpoint cluster region-related |
| ABT1 | NM_013375 | activator of basal transcription 1 |
| ABTB1 | NM_032548 | Ankyrin repeat and BTB (POZ) domain containing 1 |
| ACAD8 | NM_014384 | acyl-Coenzyme A dehydrogenase family, member 8 |
| ACADSB | NM_001609 | acyl-Coenzyme A dehydrogenase, short/branched |
| ACIN1 | NM_014977 | apoptotic chromatin condensation inducer 1 |
| ACPL2 | NM_152282 | acid phosphatase-like 2 |
| ACPP | NM_001099 | prostatic acid phosphatase precursor |
| ACSL1 | NM_001995 | acyl-CoA synthetase long-chain family member 1 |
| ACSL4 | NM_004458 | acyl-CoA synthetase long-chain family member 4 |
| ACSM1 | NM_052956 | acyl-CoA synthetase medium-chain family member |
| ACTR2 | NM_001005386 | actin-related protein 2 isoform a |
| ACVR1B | NM_004302 | activin A type IB receptor isoform a precursor |
| ADAM19 | NM_033274 | ADAM metallopeptidase domain 19 isoform 2 |
| ADAM21 | NM_003813 | ADAM metallopeptidase domain 21 preproprotein |
| ADAM33 | NM_025220 | ADAM metallopeptidase domain 33 isoform alpha |
| ADAM9 | NM_001005845 | ADAM metallopeptidase domain 9 isoform 2 |
| ADAMTS3 | NM_014243 | ADAM metallopeptidase with thrombospondin type 1 |
| ADAMTS5 | NM_007038 | ADAM metallopeptidase with thrombospondin type 1 |
| ADAMTSL2 | NM_014694 | ADAMTS-like 2 |
| ADAMTSL5 | NM_213604 | thrombospondin, type I, domain containing 6 |
| ADAR | NM_001025107 | adenosine deaminase, RNA-specific isoform d |
| ADARB1 | NM_001033049 | RNA-specific adenosine deaminase B1 isoform 4 |
| ADAT1 | NM_012091 | adenosine deaminase, tRNA-specific 1 |
| ADCY1 | NM_021116 | brain adenylate cyclase 1 |
| ADCY6 | NM_015270 | adenylate cyclase 6 isoform a |
| ADCY9 | NM_001116 | adenylate cyclase 9 |
| ADD1 | NM_001119 | Adducin 1 (alpha) isoform a |
| ADHFE1 | NM_144650 | Alcohol dehydrogenase, iron containing, 1 |
| ADIPOR2 | NM_024551 | adiponectin receptor 2 |
| ADM2 | NM_024866 | adrenomedullin 2 precusor |
| ADORA2B | NM_000676 | adenosine A2b receptor |
| ADPN | NM_025225 | Adiponutrin |
| ADPRHL2 | NM_017825 | ADP-ribosylhydrolase like 2 |
| ADRA1B | NM_000679 | alpha-1B-adrenergic receptor |
| ADRA2A | NM_000681 | alpha-2A-adrenergic receptor |
| ADRA2B | NM_000682 | alpha-2B-adrenergic receptor |
| ADRB3 | NM_000025 | adrenergic, beta-3-, receptor |
| ADSL | NM_000026 | adenylosuccinate lyase |
| AEBP2 | NM_153207 | AE binding protein 2 |
| AFAR3 | NM_201252 | aflatoxin B1 aldehyde reductase 3 |
| AFF1 | NM_005935 | Myeloid/lymphoid or mixed-lineage leukemia |
| AFF2 | NM_002025 | Fragile X mental retardation 2 |
| AFF4 | NM_014423 | ALL1 fused gene from 5q31 |
| AGA | NM_000027 | aspartylglucosaminidase precursor |
| AGBL2 | NM_024783 | ATP/GTP binding protein-like 2 |
| AGGF1 | NM_018046 | angiogenic factor VG5Q |
| AGPAT4 | NM_001012733 | 1-acylglycerol-3-phosphate O-acyltransferase 4 |
| AGPAT5 | NM_018361 | 1-acylglycerol-3-phosphate O-acyltransferase 5 |
| AGTPBP1 | NM_015239 | ATP/GTP binding protein 1 |
| AGTR2 | NM_000686 | angiotensin II receptor, type 2 |
| AGXT2L1 | NM_031279 | alanine-glyoxylate aminotransferase 2-like 1 |
| AHCTF1 | NM_015446 | Transcription factor ELYS |
| AHCY | NM_000687 | S-adenosylhomocysteine hydrolase |
| AHI1 | NM_017651 | Jouberin |
| AHNAK | NM_001620 | AHNAK nucleoprotein isoform 1 |
| AICDA | NM_020661 | activation-induced cytidine deaminase |
| AIM1 | NM_001624 | absent in melanoma 1 |
| AIPL1 | NM_001033054 | aryl hydrocarbon receptor interacting |
| AJAP1 | NM_018836 | transmembrane protein SHREW1 |
| AK1 | NM_000476 | adenylate kinase 1 |
| AK5 | NM_012093 | adenylate kinase 5 isoform 2 |
| AKAP11 | NM_016248 | A-kinase anchor protein 11 isoform 1 |
| AKAP13 | NM_006738 | A-kinase anchor protein 13 isoform 1 |
| AKAP6 | NM_004274 | A-kinase anchor protein 6 |
| AKAP9 | NM_005751 | A-kinase anchor protein 9 isoform 2 |
| AKR1D1 | NM_005989 | aldo-keto reductase family 1, member D1 |
| AKR7A2 | NM_003689 | aldo-keto reductase family 7, member A2 |
| AKT3 | NM_005465 | v-akt murine thymoma viral oncogene homolog 3 |
| ALDH1A3 | NM_000693 | aldehyde dehydrogenase 1A3 |
| ALDH3A2 | NM_000382 | aldehyde dehydrogenase 3A2 isoform 2 |
| ALDH3B1 | NM_000694 | aldehyde dehydrogenase 3B1 isoform a |
| ALDH8A1 | NM_022568 | aldehyde dehydrogenase 8A1 isoform 1 |
| ALDH9A1 | NM_000696 | aldehyde dehydrogenase 9A1 |
| ALDOC | NM_005165 | fructose-bisphosphate aldolase C |
| ALKBH4 | NM_017621 | Hypothetical protein LOC54784 |
| ALKBH5 | NM_017758 | Hypothetical protein LOC54890 |
| ALOX15B | NM_001141 | arachidonate 15-lipoxygenase, second type |
| ALPK1 | NM_025144 | alpha-kinase 1 |
| ALPP | NM_001632 | placental alkaline phosphatase precursor |
| ALS2CL | NM_147129 | ALS2 C-terminal like isoform 1 |
| ALS2CR13 | NM_173511 | Amyotrophic lateral sclerosis 2 (juvenile) |
| ALS2CR15 | NM_138468 | Ica69-related protein |
| ALS2CR19 | NM_057177 | Amyotrophic lateral sclerosis 2 (juvenile) |
| ALX4 | NM_021926 | aristaless-like homeobox 4 |
| AMELX | NM_001142 | amelogenin (X chromosome) isoform 1 precursor |
| AMELY | NM_001143 | amelogenin (Y chromosome) precursor |
| AMID | NM_032797 | apoptosis-inducing factor (AIF)-like |
| AMIGO2 | NM_181847 | amphoterin induced gene 2 |
| AMMECR1 | NM_001025580 | AMMECR1 protein isoform 2 |
| AMOTL1 | NM_130847 | angiomotin like 1 |
| AMPD2 | NM_004037 | adenosine monophosphate deaminase 2 (isoform L) |
| AMPD3 | NM_000480 | erythrocyte adenosine monophosphate deaminase |
| AMZ1 | NM_133463 | archaemetzincin-1 |
| ANAPC11 | NM_001002244 | APC11 anaphase promoting complex subunit 11 |
| ANGEL1 | NM_015305 | angel homolog 1 |
| ANGEL2 | NM_144567 | LOC90806 protein |
| ANGPTL7 | NM_021146 | Angiopoietin-like 7 |
| ANK2 | NM_001148 | Ankyrin 2 isoform 1 |
| ANKFY1 | NM_016376 | Ankyrin repeat and FYVE domain containing 1 |
| ANKH | NM_054027 | ankylosis, progressive homolog |
| ANKK1 | NM_178510 | Ankyrin repeat and kinase domain containing 1 |
| ANKRA2 | NM_023039 | Ankyrin repeat, family A (RFXANK-like), 2 |
| ANKRD10 | NM_017664 | Ankyrin repeat domain 10 |
| ANKRD11 | NM_013275 | Ankyrin repeat domain 11 |
| ANKRD12 | NM_015208 | Ankyrin repeat domain 12 |
| ANKRD13C | NM_030816 | Ankyrin repeat domain 13C |
| ANKRD15 | NM_015158 | Ankyrin repeat domain protein 15 isoform a |
| ANKRD16 | NM_019046 | Ankyrin repeat domain 16 isoform a |
| ANKRD25 | NM_015493 | Ankyrin repeat domain 25 |
| ANKRD28 | NM_015199 | Ankyrin repeat domain 28 |
| ANKRD29 | NM_173505 | Ankyrin repeat domain 29 |
| ANKRD38 | NM_181712 | Ankyrin repeat domain 38 |
| ANKRD42 | NM_182603 | Ankyrin repeat domain 42 |
| ANKRD44 | NM_153697 | Hypothetical protein DKFZp434D2328 |
| ANKRD50 | NM_020337 | Hypothetical protein LOC57182 |
| ANKRD9 | NM_152326 | Ankyrin repeat domain 9 |
| ANKS1A | NM_015245 | Ankyrin repeat and sterile alpha motif domain |
| ANKS1B | NM_020140 | Cajalin 2 isoform c |
| ANKS4B | NM_145865 | harmonin-interacting ankyrin-repeat containing |
| ANTXR1 | NM_018153 | Tumor endothelial marker 8 isoform 3 precursor |
| ANUBL1 | NM_174890 | AN1, ubiquitin-like, homolog |
| ANXA13 | NM_001003954 | Annexin A13 isoform b |
| ANXA7 | NM_001156 | Annexin VII isoform 1 |
| AOF1 | NM_153042 | Amine oxidase (flavin containing) domain 1 |
| AP1G1 | NM_001030007 | Adaptor-related protein complex 1, gamma 1 |
| AP1S2 | NM_003916 | Adaptor-related protein complex 1 sigma 2 |
| AP2B1 | NM_001030006 | Adaptor-related protein complex 2, beta 1 |
| AP3D1 | NM_003938 | Adaptor-related protein complex 3, delta 1 |
| AP4S1 | NM_007077 | Adaptor-related protein complex 4, sigma 1 |
| APBB2 | NM_173075 | Amyloid beta A4 precursor protein-binding, |
| APBB3 | NM_006051 | Amyloid beta precursor protein-binding, family |
| APC | NM_000038 | adenomatosis polyposis coli |
| APCDD1 | NM_153000 | adenomatosis polyposis coli down-regulated 1 |
| APEX1 | NM_001641 | APEX nuclease |
| API5 | NM_006595 | apoptosis inhibitor 5 |
| APOBEC3A | NM_145699 | phorbolin 1 |
| APOBEC3F | NM_001006666 | apolipoprotein B mRNA editing enzyme, catalytic |
| APOBEC4 | NM_203454 | apolipoprotein B mRNA editing enzyme, catalytic |
| APOL1 | NM_003661 | apolipoprotein L1 isoform a precursor |
| APOLD1 | NM_030817 | Hypothetical protein LOC81575 |
| APP | NM_000484 | Amyloid beta A4 protein precursor, isoform a |
| APPBP2 | NM_006380 | Amyloid beta precursor protein-binding protein |
| APPL | NM_012096 | Adaptor protein containing pH domain, PTB domain |
| APXL2 | NM_133456 | Apical protein 2 |
| AQP4 | NM_001650 | aquaporin 4 isoform a |
| AQP9 | NM_020980 | aquaporin 9 |
| ARCN1 | NM_001655 | Archain |
| ARFIP2 | NM_012402 | ADP-ribosylation factor interacting protein 2 |
| ARGFX | NM_001012659 | Hypothetical protein LOC503582 |
| ARHGAP1 | NM_004308 | Rho GTPase activating protein 1 |
| ARHGAP12 | NM_018287 | Rho GTPase activating protein 12 |
| ARHGAP18 | NM_033515 | Rho GTPase activating protein 18 |
| ARHGAP24 | NM_031305 | Rho GTPase activating protein 24 |
| ARHGAP26 | NM_015071 | GTPase regulator associated with the focal |
| ARHGAP5 | NM_001030055 | Rho GTPase activating protein 5 isoform a |
| ARHGAP6 | NM_006125 | Rho GTPase activating protein 6 isoform 3 |
| ARHGEF10 | NM_014629 | Rho guanine nucleotide exchange factor 10 |
| ARHGEF11 | NM_014784 | Rho guanine nucleotide exchange factor (GEF) 11 |
| ARHGEF18 | NM_015318 | Rho-specific guanine nucleotide exchange factor |
| ARHGEF3 | NM_019555 | Rho guanine nucleotide exchange factor 3 |
| ARHGEF6 | NM_004840 | Rac/Cdc42 guanine nucleotide exchange factor 6 |
| ARHGEF7 | NM_003899 | Rho guanine nucleotide exchange factor 7 isoform |
| ARID4A | NM_002892 | retinoblastoma-binding protein 1 isoform I |
| ARID4B | NM_016374 | AT rich interactive domain 4B isoform 1 |
| ARL1 | NM_001177 | ADP-ribosylation factor-like 1 |
| ARL10 | NM_173664 | ADP-ribosylation factor-like 10 |
| ARL13B | NM_144996 | ADP-ribosylation factor-like 2-like 1 isoform 2 |
| ARL4A | NM_005738 | ADP-ribosylation factor-like 4A |
| ARL4C | NM_005737 | ADP-ribosylation factor-like 4C |
| ARMC8 | NM_014154 | armadillo repeat containing 8 isoform 1 |
| ARNT2 | NM_014862 | aryl hydrocarbon receptor nuclear translocator |
| ARPP-19 | NM_006628 | Cyclic AMP phosphoprotein, 19 Kd |
| ARPP-21 | NM_001025068 | Cyclic AMP-regulated phosphoprotein, 21 kD |
| ARRDC1 | NM_152285 | Arrestin domain containing 1 |
| ARSB | NM_000046 | Arylsulfatase B isoform 1 precursor |
| ARSD | NM_001669 | Arylsulfatase D isoform a precursor |
| ARSJ | NM_024590 | Arylsulfatase J |
| ARTS-1 | NM_016442 | type 1 tumor necrosis factor receptor shedding |
| ASAH1 | NM_004315 | N-acylsphingosine amidohydrolase (acid |
| ASAH3L | NM_001010887 | N-acylsphingosine amidohydrolase 3-like |
| ASAHL | NM_014435 | N-acylsphingosine amidohydrolase-like protein |
| ASB1 | NM_016114 | Ankyrin repeat and SOCS box-containing protein |
| ASB13 | NM_024701 | Ankyrin repeat and SOCS box-containing protein |
| ASB5 | NM_080874 | Ankyrin repeat and SOCS box-containing protein |
| ASB6 | NM_017873 | Ankyrin repeat and SOCS box-containing 6 isoform |
| ASB7 | NM_198243 | Ankyrin repeat and SOCS box-containing protein 7 |
| ASB9 | NM_001031739 | Ankyrin repeat and SOCS box-containing 9 isoform |
| ASCIZ | NM_015251 | ATM/ATR-Substrate Chk2-Interacting Zn2+-finger |
| ASF1A | NM_014034 | ASF1 anti-silencing function 1 homolog A |
| ASL | NM_000048 | argininosuccinate lyase isoform 1 |
| ASTN | NM_004319 | astrotactin isoform 1 |
| ATAD2 | NM_014109 | two AAA domain containing protein |
| ATF5 | NM_012068 | activating transcription factor 5 |
| ATF7IP2 | NM_024997 | activating transcription factor 7 interacting |
| ATG10 | NM_031482 | APG10 autophagy 10-like |
| ATG12 | NM_004707 | APG12 autophagy 12-like |
| ATG16L1 | NM_017974 | APG16 autophagy 16-like isoform 2 |
| ATG4B | NM_013325 | APG4 autophagy 4 homolog B isoform a |
| ATG5 | NM_004849 | APG5 autophagy 5-like |
| ATM | NM_000051 | Ataxia telangiectasia mutated protein isoform 1 |
| ATOH8 | NM_032827 | Atonal homolog 8 |
| ATP11A | NM_015205 | ATPase, Class VI, type 11A isoform a |
| ATP12A | NM_001676 | ATPase, H+/K+ transporting, nongastric, alpha |
| ATP1A2 | NM_000702 | Na+/K+-ATPase alpha 2 subunit proprotein |
| ATP2B1 | NM_001001323 | plasma membrane calcium ATPase 1 isoform 1a |
| ATP2B2 | NM_001001331 | plasma membrane calcium ATPase 2 isoform a |
| ATP6V0E | NM_003945 | ATPase, H+ transporting, lysosomal, V0 subunit |
| ATP6V1D | NM_015994 | H(+)-transporting two-sector ATPase |
| ATP7B | NM_000053 | ATPase, Cu++ transporting, beta polypeptide |
| ATP8B4 | NM_024837 | ATPase class I type 8B member 4 |
| ATP9A | NM_006045 | ATPase, Class II, type 9A |
| ATPAF1 | NM_022745 | ATP synthase mitochondrial F1 complex assembly |
| ATPBD1B | NM_018066 | ATP binding domain 1 family, member B |
| ATPBD1C | NM_016301 | ATP binding domain 1 family, member C |
| ATRNL1 | NM_207303 | attractin-like 1 |
| ATXN1 | NM_000332 | Ataxin 1 |
| ATXN3 | NM_001024631 | Ataxin 3 isoform 3 |
| B2M | NM_004048 | beta-2-microglobulin precursor |
| B3GALNT2 | NM_152490 | UDP-GalNAc:betaGlcNAc beta |
| B3GALT2 | NM_003783 | UDP-Gal:betaGlcNAc beta |
| B3GALT5 | NM_006057 | UDP-Gal:betaGlcNAc beta |
| B3GNT5 | NM_032047 | beta-1,3-N-acetylglucosaminyltransferase bGnT-5 |
| B3Gn-T6 | NM_138706 | beta-1,3-N-acetylglucosaminyltransferase |
| B4GALT2 | NM_001005417 | UDP-Gal:betaGlcNAc beta 1,4- |
| B4GALT5 | NM_004776 | UDP-Gal:betaGlcNAc beta 1,4- |
| B4GALT6 | NM_004775 | UDP-Gal:betaGlcNAc beta 1,4- |
| BAALC | NM_001024372 | brain and acute leukemia, cytoplasmic isoform 2 |
| BACH2 | NM_021813 | BTB and CNC homology 1, basic leucine zipper |
| BAG1 | NM_004323 | BCL2-associated athanogene isoform 1L |
| BAG5 | NM_001015048 | BCL2-associated athanogene 5 isoform b |
| BAGE | NM_001187 | B melanoma antigen |
| BAGE4 | NM_181704 | B melanoma antigen family, member 4 |
| BAHD1 | NM_014952 | Bromo adjacent homology domain containing 1 |
| BAMBI | NM_012342 | BMP and activin membrane-bound inhibitor |
| BAPX1 | NM_001189 | Bagpipe homeobox 1 |
| BCAP29 | NM_001008405 | B-cell receptor-associated protein BAP29 isoform |
| BCAS1 | NM_003657 | Breast carcinoma amplified sequence 1 |
| BCAS2 | NM_005872 | Breast carcinoma amplified sequence 2 |
| BCL11B | NM_022898 | B-cell CLL/lymphoma 11B isoform 2 |
| BCL2 | NM_000633 | B-cell lymphoma protein 2 alpha isoform |
| BCL2L11 | NM_006538 | BCL2-like 11 isoform 6 |
| BCL2L2 | NM_004050 | BCL2-like 2 protein |
| BCL6 | NM_001706 | B-cell lymphoma 6 protein |
| BCL6B | NM_181844 | B-cell CLL/lymphoma 6, member B (zinc finger |
| BDH2 | NM_020139 | 3-hydroxybutyrate dehydrogenase, type 2 |
| BET1 | NM_005868 | Blocked early in transport 1 |
| BET1L | NM_016526 | Blocked early in transport 1 homolog (S. |
| BFAR | NM_016561 | apoptosis regulator |
| BHLHB3 | NM_030762 | basic helix-loop-helix domain containing, class |
| BHMT2 | NM_017614 | betaine-homocysteine methyltransferase 2 |
| BICD2 | NM_001003800 | bicaudal D homolog 2 isoform 1 |
| BIRC1 | NM_004536 | baculoviral IAP repeat-containing 1 |
| BIRC4 | NM_001167 | baculoviral IAP repeat-containing protein 4 |
| BIRC4BP | NM_017523 | XIAP associated factor-1 isoform 1 |
| BIRC5 | NM_001012270 | baculoviral IAP repeat-containing protein 5 |
| BLZF1 | NM_003666 | basic leucine zipper nuclear factor 1 |
| BMP8B | NM_001720 | bone morphogenetic protein 8B preproprotein |
| BMPR2 | NM_001204 | bone morphogenetic protein receptor type II |
| BMX | NM_001721 | BMX non-receptor tyrosine kinase |
| BNC2 | NM_017637 | basonuclin 2 |
| BNIP2 | NM_004330 | BCL2/adenovirus E1B 19 kD interacting protein 2 |
| BNIP3L | NM_004331 | BCL2/adenovirus E1B 19 kD-interacting protein |
| BNIPL | NM_138279 | BCL2/adenovirus E1B 19 kD interacting protein |
| BPGM | NM_001724 | 2,3-bisphosphoglycerate mutase |
| BPHL | NM_004332 | biphenyl hydrolase-like |
| BPNT1 | NM_006085 | 3′(2′),5′-bisphosphate nucleotidase 1 |
| BRCA1 | NM_007294 | Breast cancer 1, early onset isoform 1 |
| BRCA2 | NM_000059 | Breast cancer 2, early onset |
| BRD1 | NM_014577 | bromodomain containing protein 1 |
| BRMS1L | NM_032352 | Breast cancer metastasis-suppressor 1-like |
| BRWD1 | NM_001007246 | bromodomain and WD repeat domain containing 1 |
| BSCL2 | NM_032667 | Seipin |
| BSDC1 | NM_018045 | BSD domain containing 1 |
| BTBD10 | NM_032320 | K+ channel tetramerization protein |
| BTBD15 | NM_014155 | BTB (POZ) domain containing 15 |
| BTBD7 | NM_001002860 | BTB (POZ) domain containing 7 isoform 1 |
| BTG1 | NM_001731 | B-cell translocation protein 1 |
| BTG3 | NM_006806 | B-cell translocation gene 3 |
| BTN1A1 | NM_001732 | Butyrophilin, subfamily 1, member A1 |
| BTN3A1 | NM_007048 | Butyrophilin, subfamily 3, member A1 |
| BTN3A2 | NM_007047 | Butyrophilin, subfamily 3, member A2 precursor |
| BUB1 | NM_004336 | BUB1 budding uninhibited by benzimidazoles 1 |
| BVES | NM_007073 | Blood vessel epicardial substance |
| C10orf104 | NM_173473 | Hypothetical protein LOC119504 |
| C10orf114 | NM_001010911 | Hypothetical protein LOC399726 |
| C10orf118 | NM_018017 | CTCL tumor antigen L14-2 |
| C10orf129 | NM_207321 | Hypothetical protein LOC142827 |
| C10orf137 | NM_015608 | erythroid differentiation-related factor 1 |
| C10orf22 | NM_032804 | Hypothetical protein LOC84890 |
| C10orf42 | NM_138357 | Hypothetical protein LOC90550 |
| C10orf46 | NM_153810 | Hypothetical protein LOC143384 |
| C10orf54 | NM_022153 | Hypothetical protein LOC64115 |
| C10orf57 | NM_025125 | Hypothetical protein LOC80195 |
| C10orf58 | NM_032333 | Hypothetical protein LOC84293 |
| C10orf72 | NM_144984 | Hypothetical protein LOC196740 isoform 2 |
| C10orf76 | NM_024541 | Hypothetical protein LOC79591 |
| C10orf78 | NM_001002759 | Hypothetical protein LOC119392 isoform a |
| C10orf85 | NM_001012711 | Hypothetical protein LOC404216 |
| C10orf96 | NM_198515 | Hypothetical protein LOC374355 |
| C10orf97 | NM_024948 | Chromosome 10 open reading frame 97 |
| C11orf1 | NM_022761 | Hypothetical protein LOC64776 |
| C11orf30 | NM_020193 | EMSY protein |
| C11orf38 | NM_212555 | Hypothetical protein LOC399967 |
| C11orf49 | NM_001003678 | Hypothetical protein LOC79096 isoform 4 |
| C11orf54 | NM_014039 | Hypothetical protein LOC28970 |
| C11orf55 | NM_207428 | Hypothetical protein LOC399879 |
| C11orf63 | NM_199124 | Hypothetical protein LOC79864 isoform 2 |
| C11orf69 | NM_152314 | Hypothetical protein LOC120196 |
| C12orf31 | NM_032338 | Hypothetical protein LOC84298 |
| C12orf36 | NM_182558 | Hypothetical protein LOC283422 |
| C12orf44 | NM_021934 | Hypothetical protein LOC60673 |
| C12orf49 | NM_024738 | Hypothetical protein LOC79794 |
| C12orf53 | NM_153685 | Hypothetical protein LOC196500 |
| C13orf1 | NM_020456 | Hypothetical protein LOC57213 |
| C14orf101 | NM_017799 | Hypothetical protein LOC54916 |
| C14orf103 | NM_018036 | Hypothetical protein LOC55102 |
| C14orf105 | NM_018168 | Hypothetical protein LOC55195 |
| C14orf108 | NM_018229 | Hypothetical protein LOC55745 |
| C14orf111 | NM_015962 | Hypothetical protein LOC51077 |
| C14orf119 | NM_017924 | Chromosome 14 open reading frame 119 |
| C14orf126 | NM_080664 | Hypothetical protein LOC112487 |
| C14orf129 | NM_016472 | Hypothetical protein LOC51527 |
| C14orf133 | NM_022067 | Hypothetical protein LOC63894 |
| C14orf138 | NM_024558 | Hypothetical protein LOC79609 |
| C14orf143 | NM_145231 | Hypothetical protein LOC90141 |
| C14orf145 | NM_152446 | Chromosome 14 open reading frame 145 |
| C14orf150 | NM_001008726 | Hypothetical protein LOC112840 |
| C14orf153 | NM_032374 | Hypothetical protein LOC84334 |
| C14orf24 | NM_173607 | Hypothetical protein LOC283635 |
| C14orf28 | NM_001017923 | Hypothetical protein LOC122525 |
| C14orf32 | NM_144578 | MAPK-interacting and spindle-stabilizing |
| C14orf43 | NM_194278 | Hypothetical protein LOC91748 |
| C14orf44 | NM_152445 | Hypothetical protein LOC145483 |
| C15orf17 | NM_020447 | Hypothetical protein LOC57184 |
| C15orf20 | NM_025049 | DNA helicase homolog PIF1 |
| C15orf32 | NM_153040 | Hypothetical protein LOC145858 |
| C15orf40 | NM_144597 | Hypothetical protein LOC123207 |
| C15orf41 | NM_032499 | Hypothetical protein LOC84529 |
| C16orf28 | NM_023076 | Hypothetical protein LOC65259 |
| C16orf34 | NM_144570 | Chromosome 16 open reading frame 34 |
| C16orf45 | NM_033201 | Hypothetical protein LOC89927 |
| C16orf54 | NM_175900 | Hypothetical protein LOC283897 |
| C16orf58 | NM_022744 | Hypothetical protein LOC64755 |
| C16orf59 | NM_025108 | Hypothetical protein LOC80178 |
| C17orf27 | NM_020914 | Chromosome 17 open reading frame 27 |
| C17orf37 | NM_032339 | Chromosome 17 open reading frame 37 |
| C17orf39 | NM_024052 | Hypothetical protein LOC79018 |
| C17orf40 | NM_018428 | hepatocellular carcinoma-associated antigen 66 |
| C17orf53 | NM_024032 | Hypothetical protein LOC78995 |
| C17orf62 | NM_001033046 | Hypothetical protein LOC79415 |
| C17orf69 | NM_152466 | Hypothetical protein LOC147081 |
| C17orf73 | NM_017928 | Hypothetical protein LOC55018 |
| C17orf77 | NM_152460 | Hypothetical protein LOC146723 |
| C18orf1 | NM_001003674 | Hypothetical protein LOC753 isoform gamma 1 |
| C18orf16 | NM_153010 | Hypothetical protein LOC147429 |
| C18orf17 | NM_153211 | Hypothetical protein LOC125488 |
| C18orf19 | NM_152352 | Hypothetical protein LOC125228 |
| C18orf25 | NM_001008239 | Chromosome 18 open reading frame 25 isoform b |
| C18orf26 | NM_173629 | Hypothetical protein LOC284254 |
| C18orf45 | NM_032933 | Hypothetical protein LOC85019 |
| C19orf12 | NM_031448 | Hypothetical protein LOC83636 isoform 2 |
| C19orf2 | NM_003796 | RPB5-mediating protein isoform a |
| C19orf20 | NM_033513 | gene trap ROSA b-geo 22 |
| C19orf31 | NM_001014373 | Hypothetical protein LOC404664 |
| C1GALT1 | NM_020156 | core 1 synthase, |
| C1orf107 | NM_014388 | Hypothetical protein LOC27042 |
| C1orf108 | NM_024595 | Hypothetical protein LOC79647 |
| C1orf110 | NM_178550 | Hypothetical protein LOC339512 |
| C1orf116 | NM_023938 | specifically androgen-regulated protein |
| C1orf130 | NM_001010980 | Hypothetical protein LOC400746 |
| C1orf135 | NM_024037 | Hypothetical protein LOC79000 |
| C1orf138 | NM_001025493 | Hypothetical protein LOC574406 |
| C1orf150 | NM_145278 | Hypothetical protein LOC148823 |
| C1orf151 | NM_001032363 | Chromosome 1 open reading frame 151 protein |
| C1orf155 | NM_033319 | Hypothetical protein LOC91687 |
| C1orf171 | NM_138467 | Hypothetical protein LOC127253 |
| C1orf173 | NM_001002912 | Hypothetical protein LOC127254 |
| C1orf176 | NM_022774 | Hypothetical protein LOC64789 |
| C1orf178 | NM_001010922 | pro-apoptotic Bcl-2 protein isoform a |
| C1orf183 | NM_019099 | Hypothetical protein LOC55924 isoform 1 |
| C1orf19 | NM_052965 | Hypothetical protein LOC116461 |
| C1orf21 | NM_030806 | Chromosome 1 open reading frame 21 |
| C1orf24 | NM_022083 | niban protein isoform 1 |
| C1orf26 | NM_017673 | hypothetical protein LOC54823 |
| C1orf32 | NM_199351 | hypothetical protein LOC387597 |
| C1orf33 | NM_016183 | ribosomal protein P0-like protein |
| C1orf42 | NM_019060 | chromosome 1 open reading frame 42 |
| C1orf63 | NM_020317 | hypothetical protein LOC57035 isoform 2 |
| C1orf69 | NM_001010867 | hypothetical protein LOC200205 |
| C1orf76 | NM_173509 | hypothetical protein MGC16664 |
| C1orf83 | NM_153035 | hypothetical protein LOC127428 |
| C1orf84 | NM_182518 | RP11-506B15.1 protein isoform 3 |
| C1orf9 | NM_014283 | chromosome 1 open reading frame 9 protein |
| C1orf96 | NM_145257 | hypothetical protein LOC126731 |
| C1QDC1 | NM_001002259 | C1q domain containing 1 isoform 1 |
| C1QTNF7 | NM_031911 | C1q and tumor necrosis factor related protein 7 |
| C20orf103 | NM_012261 | chromosome 20 open reading frame 103 precursor |
| C20orf108 | NM_080821 | hypothetical protein LOC116151 |
| C20orf112 | NM_080616 | hypothetical protein LOC140688 |
| C20orf117 | NM_080627 | hypothetical protein LOC140710 isoform 1 |
| C20orf12 | NM_018152 | hypothetical protein LOC55184 |
| C20orf121 | NM_024331 | hypothetical protein LOC79183 |
| C20orf133 | NM_001033086 | hypothetical protein LOC140733 isoform 1 |
| C20orf161 | NM_033421 | sorting nexin 21 isoform a |
| C20orf172 | NM_024918 | hypothetical protein LOC79980 |
| C20orf175 | NM_080829 | hypothetical protein LOC140876 |
| C20orf177 | NM_022106 | hypothetical protein LOC63939 |
| C20orf29 | NM_018347 | hypothetical protein LOC55317 |
| C20orf43 | NM_016407 | hypothetical protein LOC51507 |
| C20orf51 | NM_022099 | hypothetical protein LOC63930 |
| C21orf25 | NM_199050 | hypothetical protein LOC25966 |
| C21orf49 | NM_001006116 | hypothetical protein LOC54067 |
| C21orf55 | NM_017833 | hypothetical protein LOC54943 |
| C21orf58 | NM_058180 | hypothetical protein LOC54058 isoform 1 |
| C21orf62 | NM_019596 | hypothetical protein LOC56245 |
| C21orf63 | NM_058187 | chromosome 21 open reading frame 63 |
| C21orf66 | NM_145328 | GC-rich sequence DNA-binding factor candidate |
| C21orf77 | NM_018277 | hypothetical protein LOC55264 |
| C22orf9 | NM_001009880 | hypothetical protein LOC23313 isoform b |
| C2orf13 | NM_173545 | hypothetical protein LOC200558 |
| C2orf15 | NM_144706 | hypothetical protein LOC150590 |
| C2orf17 | NM_024293 | hypothetical protein LOC79137 |
| C2orf19 | NM_001024676 | chromosome 2 open reading frame 19 |
| C2orf26 | NM_023016 | hypothetical protein LOC65124 |
| C2orf28 | NM_016085 | apoptosis related protein 3 isoform a |
| C2orf3 | NM_003203 | hypothetical protein LOC6936 |
| C3orf1 | NM_016589 | hypothetical protein LOC51300 |
| C3orf21 | NM_152531 | hypothetical protein LOC152002 |
| C3orf27 | NM_007354 | putative GR6 protein |
| C3orf34 | NM_032898 | hypothetical protein LOC84984 |
| C3orf35 | NM_178342 | AP20 region protein isoform E |
| C3orf38 | NM_173824 | hypothetical protein LOC285237 |
| C3orf52 | NM_024616 | TPA-induced transmembrane protein |
| C3orf56 | NM_001007534 | hypothetical protein LOC285311 |
| C3orf62 | NM_198562 | hypothetical protein LOC375341 |
| C3orf63 | NM_015224 | retinoblastoma-associated protein 140 |
| C3orf64 | NM_173654 | AER61 glycosyltransferase |
| C3orf9 | NM_020231 | hypothetical protein LOC56983 |
| C4orf12 | NM_205857 | FBI4 protein |
| C4orf13 | NM_001029998 | hypothetical protein LOC84068 isoform b |
| C4orf15 | NM_024511 | hypothetical protein LOC79441 |
| C5 | NM_001735 | complement component 5 |
| C5orf22 | NM_018356 | hypothetical protein LOC55322 |
| C6orf120 | NM_001029863 | hypothetical protein LOC387263 |
| C6orf128 | NM_145316 | hypothetical protein LOC221468 |
| C6orf134 | NM_024909 | hypothetical protein LOC79969 isoform 2 |
| C6orf139 | NM_018132 | hypothetical protein LOC55166 |
| C6orf15 | NM_014070 | STG protein |
| C6orf151 | NM_152551 | U11/U12 snRNP 48K |
| C6orf201 | NM_206834 | hypothetical protein LOC404220 |
| C6orf208 | NM_025002 | hypothetical protein LOC80069 |
| C6orf35 | NM_018452 | hypothetical protein LOC55836 |
| C6orf49 | NM_013397 | over-expressed breast tumor protein |
| C6orf59 | NM_024929 | hypothetical protein LOC79992 |
| C6orf69 | NM_173562 | hypothetical protein LOC222658 |
| C6orf71 | NM_203395 | chromosome 6 open reading frame 71 |
| C6orf85 | NM_021945 | ion transporter protein |
| C6orf96 | NM_017909 | hypothetical protein LOC55005 |
| C6orf97 | NM_025059 | hypothetical protein LOC80129 |
| C7 | NM_000587 | complement component 7 precursor |
| C7orf19 | NM_032831 | hypothetical protein LOC80228 |
| C7orf29 | NM_138434 | hypothetical protein LOC113763 |
| C8A | NM_000562 | complement component 8, alpha polypeptide |
| C8orf1 | NM_004337 | hypothetical protein LOC734 |
| C8orf30A | NM_016458 | brain protein 16 |
| C8orf37 | NM_177965 | hypothetical protein LOC157657 |
| C8orf38 | NM_152416 | hypothetical protein LOC137682 |
| C8orf44 | NM_019607 | hypothetical protein LOC56260 |
| C8orf45 | NM_173518 | hypothetical protein LOC157777 |
| C8orf49 | NM_001031839 | hypothetical protein LOC606553 |
| C9orf100 | NM_001031728 | hypothetical protein LOC84904 isoform 1 |
| C9orf102 | NM_020207 | stretch responsive protein 278 isoform a |
| C9orf140 | NM_178448 | hypothetical protein LOC89958 |
| C9orf40 | NM_017998 | hypothetical protein LOC55071 |
| C9orf5 | NM_032012 | hypothetical protein LOC23731 |
| C9orf64 | NM_032307 | hypothetical protein LOC84267 |
| C9orf66 | NM_152569 | hypothetical protein LOC157983 |
| C9orf72 | NM_145005 | hypothetical protein LOC203228 isoform b |
| C9orf77 | NM_001025780 | chromosome 9 open reading frame 77 isoform 2 |
| C9orf78 | NM_016482 | chromosome 9 open reading frame 78 isoform 1 |
| C9orf80 | NM_021218 | hypothetical protein LOC58493 |
| C9orf82 | NM_024828 | hypothetical protein LOC79886 |
| C9orf85 | NM_182505 | hypothetical protein LOC138241 isoform a |
| C9orf88 | NM_022833 | hypothetical protein LOC64855 |
| CA10 | NM_020178 | carbonic anhydrase X |
| CA8 | NM_004056 | carbonic anhydrase VIII |
| CABLES1 | NM_138375 | Cdk5 and Abl enzyme substrate 1 |
| CABP2 | NM_016366 | calcium binding protein 2 isoform 1 |
| CACNG4 | NM_014405 | voltage-dependent calcium channel gamma-4 |
| CALCOCO2 | NM_005831 | calcium binding and coiled-coil domain 2 |
| CALD1 | NM_004342 | caldesmon 1 isoform 2 |
| CALN1 | NM_001017440 | calneuron 1 |
| CAMK1D | NM_020397 | calcium/calmodulin-dependent protein kinase ID |
| CAMK2D | NM_172127 | calcium/calmodulin-dependent protein kinase II |
| CAMK2G | NM_001222 | calcium/calmodulin-dependent protein kinase II |
| CAMK2N1 | NM_018584 | calcium/calmodulin-dependent protein kinase II |
| CAMK2N2 | NM_033259 | CaM-KII inhibitory protein |
| CAMKK1 | NM_032294 | calcium/calmodulin-dependent protein kinase 1 |
| CAMSAP1 | NM_015447 | calmodulin regulated spectrin-associated protein |
| CAMSAP1L1 | NM_203459 | calmodulin regulated spectrin-associated protein |
| CAMTA1 | NM_015215 | calmodulin-binding transcription activator 1 |
| CAMTA2 | NM_015099 | calmodulin binding transcription activator 2 |
| CANX | NM_001024649 | calnexin precursor |
| CAPN13 | NM_144575 | calpain 13 |
| CAPN3 | NM_212464 | calpain 3 isoform g |
| CAPN7 | NM_014296 | calpain 7 |
| CAPS2 | NM_032606 | calcyphosphine 2 |
| CARD10 | NM_014550 | caspase recruitment domain protein 10 |
| CARD14 | NM_052819 | caspase recruitment domain protein 14 isoform 2 |
| CARD4 | NM_006092 | caspase recruitment domain family, member 4 |
| CARD8 | NM_014959 | caspase recruitment domain family, member 8 |
| CARKL | NM_013276 | carbohydrate kinase-like |
| CASC3 | NM_007359 | cancer susceptibility candidate 3 |
| CASC4 | NM_138423 | cancer susceptibility candidate 4 isoform a |
| CASP2 | NM_032982 | caspase 2 isoform 1 preproprotein |
| CASP6 | NM_001226 | caspase 6 isoform alpha preproprotein |
| CASP7 | NM_001227 | caspase 7 isoform alpha precursor |
| CASP8 | NM_001228 | caspase 8 isoform A |
| CATSPER2 | NM_172097 | Sperm-associated cation channel 2 isoform 4 |
| CAV1 | NM_001753 | caveolin 1 |
| CAV2 | NM_001233 | caveolin 2 isoform a and b |
| CBX1 | NM_006807 | chromobox homolog 1 (HP1 beta homolog Drosophila |
| CBX2 | NM_005189 | chromobox homolog 2 isoform 1 |
| CBX7 | NM_175709 | chromobox homolog 7 |
| CC2D1A | NM_017721 | putative NFkB activating protein |
| CC2D1B | NM_032449 | Coiled-coil and C2 domain containing 1B |
| CCBE1 | NM_133459 | collagen and calcium binding EGF domains 1 |
| CCBL1 | NM_004059 | cytoplasmic cysteine conjugate-beta lyase |
| CCDC14 | NM_022757 | Coiled-coil domain containing 14 |
| CCDC15 | NM_025004 | Coiled-coil domain containing 15 |
| CCDC16 | NM_052857 | Coiled-coil domain containing 16 |
| CCDC25 | NM_001031708 | Coiled-coil domain containing 25 isoform 1 |
| CCDC43 | NM_144609 | hypothetical protein LOC124808 |
| CCDC52 | NM_144718 | hypothetical protein LOC152185 |
| CCDC6 | NM_005436 | Coiled-coil domain containing 6 |
| CCDC68 | NM_025214 | CTCL tumor antigen se57-1 |
| CCDC69 | NM_015621 | hypothetical protein LOC26112 |
| CCL1 | NM_002981 | small inducible cytokine A1 precursor |
| CCL28 | NM_019846 | small inducible cytokine A28 precursor |
| CCL5 | NM_002985 | small inducible cytokine A5 precursor |
| CCND1 | NM_053056 | cyclin D1 |
| CCND2 | NM_001759 | cyclin D2 |
| CCNE2 | NM_057735 | cyclin E2 isoform 2 |
| CCNF | NM_001761 | cyclin F |
| CCNG2 | NM_004354 | cyclin G2 |
| CCNJ | NM_019084 | cyclin J |
| CCNT2 | NM_001241 | cyclin T2 isoform a |
| CCR6 | NM_004367 | chemokine (C-C motif) receptor 6 |
| CCRL1 | NM_016557 | chemokine (C-C motif) receptor-like 1 |
| CCS | NM_005125 | copper chaperone for superoxide dismutase |
| CD200 | NM_001004196 | CD200 antigen isoform b |
| CD28 | NM_006139 | CD28 antigen |
| CD300LG | NM_145273 | triggering receptor expressed on myeloid cells |
| CD36 | NM_000072 | CD36 antigen |
| CD38 | NM_001775 | CD38 antigen |
| CD46 | NM_002389 | CD46 antigen, complement regulatory protein |
| CD47 | NM_001025079 | CD47 molecule isoform 3 precursor |
| CD59 | NM_000611 | CD59 antigen p18-20 |
| CD68 | NM_001251 | CD68 antigen |
| CD69 | NM_001781 | CD69 antigen (p60, early T-cell activation |
| CD82 | NM_001024844 | CD82 antigen isoform 2 |
| CD84 | NM_003874 | CD84 antigen (leukocyte antigen) |
| CD96 | NM_005816 | CD96 antigen isoform 2 precursor |
| CD99L2 | NM_031462 | CD99 antigen-like 2 isoform E3′-E4′-E3-E4 |
| CDAN1 | NM_138477 | codanin 1 |
| CDC23 | NM_004661 | cell division cycle protein 23 |
| CDC37L1 | NM_017913 | cell division cycle 37 homolog (S. |
| CDC40 | NM_015891 | pre-mRNA splicing factor 17 |
| CDC42SE1 | NM_020239 | CDC42 small effector 1 |
| CDCA4 | NM_017955 | cell division cycle associated 4 |
| CDCA7 | NM_031942 | cell division cycle associated protein 7 isoform |
| CDH20 | NM_031891 | cadherin 20, type 2 preproprotein |
| CDK2AP2 | NM_005851 | CDK2-associated protein 2 |
| CDK5R1 | NM_003885 | cyclin-dependent kinase 5, regulatory subunit 1 |
| CDK6 | NM_001259 | cyclin-dependent kinase 6 |
| CDKN1A | NM_000389 | cyclin-dependent kinase inhibitor 1A |
| CDT1 | NM_030928 | DNA replication factor |
| CECR6 | NM_031890 | cat eye syndrome chromosome region, candidate 6 |
| CEECAM1 | NM_016174 | cerebral endothelial cell adhesion molecule 1 |
| CELSR2 | NM_001408 | cadherin EGF LAG seven-pass G-type receptor 2 |
| CENPF | NM_016343 | centromere protein F (350/400 kD) |
| CENTA2 | NM_018404 | centaurin-alpha 2 protein |
| CENTB2 | NM_012287 | centaurin, beta 2 |
| CENTD1 | NM_015230 | centaurin delta 1 isoform a |
| CEP135 | NM_025009 | centrosome protein 4 |
| CEP152 | NM_014985 | hypothetical protein LOC22995 |
| CEP170 | NM_014812 | centrosomal protein 170 kDa |
| CEP27 | NM_018097 | hypothetical protein LOC55142 |
| CEP57 | NM_014679 | Translokin |
| CEP70 | NM_024491 | centrosomal protein 70 kDa |
| CERK | NM_022766 | ceramide kinase isoform a |
| CES2 | NM_003869 | carboxylesterase 2 isoform 1 |
| CETN2 | NM_004344 | Caltractin |
| CFL2 | NM_021914 | cofilin 2 |
| CFLAR | NM_003879 | CASP8 and FADD-like apoptosis regulator |
| CGNL1 | NM_032866 | cingulin-like 1 |
| CHAF1A | NM_005483 | chromatin assembly factor 1, subunit A (p150) |
| CHD5 | NM_015557 | chromodomain helicase DNA binding protein 5 |
| CHD6 | NM_032221 | chromodomain helicase DNA binding protein 6 |
| CHD9 | NM_025134 | chromodomain helicase DNA binding protein 9 |
| CHES1 | NM_005197 | checkpoint suppressor 1 |
| ChGn | NM_018371 | chondroitin beta1,4 |
| CHML | NM_001821 | choroideremia-like Rab escort protein 2 |
| CHMP4C | NM_152284 | chromatin modifying protein 4C |
| CHRFAM7A | NM_139320 | CHRNA7-FAM7A fusion isoform 1 |
| CHRM2 | NM_000739 | cholinergic receptor, muscarinic 2 |
| CHRNA5 | NM_000745 | cholinergic receptor, nicotinic, alpha |
| CHRNA7 | NM_000746 | cholinergic receptor, nicotinic, alpha 7 |
| CHRNB1 | NM_000747 | nicotinic acetylcholine receptor beta 1 subunit |
| CHRNB4 | NM_000750 | cholinergic receptor, nicotinic, beta |
| CHST6 | NM_021615 | carbohydrate (N-acetylglucosamine 6-O) |
| CHSY1 | NM_014918 | carbohydrate (chondroitin) synthase 1 |
| CHURC1 | NM_145165 | churchill domain containing 1 |
| CIAPIN1 | NM_020313 | cytokine induced apoptosis inhibitor 1 |
| CIAS1 | NM_004895 | cryopyrin isoform a |
| CIC | NM_015125 | capicua homolog |
| CIT | NM_007174 | Citron |
| CITED4 | NM_133467 | Cbp/p300-interacting transactivator, with |
| CKAP2 | NM_018204 | cytoskeleton associated protein 2 |
| CLASP1 | NM_015282 | CLIP-associating protein 1 |
| CLCN6 | NM_001286 | chloride channel 6 isoform ClC-6a |
| CLDN11 | NM_005602 | claudin 11 |
| CLDN12 | NM_012129 | claudin 12 |
| CLDN15 | NM_138429 | claudin 15 isoform 2 |
| CLDN18 | NM_001002026 | claudin 18 isoform 2 |
| CLDN19 | NM_148960 | claudin 19 |
| CLDN2 | NM_020384 | claudin 2 |
| CLDND1 | NM_019895 | hypothetical protein LOC56650 |
| CLEC12B | NM_205852 | macrophage antigen h |
| CLEC2D | NM_001004419 | osteoclast inhibitory lectin isoform 2 |
| CLEC4D | NM_080387 | C-type lectin domain family 4, member D |
| CLIC4 | NM_013943 | chloride intracellular channel 4 |
| CLIC5 | NM_016929 | chloride intracellular channel 5 |
| CLN5 | NM_006493 | ceroid-lipofuscinosis, neuronal 5 |
| CLN8 | NM_018941 | CLN8 protein |
| CLOCK | NM_004898 | Clock |
| CLSTN1 | NM_001009566 | calsyntenin 1 isoform 1 |
| CLSTN2 | NM_022131 | calsyntenin 2 |
| CMPK | NM_016308 | cytidylate kinase |
| CMTM4 | NM_178818 | chemokine-like factor superfamily 4 isoform 1 |
| CMTM6 | NM_017801 | CKLF-like MARVEL transmembrane domain containing |
| CNAP1 | NM_014865 | chromosome condensation-related SMC-associated |
| CNDP2 | NM_018235 | CNDP dipeptidase 2 (metallopeptidase M20 |
| CNGB3 | NM_019098 | cyclic nucleotide gated channel beta 3 |
| CNN1 | NM_001299 | calponin 1, basic, smooth muscle |
| CNNM2 | NM_199077 | cyclin M2 isoform 3 |
| CNNM3 | NM_017623 | cyclin M3 isoform 1 |
| CNOT4 | NM_001008225 | CCR4-NOT transcription complex, subunit 4 |
| CNOT6 | NM_015455 | CCR4-NOT transcription complex, subunit 6 |
| CNOT7 | NM_013354 | CCR4-NOT transcription complex, subunit 7 |
| CNR1 | NM_016083 | central cannabinoid receptor isoform a |
| CNTF | NM_000614 | ciliary neurotrophic factor |
| CNTN3 | NM_020872 | contactin 3 |
| CNTNAP2 | NM_014141 | cell recognition molecule Caspr2 precursor |
| CNTNAP3 | NM_033655 | cell recognition molecule CASPR3 |
| COBL | NM_015198 | cordon-bleu homolog |
| COG3 | NM_031431 | component of golgi transport complex 3 |
| COG7 | NM_153603 | component of oligomeric golgi complex 7 |
| COIL | NM_004645 | Coilin |
| COL11A2 | NM_080679 | collagen, type XI, alpha 2 isoform 3 |
| COL19A1 | NM_001858 | alpha 1 type XIX collagen precursor |
| COL4A1 | NM_001845 | alpha 1 type IV collagen preproprotein |
| COL4A2 | NM_001846 | alpha 2 type IV collagen preproprotein |
| COL4A3 | NM_000091 | alpha 3 type IV collagen isoform 1 precursor |
| COL4A4 | NM_000092 | alpha 4 type IV collagen precursor |
| COL8A2 | NM_005202 | collagen, type VIII, alpha 2 |
| COLEC12 | NM_030781 | collectin sub-family member 12 isoform II |
| COLQ | NM_005677 | acetylcholinesterase collagen-like tail subunit |
| COMMD10 | NM_016144 | COMM domain containing 10 |
| COMMD2 | NM_016094 | COMM domain containing 2 |
| COMMD4 | NM_017828 | COMM domain containing 4 |
| COMMD5 | NM_014066 | hypertension-related calcium-regulated gene |
| COPA | NM_004371 | coatomer protein complex, subunit alpha |
| COPS6 | NM_006833 | COP9 signalosome subunit 6 |
| COQ2 | NM_015697 | para-hydroxybenzoate-polyprenyltransferase, |
| COQ7 | NM_016138 | COQ7 protein |
| CORIN | NM_006587 | Corin |
| CORO1C | NM_014325 | coronin, actin binding protein, 1C |
| CORO2B | NM_006091 | coronin, actin binding protein, 2B |
| COX6B2 | NM_144613 | cytochrome c oxidase subunit VIb, |
| COX7A2L | NM_004718 | cytochrome c oxidase subunit VIIa polypeptide 2 |
| COX8C | NM_182971 | cytochrome c oxidase subunit 8C |
| CP110 | NM_014711 | CP110 protein |
| CPEB3 | NM_014912 | cytoplasmic polyadenylation element binding |
| CPM | NM_001005502 | carboxypeptidase M precursor |
| CPNE1 | NM_003915 | copine I |
| CPOX | NM_000097 | coproporphyrinogen oxidase |
| CPS1 | NM_001875 | carbamoyl-phosphate synthetase 1, mitochondrial |
| CPSF6 | NM_007007 | cleavage and polyadenylation specific factor 6, |
| CR1 | NM_000573 | complement receptor 1 isoform F precursor |
| CRAMP1L | NM_020825 | Crm, cramped-like |
| CREB1 | NM_004379 | cAMP responsive element binding protein 1 |
| CREB5 | NM_001011666 | cAMP responsive element binding protein 5 |
| CREBL2 | NM_001310 | cAMP responsive element binding protein-like 2 |
| CREM | NM_181571 | cAMP responsive element modulator isoform a |
| CRIM1 | NM_016441 | cysteine-rich motor neuron 1 |
| CRIPT | NM_014171 | postsynaptic protein CRIPT |
| CRK | NM_005206 | v-crk sarcoma virus CT10 oncogene homolog |
| CRMP1 | NM_001014809 | collapsin response mediator protein 1 isoform 1 |
| CROT | NM_021151 | carnitine O-octanoyltransferase |
| CRP | NM_000567 | C-reactive protein, pentraxin-related |
| CRSP3 | NM_004830 | cofactor required for Sp1 transcriptional |
| CRSP6 | NM_004268 | cofactor required for Sp1 transcriptional |
| CRSP7 | NM_004831 | cofactor required for Sp1 transcriptional |
| CRSP9 | NM_004270 | cofactor required for Sp1 transcriptional |
| CRTAM | NM_019604 | class-I MHC-restricted T cell associated |
| CRY2 | NM_021117 | cryptochrome 2 (photolyase-like) |
| CS | NM_004077 | citrate synthase precursor, isoform a |
| CSAD | NM_015989 | cysteine sulfinic acid decarboxylase-related |
| CSDE1 | NM_001007553 | upstream of NRAS isoform 1 |
| CSF2RA | NM_006140 | colony stimulating factor 2 receptor alpha chain |
| CSMD2 | NM_052896 | CUB and Sushi multiple domains 2 |
| CSNK1G1 | NM_001011664 | casein kinase 1, gamma 1 isoform L |
| CSNK2A1 | NM_001895 | casein kinase II alpha 1 subunit isoform a |
| CSTF2T | NM_015235 | cleavage stimulation factor, 3′ pre-RNA, subunit |
| CTAGE1 | NM_022663 | cutaneous T-cell lymphoma-associated antigen 1 |
| CTDSPL | NM_001008392 | small CTD phosphatase 3 isoform 1 |
| CTDSPL2 | NM_016396 | CTD (carboxy-terminal domain, RNA polymerase II, |
| CTF1 | NM_001330 | cardiotrophin 1 |
| CTNND1 | NM_001331 | catenin (cadherin-associated protein), delta 1 |
| CTNS | NM_001031681 | cystinosis, nephropathic isoform 1 |
| CTSB | NM_001908 | cathepsin B preproprotein |
| CTSC | NM_148170 | cathepsin C isoform b precursor |
| CTSK | NM_000396 | cathepsin K preproprotein |
| CTSS | NM_004079 | cathepsin S preproprotein |
| CTTNBP2NL | NM_018704 | hypothetical protein LOC55917 |
| CUBN | NM_001081 | Cubilin |
| CUGBP2 | NM_001025076 | CUG triplet repeat, RNA binding protein 2 |
| CUL1 | NM_003592 | cullin 1 |
| CUL3 | NM_003590 | cullin 3 |
| CUTL2 | NM_015267 | cut-like 2 |
| CX3CL1 | NM_002996 | chemokine (C—X3—C motif) ligand 1 |
| CX40.1 | NM_153368 | connexin40.1 |
| CXCL14 | NM_004887 | small inducible cytokine B14 precursor |
| CXCL5 | NM_002994 | chemokine (C—X—C motif) ligand 5 precursor |
| CXCL6 | NM_002993 | chemokine (C—X—C motif) ligand 6 (granulocyte |
| CXCL9 | NM_002416 | small inducible cytokine B9 precursor |
| CXorf20 | NM_153346 | hypothetical protein LOC139105 |
| CXorf21 | NM_025159 | hypothetical protein LOC80231 |
| CXorf38 | NM_144970 | hypothetical protein LOC159013 |
| CXorf41 | NM_173494 | hypothetical protein LOC139212 |
| CXorf53 | NM_001018055 | BRCA1/BRCA2-containing complex subunit 36 |
| CXorf6 | NM_005491 | hypothetical protein LOC10046 |
| CXXC6 | NM_030625 | CXXC finger 6 |
| CYB561D1 | NM_182580 | cytochrome b-561 domain containing 1 |
| CYB5B | NM_030579 | cytochrome b5 outer mitochondrial membrane |
| CYB5D1 | NM_144607 | hypothetical protein LOC124637 |
| CYBB | NM_000397 | cytochrome b-245, beta polypeptide (chronic |
| CYBRD1 | NM_024843 | cytochrome b reductase 1 |
| CYCS | NM_018947 | cytochrome c |
| CYLD | NM_015247 | ubiquitin carboxyl-terminal hydrolase CYLD |
| CYLN2 | NM_003388 | cytoplasmic linker 2 isoform 1 |
| CYP19A1 | NM_000103 | cytochrome P450, family 19 |
| CYP26B1 | NM_019885 | cytochrome P450, family 26, subfamily b, |
| CYP2U1 | NM_183075 | cytochrome P450, family 2, subfamily U, |
| CYP2W1 | NM_017781 | cytochrome P450, family 2, subfamily W, |
| CYP4F3 | NM_000896 | cytochrome P450, family 4, subfamily F, |
| CYSLTR2 | NM_020377 | cysteinyl leukotriene receptor 2 |
| D21S2056E | NM_003683 | nucleolar protein NOP52 |
| DAPK2 | NM_014326 | death-associated protein kinase 2 |
| DAZAP2 | NM_014764 | DAZ associated protein 2 |
| DBF4 | NM_006716 | activator of S phase kinase |
| DBF4B | NM_025104 | DBF4 homolog B isoform 2 |
| DBT | NM_001918 | dihydrolipoamide branched chain transacylase |
| DCBLD2 | NM_080927 | discoidin, CUB and LCCL domain containing 2 |
| DCLRE1C | NM_001033855 | artemis protein isoform a |
| DCTN4 | NM_016221 | dynactin 4 (p62) |
| DCTN5 | NM_032486 | dynactin 4 |
| DCUN1D3 | NM_173475 | hypothetical protein LOC123879 |
| DCUN1D4 | NM_015115 | DCN1, defective in cullin neddylation 1, domain |
| DDAH1 | NM_012137 | dimethylarginine dimethylaminohydrolase 1 |
| DDB2 | NM_000107 | damage-specific DNA binding protein 2 (48 kD) |
| DDHD1 | NM_030637 | DDHD domain containing 1 |
| DDHD2 | NM_015214 | DDHD domain containing 2 |
| DDOST | NM_005216 | dolichyl-diphosphooligosaccharide-protein |
| DDX11 | NM_030655 | DEAD/H (Asp-Glu-Ala-Asp/His) box polypeptide 11 |
| DDX21 | NM_004728 | DEAD (Asp-Glu-Ala-Asp) box polypeptide 21 |
| DDX26B | NM_182540 | hypothetical protein LOC203522 |
| DDX46 | NM_014829 | DEAD (Asp-Glu-Ala-Asp) box polypeptide 46 |
| DDX5 | NM_004396 | DEAD (Asp-Glu-Ala-Asp) box polypeptide 5 |
| DDX51 | NM_175066 | DEAD (Asp-Glu-Ala-Asp) box polypeptide 51 |
| DDX55 | NM_020936 | DEAD (Asp-Glu-Ala-Asp) box polypeptide 55 |
| DDX59 | NM_031306 | DEAD (Asp-Glu-Ala-Asp) box polypeptide 59 |
| DEADC1 | NM_182503 | deaminase domain containing 1 |
| DEAF1 | NM_021008 | Suppressin |
| DECR2 | NM_020664 | 2,4-dienoyl CoA reductase 2, peroxisomal |
| DEDD | NM_032998 | death effector domain-containing protein |
| DEFB106A | NM_152251 | defensin, beta 106A |
| DEGS1 | NM_003676 | degenerative spermatocyte homolog 1, lipid |
| DENND1A | NM_024820 | hypothetical protein LOC57706 isoform 2 |
| DENND2C | NM_198459 | DENN/MADD domain containing 2C |
| DENND3 | NM_014957 | hypothetical protein LOC22898 |
| DENND4C | NM_017925 | hypothetical protein LOC55667 |
| DEPDC4 | NM_152317 | DEP domain containing 4 |
| DERL2 | NM_016041 | Der1-like domain family, member 2 |
| DFFA | NM_004401 | DNA fragmentation factor, 45 kDa, alpha |
| DFNA5 | NM_004403 | deafness, autosomal dominant 5 protein |
| DGAT2L4 | NM_001002254 | diacylglycerol O-acyltransferase 2-like 4 |
| DGCR13 | NM_001024733 | DiGeorge syndrome gene H |
| DGKQ | NM_001347 | diacylglycerol kinase, theta |
| DHDDS | NM_024887 | dehydrodolichyl diphosphate synthase isoform a |
| DHFRL1 | NM_176815 | hypothetical protein LOC200895 |
| DHODH | NM_001025193 | dihydroorotate dehydrogenase isoform 2 |
| DHTKD1 | NM_018706 | dehydrogenase E1 and transketolase domain |
| DHX34 | NM_014681 | DEAH (Asp-Glu-Ala-His) box polypeptide 34 |
| DICER1 | NM_030621 | dicer1 |
| DIDO1 | NM_033081 | death inducer-obliterator 1 isoform c |
| DIO1 | NM_000792 | deiodinase, iodothyronine, type I isoform a |
| DIP2A | NM_015151 | DIP2-like protein isoform a |
| DIP2B | NM_173602 | hypothetical protein LOC57609 |
| DIRC1 | NM_052952 | hypothetical protein LOC116093 |
| DISC1 | NM_001012957 | disrupted in schizophrenia 1 isoform Lv |
| DIXDC1 | NM_033425 | DIX domain containing 1 isoform b |
| DKFZP434B0335 | NM_015395 | hypothetical protein LOC25851 |
| DKFZp434I1020 | NM_194295 | hypothetical protein LOC196968 |
| DKFZp451A211 | NM_001003399 | hypothetical protein LOC400169 |
| DKFZP564J0863 | NM_015459 | hypothetical protein LOC25923 |
| DKFZp564K142 | NM_032121 | implantation-associated protein |
| DKFZp667M2411 | NM_207323 | hypothetical protein LOC147172 |
| DKFZP686A10121 | NM_033107 | claudin 12 |
| DKFZp686L1814 | NM_194282 | hypothetical protein LOC132660 |
| DKFZp686O24166 | NM_001009913 | hypothetical protein LOC374383 |
| DKFZp761E198 | NM_138368 | hypothetical protein LOC91056 |
| DKFZp762I137 | NM_152411 | hypothetical protein LOC136051 |
| DKFZP781I1119 | NM_152622 | hypothetical protein LOC166968 |
| DLC1 | NM_006094 | deleted in liver cancer 1 isoform 2 |
| DLEC1 | NM_005106 | deleted in lung and esophageal cancer 1 isoform |
| DLGAP2 | NM_004745 | discs large-associated protein 2 |
| DLX5 | NM_005221 | distal-less homeobox 5 |
| DMBX1 | NM_147192 | diencephalon/mesencephalon homeobox 1 isoform b |
| DMC1 | NM_007068 | DMC1 dosage suppressor of mck1 homolog |
| DMN | NM_015286 | desmuslin isoform B |
| DMP1 | NM_004407 | dentin matrix acidic phosphoprotein |
| DMRT2 | NM_006557 | doublesex and mab-3 related transcription factor |
| DMTF1 | NM_021145 | cyclin D binding myb-like transcription factor |
| DNAJA4 | NM_018602 | DnaJ (Hsp40) homolog, subfamily A, member 4 |
| DNAJA5 | NM_001012339 | DnaJ homology subfamily A member 5 isoform 2 |
| DNAJB6 | NM_005494 | DnaJ (Hsp40) homolog, subfamily B, member 6 |
| DNAJB9 | NM_012328 | DnaJ (Hsp40) homolog, subfamily B, member 9 |
| DNAJC15 | NM_013238 | DNAJ domain-containing |
| DNAJC18 | NM_152686 | DnaJ (Hsp40) homolog, subfamily C, member 18 |
| DNAJC19 | NM_145261 | translocase of the inner mitochondrial membrane |
| DNAJC5 | NM_025219 | DnaJ (Hsp40) homolog, subfamily C, member 5 |
| DNASE2 | NM_001375 | deoxyribonuclease II, lysosomal precursor |
| DNM2 | NM_001005360 | dynamin 2 isoform 1 |
| DNM3 | NM_015569 | dynamin 3 |
| DOCK9 | NM_015296 | dedicator of cytokinesis 9 |
| DOPEY2 | NM_005128 | pad-1-like |
| DPP10 | NM_001004360 | dipeptidyl peptidase 10 isoform short |
| DPP3 | NM_005700 | dipeptidyl peptidase III |
| DPP9 | NM_139159 | dipeptidylpeptidase 9 |
| DPY19L3 | NM_207325 | dpy-19-like 3 |
| DPYD | NM_000110 | dihydropyrimidine dehydrogenase |
| DPYSL5 | NM_020134 | dihydropyrimidinase-like 5 |
| DRD1 | NM_000794 | dopamine receptor D1 |
| DSC3 | NM_024423 | desmocollin 3 isoform Dsc3b preproprotein |
| DSG4 | NM_177986 | desmoglein 4 |
| DSPG3 | NM_004950 | dermatan sulfate proteoglycan 3 precursor |
| DTWD2 | NM_173666 | DTW domain containing 2 |
| DUSP10 | NM_007207 | dual specificity phosphatase 10 isoform a |
| DUSP13 | NM_001007271 | muscle-restricted dual specificity phosphatase |
| DUSP18 | NM_152511 | dual specificity phosphatase 18 |
| DUSP2 | NM_004418 | dual specificity phosphatase 2 |
| DUSP6 | NM_001946 | dual specificity phosphatase 6 isoform a |
| DUSP8 | NM_004420 | dual specificity phosphatase 8 |
| DUXA | NM_001012729 | hypothetical protein LOC503835 |
| DVL3 | NM_004423 | dishevelled 3 |
| DXS9879E | NM_006014 | ESO3 protein |
| DYNC1LI2 | NM_006141 | dynein, cytoplasmic, light intermediate |
| DYNLT1 | NM_006519 | t-complex-associated-testis-expressed 1-like 1 |
| DYRK1A | NM_001396 | dual-specificity tyrosine-(Y)-phosphorylation |
| DYRK2 | NM_003583 | dual-specificity tyrosine-(Y)-phosphorylation |
| DZIP1 | NM_014934 | DAZ interacting protein 1 isoform 1 |
| E2F1 | NM_005225 | E2F transcription factor 1 |
| E2F2 | NM_004091 | E2F transcription factor 2 |
| E2F3 | NM_001949 | E2F transcription factor 3 |
| E2F5 | NM_001951 | E2F transcription factor 5 |
| EAF1 | NM_033083 | ELL associated factor 1 |
| EBF3 | NM_001005463 | early B-cell factor 3 |
| EBI2 | NM_004951 | EBV-induced G protein-coupled receptor 2 |
| EDA | NM_001005610 | ectodysplasin A isoform EDA-B |
| EDA2R | NM_021783 | X-linked ectodysplasin receptor |
| EDD1 | NM_015902 | E3 ubiquitin protein ligase, HECT domain |
| EDEM1 | NM_014674 | ER degradation enhancer, mannosidase alpha-like |
| EDG1 | NM_001400 | endothelial differentiation, sphingolipid |
| EDG3 | NM_005226 | endothelial differentiation, sphingolipid |
| EFHA2 | NM_181723 | EF hand domain family, member A2 |
| EFNA1 | NM_004428 | ephrin A1 isoform a precursor |
| EFNB1 | NM_004429 | ephrin-B1 precursor |
| EFNB2 | NM_004093 | ephrin B2 |
| EFTUD1 | NM_024580 | elongation factor Tu GTP binding domain |
| EGFL4 | NM_001410 | EGF-like-domain, multiple 4 |
| EGLN1 | NM_022051 | egl nine homolog 1 |
| EGLN3 | NM_022073 | egl nine homolog 3 |
| EGR2 | NM_000399 | early growth response 2 protein |
| EGR3 | NM_004430 | early growth response 3 |
| EHD3 | NM_014600 | EH-domain containing 3 |
| EHHADH | NM_001966 | enoyl-Coenzyme A, hydratase/3-hydroxyacyl |
| EHMT1 | NM_024757 | euchromatic histone methyltransferase 1 |
| EI24 | NM_001007277 | etoposide induced 2.4 isoform 2 |
| EID-3 | NM_152361 | EID-2-like inhibitor of differentiation-3 |
| EIF2AK4 | NM_001013703 | eukaryotic translation initiation factor 2 alpha |
| EIF2C1 | NM_012199 | eukaryotic translation initiation factor 2C, 1 |
| EIF2S1 | NM_004094 | eukaryotic translation initiation factor 2, |
| EIF3S2 | NM_003757 | eukaryotic translation initiation factor 3, |
| EIF4EBP2 | NM_004096 | eukaryotic translation initiation factor 4E |
| EIF4G2 | NM_001418 | eukaryotic translation initiation factor 4 |
| EIF5 | NM_001969 | eukaryotic translation initiation factor 5 |
| EIF5A2 | NM_020390 | eIF-5A2 protein |
| ELK3 | NM_005230 | ELK3 protein |
| Ells1 | NM_152793 | hypothetical protein LOC222166 |
| ELMO1 | NM_014800 | engulfment and cell motility 1 isoform 1 |
| ELMOD1 | NM_018712 | ELMO domain containing 1 |
| EMR2 | NM_013447 | egf-like module containing, mucin-like, hormone |
| EMX2 | NM_004098 | empty spiracles homolog 2 |
| EN2 | NM_001427 | engrailed homolog 2 |
| ENAH | NM_001008493 | enabled homolog isoform a |
| ENAM | NM_031889 | Enamelin |
| ENO2 | NM_001975 | enolase 2 |
| ENPP4 | NM_014936 | ectonucleotide pyrophosphatase/phosphodiesterase |
| ENPP5 | NM_021572 | ectonucleotide pyrophosphatase/phosphodiesterase |
| ENSA | NM_207168 | endosulfine alpha isoform 8 |
| ENTPD4 | NM_004901 | ectonucleoside triphosphate diphosphohydrolase |
| ENTPD6 | NM_001247 | ectonucleoside triphosphate diphosphohydrolase |
| EPAS1 | NM_001430 | endothelial PAS domain protein 1 |
| EPB41 | NM_004437 | erythrocyte membrane protein band 4.1 |
| EPB41L1 | NM_012156 | erythrocyte membrane protein band 4.1-like 1 |
| EPB41L2 | NM_001431 | erythrocyte membrane protein band 4.1-like 2 |
| EPB41L4B | NM_019114 | erythrocyte membrane protein band 4.1 like 4B |
| EPB41L5 | NM_020909 | erythrocyte membrane protein band 4.1 like 5 |
| EPDR1 | NM_017549 | upregulated in colorectal cancer gene 1 protein |
| EPHA4 | NM_004438 | ephrin receptor EphA4 |
| EPHA5 | NM_004439 | ephrin receptor EphA5 isoform a |
| EPHA7 | NM_004440 | ephrin receptor EphA7 |
| EPHB1 | NM_004441 | ephrin receptor EphB1 precursor |
| EPHB4 | NM_004444 | ephrin receptor EphB4 precursor |
| EPM2A | NM_005670 | laforin isoform a |
| EPM2AIP1 | NM_014805 | EPM2A interacting protein 1 |
| ERBB2IP | NM_001006600 | ERBB2 interacting protein isoform 7 |
| ERBB3 | NM_001982 | erbB-3 isoform 1 precursor |
| EREG | NM_001432 | epiregulin precursor |
| ERG | NM_004449 | v-ets erythroblastosis virus E26 oncogene like |
| ERGIC1 | NM_020462 | endoplasmic reticulum-golgi intermediate |
| ERN1 | NM_152461 | endoplasmic reticulum to nucleus signalling 1 |
| ERO1LB | NM_019891 | endoplasmic reticulum oxidoreductin 1-Lbeta |
| ESR1 | NM_000125 | estrogen receptor 1 |
| ET | NM_024311 | hypothetical protein LOC79157 |
| ETF1 | NM_004730 | eukaryotic translation termination factor 1 |
| ETV1 | NM_004956 | ets variant gene 1 |
| ETV5 | NM_004454 | ets variant gene 5 (ets-related molecule) |
| EVA1 | NM_005797 | epithelial V-like antigen 1 precursor |
| EXOSC1 | NM_016046 | exosomal core protein CSL4 |
| EYA1 | NM_000503 | eyes absent 1 isoform b |
| EYA4 | NM_004100 | eyes absent 4 isoform a |
| EZH1 | NM_001991 | enhancer of zeste homolog 1 |
| F11R | NM_016946 | F11 receptor isoform a precursor |
| F2R | NM_001992 | coagulation factor II receptor precursor |
| F2RL1 | NM_005242 | coagulation factor II (thrombin) receptor-like 1 |
| F2RL2 | NM_004101 | coagulation factor II (thrombin) receptor-like 2 |
| F2RL3 | NM_003950 | coagulation factor II (thrombin) receptor-like 3 |
| F3 | NM_001993 | coagulation factor III precursor |
| F9 | NM_000133 | coagulation factor IX |
| FADS1 | NM_013402 | fatty acid desaturase 1 |
| FADS6 | NM_178128 | fatty acid desaturase domain family, member 6 |
| FAHD1 | NM_031208 | fumarylacetoacetate hydrolase domain containing |
| FAIM2 | NM_012306 | Fas apoptotic inhibitory molecule 2 |
| FAM102A | NM_203305 | early estrogen-induced gene 1 protein isoform b |
| FAM106A | NM_024974 | hypothetical protein LOC80039 |
| FAM107A | NM_007177 | downregulated in renal cell carcinoma |
| FAM107B | NM_031453 | hypothetical protein LOC83641 |
| FAM13A1 | NM_001015045 | family with sequence similarity 13, member A1 |
| FAM13C1 | NM_001001971 | hypothetical protein LOC220965 isoform 2 |
| FAM18B | NM_016078 | hypothetical protein LOC51030 |
| FAM19A1 | NM_213609 | family with sequence similarity 19 (chemokine |
| FAM36A | NM_198076 | family with sequence similarity 36, member A |
| FAM3A | NM_021806 | family 3, member A protein |
| FAM3C | NM_014888 | predicted osteoblast protein |
| FAM40A | NM_033088 | hypothetical protein LOC85369 |
| FAM40B | NM_020704 | hypothetical protein LOC57464 |
| FAM43A | NM_153690 | hypothetical protein LOC131583 |
| FAM45A | NM_207009 | hypothetical protein LOC404636 |
| FAM45B | NM_018472 | hypothetical protein LOC55855 |
| FAM46C | NM_017709 | hypothetical protein LOC54855 |
| FAM46D | NM_152630 | hypothetical protein LOC169966 |
| FAM53B | NM_014661 | hypothetical protein LOC9679 |
| FAM53C | NM_016605 | family 53, member C protein |
| FAM54B | NM_019557 | hypothetical protein LOC56181 |
| FAM55C | NM_145037 | hypothetical protein LOC91775 |
| FAM57A | NM_024792 | family with sequence similarity 57, member A |
| FAM60A | NM_021238 | family with sequence similarity 60, member A |
| FAM62B | NM_020728 | family with sequence similarity 62 (C2 domain |
| FAM65A | NM_024519 | hypothetical protein LOC79567 |
| FAM70A | NM_017938 | hypothetical protein LOC55026 |
| FAM73A | NM_198549 | hypothetical protein LOC374986 |
| FAM73B | NM_032809 | hypothetical protein LOC84895 |
| FAM79A | NM_182752 | hypothetical protein LOC127262 |
| FAM79B | NM_198485 | hypothetical protein LOC285386 |
| FAM82C | NM_018145 | family with sequence similarity 82, member C |
| FAM83D | NM_030919 | hypothetical protein LOC81610 |
| FAM83E | NM_017708 | hypothetical protein LOC54854 |
| FAM83H | NM_198488 | hypothetical protein LOC286077 |
| FAM84B | NM_174911 | breast cancer membrane protein 101 |
| FAM86C | NM_018172 | hypothetical protein LOC55199 isoform 1 |
| FAM8A1 | NM_016255 | Autosomal Highly Conserved Protein |
| FAM98B | NM_173611 | hypothetical protein LOC283742 |
| FANCC | NM_000136 | Fanconi anemia, complementation group C |
| FANCD2 | NM_033084 | Fanconi anemia complementation group D2 isoform |
| FARSLB | NM_005687 | phenylalanine-tRNA synthetase-like, beta |
| FASLG | NM_000639 | fas ligand |
| FASTK | NM_006712 | Fas-activated serine/threonine kinase isoform 1 |
| FAT2 | NM_001447 | FAT tumor suppressor 2 precursor |
| FBLIM1 | NM_001024216 | filamin-binding LIM protein-1 isoform c |
| FBLN1 | NM_006486 | fibulin 1 isoform D |
| FBN2 | NM_001999 | fibrillin 2 precursor |
| FBXL11 | NM_012308 | F-box and leucine-rich repeat protein 11 |
| FBXL18 | NM_024963 | F-box and leucine-rich repeat protein 18 |
| FBXL22 | NM_203373 | hypothetical protein LOC283807 |
| FBXL3 | NM_012158 | F-box and leucine-rich repeat protein 3 |
| FBXL5 | NM_012161 | F-box and leucine-rich repeat protein 5 isoform |
| FBXL7 | NM_012304 | F-box and leucine-rich repeat protein 7 |
| FBXO11 | NM_025133 | F-box only protein 11 isoform 1 |
| FBXO18 | NM_032807 | F-box only protein, helicase, 18 isoform 1 |
| FBXO21 | NM_015002 | F-box only protein 21 isoform 2 |
| FBXO27 | NM_178820 | F-box protein 27 |
| FBXO31 | NM_024735 | F-box protein 31 |
| FBXO39 | NM_153230 | F-box protein 39 |
| FBXO40 | NM_016298 | F-box protein 40 |
| FBXO6 | NM_018438 | F-box only protein 6 |
| FBXO9 | NM_012347 | F-box only protein 9 isoform 1 |
| FBXW11 | NM_012300 | F-box and WD-40 domain protein 1B isoform C |
| FCHO2 | NM_138782 | FCH domain only 2 |
| FCMD | NM_006731 | Fukutin |
| FEM1C | NM_020177 | feminization 1 homolog a |
| FEZ2 | NM_005102 | zygin 2 |
| FGD1 | NM_004463 | faciogenital dysplasia protein |
| FGD4 | NM_139241 | FYVE, RhoGEF and PH domain containing 4 |
| FGD5 | NM_152536 | FYVE, RhoGEF and PH domain containing 5 |
| FGF2 | NM_002006 | fibroblast growth factor 2 |
| FGF4 | NM_002007 | fibroblast growth factor 4 precursor |
| FGF5 | NM_004464 | fibroblast growth factor 5 isoform 1 precursor |
| FGF7 | NM_002009 | fibroblast growth factor 7 precursor |
| FGFR1 | NM_023107 | fibroblast growth factor receptor 1 isoform 5 |
| FGFR2 | NM_022973 | fibroblast growth factor receptor 2 isoform 6 |
| FGL2 | NM_006682 | fibrinogen-like 2 |
| FIGNL1 | NM_022116 | fidgetin-like 1 |
| FJX1 | NM_014344 | four jointed box 1 |
| FKBP14 | NM_017946 | FK506 binding protein 14, 22 kDa |
| FKBP1B | NM_004116 | FK506-binding protein 1B isoform a |
| FKBP5 | NM_004117 | FK506 binding protein 5 |
| FKRP | NM_024301 | fukutin-related protein |
| FLG2 | NM_001014342 | filaggrin 2 |
| FLJ10159 | NM_018013 | hypothetical protein LOC55084 |
| FLJ10241 | NM_018035 | hypothetical protein LOC55101 |
| FLJ10357 | NM_018071 | hypothetical protein LOC55701 |
| FLJ10781 | NM_018215 | hypothetical protein LOC55228 |
| FLJ10803 | NM_018224 | hypothetical protein LOC55744 |
| FLJ10925 | NM_018275 | hypothetical protein LOC55262 |
| FLJ11021 | NM_023012 | hypothetical protein LOC65117 isoform a |
| FLJ11151 | NM_018340 | hypothetical protein LOC55313 |
| FLJ11171 | NM_018348 | hypothetical protein LOC55783 |
| FLJ11259 | NM_018370 | hypothetical protein LOC55332 |
| FLJ11292 | NM_018382 | hypothetical protein LOC55338 |
| FLJ11806 | NM_024824 | nuclear protein UKp68 isoform 1 |
| FLJ12331 | NM_024986 | hypothetical protein LOC80052 |
| FLJ12505 | NM_024749 | hypothetical protein LOC79805 |
| FLJ12949 | NM_023008 | hypothetical protein LOC65095 isoform 1 |
| FLJ13236 | NM_024902 | hypothetical protein FLJ13236 |
| FLJ13576 | NM_022484 | hypothetical protein LOC64418 |
| FLJ13639 | NM_024705 | hypothetical protein FLJ13639 isoform 2 |
| FLJ13646 | NM_024584 | hypothetical protein LOC79635 |
| FLJ13841 | NM_024702 | hypothetical protein LOC79755 |
| FLJ13946 | NM_152275 | hypothetical protein LOC92104 |
| FLJ13984 | NM_024770 | hypothetical protein LOC79828 |
| FLJ14107 | NM_025026 | hypothetical protein LOC80094 |
| FLJ14213 | NM_024841 | hypothetical protein LOC79899 |
| FLJ14397 | NM_032779 | hypothetical protein LOC84865 |
| FLJ14437 | NM_032578 | Myopalladin |
| FLJ14466 | NM_032790 | hypothetical protein LOC84876 |
| FLJ14503 | NM_152780 | hypothetical protein LOC256714 |
| FLJ16008 | NM_001001665 | hypothetical protein LOC339761 |
| FLJ16237 | NM_001004320 | hypothetical protein LOC392636 |
| FLJ16542 | NM_001004301 | hypothetical protein LOC126017 |
| FLJ20032 | NM_017628 | hypothetical protein LOC54790 |
| FLJ20186 | NM_207514 | differentially expressed in FDCP 8 isoform 1 |
| FLJ20294 | NM_017749 | hypothetical protein LOC55626 |
| FLJ20298 | NM_017752 | hypothetical protein LOC54885 isoform a |
| FLJ20366 | NM_017786 | hypothetical protein FLJ20366 |
| FLJ20487 | NM_017841 | hypothetical protein LOC54949 |
| FLJ20489 | NM_017842 | hypothetical protein LOC55652 |
| FLJ20758 | NM_017952 | hypothetical protein LOC55037 |
| FLJ20972 | NM_025030 | hypothetical protein LOC80098 |
| FLJ21125 | NM_024627 | hypothetical protein LOC79680 |
| FLJ21657 | NM_022483 | hypothetical protein LOC64417 |
| FLJ21687 | NM_024859 | PDZ domain containing, X chromosome |
| FLJ21736 | NM_024922 | esterase 31 |
| FLJ21945 | NM_025203 | hypothetical protein LOC80304 |
| FLJ21963 | NM_024560 | hypothetical protein LOC79611 |
| FLJ23235 | NM_024943 | hypothetical protein LOC80008 |
| FLJ23322 | NM_024955 | hypothetical protein LOC80020 |
| FLJ23447 | NM_024825 | hypothetical protein LOC79883 |
| FLJ23834 | NM_152750 | hypothetical protein LOC222256 |
| FLJ23861 | NM_152519 | hypothetical protein LOC151050 |
| FLJ25102 | NM_182626 | hypothetical protein LOC348738 |
| FLJ25328 | NM_152483 | hypothetical protein LOC148231 |
| FLJ25416 | NM_145018 | hypothetical protein LOC220042 |
| FLJ25476 | NM_152493 | hypothetical protein LOC149076 |
| FLJ25477 | NM_152704 | hypothetical protein LOC219287 isoform 1 |
| FLJ25530 | NM_152722 | hepatocyte cell adhesion molecule |
| FLJ25773 | NM_182560 | hypothetical protein LOC283598 |
| FLJ27365 | NM_207477 | hypothetical protein LOC400931 |
| FLJ30294 | NM_144632 | hypothetical protein LOC130827 |
| FLJ31132 | NM_001004355 | hypothetical protein LOC441522 |
| FLJ31568 | NM_152509 | hypothetical protein LOC150244 |
| FLJ31659 | NM_153027 | hypothetical protein LOC152756 |
| FLJ31818 | NM_152556 | hypothetical protein LOC154743 |
| FLJ31951 | NM_144726 | hypothetical protein LOC153830 |
| FLJ32028 | NM_152680 | hypothetical protein LOC201799 |
| FLJ32214 | NM_152473 | hypothetical protein LOC147664 |
| FLJ32549 | NM_152440 | hypothetical protein LOC144577 |
| FLJ32675 | NM_173811 | hypothetical protein LOC283254 |
| FLJ33860 | NM_173644 | hypothetical protein LOC284756 |
| FLJ34969 | NM_152678 | hypothetical protein LOC201627 |
| FLJ35119 | NM_175871 | hypothetical protein LOC126074 |
| FLJ35429 | NM_001003807 | hypothetical protein LOC285830 |
| FLJ35530 | NM_207467 | hypothetical protein LOC400798 |
| FLJ35695 | NM_207444 | hypothetical protein LOC400359 |
| FLJ35848 | NM_001033659 | hypothetical protein LOC284071 |
| FLJ35934 | NM_207453 | hypothetical protein LOC400579 |
| FLJ36031 | NM_175884 | hypothetical protein LOC168455 |
| FLJ36090 | NM_153223 | hypothetical protein LOC153241 |
| FLJ36268 | NM_207511 | hypothetical protein LOC401563 |
| FLJ37543 | NM_173667 | hypothetical protein LOC285668 |
| FLJ37562 | NM_152409 | hypothetical protein LOC134553 |
| FLJ38101 | NM_153261 | hypothetical protein LOC255919 |
| FLJ38288 | NM_173632 | hypothetical protein LOC284309 |
| FLJ38663 | NM_152269 | hypothetical protein LOC91574 |
| FLJ38717 | NM_001004322 | hypothetical protein LOC401261 |
| FLJ38973 | NM_153689 | hypothetical protein LOC205327 |
| FLJ38991 | NM_173827 | mitochondrial COX18 isoform 6 |
| FLJ39237 | NM_198571 | hypothetical protein LOC375607 |
| FLJ39502 | NM_173648 | hypothetical protein LOC285025 |
| FLJ39653 | NM_152684 | hypothetical protein LOC202020 |
| FLJ40172 | NM_173649 | hypothetical protein LOC285051 |
| FLJ40194 | NM_001007529 | hypothetical protein LOC124871 |
| FLJ40453 | NM_001007542 | hypothetical protein LOC401217 |
| FLJ40919 | NM_182508 | hypothetical protein LOC144809 |
| FLJ41170 | NM_001004332 | hypothetical protein LOC440200 |
| FLJ41821 | NM_001001697 | hypothetical protein LOC401011 |
| FLJ42102 | NM_001001680 | hypothetical protein LOC399923 |
| FLJ42133 | NM_001001690 | hypothetical protein LOC400844 |
| FLJ42289 | NM_207383 | hypothetical protein LOC388182 |
| FLJ42842 | NM_001004335 | hypothetical protein LOC440446 |
| FLJ42957 | NM_207436 | hypothetical protein LOC400077 |
| FLJ43582 | NM_207412 | hypothetical protein LOC389649 |
| FLJ44006 | NM_001001696 | hypothetical protein LOC400997 |
| FLJ44060 | NM_207366 | hypothetical protein LOC346288 |
| FLJ44290 | NM_198564 | hypothetical protein LOC375347 |
| FLJ44385 | NM_207478 | hypothetical protein LOC400934 |
| FLJ44790 | NM_001001691 | hypothetical protein LOC400850 |
| FLJ44815 | NM_207454 | hypothetical protein LOC400591 |
| FLJ45187 | NM_207371 | hypothetical protein LOC387640 |
| FLJ45202 | NM_207507 | hypothetical protein LOC401508 |
| FLJ45224 | NM_207510 | hypothetical protein LOC401562 |
| FLJ45248 | NM_207505 | hypothetical protein LOC401472 |
| FLJ45256 | NM_207448 | hypothetical protein LOC400511 |
| FLJ45337 | NM_207465 | hypothetical protein LOC400754 |
| FLJ45422 | NM_001004349 | hypothetical protein LOC441140 |
| FLJ45645 | NM_198557 | hypothetical protein LOC375287 |
| FLJ45909 | NM_198445 | hypothetical protein LOC126432 |
| FLJ46247 | NM_198529 | hypothetical protein LOC374786 isoform 1 |
| FLJ46363 | NM_207434 | hypothetical protein LOC400002 |
| FLJ46385 | NM_001001675 | hypothetical protein LOC390963 |
| FLJ90013 | NM_153365 | hypothetical protein LOC202018 |
| FLJ90396 | NM_153358 | hypothetical protein LOC163049 |
| FLJ90579 | NM_173591 | hypothetical protein LOC283310 |
| FLJ90757 | NM_001004336 | hypothetical protein LOC440465 |
| FLRT2 | NM_013231 | fibronectin leucine rich transmembrane protein |
| FLT1 | NM_002019 | fms-related tyrosine kinase 1 (vascular |
| FLYWCH1 | NM_032296 | FLYWCH-type zinc finger 1 isoform a |
| FMNL2 | NM_001004417 | formin-like 2 isoform D |
| FMNL3 | NM_175736 | formin-like 3 isoform 1 |
| FMO3 | NM_001002294 | flavin containing monooxygenase 3 isoform 2 |
| FMOD | NM_002023 | fibromodulin precursor |
| FNBP1 | NM_015033 | formin binding protein 1 |
| FNBP1L | NM_001024948 | formin binding protein 1-like isoform 1 |
| FNBP4 | NM_015308 | formin binding protein 4 |
| FNDC3A | NM_014923 | fibronectin type III domain containing 3A |
| FNDC3B | NM_022763 | fibronectin type III domain containing 3B |
| FNDC5 | NM_153756 | fibronectin type III domain containing 5 |
| FOSL1 | NM_005438 | FOS-like antigen 1 |
| FOXA1 | NM_004496 | forkhead box A1 |
| FOXF1 | NM_001451 | forkhead box F1 |
| FOXJ2 | NM_018416 | forkhead box J2 |
| FOXJ3 | NM_014947 | forkhead box J3 |
| FOXL2 | NM_023067 | forkhead box L2 |
| FOXQ1 | NM_033260 | forkhead box Q1 |
| FPGT | NM_003838 | fucose-1-phosphate guanyltransferase |
| FRAT2 | NM_012083 | GSK-3 binding protein FRAT2 |
| FREQ | NM_014286 | frequenin homolog |
| FRMD4A | NM_018027 | FERM domain containing 4A |
| FRMD6 | NM_152330 | FERM domain containing 6 |
| FTS | NM_001012398 | fused toes homolog |
| FUBP1 | NM_003902 | far upstream element-binding protein |
| FUCA2 | NM_032020 | fucosidase, alpha-L-2, plasma |
| FUNDC2 | NM_023934 | FUN14 domain containing 2 |
| FURIN | NM_002569 | furin preproprotein |
| FUSIP1 | NM_054016 | FUS interacting protein (serine-arginine rich) 1 |
| FUT2 | NM_000511 | fucosyltransferase 2 (secretor status included) |
| FUT3 | NM_000149 | fucosyltransferase 3 (galactoside |
| FUT4 | NM_002033 | fucosyltransferase 4 |
| FUT5 | NM_002034 | fucosyltransferase 5 |
| FUT6 | NM_000150 | fucosyltransferase 6 (alpha (1,3) |
| FXN | NM_000144 | frataxin isoform 1 preproprotein |
| FXR1 | NM_001013438 | fragile X mental retardation-related protein 1 |
| FXYD6 | NM_022003 | FXYD domain-containing ion transport regulator |
| FYCO1 | NM_024513 | FYVE and coiled-coil domain containing 1 |
| FZD10 | NM_007197 | frizzled 10 |
| FZD4 | NM_012193 | frizzled 4 |
| FZD6 | NM_003506 | frizzled 6 |
| FZD7 | NM_003507 | frizzled 7 |
| GAB1 | NM_002039 | GRB2-associated binding protein 1 isoform b |
| GAB2 | NM_012296 | GRB2-associated binding protein 2 isoform b |
| GAB3 | NM_080612 | Gab3 protein |
| GABBR1 | NM_001470 | gamma-aminobutyric acid (GABA) B receptor 1 |
| GABBR2 | NM_005458 | G protein-coupled receptor 51 |
| GABPB2 | NM_005254 | GA binding protein transcription factor, beta |
| GABRE | NM_004961 | gamma-aminobutyric acid (GABA) A receptor, |
| GABRG1 | NM_173536 | gamma-aminobutyric acid A receptor, gamma 1 |
| GABRG2 | NM_198904 | gamma-aminobutyric acid A receptor, gamma 2 |
| GAK | NM_005255 | cyclin G associated kinase |
| GALIG | NM_194327 | galectin-3 internal gene |
| GALK2 | NM_001001556 | galactokinase 2 isoform 2 |
| GALM | NM_138801 | galactose mutarotase (aldose 1-epimerase) |
| GALNT3 | NM_004482 | polypeptide N-acetylgalactosaminyltransferase 3 |
| GALNT4 | NM_003774 | polypeptide N-acetylgalactosaminyltransferase 4 |
| GALNT6 | NM_007210 | polypeptide N-acetylgalactosaminyltransferase 6 |
| GALNTL2 | NM_054110 | UDP-N-acetyl-alpha-D-galactosamine:polypeptide |
| GAN | NM_022041 | Gigaxonin |
| GARS | NM_002047 | glycyl-tRNA synthetase |
| GAS7 | NM_003644 | growth arrest-specific 7 isoform a |
| GAS8 | NM_001481 | growth arrest-specific 8 |
| GATA6 | NM_005257 | GATA binding protein 6 |
| GATAD1 | NM_021167 | GATA zinc finger domain containing 1 |
| GATS | NM_178831 | opposite strand transcription unit to STAG3 |
| GBF1 | NM_004193 | golgi-specific brefeldin A resistance factor 1 |
| GBP1 | NM_002053 | guanylate binding protein 1, |
| GBP3 | NM_018284 | guanylate binding protein 3 |
| GBP4 | NM_052941 | guanylate binding protein 4 |
| GCC2 | NM_014635 | GRIP and coiled-coil domain-containing 2 isoform |
| GCET2 | NM_001008756 | germinal center expressed transcript 2 isoform |
| GCLM | NM_002061 | glutamate-cysteine ligase regulatory protein |
| GCNT2 | NM_001491 | glucosaminyl (N-acetyl) transferase 2, |
| GCNT4 | NM_016591 | core 2 beta-1,6-N-acetylglucosaminyltransferase |
| Gcom1 | NM_001018100 | GRINL1A upstream protein isoform 7 |
| GDA | NM_004293 | guanine deaminase |
| GDPD1 | NM_182569 | glycerophosphodiester phosphodiesterase domain |
| GEMIN7 | NM_001007269 | gemin 7 |
| GENX-3414 | NM_003943 | genethonin 1 |
| GFER | NM_005262 | erv1-like growth factor |
| GGA1 | NM_001001561 | golgi associated, gamma adaptin ear containing, |
| GGT6 | NM_153338 | gamma-glutamyltransferase 6 homolog |
| GIMAP8 | NM_175571 | GTPase, IMAP family member 8 |
| GIOT-1 | NM_153257 | gonadotropin inducible transcription repressor |
| GIPC2 | NM_017655 | PDZ domain protein GIPC2 |
| GIT2 | NM_014776 | G protein-coupled receptor kinase-interactor 2 |
| GJA1 | NM_000165 | connexin 43 |
| GJB7 | NM_198568 | hypothetical protein LOC375519 |
| GKAP1 | NM_025211 | G kinase anchoring protein 1 |
| GLB1L | NM_024506 | galactosidase, beta 1-like |
| GLDN | NM_181789 | Collomin |
| GLO1 | NM_006708 | glyoxalase I |
| GLT25D2 | NM_015101 | glycosyltransferase 25 domain containing 2 |
| GLTP | NM_016433 | glycolipid transfer protein |
| GM632 | NM_020713 | hypothetical protein LOC57473 |
| GMCL1 | NM_178439 | germ cell-less |
| GMCL1L | NM_022471 | germ cell-less homolog 1 (Drosophila)-like |
| GMFB | NM_004124 | glia maturation factor, beta |
| GNAI1 | NM_002069 | guanine nucleotide binding protein (G protein), |
| GNAZ | NM_002073 | guanine nucleotide binding protein, alpha z |
| GNB5 | NM_006578 | guanine nucleotide-binding protein, beta-5 |
| GNE | NM_005476 | UDP-N-acetylglucosamine-2-epimerase/N- |
| GNPDA2 | NM_138335 | glucosamine-6-phosphate deaminase 2 |
| GNPNAT1 | NM_198066 | glucosamine-phosphate N-acetyltransferase 1 |
| GNPTAB | NM_024312 | N-acetylglucosamine-1-phosphate transferase |
| GNS | NM_002076 | glucosamine (N-acetyl)-6-sulfatase precursor |
| GOLGA1 | NM_002077 | golgin 97 |
| GOLGA2 | NM_004486 | Golgi autoantigen, golgin subfamily a, 2 |
| GOLPH2 | NM_016548 | golgi phosphoprotein 2 |
| GOLPH3 | NM_022130 | golgi phosphoprotein 3 |
| GORASP1 | NM_031899 | Golgi reassembly stacking protein 1 |
| GOSR1 | NM_001007024 | golgi SNAP receptor complex member 1 isoform 3 |
| GP5 | NM_004488 | glycoprotein V (platelet) |
| GPAM | NM_020918 | mitochondrial glycerol 3-phosphate |
| GPATC2 | NM_018040 | G patch domain containing 2 |
| GPD1 | NM_005276 | glycerol-3-phosphate dehydrogenase 1 (soluble) |
| GPIAP1 | NM_005898 | membrane component chromosome 11 surface marker |
| GPR1 | NM_005279 | G protein-coupled receptor 1 |
| GPR114 | NM_153837 | G-protein coupled receptor 114 |
| GPR126 | NM_001032394 | G protein-coupled receptor 126 alpha 2 |
| GPR132 | NM_013345 | G protein-coupled receptor 132 |
| GPR135 | NM_022571 | G protein-coupled receptor 135 |
| GPR137B | NM_003272 | transmembrane 7 superfamily member 1 |
| GPR155 | NM_001033045 | G protein-coupled receptor 155 |
| GPR176 | NM_007223 | putative G protein coupled receptor |
| GPR180 | NM_180989 | G protein-coupled receptor 180 precursor |
| GPR26 | NM_153442 | G protein-coupled receptor 26 |
| GPR3 | NM_005281 | G protein-coupled receptor 3 |
| GPR37 | NM_005302 | G protein-coupled receptor 37 |
| GPR45 | NM_007227 | G protein-coupled receptor 45 |
| GPR6 | NM_005284 | G protein-coupled receptor 6 |
| GPR81 | NM_032554 | G protein-coupled receptor 81 |
| GPR83 | NM_016540 | G protein-coupled receptor 83 |
| GPR85 | NM_018970 | G protein-coupled receptor 85 |
| GRAMD1A | NM_020895 | hypothetical protein LOC57655 |
| GRB2 | NM_002086 | growth factor receptor-bound protein 2 isoform |
| GREB1 | NM_148903 | GREB1 protein isoform c |
| GRHL2 | NM_024915 | transcription factor CP2-like 3 |
| GRIN3A | NM_133445 | glutamate receptor, ionotropic, |
| GRIPAP1 | NM_207672 | GRIP1 associated protein 1 isoform 2 |
| GRM1 | NM_000838 | glutamate receptor, metabotropic 1 |
| GRM6 | NM_000843 | glutamate receptor, metabotropic 6 precursor |
| GRM7 | NM_000844 | glutamate receptor, metabotropic 7 isoform a |
| GRPEL2 | NM_152407 | GrpE-like 2, mitochondrial |
| GRTP1 | NM_024719 | growth hormone regulated TBC protein 1 |
| GSTM3 | NM_000849 | glutathione S-transferase M3 |
| GTDC1 | NM_001006636 | glycosyltransferase-like domain containing 1 |
| GTF2H2 | NM_001515 | general transcription factor IIH, polypeptide 2, |
| GTPBP5 | NM_015666 | GTP binding protein 5 |
| GUCA1B | NM_002098 | guanylate cyclase activator 1B (retina) |
| GUCY1A3 | NM_000856 | guanylate cyclase 1, soluble, alpha 3 |
| GUCY1B2 | NM_004129 | guanylate cyclase 1, soluble, beta 2 |
| GYS1 | NM_002103 | glycogen synthase 1 (muscle) |
| H2AFJ | NM_018267 | H2A histone family, member J isoform 1 |
| H2AFY2 | NM_018649 | core histone macroH2A2.2 |
| H6PD | NM_004285 | hexose-6-phosphate dehydrogenase precursor |
| HARS | NM_002109 | histidyl-tRNA synthetase |
| HBP1 | NM_012257 | HMG-box transcription factor 1 |
| HBS1L | NM_006620 | HBS1-like |
| HBXIP | NM_006402 | hepatitis B virus x-interacting protein |
| HCCS | NM_005333 | holocytochrome c synthase (cytochrome c |
| HCLS1 | NM_005335 | hematopoietic cell-specific Lyn substrate 1 |
| HCP5 | NM_006674 | HLA complex P5 |
| HDAC4 | NM_006037 | histone deacetylase 4 |
| HDCMA18P | NM_016648 | hypothetical protein LOC51574 |
| HDHD1A | NM_012080 | haloacid dehalogenase-like hydrolase domain |
| HECA | NM_016217 | Headcase |
| HECTD2 | NM_182765 | HECT domain containing 2 isoform a |
| HEMK1 | NM_016173 | HemK methyltransferase family member 1 |
| HERPUD2 | NM_022373 | hypothetical protein LOC64224 |
| HERV-FRD | NM_207582 | HERV-FRD provirus ancestral Env polyprotein |
| HES2 | NM_019089 | hairy and enhancer of split homolog 2 |
| HEY2 | NM_012259 | hairy/enhancer-of-split related with YRPW motif |
| HIAT1 | NM_033055 | hippocampus abundant transcript 1 |
| HIC2 | NM_015094 | hypermethylated in cancer 2 |
| HIF1A | NM_001530 | hypoxia-inducible factor 1, alpha subunit |
| HIG2 | NM_013332 | hypoxia-inducible protein 2 |
| HIP1 | NM_005338 | huntingtin interacting protein 1 |
| HIP1R | NM_003959 | huntingtin interacting protein-1-related |
| HIST1H2AG | NM_021064 | H2A histone family, member P |
| HK1 | NM_000188 | hexokinase 1 isoform HKI |
| HLA-DOA | NM_002119 | major histocompatibility complex, class II, DO |
| HLCS | NM_000411 | holocarboxylase synthetase |
| HLF | NM_002126 | hepatic leukemia factor |
| HM13 | NM_178582 | minor histocompatibility antigen 13 isoform 4 |
| HMGA2 | NM_001015886 | high mobility group AT-hook 2 isoform c |
| HMGB3 | NM_005342 | high-mobility group box 3 |
| HMGCLL1 | NM_019036 | 3-hydroxymethyl-3-methylglutaryl-Coenzyme A |
| HMGN4 | NM_006353 | high mobility group nucleosomal binding domain |
| HMOX1 | NM_002133 | heme oxygenase (decyclizing) 1 |
| HN1 | NM_001002032 | hematological and neurological expressed 1 |
| HNF4G | NM_004133 | hepatocyte nuclear factor 4, gamma |
| HNMT | NM_006895 | histamine N-methyltransferase isoform 1 |
| HNRPH2 | NM_001032393 | heterogeneous nuclear ribonucleoprotein H2 |
| HNRPU | NM_004501 | heterogeneous nuclear ribonucleoprotein U |
| HNT | NM_016522 | Neurotrimin |
| HOOK3 | NM_032410 | golgi-associated microtubule-binding protein |
| HOXA3 | NM_030661 | homeobox A3 isoform a |
| HOXB13 | NM_006361 | homeobox B13 |
| HOXB4 | NM_024015 | homeobox B4 |
| HP1BP3 | NM_016287 | HP1-BP74 |
| HPCAL4 | NM_016257 | hippocalcin-like protein 4 |
| HPGD | NM_000860 | hydroxyprostaglandin dehydrogenase 15-(NAD) |
| HPS5 | NM_007216 | Hermansky-Pudlak syndrome 5 isoform b |
| HRB | NM_004504 | HIV-1 Rev binding protein |
| HRB2 | NM_007043 | HIV-1 rev binding protein 2 |
| HRBL | NM_006076 | HIV-1 Rev-binding protein-like protein |
| HRH2 | NM_022304 | histamine receptor H2 |
| HRH4 | NM_021624 | histamine H4 receptor |
| HS2ST1 | NM_012262 | heparan sulfate 2-O-sulfotransferase 1 |
| HS3ST4 | NM_006040 | heparan sulfate D-glucosaminyl |
| HSC20 | NM_172002 | J-type co-chaperone HSC20 |
| HSD17B7 | NM_016371 | hydroxysteroid (17-beta) dehydrogenase 7 |
| HSPA5 | NM_005347 | heat shock 70 kDa protein 5 (glucose-regulated |
| HSPA6 | NM_002155 | heat shock 70 kDa protein 6 (HSP70B′) |
| HSPA8 | NM_006597 | heat shock 70 kDa protein 8 isoform 1 |
| HSPC047 | NM_014147 | hypothetical protein LOC29060 |
| HSPC065 | NM_014157 | hypothetical protein LOC29070 |
| HSPC268 | NM_197964 | hypothetical protein LOC154791 |
| HSPH1 | NM_006644 | heat shock 105 kD |
| HTR2A | NM_000621 | 5-hydroxytryptamine (serotonin) receptor 2A |
| HUNK | NM_014586 | hormonally upregulated Neu-associated kinase |
| HYAL3 | NM_003549 | hyaluronoglucosaminidase 3 |
| HYPK | NM_016400 | Huntingtin interacting protein K |
| IAPP | NM_000415 | islet amyloid polypeptide precursor |
| ICAM4 | NM_001544 | intercellular adhesion molecule 4 isoform 1 |
| ICMT | NM_012405 | isoprenylcysteine carboxyl methyltransferase |
| IFIT1 | NM_001548 | interferon-induced protein with |
| IFIT3 | NM_001549 | interferon-induced protein with |
| IFIT5 | NM_012420 | interferon-induced protein with |
| IFNAR1 | NM_000629 | interferon-alpha receptor 1 precursor |
| IFNAR2 | NM_207585 | interferon alpha/beta receptor 2 isoform a |
| IFRD2 | NM_006764 | interferon-related developmental regulator 2 |
| IFT80 | NM_020800 | WD repeat domain 56 |
| IGF2BP1 | NM_006546 | insulin-like growth factor 2 mRNA binding |
| IGFBP5 | NM_000599 | insulin-like growth factor binding protein 5 |
| IGFBP7 | NM_001553 | insulin-like growth factor binding protein 7 |
| IGFL3 | NM_207393 | insulin growth factor-like family member 3 |
| IHPK1 | NM_001006115 | inositol hexaphosphate kinase 1 isoform 2 |
| IKBKB | NM_001556 | inhibitor of kappa light polypeptide gene |
| IKIP | NM_153687 | IKK interacting protein isoform 1 |
| IL10 | NM_000572 | interleukin 10 precursor |
| IL10RA | NM_001558 | interleukin 10 receptor, alpha precursor |
| IL11 | NM_000641 | interleukin 11 precursor |
| IL12RB2 | NM_001559 | interleukin 12 receptor, beta 2 precursor |
| IL17E | NM_022789 | interleukin 17E isoform 1 precursor |
| IL17F | NM_052872 | interleukin 17F precursor |
| IL17RB | NM_172234 | interleukin 17B receptor isoform 2 precursor |
| IL17RD | NM_017563 | interleukin 17 receptor D |
| IL1F5 | NM_012275 | interleukin 1 family, member 5 |
| IL1R1 | NM_000877 | interleukin 1 receptor, type I precursor |
| IL1RAP | NM_002182 | interleukin 1 receptor accessory protein isoform |
| IL1RL1 | NM_003856 | interleukin 1 receptor-like 1 isoform 2 |
| IL23R | NM_144701 | interleukin 23 receptor precursor |
| IL27RA | NM_004843 | class I cytokine receptor |
| IL28RA | NM_173065 | interleukin 28 receptor, alpha isoform 3 |
| IL6R | NM_000565 | interleukin 6 receptor isoform 1 precursor |
| IL8 | NM_000584 | interleukin 8 precursor |
| ILDR1 | NM_175924 | immunoglobulin-like domain containing receptor |
| ILKAP | NM_176799 | integrin-linked kinase-associated protein |
| IMPAD1 | NM_017813 | myo-inositol monophosphatase A3 |
| INHBA | NM_002192 | inhibin beta A precursor |
| INHBE | NM_031479 | activin beta B |
| INOC1 | NM_017553 | INO80 complex homolog 1 |
| INPP5B | NM_005540 | inositol polyphosphate-5-phosphatase, 75 kDa |
| INPP5F | NM_014937 | inositol polyphosphate-5-phosphatase F isoform |
| INTS5 | NM_030628 | integrator complex subunit 5 |
| INTS7 | NM_015434 | integrator complex subunit 7 |
| IPO8 | NM_006390 | importin 8 |
| IPP | NM_005897 | intracisternal A particle-promoted polypeptide |
| IPPK | NM_022755 | inositol 1,3,4,5,6-pentakisphosphate 2-kinase |
| IQCC | NM_018134 | IQ motif containing C |
| IQSEC1 | NM_014869 | IQ motif and Sec7 domain 1 |
| IQSEC2 | NM_015075 | IQ motif and Sec7 domain 2 |
| IRAK1 | NM_001025242 | interleukin-1 receptor-associated kinase 1 |
| IRAK4 | NM_016123 | interleukin-1 receptor-associated kinase 4 |
| IRF1 | NM_002198 | interferon regulatory factor 1 |
| IRXL1 | NM_173576 | hypothetical protein LOC283078 |
| ISG20L1 | NM_022767 | interferon stimulated exonuclease gene |
| ISGF3G | NM_006084 | interferon-stimulated transcription factor 3, |
| ITCH | NM_031483 | itchy homolog E3 ubiquitin protein ligase |
| ITFG1 | NM_030790 | T-cell immunomodulatory protein |
| ITGA10 | NM_003637 | integrin, alpha 10 precursor |
| ITGA4 | NM_000885 | integrin alpha 4 precursor |
| ITGAL | NM_002209 | integrin alpha L precursor |
| ITGB1 | NM_002211 | integrin beta 1 isoform 1A precursor |
| ITGB8 | NM_002214 | integrin, beta 8 |
| ITGBL1 | NM_004791 | integrin, beta-like 1 (with EGF-like repeat |
| ITIH5 | NM_001001851 | inter-alpha trypsin inhibitor heavy chain |
| ITIH5L | NM_198510 | hypothetical protein LOC347365 |
| ITPK1 | NM_014216 | inositol 1,3,4-triphosphate 5/6 kinase |
| ITPKB | NM_002221 | 1D-myo-inositol-trisphosphate 3-kinase B |
| ITSN2 | NM_147152 | intersectin 2 isoform 2 |
| IVNS1ABP | NM_006469 | influenza virus NS1A binding protein isoform a |
| IXL | NM_017592 | intersex-like |
| JAZF1 | NM_175061 | juxtaposed with another zinc finger gene 1 |
| JOSD1 | NM_014876 | hypothetical protein LOC9929 |
| JRKL | NM_003772 | jerky homolog-like |
| JUB | NM_032876 | jub, ajuba homolog isoform 1 |
| KAL1 | NM_000216 | Kallmann syndrome 1 protein |
| KATNAL1 | NM_001014380 | katanin p60 subunit A-like 1 |
| KBTBD6 | NM_152903 | kelch repeat and BTB (POZ) domain-containing 6 |
| KBTBD8 | NM_032505 | T-cell activation kelch repeat protein |
| KCNA7 | NM_031886 | potassium voltage-gated channel, shaker-related |
| KCNB1 | NM_004975 | potassium voltage-gated channel, Shab-related |
| KCNH5 | NM_139318 | potassium voltage-gated channel, subfamily H, |
| KCNH6 | NM_030779 | potassium voltage-gated channel, subfamily H, |
| KCNH8 | NM_144633 | potassium voltage-gated channel, subfamily H, |
| KCNJ10 | NM_002241 | potassium inwardly-rectifying channel, subfamily |
| KCNJ16 | NM_018658 | potassium inwardly-rectifying channel J16 |
| KCNJ8 | NM_004982 | potassium inwardly-rectifying channel J8 |
| KCNJ9 | NM_004983 | potassium inwardly-rectifying channel subfamily |
| KCNK1 | NM_002245 | potassium channel, subfamily K, member 1 |
| KCNK2 | NM_001017424 | potassium channel, subfamily K, member 2 isoform |
| KCNK3 | NM_002246 | potassium channel, subfamily K, member 3 |
| KCNK6 | NM_004823 | potassium channel, subfamily K, member 6 |
| KCNMA1 | NM_001014797 | large conductance calcium-activated potassium |
| KCNQ2 | NM_004518 | potassium voltage-gated channel KQT-like protein |
| KCNRG | NM_199464 | potassium channel regulator isoform 2 |
| KCNT2 | NM_198503 | potassium channel, subfamily T, member 2 |
| KCTD18 | NM_152387 | potassium channel tetramerisation domain |
| KDELC2 | NM_153705 | KDEL (Lys-Asp-Glu-Leu) containing 2 |
| KEAP1 | NM_012289 | kelch-like ECH-associated protein 1 |
| KIAA0082 | NM_015050 | hypothetical protein LOC23070 |
| KIAA0143 | NM_015137 | hypothetical protein LOC23167 |
| KIAA0157 | NM_032182 | hypothetical protein LOC23172 |
| KIAA0240 | NM_015349 | hypothetical protein LOC23506 |
| KIAA0247 | NM_014734 | hypothetical protein LOC9766 |
| KIAA0319 | NM_014809 | KIAA0319 |
| KIAA0319L | NM_024874 | polycystic kidney disease 1-like isoform a |
| KIAA0355 | NM_014686 | hypothetical protein LOC9710 |
| KIAA0367 | NM_015225 | BNIP2 motif containing molecule at the carboxyl |
| KIAA0404 | NM_015104 | hypothetical protein LOC23130 |
| KIAA0427 | NM_014772 | hypothetical protein LOC9811 |
| KIAA0446 | NM_014655 | hypothetical protein LOC9673 |
| KIAA0467 | NM_015284 | KIAA0467 protein |
| KIAA0494 | NM_014774 | hypothetical protein LOC9813 |
| KIAA0495 | NM_207306 | KIAA0495 |
| KIAA0513 | NM_014732 | hypothetical protein LOC9764 |
| KIAA0562 | NM_014704 | glycine-, glutamate-, |
| KIAA0564 | NM_015058 | hypothetical protein LOC23078 isoform a |
| KIAA0649 | NM_014811 | 1A6/DRIM (down-regulated in metastasis) |
| KIAA0664 | NM_015229 | hypothetical protein LOC23277 |
| KIAA0773 | NM_001031690 | hypothetical protein LOC9715 |
| KIAA0828 | NM_015328 | KIAA0828 protein |
| KIAA0831 | NM_014924 | hypothetical protein LOC22863 |
| KIAA0889 | NM_152257 | hypothetical protein LOC25781 |
| KIAA0892 | NM_015329 | hypothetical protein LOC23383 |
| KIAA0895 | NM_015314 | hypothetical protein LOC23366 |
| KIAA0922 | NM_015196 | KIAA0922 protein |
| KIAA0980 | NM_025176 | hypothetical protein LOC22981 |
| KIAA0999 | NM_025164 | KIAA0999 protein |
| KIAA1128 | NM_018999 | granule cell antiserum positive 14 |
| KIAA1160 | NM_020701 | hypothetical protein LOC57461 |
| KIAA1191 | NM_020444 | hypothetical protein LOC57179 |
| KIAA1193 | NM_017550 | hypothetical protein LOC54531 |
| KIAA1199 | NM_018689 | KIAA1199 |
| KIAA1202 | NM_020717 | hypothetical protein LOC57477 |
| KIAA1324 | NM_020775 | hypothetical protein LOC57535 |
| KIAA1377 | NM_020802 | hypothetical protein LOC57562 |
| KIAA1434 | NM_019593 | hypothetical protein LOC56261 |
| KIAA1456 | NM_020844 | hypothetical protein LOC57604 |
| KIAA1522 | NM_020888 | hypothetical protein LOC57648 |
| KIAA1530 | NM_020894 | hypothetical protein LOC57654 |
| KIAA1559 | NM_020917 | zinc finger protein 14-like |
| KIAA1598 | NM_018330 | hypothetical protein LOC57698 |
| KIAA1600 | NM_020940 | hypothetical protein LOC57700 |
| KIAA1609 | NM_020947 | hypothetical protein LOC57707 |
| KIAA1618 | NM_020954 | hypothetical protein LOC57714 |
| KIAA1627 | NM_020961 | hypothetical protein LOC57721 |
| KIAA1641 | NM_020970 | hypothetical protein LOC57730 |
| KIAA1706 | NM_030636 | hypothetical protein LOC80820 |
| KIAA1727 | NM_033393 | hypothetical protein LOC85462 |
| KIAA1826 | NM_032424 | KIAA1826 protein |
| KIAA1909 | NM_052909 | hypothetical protein LOC153478 |
| KIF11 | NM_004523 | kinesin family member 11 |
| KIF13B | NM_015254 | kinesin family member 13B |
| KIF14 | NM_014875 | kinesin family member 14 |
| KIF1A | NM_004321 | axonal transport of synaptic vesicles |
| KIF1B | NM_015074 | kinesin family member 1B isoform b |
| KIF23 | NM_004856 | kinesin family member 23 isoform 2 |
| KIF3B | NM_004798 | kinesin family member 3B |
| KIF3C | NM_002254 | kinesin family member 3C |
| KIF5A | NM_004984 | kinesin family member 5A |
| KIF9 | NM_022342 | kinesin family member 9 isoform 1 |
| KIRREL | NM_018240 | kin of IRRE like |
| KIT | NM_000222 | v-kit Hardy-Zuckerman 4 feline sarcoma viral |
| KLC3 | NM_145275 | kinesin light chain 2-like isoform b |
| KLF10 | NM_001032282 | Kruppel-like factor 10 isoform b |
| KLF11 | NM_003597 | Kruppel-like factor 11 |
| KLF12 | NM_007249 | Kruppel-like factor 12 isoform a |
| KLF13 | NM_015995 | Kruppel-like factor 13 |
| KLF17 | NM_173484 | zinc finger protein 393 |
| KLF9 | NM_001206 | Kruppel-like factor 9 |
| KLHDC5 | NM_020782 | kelch domain containing 5 |
| KLHDC6 | NM_207335 | hypothetical protein LOC166348 |
| KLHL12 | NM_021633 | kelch-like 12 |
| KLHL2 | NM_007246 | kelch-like 2, Mayven |
| KLHL20 | NM_014458 | kelch-like 20 |
| KLHL21 | NM_014851 | kelch-like 21 |
| KLHL22 | NM_032775 | kelch-like |
| KLHL23 | NM_144711 | kelch-like 23 |
| KLHL3 | NM_017415 | kelch-like 3 (Drosophila) |
| KLHL8 | NM_020803 | kelch-like 8 |
| KLK5 | NM_012427 | kallikrein 5 preproprotein |
| KLK7 | NM_005046 | stratum corneum chymotryptic enzyme |
| KLRK1 | NM_007360 | NKG2-D type II integral membrane protein |
| KMO | NM_003679 | kynurenine 3-monooxygenase |
| KPNA2 | NM_002266 | karyopherin alpha 2 |
| KPNA3 | NM_002267 | karyopherin alpha 3 |
| KPNA4 | NM_002268 | karyopherin alpha 4 |
| KRIT1 | NM_001013406 | krev interaction trapped 1 isoform 2 |
| KRT10 | NM_000421 | keratin 10 |
| KRT23 | NM_015515 | keratin 23 |
| KRT2A | NM_000423 | keratin 2a |
| KRT2B | NM_015848 | cytokeratin 2 |
| KRT6IRS | NM_033448 | keratin 6 irs |
| KRTAP10-4 | NM_198687 | keratin associated protein 10-4 |
| KRTHB1 | NM_002281 | keratin, hair, basic, 1 |
| KRTHB5 | NM_002283 | keratin, hair, basic, 5 |
| KTI12 | NM_138417 | KTI12 homolog, chromatin associated |
| L2HGDH | NM_024884 | hypothetical protein LOC79944 |
| L3MBTL2 | NM_001003689 | l(3)mbt-like 2 isoform b |
| L3MBTL3 | NM_001007102 | l(3)mbt-like 3 isoform b |
| L3MBTL4 | NM_173464 | hypothetical protein LOC91133 |
| LACE1 | NM_145315 | lactation elevated 1 |
| LALBA | NM_002289 | lactalbumin, alpha-precursor |
| LAMA3 | NM_000227 | laminin alpha 3 subunit isoform 2 |
| LAMC1 | NM_002293 | laminin, gamma 1 precursor |
| LAMC2 | NM_018891 | laminin, gamma 2 isoform b precursor |
| LAMP1 | NM_005561 | lysosomal-associated membrane protein 1 |
| LAMP2 | NM_013995 | lysosomal-associated membrane protein 2 |
| LAMP3 | NM_014398 | lysosomal-associated membrane protein 3 |
| LAPTM4A | NM_014713 | lysosomal-associated protein transmembrane 4 |
| LARP2 | NM_018078 | La ribonucleoprotein domain family member 2 |
| LARP5 | NM_015155 | La ribonucleoprotein domain family, member 5 |
| LASP1 | NM_006148 | LIM and SH3 protein 1 |
| LASS2 | NM_013384 | LAG1 longevity assurance homolog 2 isoform 2 |
| LASS3 | NM_178842 | hypothetical protein LOC204219 |
| LASS6 | NM_203463 | longevity assurance homolog 6 |
| LAX1 | NM_017773 | lymphocyte transmembrane adaptor 1 |
| LBH | NM_030915 | hypothetical protein DKFZp566J091 |
| LDLR | NM_000527 | low density lipoprotein receptor precursor |
| LDLRAD3 | NM_174902 | hypothetical protein LOC143458 |
| LDLRAP1 | NM_015627 | low density lipoprotein receptor adaptor protein |
| LDOC1L | NM_032287 | hypothetical protein LOC84247 |
| LEP | NM_000230 | leptin precursor |
| LEPREL1 | NM_018192 | leprecan-like 1 |
| LEPROT | NM_017526 | leptin receptor gene-related protein |
| LEPROTL1 | NM_015344 | leptin receptor overlapping transcript-like 1 |
| LETM1 | NM_012318 | leucine zipper-EF-hand containing transmembrane |
| LGALS8 | NM_006499 | galectin 8 isoform a |
| LHFP | NM_005780 | lipoma HMGIC fusion partner |
| LHFPL2 | NM_005779 | lipoma HMGIC fusion partner-like 2 |
| LHFPL3 | NM_199000 | lipoma HMGIC fusion partner-like 3 |
| LHX6 | NM_014368 | LIM homeobox protein 6 isoform 1 |
| LHX8 | NM_001001933 | LIM homeobox 8 |
| LIAS | NM_006859 | lipoic acid synthetase isoform 1 precursor |
| LIF | NM_002309 | leukemia inhibitory factor (cholinergic |
| LILRA5 | NM_181986 | leukocyte immunoglobulin-like receptor subfamily |
| LILRB1 | NM_006669 | leukocyte immunoglobulin-like receptor, |
| LIMA1 | NM_016357 | epithelial protein lost in neoplasm beta |
| LIMK1 | NM_002314 | LIM domain kinase 1 isoform 1 |
| LIN10 | NM_025187 | lin-10 |
| LIN28 | NM_024674 | lin-28 homolog |
| LIN7B | NM_022165 | lin-7 homolog B |
| LINS1 | NM_181740 | lines homolog 1 isoform 3 |
| LIPH | NM_139248 | lipase, member H precursor |
| LITAF | NM_004862 | LPS-induced TNF-alpha factor |
| LMO2 | NM_005574 | LIM domain only 2 |
| LMO3 | NM_001001395 | LIM domain only 3 |
| LMOD3 | NM_198271 | leiomodin 3 (fetal) |
| LNK | NM_005475 | lymphocyte adaptor protein |
| LOC116143 | NM_138458 | Monad |
| LOC116236 | NM_198147 | hypothetical protein LOC116236 |
| LOC123688 | NM_001013619 | hypothetical protein LOC123688 |
| LOC124751 | NM_213597 | hypothetical protein LOC124751 |
| LOC126248 | NM_173479 | hypothetical protein LOC126248 |
| LOC128439 | NM_139016 | hypothetical protein LOC128439 |
| LOC129285 | NM_152994 | smooth muscle myosin heavy chain 11 isoform |
| LOC130951 | NM_138804 | hypothetical protein LOC130951 |
| LOC133619 | NM_130809 | hypothetical protein LOC133619 |
| LOC134147 | NM_138809 | hypothetical protein LOC134147 |
| LOC136263 | NM_145268 | hypothetical protein LOC136263 |
| LOC148137 | NM_144692 | hypothetical protein LOC148137 |
| LOC149620 | NM_001013621 | hypothetical protein LOC149620 |
| LOC151194 | NM_145280 | hypothetical protein LOC151194 |
| LOC153222 | NM_153607 | hypothetical protein LOC153222 |
| LOC153561 | NM_207331 | hypothetical protein LOC153561 |
| LOC158160 | NM_001031744 | 17-beta-hydroxysteroid dehydrogenase type |
| LOC162427 | NM_178126 | hypothetical protein LOC162427 |
| LOC168850 | NM_176814 | hypothetical protein LOC168850 |
| LOC201895 | NM_174921 | hypothetical protein LOC201895 |
| LOC203427 | NM_145305 | mitochondrial solute carrier protein |
| LOC203547 | NM_001017980 | hypothetical protein LOC203547 |
| LOC220594 | NM_145809 | TL132 protein |
| LOC221091 | NM_203422 | hypothetical protein LOC221091 |
| LOC222171 | NM_175887 | hypothetical protein LOC222171 |
| LOC223075 | NM_194300 | hypothetical protein LOC223075 |
| LOC283537 | NM_181785 | hypothetical protein LOC283537 |
| LOC283551 | NM_001012706 | hypothetical protein LOC283551 |
| LOC283849 | NM_178516 | hypothetical protein LOC283849 |
| LOC284434 | NM_001007525 | hypothetical protein LOC284434 |
| LOC284912 | NM_203375 | hypothetical protein LOC284912 |
| LOC285382 | NM_001025266 | hypothetical protein LOC285382 |
| LOC285636 | NM_175921 | hypothetical protein LOC285636 |
| LOC338328 | NM_178172 | high density lipoprotein-binding protein |
| LOC339745 | NM_001001664 | hypothetical protein LOC339745 |
| LOC340843 | NM_001013629 | hypothetical protein LOC340843 |
| LOC347273 | NM_001018116 | hypothetical protein LOC347273 |
| LOC348262 | NM_207368 | hypothetical protein LOC348262 |
| LOC387758 | NM_203371 | hypothetical protein LOC387758 |
| LOC387790 | NM_001013634 | hypothetical protein LOC387790 |
| LOC387873 | NM_001013636 | hypothetical protein LOC387873 |
| LOC387882 | NM_207376 | hypothetical protein LOC387882 |
| LOC387921 | NM_001012754 | hypothetical protein LOC387921 isoform a |
| LOC388335 | NM_001004313 | hypothetical protein LOC388335 |
| LOC388610 | NM_001013642 | hypothetical protein LOC388610 |
| LOC388969 | NM_001013649 | hypothetical protein LOC388969 |
| LOC389432 | NM_001030060 | hypothetical protein LOC389432 |
| LOC389634 | NM_001012988 | hypothetical protein LOC389634 |
| LOC389936 | NM_001013656 | hypothetical protein LOC389936 |
| LOC390980 | NM_001023563 | similar to Zinc finger protein 264 |
| LOC399706 | NM_001010910 | hypothetical protein LOC399706 |
| LOC400464 | NM_001013670 | hypothetical protein LOC400464 |
| LOC400499 | NM_001013671 | hypothetical protein LOC400499 |
| LOC401137 | NM_214711 | hypothetical protein LOC401137 |
| LOC401152 | NM_001001701 | hypothetical protein LOC401152 |
| LOC401410 | NM_001008742 | hypothetical protein LOC401410 |
| LOC401431 | NM_001008745 | hypothetical protein LOC401431 |
| LOC401507 | NM_001012278 | hypothetical protein LOC401507 |
| LOC401589 | NM_001013687 | hypothetical protein LOC401589 |
| LOC401620 | NM_001013688 | hypothetical protein LOC401620 |
| LOC402176 | NM_001011538 | hypothetical protein LOC402176 |
| LOC440248 | NM_199045 | hypothetical protein LOC440248 |
| LOC440742 | NM_001013710 | hypothetical protein LOC440742 |
| LOC441136 | NM_001013719 | hypothetical protein LOC441136 |
| LOC441208 | NM_001013723 | hypothetical protein LOC441208 |
| LOC441268 | NM_001013725 | hypothetical protein LOC441268 |
| LOC441376 | NM_001025357 | hypothetical protein LOC441376 |
| LOC442578 | NM_001013739 | hypothetical protein LOC442578 |
| LOC493829 | NM_001008274 | hypothetical protein LOC493829 |
| LOC51149 | NM_001017987 | hypothetical protein LOC51149 isoform 2 |
| LOC51333 | NM_016643 | mesenchymal stem cell protein DSC43 |
| LOC51334 | NM_016644 | mesenchymal stem cell protein DSC54 |
| LOC554251 | NM_001024680 | hypothetical protein LOC554251 |
| LOC57149 | NM_020424 | hypothetical protein LOC57149 |
| LOC613206 | NM_001033016 | myeloproliferative disease associated tumor |
| LOC613266 | NM_001033516 | hypothetical protein LOC613266 |
| LOC619208 | NM_001033564 | hypothetical protein LOC619208 |
| LOC63928 | NM_022097 | hepatocellular carcinoma antigen gene 520 |
| LOC63929 | NM_022098 | hypothetical protein LOC63929 |
| LOC81558 | NM_030802 | C/EBP-induced protein |
| LOC90321 | NM_001010851 | hypothetical protein LOC90321 |
| LOC90624 | NM_181705 | hypothetical protein LOC90624 |
| LOC90639 | NM_001031617 | hypothetical protein LOC90639 |
| LOC94431 | NM_145237 | hypothetical protein LOC94431 |
| LONPL | NM_031490 | peroxisomal LON protease-like |
| LPGAT1 | NM_014873 | lysophosphatidylglycerol acyltransferase 1 |
| LPHN3 | NM_015236 | latrophilin 3 precursor |
| LPIN1 | NM_145693 | lipin 1 |
| LPIN3 | NM_022896 | lipin 3 |
| LRAP | NM_022350 | leukocyte-derived arginine aminopeptidase |
| LRAT | NM_004744 | lecithin retinol acyltransferase |
| LRFN5 | NM_152447 | leucine rich repeat and fibronectin type III |
| LRG1 | NM_052972 | leucine-rich alpha-2-glycoprotein 1 |
| LRIG1 | NM_015541 | leucine-rich repeats and immunoglobulin-like |
| LRP1 | NM_002332 | low density lipoprotein-related protein 1 |
| LRP12 | NM_013437 | suppression of tumorigenicity |
| LRP1B | NM_018557 | low density lipoprotein-related protein 1B |
| LRP2BP | NM_018409 | LRP2 binding protein |
| LRP4 | NM_002334 | low density lipoprotein receptor-related protein |
| LRRC10 | NM_201550 | leucine rich repeat containing 10 |
| LRRC15 | NM_130830 | leucine rich repeat containing 15 |
| LRRC2 | NM_024512 | leucine rich repeat containing 2 |
| LRRC20 | NM_018205 | leucine rich repeat containing 20 isoform 3 |
| LRRC27 | NM_030626 | leucine rich repeat containing 27 |
| LRRC32 | NM_005512 | leucine rich repeat containing 32 precursor |
| LRRC44 | NM_145258 | leucine rich repeat containing 44 |
| LRRC45 | NM_144999 | leucine rich repeat containing 45 |
| LRRC54 | NM_015516 | Tsukushi |
| LRRC55 | NM_001005210 | hypothetical protein LOC219527 |
| LRRC57 | NM_153260 | hypothetical protein LOC255252 |
| LRRC8A | NM_019594 | leucine-rich repeat-containing 8 |
| LRRC8B | NM_015350 | T-cell activation leucine repeat-rich protein |
| LRRIQ2 | NM_024548 | leucine-rich repeats and IQ motif containing 2 |
| LRRN6A | NM_032808 | leucine-rich repeat neuronal 6A |
| LSM11 | NM_173491 | LSM11, U7 small nuclear RNA associated |
| LSM12 | NM_152344 | hypothetical protein LOC124801 |
| LTB4R | NM_181657 | leukotriene B4 receptor |
| LTBP2 | NM_000428 | latent transforming growth factor beta binding |
| LTBR | NM_002342 | lymphotoxin beta receptor |
| LTV1 | NM_032860 | hypothetical protein LOC84946 |
| LUZP1 | NM_033631 | leucine zipper protein 1 |
| LY75 | NM_002349 | lymphocyte antigen 75 |
| LYCAT | NM_001002257 | lysocardiolipin acyltransferase isoform 2 |
| LYST | NM_000081 | lysosomal trafficking regulator isoform 1 |
| M6PR | NM_002355 | cation-dependent mannose-6-phosphate receptor |
| M6PRBP1 | NM_005817 | mannose 6 phosphate receptor binding protein 1 |
| MAF1 | NM_032272 | MAF1 protein |
| MAFF | NM_012323 | transcription factor MAFF |
| MAGI2 | NM_012301 | membrane associated guanylate kinase, WW and PDZ |
| MAK | NM_005906 | male germ cell-associated kinase |
| MAL2 | NM_052886 | mal, T-cell differentiation protein 2 |
| MAN1A2 | NM_006699 | mannosidase, alpha, class 1A, member 2 |
| MAN2A2 | NM_006122 | mannosidase, alpha, class 2A, member 2 |
| MANEAL | NM_152496 | hypothetical protein LOC149175 isoform 2 |
| MAP1LC3B | NM_022818 | microtubule-associated proteins 1A/1B light |
| MAP3K11 | NM_002419 | mitogen-activated protein kinase kinase kinase |
| MAP3K12 | NM_006301 | mitogen-activated protein kinase kinase kinase |
| MAP3K14 | NM_003954 | mitogen-activated protein kinase kinase kinase |
| MAP3K2 | NM_006609 | mitogen-activated protein kinase kinase kinase |
| MAP3K3 | NM_002401 | mitogen-activated protein kinase kinase kinase 3 |
| MAP3K5 | NM_005923 | mitogen-activated protein kinase kinase kinase |
| MAP3K7 | NM_003188 | mitogen-activated protein kinase kinase kinase 7 |
| MAP3K8 | NM_005204 | mitogen-activated protein kinase kinase kinase |
| MAP3K9 | NM_033141 | mitogen-activated protein kinase kinase kinase |
| MAP6 | NM_207577 | microtubule-associated protein 6 isoform 2 |
| MAP7 | NM_003980 | microtubule-associated protein 7 |
| MAPK1 | NM_002745 | mitogen-activated protein kinase 1 |
| MAPK4 | NM_002747 | mitogen-activated protein kinase 4 |
| MAPK9 | NM_002752 | mitogen-activated protein kinase 9 isoform 1 |
| MAPKBP1 | NM_014994 | mitogen-activated protein kinase binding protein |
| MAPRE1 | NM_012325 | microtubule-associated protein, RP/EB family, |
| MAPRE3 | NM_012326 | microtubule-associated protein, RP/EB family, |
| MARCH2 | NM_001005415 | membrane-associated ring finger (C3HC4) 2 |
| MARCH5 | NM_017824 | ring finger protein 153 |
| MARCH6 | NM_005885 | membrane-associated ring finger (C3HC4) 6 |
| MARCH7 | NM_022826 | Axotrophin |
| MARCH8 | NM_001002265 | cellular modulator of immune recognition |
| MARK1 | NM_018650 | MAP/microtubule affinity-regulating kinase 1 |
| MARK4 | NM_031417 | MAP/microtubule affinity-regulating kinase 4 |
| MARS | NM_004990 | methionine-tRNA synthetase |
| MARVELD3 | NM_001017967 | MARVEL domain containing 3 isoform 1 |
| MASTL | NM_032844 | microtubule associated serine/threonine |
| MAT2B | NM_013283 | methionine adenosyltransferase II, beta isoform |
| MAWBP | NM_022129 | MAWD binding protein isoform a |
| MBD5 | NM_018328 | methyl-CpG binding domain protein 5 |
| MBNL1 | NM_021038 | muscleblind-like 1 isoform a |
| MBNL3 | NM_018388 | muscleblind-like 3 isoform G |
| MBP | NM_001025100 | Golli-mbp isoform 2 |
| MBTD1 | NM_017643 | mbt domain containing 1 |
| MBTPS1 | NM_003791 | membrane-bound transcription factor site-1 |
| MCAM | NM_006500 | melanoma cell adhesion molecule |
| MCF2L2 | NM_015078 | Rho family guanine-nucleotide exchange factor |
| MCFD2 | NM_139279 | multiple coagulation factor deficiency 2 |
| MCL1 | NM_021960 | myeloid cell leukemia sequence 1 isoform 1 |
| MCM3 | NM_002388 | minichromosome maintenance protein 3 |
| MCM4 | NM_005914 | minichromosome maintenance protein 4 |
| MCMDC1 | NM_153255 | minichromosome maintenance protein domain |
| MCOLN2 | NM_153259 | mucolipin 2 |
| MDFIC | NM_199072 | MyoD family inhibitor domain containing isoform |
| MDM4 | NM_002393 | mouse double minute 4 homolog |
| MECP2 | NM_004992 | methyl CpG binding protein 2 |
| MECR | NM_001024732 | nuclear receptor-binding factor 1 isoform b |
| MED12L | NM_053002 | hypothetical protein LOC116931 |
| MED18 | NM_017638 | mediator of RNA polymerase II transcription, |
| MED6 | NM_005466 | mediator of RNA polymerase II transcription, |
| METAP1 | NM_015143 | methionyl aminopeptidase 1 |
| METT5D1 | NM_152636 | methyltransferase 5 domain containing 1 |
| METTL2A | NM_001005372 | hypothetical protein LOC339175 |
| METTL4 | NM_022840 | methyltransferase like 4 |
| MFAP3L | NM_001009554 | microfibrillar-associated protein 3-like isoform |
| MFAP5 | NM_003480 | microfibrillar associated protein 5 |
| MFN2 | NM_014874 | mitofusin 2 |
| MFSD4 | NM_181644 | hypothetical protein DKFZp761N1114 |
| MGC11266 | NM_024322 | hypothetical protein LOC79172 |
| MGC11332 | NM_032718 | hypothetical protein LOC84804 |
| MGC13017 | NM_080656 | hypothetical protein LOC91368 |
| MGC15476 | NM_145056 | thymus expressed gene 3-like |
| MGC15619 | NM_032369 | hypothetical protein LOC84329 |
| MGC16291 | NM_032770 | hypothetical protein LOC84856 |
| MGC16385 | NM_145039 | hypothetical protein LOC92806 |
| MGC16703 | NM_145042 | hypothetical protein LOC113691 |
| MGC19604 | NM_001031734 | hypothetical protein LOC112812 isoform 1 |
| MGC22001 | NM_153238 | hypothetical protein LOC197196 |
| MGC24039 | NM_144973 | hypothetical protein LOC160518 |
| MGC26718 | NM_001029999 | hypothetical protein LOC440482 |
| MGC26733 | NM_144992 | hypothetical protein LOC200403 |
| MGC26816 | NM_152613 | hypothetical protein LOC164684 |
| MGC2752 | NM_023939 | hypothetical protein LOC65996 |
| MGC29891 | NM_144618 | GA repeat binding protein, beta 2 |
| MGC3123 | NM_024107 | hypothetical protein LOC79089 isoform 1 |
| MGC32020 | NM_152266 | hypothetical protein LOC91442 |
| MGC3207 | NM_001031727 | hypothetical protein LOC84245 isoform 1 |
| MGC34646 | NM_173519 | hypothetical protein LOC157807 |
| MGC34821 | NM_173586 | hypothetical protein LOC283238 |
| MGC35048 | NM_153208 | hypothetical protein LOC124152 |
| MGC35440 | NM_153220 | hypothetical protein LOC147990 |
| MGC39518 | NM_173822 | hypothetical protein LOC285172 |
| MGC40069 | NM_182615 | hypothetical protein LOC348035 |
| MGC40405 | NM_152789 | hypothetical protein LOC257415 |
| MGC42090 | NM_152774 | hypothetical protein LOC256130 |
| MGC4268 | NM_031445 | hypothetical protein LOC83607 |
| MGC45438 | NM_152459 | hypothetical protein LOC146556 |
| MGC4562 | NM_133375 | hypothetical protein LOC115752 |
| MGC4655 | NM_033309 | hypothetical protein LOC84752 |
| MGC48628 | NM_207491 | hypothetical protein LOC401145 |
| MGC50372 | NM_173566 | hypothetical protein LOC253143 |
| MGC52057 | NM_194317 | hypothetical protein LOC130574 |
| MGC52110 | NM_001008215 | hypothetical protein LOC493753 |
| MGC52498 | NM_182621 | hypothetical protein LOC348378 |
| MGC70857 | NM_001001795 | hypothetical protein LOC414919 |
| MGC87631 | NM_001004306 | hypothetical protein LOC339184 |
| MGC9712 | NM_152689 | hypothetical protein LOC202915 |
| MGEA5 | NM_012215 | meningioma expressed antigen 5 (hyaluronidase) |
| MGLL | NM_001003794 | monoglyceride lipase isoform 2 |
| MICA | NM_000247 | MHC class I chain-related gene A protein |
| MICB | NM_005931 | MHC class I polypeptide-related sequence B |
| MIDN | NM_177401 | Midnolin |
| MINK1 | NM_001024937 | misshapen/NIK-related kinase isoform 4 |
| MKI67 | NM_002417 | antigen identified by monoclonal antibody Ki-67 |
| MKL2 | NM_014048 | megakaryoblastic leukemia 2 protein |
| MKLN1 | NM_013255 | muskelin 1, intracellular mediator containing |
| MKNK2 | NM_017572 | MAP kinase-interacting serine/threonine kinase 2 |
| MKRN1 | NM_013446 | makorin, ring finger protein, 1 |
| MLC1 | NM_015166 | megalencephalic leukoencephalopathy with |
| MLL3 | NM_021230 | myeloid/lymphoid or mixed-lineage leukemia 3 |
| MLL4 | NM_014727 | myeloid/lymphoid or mixed-lineage leukemia 4 |
| MLLT10 | NM_001009569 | myeloid/lymphoid or mixed-lineage leukemia |
| MLLT11 | NM_006818 | MLLT11 protein |
| MLLT6 | NM_005937 | myeloid/lymphoid or mixed-lineage leukemia |
| MLR1 | NM_153686 | transcription factor MLR1 |
| MMACHC | NM_015506 | hypothetical protein LOC25974 |
| MME | NM_000902 | membrane metallo-endopeptidase |
| MMP19 | NM_001032360 | matrix metalloproteinase 19 isoform 2 precursor |
| MMP2 | NM_004530 | matrix metalloproteinase 2 preproprotein |
| MMP23A | NM_004659 | matrix metalloproteinase 23A precursor |
| MMP23B | NM_006983 | matrix metalloproteinase 23B precursor |
| MMP24 | NM_006690 | matrix metalloproteinase 24 preproprotein |
| MMP3 | NM_002422 | matrix metalloproteinase 3 preproprotein |
| MMRN2 | NM_024756 | multimerin 2 |
| MOBKL1A | NM_173468 | MOB1, Mps One Binder kinase activator-like 1A |
| MOCS1 | NM_005943 | molybdenum cofactor synthesis-step 1 protein |
| MOG | NM_001008228 | myelin oligodendrocyte glycoprotein isoform |
| MOGAT3 | NM_178176 | monoacylglycerol O-acyltransferase 3 |
| MORF4L1 | NM_006791 | MORF-related gene 15 isoform 1 |
| MORF4L2 | NM_012286 | MORF-related gene X |
| MPPE1 | NM_023075 | metallophosphoesterase 1 isoform a precursor |
| MPZ | NM_000530 | myelin protein zero |
| MRAS | NM_012219 | muscle RAS oncogene homolog |
| MRCL3 | NM_006471 | myosin regulatory light chain MRCL3 |
| MRE11A | NM_005590 | meiotic recombination 11 homolog A isoform 2 |
| MRGPRX3 | NM_054031 | G protein-coupled receptor MRGX3 |
| MRP63 | NM_024026 | mitochondrial ribosomal protein 63 |
| MRPL17 | NM_022061 | mitochondrial ribosomal protein L17 |
| MRPL24 | NM_024540 | mitochondrial ribosomal protein L24 |
| MRPL30 | NM_145212 | mitochondrial ribosomal protein L30 |
| MRPL43 | NM_032112 | mitochondrial ribosomal protein L43 isoform a |
| MRPL47 | NM_020409 | mitochondrial ribosomal protein L47 isoform a |
| MRPL49 | NM_004927 | mitochondrial ribosomal protein L49 |
| MRPL52 | NM_178336 | mitochondrial ribosomal protein L52 isoform a |
| MRPS10 | NM_018141 | mitochondrial ribosomal protein S10 |
| MRPS16 | NM_016065 | mitochondrial ribosomal protein S16 |
| MRPS18B | NM_014046 | mitochondrial ribosomal protein S18B |
| MRPS25 | NM_022497 | mitochondrial ribosomal protein S25 |
| MRPS36 | NM_033281 | mitochondrial ribosomal protein S36 |
| MRRF | NM_138777 | mitochondrial ribosome recycling factor isoform |
| MS4A10 | NM_206893 | membrane-spanning 4-domains, subfamily A, member |
| MS4A7 | NM_021201 | membrane-spanning 4-domains, subfamily A, member |
| MSH3 | NM_002439 | mutS homolog 3 |
| MSL2L1 | NM_018133 | ring finger protein 184 |
| MSR1 | NM_138715 | macrophage scavenger receptor 1 isoform type 1 |
| MSRB3 | NM_001031679 | methionine sulfoxide reductase B3 isoform 2 |
| MST150 | NM_032947 | putative small membrane protein NID67 |
| MSTO1 | NM_018116 | Misato |
| MTAC2D1 | NM_152332 | membrane targeting (tandem) C2 domain containing |
| MTCH2 | NM_014342 | mitochondrial carrier homolog 2 |
| MTERFD2 | NM_182501 | MTERF domain containing 2 |
| MTF1 | NM_005955 | metal-regulatory transcription factor 1 |
| MTFMT | NM_139242 | methionyl-tRNA formyltransferase, mitochondrial |
| MTHFD1L | NM_015440 | methylenetetrahydrofolate dehydrogenase (NADP+ |
| MTHFD2 | NM_006636 | methylene tetrahydrofolate dehydrogenase 2 |
| MTMR12 | NM_019061 | myotubularin related protein 12 |
| MTMR3 | NM_021090 | myotubularin-related protein 3 isoform c |
| MTMR7 | NM_004686 | myotubularin related protein 7 |
| MTMR9 | NM_015458 | myotubularin-related protein 9 |
| MUC17 | NM_001004430 | mucin 17 |
| MUCDHL | NM_017717 | mu-protocadherin isoform 2 |
| MULK | NM_018238 | multiple substrate lipid kinase |
| MUM1L1 | NM_152423 | melanoma associated antigen (mutated) 1-like 1 |
| MUTED | NM_201280 | Muted |
| MVK | NM_000431 | mevalonate kinase |
| MXD1 | NM_002357 | MAX dimerization protein 1 |
| MXI1 | NM_001008541 | MAX interactor 1 isoform c |
| MXRA7 | NM_001008529 | transmembrane anchor protein 1 isoform 2 |
| MYADM | NM_001020818 | myeloid-associated differentiation marker |
| MYCL1 | NM_001033081 | l-myc-1 proto-oncogene isoform 1 |
| MYCN | NM_005378 | v-myc myelocytomatosis viral related oncogene, |
| MYF5 | NM_005593 | myogenic factor 5 |
| MYF6 | NM_002469 | myogenic factor 6 (herculin) |
| MYLIP | NM_013262 | myosin regulatory light chain interacting |
| MYLK | NM_005965 | myosin light chain kinase isoform 6 |
| MYNN | NM_018657 | Myoneurin |
| MYO10 | NM_012334 | myosin X |
| MYO18A | NM_078471 | myosin 18A isoform a |
| MYO1C | NM_033375 | myosin IC |
| MYO1D | NM_015194 | myosin ID |
| MYO3B | NM_138995 | myosin IIIB |
| MYOHD1 | NM_001033579 | myosin head domain containing 1 isoform 2 |
| MYOM1 | NM_003803 | myomesin 1 |
| MYOZ2 | NM_016599 | myozenin 2 |
| MYOZ3 | NM_133371 | myozenin 3 |
| MYT1L | NM_015025 | myelin transcription factor 1-like |
| N4BP1 | NM_153029 | Nedd4 binding protein 1 |
| N4BP2 | NM_018177 | Nedd4 binding protein 2 |
| NAGK | NM_017567 | N-Acetylglucosamine kinase |
| NANOS1 | NM_001009553 | nanos homolog 1 isoform 2 |
| NAPB | NM_022080 | N-ethylmaleimide-sensitive factor attachment |
| NAPE-PLD | NM_198990 | N-acyl-phosphatidylethanolamine-hydrolyzing |
| NARG1L | NM_018527 | NMDA receptor regulated 1-like protein isoform |
| NARS | NM_004539 | asparaginyl-tRNA synthetase |
| NAT11 | NM_024771 | hypothetical protein LOC79829 |
| NAT12 | NM_001011713 | hypothetical protein LOC122830 |
| NAV2 | NM_145117 | neuron navigator 2 isoform 2 |
| NBEA | NM_015678 | Neurobeachin |
| NBEAL1 | NM_198945 | neurobeachin-like 1 |
| NBL1 | NM_005380 | neuroblastoma, suppression of tumorigenicity 1 |
| NBPF4 | NM_152488 | hypothetical protein LOC148545 |
| NBR1 | NM_005899 | neighbor of BRCA1 gene 1 |
| NBR2 | NM_005821 | hypothetical protein LOC10230 |
| NCF2 | NM_000433 | neutrophil cytosolic factor 2 |
| NCK2 | NM_001004720 | NCK adaptor protein 2 isoform A |
| NCKAP1L | NM_005337 | hematopoietic protein 1 |
| NCOA3 | NM_006534 | nuclear receptor coactivator 3 isoform b |
| NCOA7 | NM_181782 | nuclear receptor coactivator 7 |
| NCR1 | NM_004829 | natural cytotoxicity triggering receptor 1 |
| NDEL1 | NM_001025579 | nudE nuclear distribution gene E homolog like 1 |
| NDN | NM_002487 | Necdin |
| NDUFA6 | NM_002490 | NADH dehydrogenase (ubiquinone) 1 alpha |
| NDUFC2 | NM_004549 | NADH dehydrogenase (ubiquinone) 1, subcomplex |
| NDUFV3 | NM_001001503 | NADH-ubiquinone oxidoreductase flavoprotein 3 |
| NEB | NM_004543 | Nebulin |
| NEBL | NM_006393 | nebulette sarcomeric isoform |
| NEDD4L | NM_015277 | ubiquitin-protein ligase NEDD4-like |
| NEFH | NM_021076 | neurofilament, heavy polypeptide 200 kDa |
| NEK8 | NM_178170 | NIMA-related kinase 8 |
| NEK9 | NM_033116 | NIMA related kinase 9 |
| NENF | NM_013349 | SCIRP10-related protein |
| NEO1 | NM_002499 | neogenin homolog 1 |
| NETO2 | NM_018092 | neuropilin- and tolloid-like protein 2 |
| NEUROG1 | NM_006161 | neurogenin 1 |
| NEUROG2 | NM_024019 | neurogenin 2 |
| NF2 | NM_000268 | neurofibromin 2 isoform 1 |
| NFASC | NM_015090 | neurofascin precursor |
| NFAT5 | NM_006599 | nuclear factor of activated T-cells 5 isoform c |
| NFATC1 | NM_172387 | nuclear factor of activated T-cells, cytosolic |
| NFATC2IP | NM_032815 | nuclear factor of activated T-cells, |
| NFATC3 | NM_173164 | cytoplasmic nuclear factor of activated T-cells |
| NFATC4 | NM_004554 | cytoplasmic nuclear factor of activated T-cells |
| NFE2L2 | NM_006164 | nuclear factor (erythroid-derived 2)-like 2 |
| NFIA | NM_005595 | nuclear factor I/A |
| NFKBIB | NM_001001716 | nuclear factor of kappa light polypeptide gene |
| NFKBIL2 | NM_013432 | I-kappa-B-related protein |
| NFX1 | NM_147134 | nuclear transcription factor, X-box binding 1 |
| NFYB | NM_006166 | nuclear transcription factor Y, beta |
| NGEF | NM_019850 | neuronal guanine nucleotide exchange factor |
| NHLH1 | NM_005598 | nescient helix loop helix 1 |
| NHS | NM_198270 | Nance-Horan syndrome protein |
| NIN | NM_020921 | ninein isoform 2 |
| NINJ2 | NM_016533 | ninjurin 2 |
| NIP30 | NM_024946 | hypothetical protein LOC80011 |
| NIP7 | NM_016101 | 60S ribosome subunit biogenesis protein NIP7 |
| NIPA1 | NM_144599 | non-imprinted in Prader-Willi/Angelman syndrome |
| NKIRAS1 | NM_020345 | kappa B-ras 1 |
| NKIRAS2 | NM_001001349 | NFKB inhibitor interacting Ras-like 2 |
| NKX2-2 | NM_002509 | NK2 transcription factor related, locus 2 |
| NKX3-1 | NM_006167 | NK3 transcription factor related, locus 1 |
| NLK | NM_016231 | nemo like kinase |
| NMD3 | NM_015938 | NMD3 homolog |
| NME6 | NM_005793 | nucleoside diphosphate kinase type 6 |
| NMNAT1 | NM_022787 | nicotinamide nucleotide adenylyltransferase 1 |
| NMT1 | NM_021079 | N-myristoyltransferase 1 |
| NMT2 | NM_004808 | glycylpeptide N-tetradecanoyltransferase 2 |
| NMUR1 | NM_006056 | neuromedin U receptor 1 |
| NMUR2 | NM_020167 | neuromedin U receptor 2 |
| NOL9 | NM_024654 | hypothetical protein LOC79707 |
| NOM1 | NM_138400 | nucleolar protein with MIF4G domain 1 |
| NOS1AP | NM_014697 | nitric oxide synthase 1 (neuronal) adaptor |
| NOTCH2NL | NM_203458 | Notch homolog 2 N-terminal like protein |
| NPAL2 | NM_024759 | NIPA-like domain containing 2 |
| NPAL3 | NM_020448 | NIPA-like domain containing 3 |
| NPAS2 | NM_002518 | neuronal PAS domain protein 2 |
| NPAS3 | NM_022123 | neuronal PAS domain protein 3 isoform 1 |
| NPAT | NM_002519 | nuclear protein, ataxia-telangiectasia locus |
| NPC1 | NM_000271 | Niemann-Pick disease, type C1 |
| NPEPL1 | NM_024663 | aminopeptidase-like 1 |
| NPHP1 | NM_000272 | nephrocystin isoform 1 |
| NPHP3 | NM_153240 | nephronophthisis 3 |
| NPHS1 | NM_004646 | Nephrin |
| NPL | NM_030769 | N-acetylneuraminate pyruvate lyase |
| NPLOC4 | NM_017921 | nuclear protein localization 4 |
| NPNT | NM_001033047 | Nephronectin |
| NPTX1 | NM_002522 | neuronal pentraxin I precursor |
| NPTXR | NM_014293 | neuronal pentraxin receptor isoform 1 |
| NPY5R | NM_006174 | neuropeptide Y receptor Y5 |
| NR2E1 | NM_003269 | nuclear receptor subfamily 2, group E, member 1 |
| NR2E3 | NM_014249 | photoreceptor-specific nuclear receptor isoform |
| NR3C1 | NM_000176 | nuclear receptor subfamily 3, group C, member 1 |
| NR4A2 | NM_006186 | nuclear receptor subfamily 4, group A, member 2 |
| NR4A3 | NM_006981 | nuclear receptor subfamily 4, group A, member 3 |
| NRBF2 | NM_030759 | nuclear receptor binding factor 2 |
| NRBP1 | NM_013392 | nuclear receptor binding protein |
| NRIP2 | NM_031474 | nuclear receptor interacting protein 2 |
| NRP2 | NM_018534 | neuropilin 2 isoform 4 precursor |
| NSUN4 | NM_199044 | NOL1/NOP2/Sun domain family 4 protein |
| NT5C2 | NM_012229 | 5′-nucleotidase, cytosolic II |
| NT5DC3 | NM_016575 | hypothetical protein LOC51559 isoform 2 |
| NT5E | NM_002526 | 5′ nucleotidase, ecto |
| NTN4 | NM_021229 | netrin 4 |
| NTRK2 | NM_001007097 | neurotrophic tyrosine kinase, receptor, type 2 |
| NTSR1 | NM_002531 | neurotensin receptor 1 |
| NUAK1 | NM_014840 | AMPK-related protein kinase 5 |
| NUBP1 | NM_002484 | nucleotide binding protein 1 (MinD homolog, E. |
| NUBPL | NM_025152 | nucleotide binding protein-like |
| NUDT4 | NM_019094 | nudix-type motif 4 isoform alpha |
| NUFIP2 | NM_020772 | 82-kD FMRP Interacting Protein |
| NUP160 | NM_015231 | nucleoporin 160 kDa |
| NUP35 | NM_001008544 | nucleoporin 35 kDa isoform b |
| NUP43 | NM_198887 | nucleoporin 43 kDa |
| NUP62 | NM_012346 | nucleoporin 62 kDa |
| NUP98 | NM_016320 | nucleoporin 98 kD isoform 1 |
| NUPL1 | NM_001008564 | nucleoporin like 1 isoform b |
| NUSAP1 | NM_016359 | nucleolar and spindle associated protein 1 |
| NY-SAR-48 | NM_001011699 | sarcoma antigen NY-SAR-48 isoform b |
| OACT2 | NM_138799 | O-acyltransferase (membrane bound) domain |
| OACT5 | NM_005768 | gene rich cluster, C3f gene |
| OAS2 | NM_016817 | 2′-5′-oligoadenylate synthetase 2 isoform 1 |
| OATL1 | NM_001006113 | ornithine aminotransferase-like 1 isoform 1 |
| OBFC2A | NM_001031716 | hypothetical protein LOC64859 |
| OBFC2B | NM_024068 | hypothetical protein LOC79035 |
| OCLN | NM_002538 | Occludin |
| OCRL | NM_000276 | phosphatidylinositol polyphosphate 5-phosphatase |
| OGDH | NM_001003941 | oxoglutarate (alpha-ketoglutarate) dehydrogenase |
| OGG1 | NM_016827 | 8-oxoguanine DNA glycosylase isoform 2c |
| OGT | NM_003605 | O-linked GlcNAc transferase isoform 3 |
| OLIG1 | NM_138983 | oligodendrocyte transcription factor 1 |
| OPA3 | NM_001017989 | OPA3 protein isoform a |
| OPHN1 | NM_002547 | oligophrenin 1 |
| OPTN | NM_001008211 | Optineurin |
| OR7D2 | NM_175883 | hypothetical protein LOC162998 |
| ORC6L | NM_014321 | origin recognition complex subunit 6 |
| ORMDL3 | NM_139280 | ORM1-like 3 |
| OSBPL2 | NM_014835 | oxysterol-binding protein-like protein 2 isoform |
| OSBPL5 | NM_020896 | oxysterol-binding protein-like protein 5 isoform |
| OSCAR | NM_206817 | osteoclast-associated receptor isoform 2 |
| OSM | NM_020530 | oncostatin M precursor |
| OSR1 | NM_145260 | odd-skipped related 1 |
| OSTM1 | NM_014028 | osteopetrosis associated transmembrane protein |
| OTUD4 | NM_199324 | OTU domain containing 4 protein isoform 1 |
| OTUD6A | NM_207320 | HIN-6 protease |
| OTX1 | NM_014562 | orthodenticle 1 |
| OXR1 | NM_181354 | oxidation resistance 1 |
| P2RX4 | NM_002560 | purinergic receptor P2X4 isoform a |
| P2RX7 | NM_002562 | purinergic receptor P2X7 isoform a |
| P2RY13 | NM_023914 | purinergic receptor P2Y, G-protein coupled, 13 |
| P2RY14 | NM_014879 | purinergic receptor P2Y, G-protein coupled, 14 |
| P2RY6 | NM_004154 | pyrimidinergic receptor P2Y6 |
| P2RY8 | NM_178129 | G-protein coupled purinergic receptor P2Y8 |
| P4HA3 | NM_182904 | prolyl 4-hydroxylase, alpha III subunit |
| PABPC5 | NM_080832 | poly(A) binding protein, cytoplasmic 5 |
| PACSIN1 | NM_020804 | protein kinase C and casein kinase substrate in |
| PADI1 | NM_013358 | peptidylarginine deiminase type I |
| PAF1 | NM_019088 | Paf1, RNA polymerase II associated factor, |
| PAFAH1B1 | NM_000430 | platelet-activating factor acetylhydrolase, |
| PAFAH1B2 | NM_002572 | platelet-activating factor acetylhydrolase, |
| PAFAH2 | NM_000437 | platelet-activating factor acetylhydrolase 2 |
| PAG1 | NM_018440 | phosphoprotein associated with glycosphingolipid |
| PAICS | NM_006452 | phosphoribosylaminoimidazole carboxylase |
| PAK2 | NM_002577 | p21-activated kinase 2 |
| PALLD | NM_016081 | Palladin |
| PALM2-AKAP2 | NM_007203 | PALM2-AKAP2 protein isoform 1 |
| PAM | NM_000919 | peptidylglycine alpha-amidating monooxygenase |
| PANK1 | NM_138316 | pantothenate kinase 1 isoform gamma |
| PANK3 | NM_024594 | pantothenate kinase 3 |
| PANX1 | NM_015368 | pannexin 1 |
| PANX2 | NM_052839 | pannexin 2 |
| PAPD1 | NM_018109 | PAP associated domain containing 1 |
| PAPOLA | NM_032632 | poly(A) polymerase alpha |
| PAPOLB | NM_020144 | poly(A) polymerase beta (testis specific) |
| PAPOLG | NM_022894 | poly(A) polymerase gamma |
| PAPPA | NM_002581 | pregnancy-associated plasma protein A |
| PAQR5 | NM_017705 | membrane progestin receptor gamma |
| PARD6B | NM_032521 | PAR-6 beta |
| PARD6G | NM_032510 | PAR-6 gamma protein |
| PARN | NM_002582 | poly(A)-specific ribonuclease (deadenylation |
| PARP14 | NM_017554 | poly (ADP-ribose) polymerase family, member 14 |
| PARP6 | NM_020213 | poly (ADP-ribose) polymerase family, member 6 |
| PBK | NM_018492 | T-LAK cell-originated protein kinase |
| PBOV1 | NM_021635 | prostate and breast cancer overexpressed 1 |
| PBX3 | NM_006195 | pre-B-cell leukemia transcription factor 3 |
| PBXIP1 | NM_020524 | pre-B-cell leukemia transcription factor |
| PCAF | NM_003884 | p300/CBP-associated factor |
| PCDH11X | NM_032967 | protocadherin 11 X-linked isoform b precursor |
| PCDH11Y | NM_032971 | protocadherin 11 Y-linked isoform a |
| PCDH20 | NM_022843 | protocadherin 20 |
| PCDHA1 | NM_018900 | protocadherin alpha 1 isoform 1 precursor |
| PCDHA10 | NM_018901 | protocadherin alpha 10 isoform 1 precursor |
| PCDHA11 | NM_018902 | protocadherin alpha 11 isoform 1 precursor |
| PCDHA12 | NM_018903 | protocadherin alpha 12 isoform 1 precursor |
| PCDHA13 | NM_018904 | protocadherin alpha 13 isoform 1 precursor |
| PCDHA2 | NM_018905 | protocadherin alpha 2 isoform 1 precursor |
| PCDHA3 | NM_018906 | protocadherin alpha 3 isoform 1 precursor |
| PCDHA4 | NM_018907 | protocadherin alpha 4 isoform 1 precursor |
| PCDHA5 | NM_018908 | protocadherin alpha 5 isoform 1 precursor |
| PCDHA6 | NM_018909 | protocadherin alpha 6 isoform 1 precursor |
| PCDHA7 | NM_018910 | protocadherin alpha 7 isoform 1 precursor |
| PCDHA8 | NM_018911 | protocadherin alpha 8 isoform 1 precursor |
| PCDHA9 | NM_031857 | protocadherin alpha 9 isoform 1 precursor |
| PCDHAC1 | NM_018898 | protocadherin alpha subfamily C, 1 isoform 1 |
| PCDHAC2 | NM_018899 | protocadherin alpha subfamily C, 2 isoform 1 |
| PCDHB9 | NM_019119 | protocadherin beta 9 precursor |
| PCDHGA7 | NM_032087 | protocadherin gamma subfamily A, 7 isoform 2 |
| PCGF6 | NM_001011663 | polycomb group ring finger 6 isoform a |
| PCK1 | NM_002591 | cytosolic phosphoenolpyruvate carboxykinase 1 |
| PCMTD1 | NM_052937 | hypothetical protein LOC115294 |
| PCNP | NM_020357 | PEST-containing nuclear protein |
| PCNX | NM_014982 | pecanex homolog |
| PCNXL2 | NM_014801 | pecanex-like 2 |
| PCSK2 | NM_002594 | proprotein convertase subtilisin/kexin type 2 |
| PCSK6 | NM_138323 | paired basic amino acid cleaving system 4 |
| PCYOX1 | NM_016297 | prenylcysteine oxidase 1 |
| PCYT1B | NM_004845 | phosphate cytidylyltransferase 1, choline, beta |
| PDAP1 | NM_014891 | PDGFA associated protein 1 |
| PDCD1LG2 | NM_025239 | programmed cell death 1 ligand 2 |
| PDCD4 | NM_014456 | programmed cell death 4 isoform 1 |
| PDCD7 | NM_005707 | programmed cell death 7 |
| PDDC1 | NM_182612 | hypothetical protein LOC347862 |
| PDE1B | NM_000924 | phosphodiesterase 1B, calmodulin-dependent |
| PDE3B | NM_000922 | phosphodiesterase 3B, cGMP-inhibited |
| PDE4A | NM_006202 | phosphodiesterase 4A, cAMP-specific |
| PDE4B | NM_002600 | phosphodiesterase 4B, cAMP-specific isoform 1 |
| PDE4C | NM_000923 | phosphodiesterase 4C, cAMP-specific |
| PDE4DIP | NM_001002811 | phosphodiesterase 4D interacting protein isoform |
| PDE5A | NM_001083 | phosphodiesterase 5A isoform 1 |
| PDE7A | NM_002604 | phosphodiesterase 7A isoform b |
| PDE7B | NM_018945 | phosphodiesterase 7B |
| PDE8A | NM_002605 | phosphodiesterase 8A isoform 1 |
| PDGFB | NM_002608 | platelet-derived growth factor beta isoform 1, |
| PDGFC | NM_016205 | platelet-derived growth factor C precursor |
| PDGFD | NM_025208 | platelet derived growth factor D isoform 1 |
| PDGFRA | NM_006206 | platelet-derived growth factor receptor alpha |
| PDGFRB | NM_002609 | platelet-derived growth factor receptor beta |
| PDHX | NM_003477 | pyruvate dehydrogenase complex, component X |
| PDIK1L | NM_152835 | PDLIM1 interacting kinase 1 like |
| PDK1 | NM_002610 | pyruvate dehydrogenase kinase, isozyme 1 |
| PDK4 | NM_002612 | pyruvate dehydrogenase kinase, isoenzyme 4 |
| PDLIM4 | NM_003687 | PDZ and LIM domain 4 |
| PDLIM5 | NM_001011513 | PDZ and LIM domain 5 isoform b |
| PDPK1 | NM_002613 | 3-phosphoinositide dependent protein kinase-1 |
| PDPN | NM_001006624 | lung type-I cell membrane-associated |
| PDPR | NM_017990 | pyruvate dehydrogenase phosphatase regulatory |
| PDRG1 | NM_030815 | p53 and DNA damage-regulated protein |
| PDZD11 | NM_016484 | PDZ domain containing 11 |
| PECR | NM_018441 | peroxisomal trans-2-enoyl-CoA reductase |
| PEG3 | NM_006210 | paternally expressed 3 |
| PELI2 | NM_021255 | pellino 2 |
| PELO | NM_015946 | pelota homolog |
| PER2 | NM_022817 | period 2 isoform 1 |
| PERP | NM_022121 | PERP, TPS3 apoptosis effector |
| PERQ1 | NM_022574 | PERQ amino acid rich, with GYF domain 1 |
| PEX16 | NM_057174 | peroxisomal biogenesis factor 16 isoform 2 |
| PEX19 | NM_002857 | peroxisomal biogenesis factor 19 |
| PEX26 | NM_017929 | peroxisome biogenesis factor 26 |
| PEX5L | NM_016559 | PXR2b protein |
| PF4V1 | NM_002620 | platelet factor 4 variant 1 |
| PFKFB2 | NM_006212 | 6-phosphofructo-2-kinase/fructose-2, |
| PFKFB3 | NM_004566 | 6-phosphofructo-2-kinase/fructose-2, |
| PFKP | NM_002627 | phosphofructokinase, platelet |
| PFN2 | NM_002628 | profilin 2 isoform b |
| PGA5 | NM_014224 | pepsinogen 5, group I (pepsinogen A) |
| PGAP1 | NM_024989 | GPI deacylase |
| PGBD4 | NM_152595 | piggyBac transposable element derived 4 |
| PGBD5 | NM_024554 | piggyBac transposable element derived 5 |
| PGDS | NM_014485 | prostaglandin-D synthase |
| PGF | NM_002632 | placental growth factor, vascular endothelial |
| PGM2L1 | NM_173582 | phosphoglucomutase 2-like 1 |
| PGM5 | NM_021965 | phosphoglucomutase 5 |
| PHACTR4 | NM_023923 | phosphatase and actin regulator 4 |
| PHF1 | NM_002636 | PHD finger protein 1 isoform a |
| PHF11 | NM_016119 | PHD finger protein 11 |
| PHF15 | NM_015288 | PHD finger protein 15 |
| PHF17 | NM_024900 | Jade1 protein short isoform |
| PHF2 | NM_005392 | PHD finger protein 2 isoform a |
| PHF20 | NM_016436 | PHD finger protein 20 |
| PHF23 | NM_024297 | PHD finger protein 23 |
| PHF6 | NM_001015877 | PHD finger protein 6 isoform 1 |
| PHF8 | NM_015107 | PHD finger protein 8 |
| PHKG1 | NM_006213 | phosphorylase kinase, gamma 1 (muscle) |
| PHLDB3 | NM_198850 | pleckstrin homology-like domain, family B, |
| PHLPPL | NM_015020 | PH domain and leucine rich repeat protein |
| PHTF2 | NM_020432 | putative homeodomain transcription factor 2 |
| PHYHIP | NM_014759 | phytanoyl-CoA hydroxylase interacting protein |
| PI15 | NM_015886 | protease inhibitor 15 preproprotein |
| PIGK | NM_005482 | phosphatidylinositol glycan, class K precursor |
| PIGM | NM_145167 | PIG-M mannosyltransferase |
| PIGO | NM_032634 | phosphatidylinositol glycan, class O isoform 1 |
| PIGX | NM_017861 | GPI-mannosyltransferase subunit |
| PIK3CD | NM_005026 | phosphoinositide-3-kinase, catalytic, delta |
| PIK3R1 | NM_181504 | phosphoinositide-3-kinase, regulatory subunit, |
| PIK3R2 | NM_005027 | phosphoinositide-3-kinase, regulatory subunit 2 |
| PIP3-E | NM_015553 | phosphoinositide-binding protein PIP3-E |
| PIP5K1B | NM_001031687 | phosphatidylinositol-4-phosphate 5-kinase, type |
| PIP5K2C | NM_024779 | phosphatidylinositol-4-phosphate 5-kinase, type |
| PIP5K3 | NM_001002881 | phosphatidylinositol-3- |
| PIPOX | NM_016518 | L-pipecolic acid oxidase |
| PITPNA | NM_006224 | phosphatidylinositol transfer protein, alpha |
| PITX1 | NM_002653 | paired-like homeodomain transcription factor 1 |
| PIWIL2 | NM_018068 | piwi-like 2 |
| PKD1 | NM_000296 | polycystin 1 isoform 2 precursor |
| PKD2 | NM_000297 | polycystin 2 |
| PKDREJ | NM_006071 | receptor for egg jelly-like protein precursor |
| PKHD1 | NM_138694 | polyductin isoform 1 |
| PKIA | NM_006823 | cAMP-dependent protein kinase inhibitor alpha |
| PKIG | NM_007066 | cAMP-dependent protein kinase inhibitor gamma |
| PKMYT1 | NM_004203 | protein kinase Myt1 isoform 1 |
| PKNOX1 | NM_004571 | PBX/knotted 1 homeobox 1 isoform 1 |
| PKP1 | NM_000299 | plakophilin 1 isoform 1b |
| PLA2G6 | NM_001004426 | phospholipase A2, group VI isoform b |
| PLAC1 | NM_021796 | placenta-specific 1 |
| PLAC2 | NM_153375 | placenta-specific 2 |
| PLAC4 | NM_182832 | placenta-specific 4 |
| PLAG1 | NM_002655 | pleiomorphic adenoma gene 1 |
| PLAGL2 | NM_002657 | pleiomorphic adenoma gene-like 2 |
| PLAU | NM_002658 | urokinase plasminogen activator preproprotein |
| PLAUR | NM_001005376 | plasminogen activator, urokinase receptor |
| PLB1 | NM_153021 | phospholipase B1 |
| PLCB1 | NM_015192 | phosphoinositide-specific phospholipase C beta 1 |
| PLCH1 | NM_014996 | phospholipase C-like 3 |
| PLCXD3 | NM_001005473 | phosphatidylinositol-specific phospholipase C, X |
| PLD1 | NM_002662 | phospholipase D1, phophatidylcholine-specific |
| PLDN | NM_012388 | Pallidin |
| PLEKHA1 | NM_001001974 | pleckstrin homology domain containing, family A |
| PLEKHA3 | NM_019091 | pleckstrin homology domain containing, family A |
| PLEKHA6 | NM_014935 | phosphoinositol 3-phosphate-binding protein-3 |
| PLEKHB2 | NM_017958 | pleckstrin homology domain containing, family B |
| PLEKHF2 | NM_024613 | phafin 2 |
| PLEKHG1 | NM_001029884 | pleckstrin homology domain containing, family G |
| PLEKHG6 | NM_018173 | pleckstrin homology domain containing, family G |
| PLEKHM1 | NM_014798 | pleckstrin homology domain containing, family M |
| PLEKHQ1 | NM_025201 | PH domain-containing protein |
| PLIN | NM_002666 | Perilipin |
| PLS1 | NM_002670 | plastin 1 |
| PLSCR3 | NM_020360 | phospholipid scramblase 3 |
| PLSCR4 | NM_020353 | phospholipid scramblase 4 |
| PLXDC1 | NM_020405 | plexin domain containing 1 precursor |
| PLXNA1 | NM_032242 | plexin A1 |
| PLXNA4B | NM_181775 | hypothetical protein LOC91584 |
| PLXNC1 | NM_005761 | plexin C1 |
| PMAIP1 | NM_021127 | phorbol-12-myristate-13-acetate-induced protein |
| PNKD | NM_015488 | myofibrillogenesis regulator 1 isoform 1 |
| PNPLA1 | NM_173676 | patatin-like phospholipase domain containing 1 |
| PNPLA4 | NM_004650 | GS2 gene |
| PODN | NM_153703 | Podocan |
| POFUT1 | NM_015352 | protein O-fucosyltransferase 1 isoform 1 |
| POLDIP2 | NM_015584 | DNA polymerase delta interacting protein 2 |
| POLH | NM_006502 | polymerase (DNA directed), eta |
| POLQ | NM_199420 | DNA polymerase theta |
| POLR1E | NM_022490 | RNA polymerase I associated factor 53 |
| POLR3A | NM_007055 | polymerase (RNA) III (DNA directed) polypeptide |
| POLR3K | NM_016310 | DNA directed RNA polymerase III polypeptide K |
| PON2 | NM_000305 | paraoxonase 2 isoform 1 |
| POU3F2 | NM_005604 | POU domain, class 3, transcription factor 2 |
| POU4F2 | NM_004575 | POU domain, class 4, transcription factor 2 |
| POU6F1 | NM_002702 | POU domain, class 6, transcription factor 1 |
| PPAPDC3 | NM_032728 | phosphatidic acid phosphatase type 2 domain |
| PPARA | NM_001001928 | peroxisome proliferative activated receptor, |
| PPARD | NM_006238 | peroxisome proliferative activated receptor, |
| PPGB | NM_000308 | protective protein for beta-galactosidase |
| PPM1A | NM_177951 | protein phosphatase 1A isoform 2 |
| PPM1B | NM_001033556 | protein phosphatase 1B isoform 4 |
| PPM1E | NM_014906 | protein phosphatase 1E |
| PPM1K | NM_152542 | protein phosphatase 1K (PP2C domain containing) |
| PPP1R12B | NM_002481 | protein phosphatase 1, regulatory (inhibitor) |
| PPP1R13B | NM_015316 | protein phosphatase 1, regulatory (inhibitor) |
| PPP1R14C | NM_030949 | protein phosphatase 1, regulatory (inhibitor) |
| PPP1R15B | NM_032833 | protein phosphatase 1, regulatory subunit 15B |
| PPP1R1A | NM_006741 | protein phosphatase 1, regulatory (inhibitor) |
| PPP1R3B | NM_024607 | protein phosphatase 1, regulatory (inhibitor) |
| PPP1R3C | NM_005398 | protein phosphatase 1, regulatory (inhibitor) |
| PPP2CA | NM_002715 | protein phosphatase 2, catalytic subunit, alpha |
| PPP2R1B | NM_002716 | beta isoform of regulatory subunit A, protein |
| PPP2R2A | NM_002717 | alpha isoform of regulatory subunit B55, protein |
| PPP2R3A | NM_002718 | protein phosphatase 2, regulatory subunit B″, |
| PPP3CA | NM_000944 | protein phosphatase 3 (formerly 2B), catalytic |
| PPP3R1 | NM_000945 | protein phosphatase 3, regulatory subunit B, |
| PPP6C | NM_002721 | protein phosphatase 6, catalytic subunit |
| PPTC7 | NM_139283 | T-cell activation protein phosphatase 2C |
| PQLC2 | NM_017765 | PQ loop repeat containing 2 |
| PRAP1 | NM_145202 | proline-rich acidic protein 1 |
| PRDM10 | NM_020228 | PR domain containing 10 isoform 1 |
| PRDM12 | NM_021619 | PR domain containing 12 |
| PRDM14 | NM_024504 | PR domain containing 14 |
| PRDM2 | NM_001007257 | retinoblastoma protein-binding zinc finger |
| PRELP | NM_002725 | proline arginine-rich end leucine-rich repeat |
| PREPL | NM_006036 | prolyl endopeptidase-like |
| PREX1 | NM_020820 | PREX1 protein |
| PRH2 | NM_005042 | proline-rich protein HaeIII subfamily 2 |
| PRIC285 | NM_033405 | PPAR-alpha interacting complex protein 285 |
| PRICKLE2 | NM_198859 | prickle-like 2 |
| PRKAB2 | NM_005399 | AMP-activated protein kinase beta 2 |
| PRKACB | NM_002731 | cAMP-dependent protein kinase catalytic subunit |
| PRKAR1A | NM_002734 | cAMP-dependent protein kinase, regulatory |
| PRKCH | NM_006255 | protein kinase C, eta |
| PRKD3 | NM_005813 | protein kinase D3 |
| PRKX | NM_005044 | protein kinase, X-linked |
| PRKY | NM_002760 | protein kinase, Y-linked |
| PRND | NM_012409 | prion-like protein doppel preproprotein |
| PRNP | NM_000311 | prion protein preproprotein |
| PRO0149 | NM_014117 | hypothetical protein LOC29035 |
| PRO1853 | NM_018607 | hypothetical protein LOC55471 isoform 2 |
| PROK1 | NM_032414 | prokineticin 1 |
| PROSC | NM_007198 | proline synthetase co-transcribed homolog |
| PRPF4 | NM_004697 | PRP4 pre-mRNA processing factor 4 homolog |
| PRPF40B | NM_001031698 | Huntingtin interacting protein C isoform 1 |
| PRPF4B | NM_003913 | serine/threonine-protein kinase PRP4K |
| PRR11 | NM_018304 | hypothetical protein LOC55771 |
| PRR5 | NM_001017528 | proline rich 5 (renal) isoform 2 |
| PRRG1 | NM_000950 | proline rich Gla (G-carboxyglutamic acid) 1 |
| PRRG4 | NM_024081 | proline rich Gla (G-carboxyglutamic acid) 4 |
| PRRT3 | NM_207351 | hypothetical protein LOC285368 |
| PRRX1 | NM_006902 | paired mesoderm homeobox 1 isoform pmx-1a |
| PRSS35 | NM_153362 | protease, serine, 35 |
| PSCD1 | NM_004762 | pleckstrin homology, Sec7 and coiled/coil |
| PSD | NM_002779 | pleckstrin and Sec7 domain containing |
| PSD3 | NM_015310 | ADP-ribosylation factor guanine nucleotide |
| PSG3 | NM_021016 | pregnancy specific beta-1-glycoprotein 3 |
| PSMC3IP | NM_016556 | TBP-1 interacting protein isoform 2 |
| PSMD12 | NM_002816 | proteasome 26S non-ATPase subunit 12 isoform 1 |
| PSMD5 | NM_005047 | proteasome 26S non-ATPase subunit 5 |
| PSTPIP2 | NM_024430 | proline-serine-threonine phosphatase interacting |
| PTAFR | NM_000952 | platelet-activating factor receptor |
| PTDSS1 | NM_014754 | phosphatidylserine synthase 1 |
| PTGDR | NM_000953 | prostaglandin D2 receptor |
| PTGER3 | NM_198718 | prostaglandin E receptor 3, subtype EP3 isoform |
| PTGER4 | NM_000958 | prostaglandin E receptor 4, subtype EP4 |
| PTGFRN | NM_020440 | prostaglandin F2 receptor negative regulator |
| PTGIS | NM_000961 | prostaglandin I2 (prostacyclin) synthase |
| PTGS1 | NM_000962 | prostaglandin-endoperoxide synthase 1 isoform 1 |
| PTHLH | NM_198965 | parathyroid hormone-like hormone isoform 1 |
| PTK2 | NM_005607 | PTK2 protein tyrosine kinase 2 isoform b |
| PTK6 | NM_005975 | PTK6 protein tyrosine kinase 6 |
| PTK9 | NM_002822 | twinfilin isoform 1 |
| PTP4A1 | NM_003463 | protein tyrosine phosphatase type IVA, member 1 |
| PTPDC1 | NM_152422 | protein tyrosine phosphatase domain containing 1 |
| PTPN1 | NM_002827 | protein tyrosine phosphatase, non-receptor type |
| PTPN12 | NM_002835 | protein tyrosine phosphatase, non-receptor type |
| PTPN3 | NM_002829 | protein tyrosine phosphatase, non-receptor type |
| PTPN4 | NM_002830 | protein tyrosine phosphatase, non-receptor type |
| PTPNS1 | NM_080792 | protein tyrosine phosphatase, non-receptor type |
| PTPRO | NM_002848 | receptor-type protein tyrosine phosphatase O |
| PTPRT | NM_007050 | protein tyrosine phosphatase, receptor type, T |
| PTRF | NM_012232 | polymerase I and transcript release factor |
| PURA | NM_005859 | purine-rich element binding protein A |
| PURB | NM_033224 | purine-rich element binding protein B |
| PVR | NM_006505 | poliovirus receptor |
| PVRL2 | NM_002856 | poliovirus receptor-related 2 (herpesvirus entry |
| PYGB | NM_002862 | brain glycogen phosphorylase |
| QKI | NM_206853 | quaking homolog, KH domain RNA binding isoform |
| QPRT | NM_014298 | quinolinate phosphoribosyltransferase |
| QRSL1 | NM_018292 | glutaminyl-tRNA synthase |
| R7BP | NM_001029875 | R7 binding protein |
| RAB10 | NM_016131 | ras-related GTP-binding protein RAB10 |
| RAB11A | NM_004663 | Ras-related protein Rab-11A |
| RAB11FIP1 | NM_001002233 | Rab coupling protein isoform 2 |
| RAB11FIP4 | NM_032932 | RAB11 family interacting protein 4 (class II) |
| RAB11FIP5 | NM_015470 | RAB11 family interacting protein 5 (class I) |
| RAB21 | NM_014999 | RAB21, member RAS oncogene family |
| RAB22A | NM_020673 | RAS-related protein RAB-22A |
| RAB23 | NM_016277 | Ras-related protein Rab-23 |
| RAB27A | NM_004580 | Ras-related protein Rab-27A |
| RAB28 | NM_001017979 | RAB28, member RAS oncogene family isoform 1 |
| RAB30 | NM_014488 | RAB30, member RAS oncogene family |
| RAB31 | NM_006868 | RAB31, member RAS oncogene family |
| RAB35 | NM_006861 | RAB35, member RAS oncogene family |
| RAB37 | NM_001006638 | RAB37, member RAS oncogene family isoform 2 |
| RAB3B | NM_002867 | RAB3B, member RAS oncogene family |
| RAB3IL1 | NM_013401 | RAB3A interacting protein (rabin3)-like 1 |
| RAB43 | NM_198490 | RAB43 protein |
| RAB5B | NM_002868 | RAB5B, member RAS oncogene family |
| RAB7L1 | NM_003929 | RAB7, member RAS oncogene family-like 1 |
| RAB8B | NM_016530 | RAB8B, member RAS oncogene family |
| RABEP1 | NM_004703 | rabaptin, RAB GTPase binding effector protein 1 |
| RABGAP1 | NM_012197 | RAB GTPase activating protein 1 |
| RABL2A | NM_013412 | RAB, member of RAS oncogene family-like 2A |
| RABL2B | NM_001003789 | RAB, member of RAS oncogene family-like 2B |
| RABL5 | NM_022777 | RAB, member RAS oncogene family-like 5 |
| RACGAP1 | NM_013277 | Rac GTPase activating protein 1 |
| RAD1 | NM_002853 | RAD1 homolog isoform 1 |
| RAD17 | NM_002873 | RAD17 homolog isoform 1 |
| RAD18 | NM_020165 | postreplication repair protein hRAD18p |
| RAD23B | NM_002874 | UV excision repair protein RAD23 homolog B |
| RAD51 | NM_002875 | RAD51 homolog protein isoform 1 |
| RAD52 | NM_002879 | RAD52 homolog isoform alpha |
| RAG1AP1 | NM_018845 | stromal cell protein |
| RAI16 | NM_022749 | retinoic acid induced 16 |
| RAI17 | NM_020338 | retinoic acid induced 17 |
| RALBP1 | NM_006788 | ralA binding protein 1 |
| RALGPS1 | NM_014636 | Ral GEF with PH domain and SH3 binding motif 1 |
| RALGPS2 | NM_018037 | Ral GEF with PH domain and SH3 binding motif 2 |
| RAN | NM_006325 | ras-related nuclear protein |
| RANBP5 | NM_002271 | RAN binding protein 5 |
| RAP1A | NM_001010935 | RAP1A, member of RAS oncogene family |
| RAP2B | NM_002886 | RAP2B, member of RAS oncogene family |
| RAP2C | NM_021183 | RAP2C, member of RAS oncogene family |
| RAPGEF1 | NM_005312 | guanine nucleotide-releasing factor 2 isoform a |
| RAPGEF4 | NM_007023 | Rap guanine nucleotide exchange factor (GEF) 4 |
| RAPGEFL1 | NM_016339 | Rap guanine nucleotide exchange factor |
| RAPH1 | NM_213589 | Ras association and pleckstrin homology domains |
| Raptor | NM_020761 | Raptor |
| RARB | NM_000965 | retinoic acid receptor, beta isoform 1 |
| RASD1 | NM_016084 | RAS, dexamethasone-induced 1 |
| RASGEF1A | NM_145313 | RasGEF domain family, member 1A |
| RASL11B | NM_023940 | RAS-like family 11 member B |
| RASL12 | NM_016563 | RAS-like, family 12 protein |
| RASSF2 | NM_014737 | Ras association domain family 2 |
| RASSF6 | NM_177532 | Ras association (RalGDS/AF-6) domain family 6 |
| RASSF8 | NM_007211 | Ras association (RalGDS/AF-6) domain family 8 |
| RAVER2 | NM_018211 | ribonucleoprotein, PTB-binding 2 |
| RB1 | NM_000321 | retinoblastoma 1 |
| RB1CC1 | NM_014781 | Rb1-inducible coiled coil protein 1 |
| RBBP5 | NM_005057 | retinoblastoma binding protein 5 |
| RBBP7 | NM_002893 | retinoblastoma binding protein 7 |
| RBJ | NM_016544 | Ras-associated protein Rap1 |
| RBL1 | NM_002895 | retinoblastoma-like protein 1 isoform a |
| RBL2 | NM_005611 | retinoblastoma-like 2 (p130) |
| RBM12 | NM_006047 | RNA binding motif protein 12 |
| RBM12B | NM_203390 | hypothetical protein LOC389677 |
| RBM13 | NM_032509 | RNA binding motif protein 13 |
| RBM15B | NM_013286 | RNA binding motif protein 15B |
| RBM7 | NM_016090 | RNA binding motif protein 7 |
| RBPMS | NM_006867 | RNA-binding protein with multiple splicing |
| RCCD1 | NM_001017919 | hypothetical protein LOC91433 |
| RCOR2 | NM_173587 | REST corepressor 2 |
| RECQL5 | NM_001003715 | RecQ protein-like 5 isoform 2 |
| REEP1 | NM_022912 | receptor expression enhancing protein 1 |
| REEP3 | NM_001001330 | receptor expression enhancing protein 3 |
| REEP5 | NM_005669 | receptor accessory protein 5 |
| REL | NM_002908 | v-rel reticuloendotheliosis viral oncogene |
| RERE | NM_012102 | atrophin-1 like protein |
| RET | NM_020975 | ret proto-oncogene isoform a |
| REXO1L1 | NM_172239 | exonuclease GOR |
| RFC2 | NM_002914 | replication factor C 2 (40 kD) isoform 2 |
| RFK | NM_018339 | riboflavin kinase |
| RFX2 | NM_000635 | regulatory factor X2 isoform a |
| RFX5 | NM_000449 | regulatory factor X, 5 |
| RFXAP | NM_000538 | regulatory factor X-associated protein |
| RG9MTD3 | NM_144964 | RNA (guanine-9-) methyltransferase domain |
| RGL1 | NM_015149 | ral guanine nucleotide dissociation |
| RGMA | NM_020211 | RGM domain family, member A |
| RGMB | NM_001012761 | RGM domain family, member B isoform 1 precursor |
| RGPD5 | NM_005054 | RANBP2-like and GRIP domain containing 5 isoform |
| RGS3 | NM_021106 | regulator of G-protein signalling 3 isoform 2 |
| RGS4 | NM_005613 | regulator of G-protein signaling 4 |
| RGS5 | NM_003617 | regulator of G-protein signalling 5 |
| RGS9BP | NM_207391 | RGS9 anchor protein |
| RHBDL2 | NM_017821 | rhomboid-related protein 2 |
| RHO | NM_000539 | Rhodopsin |
| RHOA | NM_001664 | ras homolog gene family, member A |
| RHOBTB1 | NM_001032380 | Rho-related BTB domain containing 1 |
| RHOBTB3 | NM_014899 | rho-related BTB domain containing 3 |
| RHOC | NM_175744 | ras homolog gene family, member C |
| RHOT1 | NM_001033566 | ras homolog gene family, member T1 isoform 2 |
| RHOV | NM_133639 | ras homolog gene family, member V |
| RIC3 | NM_024557 | resistance to inhibitors of cholinesterase 3 |
| RIMBP2 | NM_015347 | RIM-binding protein 2 |
| RIMS4 | NM_182970 | regulating synaptic membrane exocytosis 4 |
| RIOK3 | NM_003831 | sudD suppressor of bimD6 homolog isoform 1 |
| RIPK5 | NM_015375 | receptor interacting protein kinase 5 isoform 1 |
| RKHD1 | NM_203304 | ring finger and KH domain containing 1 |
| RKHD2 | NM_016626 | ring finger and KH domain containing 2 |
| RNASEL | NM_021133 | ribonuclease L |
| RND3 | NM_005168 | ras homolog gene family, member E |
| RNF11 | NM_014372 | ring finger protein 11 |
| RNF12 | NM_016120 | ring finger protein 12 |
| RNF125 | NM_017831 | ring finger protein 125 |
| RNF128 | NM_024539 | ring finger protein 128 isoform 2 |
| RNF141 | NM_016422 | ring finger protein 141 |
| RNF144 | NM_014746 | ring finger protein 144 |
| RNF149 | NM_173647 | ring finger protein 149 |
| RNF157 | NM_052916 | ring finger protein 157 |
| RNF170 | NM_030954 | ring finger protein 170 |
| RNF180 | NM_178532 | ring finger protein 180 |
| RNF19 | NM_015435 | ring finger protein 19 |
| RNF2 | NM_007212 | ring finger protein 2 |
| RNF24 | NM_007219 | ring finger protein 24 |
| RNF31 | NM_017999 | ring finger protein 31 |
| RNF38 | NM_022781 | ring finger protein 38 isoform 1 |
| RNF4 | NM_002938 | ring finger protein 4 |
| RNF6 | NM_005977 | ring finger protein 6 isoform 1 |
| RNF8 | NM_003958 | ring finger protein 8 isoform 1 |
| RNH1 | NM_002939 | ribonuclease/angiogenin inhibitor |
| RNMT | NM_003799 | RNA (guanine-7-) methyltransferase |
| RNMTL1 | NM_018146 | RNA methyltransferase like 1 |
| RNPC1 | NM_183425 | RNA-binding region containing protein 1 isoform |
| RNPS1 | NM_006711 | RNA-binding protein S1, serine-rich domain |
| RNUXA | NM_032177 | RNA U, small nuclear RNA export adaptor |
| ROCK2 | NM_004850 | Rho-associated, coiled-coil containing protein |
| RORA | NM_002943 | RAR-related orphan receptor A isoform c |
| RORC | NM_001001523 | RAR-related orphan receptor C isoform b |
| RPA2 | NM_002946 | replication protein A2, 32 kDa |
| RPIP8 | NM_006695 | RaP2 interacting protein 8 |
| RPL13 | NM_000977 | ribosomal protein L13 |
| RPL15 | NM_002948 | ribosomal protein L15 |
| RPL32 | NM_000994 | ribosomal protein L32 |
| RPL37 | NM_000997 | ribosomal protein L37 |
| RPL37A | NM_000998 | ribosomal protein L37a |
| RPL7L1 | NM_198486 | ribosomal protein L7-like 1 |
| RPN1 | NM_002950 | ribophorin I precursor |
| RPP14 | NM_007042 | ribonuclease P 14 kDa subunit |
| RPRM | NM_019845 | reprimo, TP53 dependant G2 arrest mediator |
| RPS23 | NM_001025 | ribosomal protein S23 |
| RPS6KA1 | NM_001006665 | ribosomal protein S6 kinase, 90 kDa, polypeptide |
| RPS6KA2 | NM_001006932 | ribosomal protein S6 kinase, 90 kDa, polypeptide |
| RPS6KA3 | NM_004586 | ribosomal protein S6 kinase, 90 kDa, polypeptide |
| RPS6KA4 | NM_001006944 | ribosomal protein S6 kinase, 90 kDa, polypeptide |
| RPS6KA5 | NM_004755 | ribosomal protein S6 kinase, 90 kDa, polypeptide |
| RRH | NM_006583 | Peropsin |
| RRM2 | NM_001034 | ribonucleotide reductase M2 polypeptide |
| RRN3 | NM_018427 | RRN3 RNA polymerase I transcription factor |
| RSAD2 | NM_080657 | radical S-adenosyl methionine domain containing |
| RSBN1 | NM_018364 | round spermatid basic protein 1 |
| RSL1D1 | NM_015659 | ribosomal L1 domain containing 1 |
| RSNL2 | NM_024692 | restin-like 2 |
| RSPO2 | NM_178565 | R-spondin family, member 2 |
| RSPO4 | NM_001029871 | R-spondin family, member 4 isoform 1 precursor |
| RSU1 | NM_012425 | ras suppressor protein 1 isoform 1 |
| RTF1 | NM_015138 | Paf1/RNA polymerase II complex component |
| RTN2 | NM_206902 | reticulon 2 isoform D |
| RTP1 | NM_153708 | receptor transporting protein 1 |
| RTP4 | NM_022147 | 28 kD interferon responsive protein |
| RUNDC1 | NM_173079 | RUN domain containing 1 |
| RUNDC2A | NM_032167 | RUN domain containing 2A |
| RUNX1 | NM_001001890 | runt-related transcription factor 1 isoform b |
| RUNX2 | NM_001015051 | runt-related transcription factor 2 isoform b |
| RUNX3 | NM_001031680 | runt-related transcription factor 3 isoform 1 |
| RXRG | NM_006917 | retinoid X receptor, gamma isoform a |
| S100A16 | NM_080388 | S100 calcium binding protein A16 |
| S100A4 | NM_002961 | S100 calcium-binding protein A4 |
| S100PBP | NM_001017406 | S100P binding protein Riken isoform b |
| SACS | NM_014363 | Sacsin |
| SAMD10 | NM_080621 | sterile alpha motif domain containing 10 |
| SAMD12 | NM_207506 | sterile alpha motif domain containing 12 |
| SAMD8 | NM_144660 | sterile alpha motif domain containing 8 |
| SAMD9L | NM_152703 | sterile alpha motif domain containing 9-like |
| SAPS1 | NM_014931 | hypothetical protein LOC22870 |
| SAPS2 | NM_014678 | hypothetical protein LOC9701 |
| SAPS3 | NM_018312 | SAPS domain family, member 3 |
| SAR1B | NM_001033503 | SAR1a gene homolog 2 |
| SART1 | NM_005146 | squamous cell carcinoma antigen recognized by T |
| SASH1 | NM_015278 | SAM and SH3 domain containing 1 |
| SATB2 | NM_015265 | SATB family member 2 |
| SATL1 | NM_001012980 | spermidine/spermine N1-acetyl transferase-like |
| SBK1 | NM_001024401 | SH3-binding domain kinase 1 |
| SC4MOL | NM_001017369 | sterol-C4-methyl oxidase-like isoform 2 |
| SCAMP1 | NM_052822 | secretory carrier membrane protein 1 isoform 2 |
| SCAMP2 | NM_005697 | secretory carrier membrane protein 2 |
| SCAMP5 | NM_138967 | secretory carrier membrane protein 5 |
| SCAND2 | NM_022050 | SCAN domain-containing protein 2 isoform 1 |
| SCARA5 | NM_173833 | hypothetical protein LOC286133 |
| SCC-112 | NM_015200 | SCC-112 protein |
| SCCPDH | NM_016002 | saccharopine dehydrogenase (putative) |
| SCIN | NM_033128 | Scinderin |
| SCMH1 | NM_001031694 | sex comb on midleg homolog 1 isoform 1 |
| SCML2 | NM_006089 | sex comb on midleg-like 2 |
| SCN11A | NM_014139 | sodium channel, voltage-gated, type XI, alpha |
| SCN2B | NM_004588 | sodium channel, voltage-gated, type II, beta |
| SCN3A | NM_006922 | sodium channel, voltage-gated, type III, alpha |
| SCRN3 | NM_024583 | secernin 3 |
| SCRT2 | NM_033129 | scratch 2 protein |
| SDC2 | NM_002998 | syndecan 2 precursor |
| SDPR | NM_004657 | serum deprivation response protein |
| SEC14L2 | NM_012429 | SEC14-like 2 |
| SEC31L2 | NM_198138 | S. cerevisiae SEC31-like 2 isoform b |
| SEL1L | NM_005065 | sel-1 suppressor of lin-12-like |
| SELE | NM_000450 | selectin E precursor |
| SELI | NM_033505 | selenoprotein I |
| SELPLG | NM_003006 | selectin P ligand |
| SEMA3E | NM_012431 | semaphorin 3E |
| SEMA4B | NM_020210 | semaphorin 4B precursor |
| SEMA4G | NM_017893 | semaphorin 4G |
| SEMA5A | NM_003966 | semaphorin 5A |
| SENP1 | NM_014554 | sentrin/SUMO-specific protease 1 |
| SENP8 | NM_145204 | SUMO/sentrin specific protease family member 8 |
| SEPT11 | NM_018243 | septin 11 |
| SEPT2 | NM_001008491 | septin 2 |
| SEPT6 | NM_015129 | septin 6 isoform B |
| SERF1A | NM_021967 | small EDRK-rich factor 1A, telomeric |
| SERF1B | NM_022978 | small EDRK-rich factor 1B, centromeric |
| SERINC1 | NM_020755 | tumor differentially expressed 2 |
| SERP1 | NM_014445 | stress-associated endoplasmic reticulum protein |
| SERPINB8 | NM_002640 | serine (or cysteine) proteinase inhibitor, clade |
| SERPINE1 | NM_000602 | plasminogen activator inhibitor-1 |
| SERTAD2 | NM_014755 | SERTA domain containing 2 |
| SESN1 | NM_014454 | sestrin 1 |
| SESN2 | NM_031459 | sestrin 2 |
| SET | NM_003011 | SET translocation (myeloid leukemia-associated) |
| SETD2 | NM_014159 | huntingtin interacting protein B |
| SETD4 | NM_001007258 | hypothetical protein LOC54093 isoform b |
| SEZ6 | NM_178860 | seizure related 6 homolog |
| SF4 | NM_182812 | splicing factor 4 isoform c |
| SFMBT1 | NM_001005158 | Scm-like with four mbt domains 1 |
| SFRP1 | NM_003012 | secreted frizzled-related protein 1 |
| SFRP2 | NM_003013 | secreted frizzled-related protein 2 precursor |
| SFRS14 | NM_001017392 | splicing factor, arginine/serine-rich 14 |
| SFRS2 | NM_003016 | splicing factor, arginine/serine-rich 2 |
| SFT2D2 | NM_199344 | SFT2 domain containing 2 |
| SFXN2 | NM_178858 | sideroflexin 2 |
| SFXN5 | NM_144579 | sideroflexin 5 |
| SGCD | NM_000337 | delta-sarcoglycan isoform 1 |
| SGK3 | NM_001033578 | serum/glucocorticoid regulated kinase 3 isoform |
| SGPL1 | NM_003901 | sphingosine-1-phosphate lyase 1 |
| SH2D3A | NM_005490 | SH2 domain containing 3A |
| SH3BGRL2 | NM_031469 | SH3 domain binding glutamic acid-rich protein |
| SH3BP5 | NM_001018009 | SH3-domain binding protein 5 (BTK-associated) |
| SH3GL3 | NM_003027 | SH3-domain GRB2-like 3 |
| SH3PX3 | NM_153271 | SH3 and PX domain containing 3 |
| SH3PXD2A | NM_014631 | SH3 multiple domains 1 |
| SH3RF2 | NM_152550 | SH3 domain containing ring finger 2 |
| SHANK2 | NM_012309 | SH3 and multiple ankyrin repeat domains 2 |
| SHCBP1 | NM_024745 | SHC SH2-domain binding protein 1 |
| SHE | NM_001010846 | Src homology 2 domain containing E |
| SHF | NM_138356 | hypothetical protein LOC90525 |
| SHMT2 | NM_005412 | serine hydroxymethyltransferase 2 |
| SHOC2 | NM_007373 | soc-2 suppressor of clear homolog |
| SIAE | NM_170601 | cytosolic sialic acid 9-O-acetylesterase |
| SIDT1 | NM_017699 | SID1 transmembrane family, member 1 |
| SIGLEC10 | NM_033130 | sialic acid binding Ig-like lectin 10 |
| SIGLEC11 | NM_052884 | sialic acid binding Ig-like lectin 11 |
| SIKE | NM_025073 | suppressor of IKK epsilon |
| SIM2 | NM_009586 | single-minded homolog 2 short isoform |
| SIN3B | NM_015260 | SIN3 homolog B, transcription regulator |
| SIPA1L3 | NM_015073 | signal-induced proliferation-associated 1 like |
| SIRT3 | NM_001017524 | sirtuin 3 isoform b |
| SIRT7 | NM_016538 | sirtuin 7 |
| SKI | NM_003036 | v-ski sarcoma viral oncogene homolog |
| SLAMF7 | NM_021181 | SLAM family member 7 |
| SLC11A1 | NM_000578 | solute carrier family 11 (proton-coupled |
| SLC12A7 | NM_006598 | solute carrier family 12 (potassium/chloride |
| SLC13A1 | NM_022444 | solute carrier family 13 (sodium/sulfate |
| SLC14A1 | NM_015865 | RACH1 |
| SLC14A2 | NM_007163 | solute carrier family 14 (urea transporter), |
| SLC16A12 | NM_213606 | solute carrier family 16 (monocarboxylic acid |
| SLC16A14 | NM_152527 | solute carrier family 16 (monocarboxylic acid |
| SLC16A2 | NM_006517 | solute carrier family 16, member 2 |
| SLC16A7 | NM_004731 | solute carrier family 16, member 7 |
| SLC16A9 | NM_194298 | solute carrier family 16 (monocarboxylic acid |
| SLC17A5 | NM_012434 | solute carrier family 17 (anion/sugar |
| SLC17A7 | NM_020309 | solute carrier family 17, member 7 |
| SLC17A8 | NM_139319 | solute carrier family 17 (sodium-dependent |
| SLC19A3 | NM_025243 | solute carrier family 19, member 3 |
| SLC1A2 | NM_004171 | solute carrier family 1, member 2 |
| SLC1A4 | NM_003038 | solute carrier family 1, member 4 |
| SLC22A15 | NM_018420 | solute carrier family 22 (organic cation |
| SLC22A2 | NM_003058 | solute carrier family 22 member 2 isoform a |
| SLC22A3 | NM_021977 | solute carrier family 22 member 3 |
| SLC22A5 | NM_003060 | solute carrier family 22 member 5 |
| SLC23A3 | NM_144712 | solute carrier family 23 (nucleobase |
| SLC24A3 | NM_020689 | solute carrier family 24 |
| SLC24A4 | NM_153646 | solute carrier family 24 member 4 isoform 1 |
| SLC25A10 | NM_012140 | solute carrier family 25 (mitochondrial carrier; |
| SLC25A13 | NM_014251 | solute carrier family 25, member 13 (citrin) |
| SLC25A24 | NM_013386 | solute carrier family 25 member 24 isoform 1 |
| SLC25A27 | NM_004277 | solute carrier family 25, member 27 |
| SLC25A34 | NM_207348 | solute carrier family 25, member 34 |
| SLC26A4 | NM_000441 | Pendrin |
| SLC26A7 | NM_052832 | solute carrier family 26, member 7 isoform a |
| SLC29A2 | NM_001532 | solute carrier family 29 (nucleoside |
| SLC2A11 | NM_030807 | glucose transporter protein 10 isoform a |
| SLC2A2 | NM_000340 | solute carrier family 2 (facilitated glucose |
| SLC2A3 | NM_006931 | solute carrier family 2 (facilitated glucose |
| SLC2A4 | NM_001042 | glucose transporter 4 |
| SLC2A4RG | NM_020062 | SLC2A4 regulator |
| SLC2A5 | NM_003039 | solute carrier family 2 (facilitated |
| SLC2A6 | NM_017585 | solute carrier family 2 (facilitated glucose |
| SLC30A10 | NM_001004433 | solute carrier family 30 (zinc transporter), |
| SLC30A3 | NM_003459 | solute carrier family 30 (zinc transporter), |
| SLC30A7 | NM_133496 | zinc transporter like 2 |
| SLC31A1 | NM_001859 | solute carrier family 31 (copper transporters), |
| SLC35A5 | NM_017945 | solute carrier family 35, member A5 |
| SLC35B4 | NM_032826 | solute carrier family 35, member B4 |
| SLC35E1 | NM_024881 | solute carrier family 35, member E1 |
| SLC35E3 | NM_018656 | solute carrier family 35, member E2 |
| SLC35F3 | NM_173508 | solute carrier family 35, member F3 |
| SLC35F5 | NM_025181 | solute carrier family 35, member F5 |
| SLC36A1 | NM_078483 | solute carrier family 36 member 1 |
| SLC36A2 | NM_181776 | solute carrier family 36 (proton/amino acid |
| SLC37A4 | NM_001467 | solute carrier family 37 (glycerol-6-phosphate |
| SLC40A1 | NM_014585 | solute carrier family 40 (iron-regulated |
| SLC41A1 | NM_173854 | solute carrier family 41 member 1 |
| SLC44A4 | NM_025257 | NG22 protein isoform 1 |
| SLC45A2 | NM_001012509 | membrane-associated transporter protein isoform |
| SLC4A7 | NM_003615 | solute carrier family 4, sodium bicarbonate |
| SLC5A12 | NM_178498 | solute carrier family 5 (sodium/glucose |
| SLC5A7 | NM_021815 | solute carrier family 5 (choline transporter), |
| SLC6A8 | NM_005629 | solute carrier family 6 (neurotransmitter |
| SLC6A9 | NM_001024845 | solute carrier family 6 member 9 isoform 3 |
| SLC7A11 | NM_014331 | solute carrier family 7, (cationic amino acid |
| SLC7A2 | NM_001008539 | solute carrier family 7, member 2 isoform 1 |
| SLC7A6 | NM_003983 | solute carrier family 7 (cationic amino acid |
| SLC9A2 | NM_003048 | solute carrier family 9 (sodium/hydrogen |
| SLC9A3R1 | NM_004252 | solute carrier family 9 (sodium/hydrogen |
| SLC9A6 | NM_006359 | solute carrier family 9 (sodium/hydrogen |
| SLC9A8 | NM_015266 | Na+/H+ exchanger isoform 8 |
| SLC9A9 | NM_173653 | solute carrier family 9 (sodium/hydrogen |
| SLCO1C1 | NM_017435 | solute carrier organic anion transporter family, |
| SLCO4C1 | NM_180991 | solute carrier organic anion transporter family, |
| SLD5 | NM_032336 | SLD5 |
| SLITRK2 | NM_032539 | SLIT and NTRK-like family, member 2 |
| SLITRK3 | NM_014926 | slit and trk like 3 protein |
| SLK | NM_014720 | serine/threonine kinase 2 |
| SLTM | NM_001013843 | modulator of estrogen induced transcription |
| SMA4 | NM_021652 | SMA4 |
| SMA5 | NM_021036 | SMA5 |
| SMAD1 | NM_001003688 | Sma- and Mad-related protein 1 |
| SMAD2 | NM_001003652 | Sma- and Mad-related protein 2 |
| SMAD5 | NM_001001419 | SMAD, mothers against DPP homolog 5 |
| SMAD6 | NM_005585 | MAD, mothers against decapentaplegic homolog 6 |
| SMAD7 | NM_005904 | MAD, mothers against decapentaplegic homolog 7 |
| SMC1L1 | NM_006306 | SMC1 structural maintenance of chromosomes |
| SMC1L2 | NM_148674 | SMC1 structural maintenance of chromosomes |
| SMC4L1 | NM_001002799 | SMC4 structural maintenance of chromosomes |
| SMEK2 | NM_020463 | hypothetical protein LOC57223 |
| SMG1 | NM_015092 | PI-3-kinase-related kinase SMG-1 |
| SMOC1 | NM_022137 | secreted modular calcium-binding protein 1 |
| SMOC2 | NM_022138 | secreted modular calcium-binding protein 2 |
| SMYD1 | NM_198274 | SET and MYND domain containing 1 |
| SMYD4 | NM_052928 | SET and MYND domain containing 4 |
| SNAP23 | NM_003825 | synaptosomal-associated protein 23 isoform |
| SNAPC4 | NM_003086 | small nuclear RNA activating complex, |
| SNF1LK | NM_173354 | SNF1-like kinase |
| SNPH | NM_014723 | Syntaphilin |
| SNRK | NM_017719 | SNF related kinase |
| SNRPD3 | NM_004175 | small nuclear ribonucleoprotein polypeptide D3 |
| SNX11 | NM_013323 | sorting nexin 11 |
| SNX16 | NM_022133 | sorting nexin 16 isoform a |
| SNX19 | NM_014758 | sorting nexin 19 |
| SNX22 | NM_024798 | sorting nexin 22 |
| SNX27 | NM_030918 | sorting nexin family member 27 |
| SNX9 | NM_016224 | sorting nexin 9 |
| SOCS6 | NM_004232 | suppressor of cytokine signaling 6 |
| SOD2 | NM_000636 | manganese superoxide dismutase isoform A |
| SOLH | NM_005632 | small optic lobes |
| SORBS2 | NM_003603 | sorbin and SH3 domain containing 2 isoform 1 |
| SORL1 | NM_003105 | sortilin-related receptor containing LDLR class |
| SORT1 | NM_002959 | sortilin 1 preproprotein |
| SOX1 | NM_005986 | SRY (sex determining region Y)-box 1 |
| SOX12 | NM_006943 | SRY (sex determining region Y)-box 12 |
| SOX4 | NM_003107 | SRY (sex determining region Y)-box 4 |
| SOX7 | NM_031439 | SRY-box 7 |
| SP140 | NM_007237 | SP140 nuclear body protein isoform 1 |
| SP4 | NM_003112 | Sp4 transcription factor |
| SP6 | NM_199262 | Sp6 transcription factor |
| SP8 | NM_182700 | Sp8 transcription factor isoform 1 |
| SPACA1 | NM_030960 | sperm acrosome associated 1 |
| SPACA4 | NM_133498 | sperm acrosomal membrane protein 14 |
| SPARC | NM_003118 | secreted protein, acidic, cysteine-rich |
| SPATA13 | NM_153023 | spermatogenesis associated 13 |
| SPATA3 | NM_139073 | testis and spermatogenesis cell apoptosis |
| SPATS2 | NM_023071 | spermatogenesis associated, serine-rich 2 |
| SPBC24 | NM_182513 | spindle pole body component 24 homolog |
| SPECC1 | NM_001033553 | spectrin domain with coiled-coils 1 NSP5b3b |
| SPFH1 | NM_006459 | SPFH domain family, member 1 |
| SPFH2 | NM_007175 | SPFH domain family, member 2 isoform 1 |
| SPG20 | NM_015087 | Spartin |
| SPIRE2 | NM_032451 | spire homolog 2 |
| SPN | NM_001030288 | Sialophorin |
| SPOCK1 | NM_004598 | sparc/osteonectin, cwcv and kazal-like domains |
| SPOCK2 | NM_014767 | sparc/osteonectin, cwcv and kazal-like domains |
| SPRN | NM_001012508 | shadow of prion protein |
| SPRR2B | NM_001017418 | small proline-rich protein 2B |
| SPRR2E | NM_001024209 | small proline-rich protein 2E |
| SPRR2F | NM_001014450 | small proline-rich protein 2F |
| SPRY4 | NM_030964 | sprouty homolog 4 |
| SPSB4 | NM_080862 | SPRY domain-containing SOCS box protein SSB-4 |
| SPTBN4 | NM_020971 | spectrin, beta, non-erythrocytic 4 isoform 1 |
| SPTLC1 | NM_178324 | serine palmitoyltransferase subunit 1 isoform b |
| SPTLC2 | NM_004863 | serine palmitoyltransferase, long chain base |
| SPTY2D1 | NM_194285 | hypothetical protein LOC144108 |
| SQSTM1 | NM_003900 | sequestosome 1 |
| SRD5A2L2 | NM_001010874 | steroid 5 alpha-reductase 2-like 2 |
| SRGAP3 | NM_001033116 | SLIT-ROBO Rho GTPase activating protein 3 |
| SRI | NM_003130 | sorcin isoform a |
| SRP72 | NM_006947 | signal recognition particle 72 kDa |
| SRPK1 | NM_003137 | SFRS protein kinase 1 |
| SRPK2 | NM_182691 | SFRS protein kinase 2 isoform b |
| SRXN1 | NM_080725 | sulfiredoxin 1 homolog |
| SS18L1 | NM_015558 | SS18-like protein 1 |
| SSBP3 | NM_001009955 | single stranded DNA binding protein 3 isoform c |
| SSFA2 | NM_006751 | sperm specific antigen 2 |
| SSH2 | NM_033389 | slingshot 2 |
| SSPN | NM_005086 | Sarcospan |
| SSR3 | NM_007107 | signal sequence receptor gamma subunit |
| SSTR2 | NM_001050 | somatostatin receptor 2 |
| SSX2IP | NM_014021 | synovial sarcoma, X breakpoint 2 interacting |
| ST3GAL1 | NM_003033 | sialyltransferase 4A |
| ST6GAL1 | NM_003032 | sialyltransferase 1 isoform a |
| ST6GALNAC3 | NM_152996 | ST6 |
| ST6GALNAC6 | NM_013443 | ST6 |
| ST8SIA2 | NM_006011 | ST8 alpha-N-acetyl-neuraminide |
| ST8SIA4 | NM_005668 | ST8 alpha-N-acetyl-neuraminide |
| STAC | NM_003149 | SH3 and cysteine rich domain |
| STAC2 | NM_198993 | SH3 and cysteine rich domain 2 |
| STAM2 | NM_005843 | signal transducing adaptor molecule 2 |
| STAR | NM_000349 | steroidogenic acute regulator isoform 1 |
| STARD8 | NM_014725 | START domain containing 8 |
| STAT1 | NM_007315 | signal transducer and activator of transcription |
| STAT3 | NM_003150 | signal transducer and activator of transcription |
| STAT5B | NM_012448 | signal transducer and activator of transcription |
| STC1 | NM_003155 | stanniocalcin 1 precursor |
| STIM1 | NM_003156 | stromal interaction molecule 1 precursor |
| STIM2 | NM_020860 | stromal interaction molecule 2 |
| STK11 | NM_000455 | serine/threonine protein kinase 11 |
| STK11IP | NM_052902 | LKB1 interacting protein |
| STK17B | NM_004226 | serine/threonine kinase 17b |
| STK33 | NM_030906 | serine/threonine kinase 33 |
| STK38 | NM_007271 | serine/threonine kinase 38 |
| STK4 | NM_006282 | serine/threonine kinase 4 |
| STOM | NM_004099 | stomatin isoform a |
| STRBP | NM_018387 | spermatid perinuclear RNA-binding protein |
| STRN3 | NM_014574 | nuclear autoantigen |
| STS-1 | NM_032873 | Cbl-interacting protein Sts-1 |
| STX6 | NM_005819 | syntaxin 6 |
| STYX | NM_145251 | serine/threonine/tyrosine interacting protein |
| SUDS3 | NM_022491 | suppressor of defective silencing 3 |
| SUHW2 | NM_080764 | suppressor of hairy wing homolog 2 |
| SUHW3 | NM_017666 | suppressor of hairy wing homolog 3 |
| SUHW4 | NM_001002843 | suppressor of hairy wing homolog 4 isoform 2 |
| SULF1 | NM_015170 | sulfatase 1 |
| SULT2A1 | NM_003167 | sulfotransferase family, cytosolic, 2A, |
| SUMF1 | NM_182760 | sulfatase modifying factor 1 |
| SUPT7L | NM_014860 | SPTF-associated factor 65 gamma |
| SUSD1 | NM_022486 | sushi domain containing 1 |
| SUV39H2 | NM_024670 | suppressor of variegation 3-9 homolog 2 |
| SUV420H1 | NM_017635 | suppressor of variegation 4-20 homolog 1 isoform |
| SVH | NM_031905 | SVH protein |
| SWAP70 | NM_015055 | SWAP-70 protein |
| SYAP1 | NM_032796 | SYAP1 protein |
| SYNE1 | NM_015293 | nesprin 1 isoform beta |
| SYNE2 | NM_015180 | spectrin repeat containing, nuclear envelope 2 |
| SYNGR2 | NM_004710 | synaptogyrin 2 |
| SYNJ2BP | NM_018373 | synaptojanin 2 binding protein |
| SYNPO2 | NM_133477 | synaptopodin 2 |
| SYNPO2L | NM_024875 | synaptopodin 2-like |
| SYT10 | NM_198992 | synaptotagmin 10 |
| SYT13 | NM_020826 | synaptotagmin XIII |
| SYT15 | NM_031912 | synaptotagmin XV isoform a |
| SYT7 | NM_004200 | synaptotagmin VII |
| SYTL4 | NM_080737 | synaptotagmin-like 4 (granuphilin-a) |
| TACC1 | NM_006283 | transforming, acidic coiled-coil containing |
| TAF7 | NM_005642 | TATA box-binding protein-associated factor 2F |
| TAF9B | NM_015975 | transcription associated factor 9B |
| TAGAP | NM_054114 | T-cell activation Rho GTPase-activating protein |
| TAIP-2 | NM_024969 | TGF-beta induced apoptosis protein 2 |
| TAL1 | NM_003189 | T-cell acute lymphocytic leukemia 1 |
| TANC1 | NM_033394 | TPR domain, ankyrin-repeat and |
| TAOK2 | NM_016151 | TAO kinase 2 isoform 2 |
| TAOK3 | NM_016281 | TAO kinase 3 |
| TAPBP | NM_172208 | tapasin isoform 2 precursor |
| TAT | NM_000353 | tyrosine aminotransferase |
| TAX1BP1 | NM_006024 | Tax1 (human T-cell leukemia virus type I) |
| TBC1D1 | NM_015173 | TBC1 (tre-2/USP6, BUB2, cdc16) domain family, |
| TBC1D10C | NM_198517 | TBC1 domain family, member 10C |
| TBC1D15 | NM_022771 | TBC1 domain family, member 15 |
| TBC1D17 | NM_024682 | TBC1 domain family, member 17 |
| TBC1D2 | NM_018421 | TBC1 domain family, member 2 |
| TBC1D4 | NM_014832 | TBC1 domain family, member 4 |
| TBC1D8 | NM_007063 | TBC1 domain family, member 8 |
| TBC1D9 | NM_015130 | hypothetical protein LOC23158 |
| TBL1X | NM_005647 | transducin beta-like 1X |
| TBRG1 | NM_032811 | transforming growth factor beta regulator 1 |
| TBX19 | NM_005149 | T-box 19 |
| TBX3 | NM_005996 | T-box 3 protein isoform 1 |
| TCEAL7 | NM_152278 | hypothetical protein LOC56849 |
| TCEB3 | NM_003198 | elongin A |
| TCF21 | NM_003206 | transcription factor 21 |
| TCF7 | NM_003202 | transcription factor 7 (T-cell specific, |
| TCF7L1 | NM_031283 | HMG-box transcription factor TCF-3 |
| TCL6 | NM_014418 | T-cell leukemia/lymphoma 6 isoform TCL6a2 |
| TCN2 | NM_000355 | transcobalamin II precursor |
| TCOF1 | NM_001008657 | Treacher Collins-Franceschetti syndrome 1 |
| TCTA | NM_022171 | T-cell leukemia translocation altered gene |
| TCTEX1D1 | NM_152665 | hypothetical protein LOC200132 |
| TDRD1 | NM_198795 | tudor domain containing 1 |
| TEGT | NM_003217 | testis enhanced gene transcript (BAX inhibitor |
| TEK | NM_000459 | TEK tyrosine kinase, endothelial precursor |
| TEP1 | NM_007110 | telomerase-associated protein 1 |
| TESC | NM_017899 | Tescalcin |
| TEX14 | NM_031272 | testis expressed sequence 14 isoform b |
| TEX15 | NM_031271 | testis expressed sequence 15 |
| TEX261 | NM_144582 | testis expressed sequence 261 |
| TFEC | NM_001018058 | transcription factor EC isoform b |
| TGFB1I1 | NM_015927 | androgen receptor coactivator ARA55 |
| TGFB2 | NM_003238 | transforming growth factor, beta 2 |
| TGFBI | NM_000358 | transforming growth factor, beta-induced, 68 kDa |
| TGFBR2 | NM_001024847 | TGF-beta type II receptor isoform A precursor |
| TGM2 | NM_198951 | transglutaminase 2 isoform b |
| TGM3 | NM_003245 | transglutaminase 3 precursor |
| TGOLN2 | NM_006464 | trans-golgi network protein 2 |
| THADA | NM_198554 | thyroid adenoma associated isoform 2 |
| THAP2 | NM_031435 | THAP domain containing, apoptosis associated |
| THAP6 | NM_144721 | THAP domain containing 6 |
| THBD | NM_000361 | thrombomodulin precursor |
| THBS2 | NM_003247 | thrombospondin 2 precursor |
| THEDC1 | NM_018324 | thioesterase domain containing 1 isoform 1 |
| THEM4 | NM_053055 | thioesterase superfamily member 4 isoform a |
| THEM5 | NM_182578 | thioesterase superfamily member 5 |
| THEX1 | NM_153332 | histone mRNA 3′ end-specific exonuclease |
| THRA | NM_199334 | thyroid hormone receptor, alpha isoform 1 |
| THSD3 | NM_182509 | thrombospondin, type I domain containing 3 |
| THUMPD1 | NM_017736 | THUMP domain containing 1 |
| TIFA | NM_052864 | TRAF-interacting protein with a |
| TIMM10 | NM_012456 | translocase of inner mitochondrial membrane 10 |
| TIMM17A | NM_006335 | translocase of inner mitochondrial membrane 17 |
| TIMM44 | NM_006351 | translocase of inner mitochondrial membrane 44 |
| TIMM50 | NM_001001563 | translocase of inner mitochondrial membrane 50 |
| TIMP2 | NM_003255 | tissue inhibitor of metalloproteinase 2 |
| TIMP3 | NM_000362 | tissue inhibitor of metalloproteinase 3 |
| TIPARP | NM_015508 | TCDD-inducible poly(ADP-ribose) polymerase |
| TJP1 | NM_003257 | tight junction protein 1 isoform a |
| TLE4 | NM_007005 | transducin-like enhancer protein 4 |
| TLL1 | NM_012464 | tolloid-like 1 |
| TLOC1 | NM_003262 | translocation protein 1 |
| TLR7 | NM_016562 | toll-like receptor 7 |
| TM2D2 | NM_001024380 | TM2 domain containing 2 isoform b |
| TMBIM4 | NM_016056 | transmembrane BAX inhibitor motif containing 4 |
| TMC7 | NM_024847 | transmembrane channel-like 7 |
| TMCC1 | NM_001017395 | transmembrane and coiled-coil domains 1 isoform |
| TMCC3 | NM_020698 | transmembrane and coiled-coil domains 3 |
| TMED5 | NM_016040 | transmembrane emp24 protein transport domain |
| TMEM1 | NM_001001723 | transmembrane protein 1 isoform b |
| TMEM105 | NM_178520 | hypothetical protein LOC284186 |
| TMEM113 | NM_025222 | hypothetical protein PRO2730 |
| TMEM123 | NM_052932 | pro-oncosis receptor inducing membrane injury |
| TMEM127 | NM_017849 | hypothetical protein LOC55654 |
| TMEM130 | NM_152913 | hypothetical protein LOC222865 |
| TMEM133 | NM_032021 | hypothetical protein LOC83935 |
| TMEM135 | NM_022918 | hypothetical protein LOC65084 |
| TMEM138 | NM_016464 | hypothetical protein LOC51524 |
| TMEM16E | NM_213599 | transmembrane protein 16E |
| TMEM16F | NM_001025356 | transmembrane protein 16F |
| TMEM17 | NM_198276 | transmembrane protein 17 |
| TMEM18 | NM_152834 | transmembrane protein 18 |
| TMEM23 | NM_147156 | phosphatidylcholine:ceramide |
| TMEM25 | NM_032780 | transmembrane protein 25 |
| TMEM26 | NM_178505 | transmembrane protein 26 |
| TMEM28 | NM_015686 | transmembrane protein 28 |
| TMEM30A | NM_018247 | transmembrane protein 30A |
| TMEM38A | NM_024074 | transmembrane protein 38A |
| TMEM40 | NM_018306 | transmembrane protein 40 |
| TMEM41A | NM_080652 | transmembrane protein 41A |
| TMEM45B | NM_138788 | transmembrane protein 45B |
| TMEM50B | NM_006134 | transmembrane protein 50B |
| TMEM56 | NM_152487 | transmembrane protein 56 |
| TMEM64 | NM_001008495 | transmembrane protein 64 |
| TMEM71 | NM_144649 | hypothetical protein LOC137835 |
| TMEM77 | NM_178454 | hypothetical protein LOC128338 |
| TMEM80 | NM_174940 | hypothetical protein LOC283232 |
| TMEM83 | NM_152454 | hypothetical protein LOC145978 |
| TMOD1 | NM_003275 | tropomodulin 1 |
| TMOD2 | NM_014548 | tropomodulin 2 (neuronal) |
| TMPO | NM_001032283 | thymopoietin isoform beta |
| TMPRSS11B | NM_182502 | transmembrane protease, serine 11B |
| TMTC2 | NM_152588 | hypothetical protein LOC160335 |
| TNFAIP1 | NM_021137 | tumor necrosis factor, alpha-induced protein 1 |
| TNFAIP3 | NM_006290 | tumor necrosis factor, alpha-induced protein 3 |
| TNFAIP8L1 | NM_152362 | tumor necrosis factor, alpha-induced protein |
| TNFAIP8L2 | NM_024575 | tumor necrosis factor, alpha-induced protein |
| TNFAIP8L3 | NM_207381 | tumor necrosis factor, alpha-induced protein |
| TNFRSF10A | NM_003844 | tumor necrosis factor receptor superfamily, |
| TNFRSF10B | NM_003842 | tumor necrosis factor receptor superfamily, |
| TNFRSF10D | NM_003840 | tumor necrosis factor receptor superfamily, |
| TNFRSF17 | NM_001192 | tumor necrosis factor receptor superfamily, |
| TNFRSF19 | NM_148957 | tumor necrosis factor receptor superfamily, |
| TNFRSF1B | NM_001066 | tumor necrosis factor receptor 2 precursor |
| TNFRSF21 | NM_014452 | tumor necrosis factor receptor superfamily, |
| TNFSF11 | NM_003701 | tumor necrosis factor ligand superfamily, member |
| TNFSF14 | NM_003807 | tumor necrosis factor ligand superfamily, member |
| TNFSF15 | NM_005118 | tumor necrosis factor (ligand) superfamily, |
| TNIP3 | NM_024873 | hypothetical protein LOC79931 |
| TNK2 | NM_001010938 | tyrosine kinase, non-receptor, 2 isoform 2 |
| TNKS1BP1 | NM_033396 | tankyrase 1-binding protein of 182 kDa |
| TNKS2 | NM_025235 | tankyrase, TRF1-interacting ankyrin-related |
| TNNI1 | NM_003281 | troponin I, skeletal, slow |
| TNRC6A | NM_014494 | trinucleotide repeat containing 6A isoform 1 |
| TNRC6B | NM_001024843 | trinucleotide repeat containing 6B isoform 2 |
| TOLLIP | NM_019009 | toll interacting protein |
| TOMM40L | NM_032174 | translocase of outer mitochondrial membrane 40 |
| TOMM7 | NM_019059 | 6.2 kd protein |
| TOMM70A | NM_014820 | translocase of outer mitochondrial membrane 70 |
| TOP3A | NM_004618 | topoisomerase (DNA) III alpha |
| TOPORS | NM_005802 | topoisomerase I binding, arginine/serine-rich |
| TOR1B | NM_014506 | torsin family 1, member B (torsin B) |
| TP53INP1 | NM_033285 | tumor protein p53 inducible nuclear protein 1 |
| TP53INP2 | NM_021202 | tumor protein p53 inducible nuclear protein 2 |
| TPD52 | NM_001025252 | tumor protein D52 isoform 1 |
| TPK1 | NM_022445 | thiamin pyrophosphokinase 1 |
| TPM4 | NM_003290 | tropomyosin 4 |
| TRA16 | NM_176880 | TR4 orphan receptor associated protein TRA16 |
| TRAK1 | NM_014965 | OGT(O-Glc-NAc transferase)-interacting protein |
| TRAM1 | NM_014294 | translocating chain-associating membrane |
| TRAM2 | NM_012288 | translocation-associated membrane protein 2 |
| TRAPPC2 | NM_001011658 | trafficking protein particle complex 2 |
| TRIAD3 | NM_019011 | TRIAD3 protein isoform c |
| TRIAP1 | NM_016399 | p53-inducible cell-survival factor |
| TRIB3 | NM_021158 | tribbles 3 |
| TRIM10 | NM_052828 | tripartite motif-containing 10 isoform 2 |
| TRIM2 | NM_015271 | tripartite motif-containing 2 |
| TRIM22 | NM_006074 | tripartite motif-containing 22 |
| TRIM26 | NM_003449 | tripartite motif-containing 26 |
| TRIM3 | NM_006458 | tripartite motif-containing 3 |
| TRIM31 | NM_052816 | tripartite motif protein 31 isoform beta |
| TRIM32 | NM_012210 | TAT-interactive protein, 72-KD |
| TRIM33 | NM_015906 | tripartite motif-containing 33 protein isoform |
| TRIM36 | NM_018700 | tripartite motif-containing 36 isoform 1 |
| TRIM37 | NM_015294 | tripartite motif-containing 37 protein |
| TRIM4 | NM_033017 | tripartite motif protein TRIM4 isoform alpha |
| TRIM55 | NM_033058 | ring finger protein 29 isoform 2 |
| TRIM56 | NM_030961 | tripartite motif-containing 56 |
| TRIM58 | NM_015431 | tripartite motif-containing 58 |
| TRIM65 | NM_173547 | tripartite motif-containing 65 |
| TRIM68 | NM_018073 | ring finger protein 137 |
| TRIM7 | NM_203293 | tripartite motif-containing 7 isoform 1 |
| TRIM8 | NM_030912 | tripartite motif-containing 8 |
| TRIM9 | NM_052978 | tripartite motif protein 9 isoform 2 |
| TRIP10 | NM_004240 | thyroid hormone receptor interactor 10 |
| TRIP11 | NM_004239 | thyroid hormone receptor interactor 11 |
| TRMU | NM_001008568 | tRNA 5-methylaminomethyl-2-thiouridylate |
| TRPA1 | NM_007332 | ankyrin-like protein 1 |
| TRPC1 | NM_003304 | transient receptor potential cation channel, |
| TRPC4 | NM_016179 | transient receptor potential 4 |
| TRPM1 | NM_002420 | transient receptor potential cation channel, |
| TRPM6 | NM_017662 | transient receptor potential cation channel, |
| TRPS1 | NM_014112 | zinc finger transcription factor TRPS1 |
| TRPV6 | NM_018646 | transient receptor potential cation channel, |
| TRSPAP1 | NM_017846 | tRNA selenocysteine associated protein |
| TRUB1 | NM_139169 | TruB pseudouridine (psi) synthase homolog 1 |
| TSC22D2 | NM_014779 | TSC22 domain family 2 |
| TSCOT | NM_033051 | thymic stromal co-transporter |
| TSEN2 | NM_025265 | tRNA splicing endonuclease 2 homolog |
| TSG101 | NM_006292 | tumor susceptibility gene 101 |
| TSHZ3 | NM_020856 | zinc finger protein 537 |
| TSNAX | NM_005999 | translin-associated factor X |
| TSPAN14 | NM_030927 | tetraspanin 14 |
| TSPAN17 | NM_001006616 | transmembrane 4 superfamily member 17 isoform c |
| TSPAN4 | NM_001025234 | tetraspanin 4 isoform a |
| TSPAN9 | NM_006675 | tetraspanin 9 |
| TSPYL1 | NM_003309 | TSPY-like 1 |
| TSR1 | NM_018128 | hypothetical protein LOC55720 |
| TTF2 | NM_003594 | transcription termination factor, RNA polymerase |
| TTL | NM_153712 | tubulin tyrosine ligase |
| TTLL6 | NM_173623 | hypothetical protein LOC284076 |
| TTN | NM_003319 | titin isoform N2-B |
| TULP3 | NM_003324 | tubby like protein 3 |
| TUSC2 | NM_007275 | tumor suppressor candidate 2 |
| TWIST1 | NM_000474 | Twist |
| TXLNA | NM_175852 | Taxilin |
| TXNDC10 | NM_019022 | thioredoxin domain containing 10 |
| TXNDC4 | NM_015051 | thioredoxin domain containing 4 (endoplasmic |
| TXNIP | NM_006472 | thioredoxin interacting protein |
| TXNL2 | NM_006541 | thioredoxin-like |
| TYRP1 | NM_000550 | tyrosinase-related protein 1 |
| UACA | NM_001008224 | uveal autoantigen with coiled-coil domains and |
| UBAP1 | NM_016525 | ubiquitin associated protein 1 |
| UBASH3A | NM_001001895 | ubiquitin associated and SH3 domain containing, |
| UBC | NM_021009 | ubiquitin C |
| UBE2B | NM_003337 | ubiquitin-conjugating enzyme E2B |
| UBE2G1 | NM_003342 | ubiquitin-conjugating enzyme E2G 1 isoform 1 |
| UBE2G2 | NM_003343 | ubiquitin-conjugating enzyme E2G 2 isoform 1 |
| UBE2J1 | NM_016021 | ubiquitin-conjugating enzyme E2, J1 |
| UBE2Q2 | NM_173469 | ubiquitin-conjugating enzyme E2Q (putative) 2 |
| UBE2W | NM_001001481 | hypothetical protein LOC55284 isoform 1 |
| UBE3A | NM_000462 | ubiquitin protein ligase E3A isoform 2 |
| UBE3C | NM_014671 | ubiquitin protein ligase E3C |
| UBE4A | NM_004788 | ubiquitination factor E4A |
| UBL4A | NM_014235 | ubiquitin-like 4 |
| UBOX5 | NM_014948 | U-box domain containing 5 isoform a |
| UBQLN1 | NM_013438 | ubiquilin 1 isoform 1 |
| UBQLN4 | NM_020131 | ataxin-1 ubiquitin-like interacting protein |
| UBXD4 | NM_181713 | UBX domain containing 4 |
| UBXD8 | NM_014613 | UBX domain containing 8 |
| UCP3 | NM_003356 | uncoupling protein 3 isoform UCP3L |
| UEV3 | NM_018314 | ubiquitin-conjugating enzyme E2-like |
| UGDH | NM_003359 | UDP-glucose dehydrogenase |
| UGP2 | NM_001001521 | UDP-glucose pyrophosphorylase 2 isoform b |
| ULK1 | NM_003565 | unc-51-like kinase 1 |
| UNC5C | NM_003728 | unc5C |
| UNC5CL | NM_173561 | unc-5 homolog C-like |
| UNC93B1 | NM_030930 | unc-93 homolog B1 |
| UNQ9370 | NM_207447 | hypothetical protein LOC400454 |
| UPF3A | NM_023011 | UPF3 regulator of nonsense transcripts homolog A |
| UPK1A | NM_007000 | uroplakin 1A |
| UPK1B | NM_006952 | uroplakin 1B |
| UPP1 | NM_003364 | uridine phosphorylase 1 |
| UQCRB | NM_006294 | ubiquinol-cytochrome c reductase binding |
| URB | NM_199511 | steroid-sensitive protein 1 |
| URG4 | NM_017920 | hypothetical protein LOC55665 |
| USP14 | NM_005151 | ubiquitin specific protease 14 isoform a |
| USP25 | NM_013396 | ubiquitin specific protease 25 |
| USP28 | NM_020886 | ubiquitin specific protease 28 |
| USP3 | NM_006537 | ubiquitin specific protease 3 |
| USP32 | NM_032582 | ubiquitin specific protease 32 |
| USP33 | NM_015017 | ubiquitin specific protease 33 isoform 1 |
| USP37 | NM_020935 | ubiquitin specific protease 37 |
| USP46 | NM_022832 | ubiquitin specific protease 46 |
| USP47 | NM_017944 | ubiquitin specific protease 47 |
| USP49 | NM_018561 | ubiquitin specific protease 49 |
| USP9Y | NM_004654 | ubiquitin specific protease 9, Y-linked |
| UST | NM_005715 | uronyl-2-sulfotransferase |
| UTP14C | NM_021645 | UTP14, U3 small nucleolar ribonucleoprotein, |
| UXS1 | NM_025076 | UDP-glucuronate decarboxylase 1 |
| VANGL1 | NM_138959 | vang-like 1 |
| VAPA | NM_003574 | vesicle-associated membrane protein-associated |
| VASP | NM_001008736 | vasodilator-stimulated phosphoprotein isoform 2 |
| VAV2 | NM_003371 | vav 2 oncogene |
| VAX1 | NM_199131 | ventral anterior homeobox 1 |
| VCL | NM_003373 | vinculin isoform VCL |
| VDAC1 | NM_003374 | voltage-dependent anion channel 1 |
| VEGF | NM_001025366 | vascular endothelial growth factor isoform a |
| VEZT | NM_017599 | transmembrane protein vezatin |
| VGLL2 | NM_153453 | vestigial-like 2 isoform 2 |
| VGLL3 | NM_016206 | colon carcinoma related protein |
| VGLL4 | NM_014667 | vestigial like 4 |
| VHL | NM_000551 | von Hippel-Lindau tumor suppressor isoform 1 |
| VLDLR | NM_001018056 | very low density lipoprotein receptor isoform b |
| VMP | NM_080723 | vesicular membrane protein p24 |
| VPS13C | NM_017684 | vacuolar protein sorting 13C protein isoform 1A |
| VPS13D | NM_015378 | vacuolar protein sorting 13D isoform 1 |
| VPS24 | NM_001005753 | vacuolar protein sorting 24 isoform 2 |
| VPS25 | NM_032353 | vacuolar protein sorting 25 |
| VPS26A | NM_004896 | vacuolar protein sorting 26 homolog A isoform 1 |
| VPS36 | NM_016075 | vacuolar protein sorting 36 |
| VPS37C | NM_017966 | vacuolar protein sorting 37C |
| VPS4B | NM_004869 | vacuolar protein sorting factor 4B |
| VRK1 | NM_003384 | vaccinia related kinase 1 |
| VSIG1 | NM_182607 | V-set and immunoglobulin domain containing 1 |
| VSX1 | NM_014588 | visual system homeobox 1 protein isoform a |
| WAC | NM_016628 | WW domain-containing adapter with a coiled-coil |
| WASF2 | NM_006990 | WAS protein family, member 2 |
| WASF3 | NM_006646 | WAS protein family, member 3 |
| WASL | NM_003941 | Wiskott-Aldrich syndrome gene-like protein |
| WBSCR1 | NM_022170 | eukaryotic translation initiation factor 4H |
| WBSCR22 | NM_017528 | Williams Beuren syndrome chromosome region 22 |
| WDFY3 | NM_014991 | WD repeat and FYVE domain containing 3 isoform |
| WDR1 | NM_005112 | WD repeat-containing protein 1 isoform 2 |
| WDR17 | NM_170710 | WD repeat domain 17 isoform 1 |
| WDR19 | NM_025132 | WD repeat domain 19 |
| WDR21C | NM_152418 | hypothetical protein LOC138009 |
| WDR23 | NM_025230 | WD repeat domain 23 isoform 1 |
| WDR26 | NM_025160 | WD repeat domain 26 |
| WDR32 | NM_024345 | WD repeat domain 32 |
| WDR33 | NM_001006623 | WD repeat domain 33 isoform 3 |
| WDR35 | NM_001006657 | WD repeat domain 35 isoform 1 |
| WDR36 | NM_139281 | WD repeat domain 36 |
| WDR37 | NM_014023 | WD repeat domain 37 |
| WDR39 | NM_004804 | WD repeat domain 39 |
| WDR4 | NM_018669 | WD repeat domain 4 protein |
| WDR40B | NM_178470 | WD repeat domain 40B |
| WDR42A | NM_015726 | H326 |
| WDR48 | NM_020839 | WD repeat domain 48 |
| WDR5B | NM_019069 | WD repeat domain 5B |
| WDR73 | NM_032856 | WD repeat domain 73 |
| WEE1 | NM_003390 | wee1 tyrosine kinase |
| WFS1 | NM_006005 | Wolframin |
| WHSC1 | NM_007331 | Wolf-Hirschhorn syndrome candidate 1 protein |
| WIG1 | NM_022470 | p53 target zinc finger protein isoform 1 |
| WIRE | NM_133264 | WIRE protein |
| WISP2 | NM_003881 | WNT1 inducible signaling pathway protein 2 |
| WNK2 | NM_006648 | WNK lysine deficient protein kinase 2 |
| WNK3 | NM_001002838 | WNK lysine deficient protein kinase 3 isoform 2 |
| WNT5A | NM_003392 | wingless-type MMTV integration site family, |
| WNT7B | NM_058238 | wingless-type MMTV integration site family, |
| WSB2 | NM_018639 | WD SOCS-box protein 2 |
| WT1 | NM_000378 | Wilms tumor 1 isoform A |
| WWP2 | NM_199423 | WW domain containing E3 ubiquitin protein ligase |
| XCL1 | NM_002995 | chemokine (C motif) ligand 1 |
| XCL2 | NM_003175 | chemokine (C motif) ligand 2 |
| XKR5 | NM_207411 | XK-related protein 5a |
| XKRX | NM_212559 | X Kell blood group precursor-related, X-linked |
| XPO5 | NM_020750 | exportin 5 |
| XRCC2 | NM_005431 | X-ray repair cross complementing protein 2 |
| XRN1 | NM_019001 | 5′-3′ exoribonuclease 1 |
| XTP7 | NM_138568 | protein 7 transactivated by hepatitis B virus X |
| YAF2 | NM_001012424 | YY1 associated factor 2 isoform b |
| YARS2 | NM_015936 | tyrosyl-tRNA synthetase 2 (mitochondrial) |
| YES1 | NM_005433 | viral oncogene yes-1 homolog 1 |
| YIPF5 | NM_001024947 | smooth muscle cell associated protein 5 |
| YME1L1 | NM_014263 | YME1-like 1 isoform 3 |
| YOD1 | NM_018566 | hypothetical protein LOC55432 |
| YPEL1 | NM_013313 | yippee-like 1 |
| YPEL2 | NM_001005404 | yippee-like 2 |
| YPEL4 | NM_145008 | yippee-like 4 |
| YTHDF3 | NM_152758 | YTH domain family, member 3 |
| YWHAQ | NM_006826 | tyrosine 3/tryptophan 5-monooxygenase |
| YWHAZ | NM_003406 | tyrosine 3/tryptophan 5-monooxygenase |
| ZADH1 | NM_152444 | zinc binding alcohol dehydrogenase, domain |
| ZADH2 | NM_175907 | zinc binding alcohol dehydrogenase, domain |
| ZAK | NM_133646 | MLK-related kinase isoform 2 |
| ZBED1 | NM_004729 | Ac-like transposable element |
| ZBP1 | NM_030776 | tumor stroma and activated macrophage protein |
| ZBTB24 | NM_014797 | zinc finger and BTB domain containing 24 |
| ZBTB33 | NM_006777 | Kaiso |
| ZBTB39 | NM_014830 | zinc finger and BTB domain containing 39 |
| ZBTB4 | NM_020899 | zinc finger and BTB domain containing 4 |
| ZBTB41 | NM_194314 | zinc finger and BTB domain containing 41 |
| ZBTB5 | NM_014872 | zinc finger and BTB domain containing 5 |
| ZBTB7A | NM_015898 | zinc finger and BTB domain containing 7A |
| ZBTB9 | NM_152735 | zinc finger and BTB domain containing 9 |
| ZC3H12B | NM_001010888 | hypothetical protein LOC340554 |
| ZCCHC16 | NM_001004308 | hypothetical protein LOC340595 |
| ZDHHC1 | NM_013304 | zinc finger, DHHC domain containing 1 |
| ZDHHC23 | NM_173570 | zinc finger, DHHC domain containing 23 |
| ZDHHC9 | NM_001008222 | zinc finger, DHHC domain containing 9 |
| ZFP106 | NM_022473 | zinc finger protein 106 homolog |
| ZFP161 | NM_003409 | zinc finger protein 161 homolog |
| ZFP30 | NM_014898 | zinc finger protein 30 homolog |
| ZFP42 | NM_174900 | zinc finger protein 42 |
| ZFP90 | NM_133458 | zinc finger protein 90 homolog |
| ZFP91 | NM_053023 | zinc finger protein 91 isoform 1 |
| ZFP95 | NM_014569 | zinc finger protein 95 homolog |
| ZFPM2 | NM_012082 | zinc finger protein, multitype 2 |
| ZFYVE20 | NM_022340 | FYVE-finger-containing Rab5 effector protein |
| ZFYVE21 | NM_024071 | zinc finger, FYVE domain containing 21 |
| ZFYVE26 | NM_015346 | zinc finger, FYVE domain containing 26 |
| ZFYVE9 | NM_004799 | zinc finger, FYVE domain containing 9 isoform 3 |
| ZHX2 | NM_014943 | zinc fingers and homeoboxes 2 |
| ZHX3 | NM_015035 | zinc fingers and homeoboxes 3 |
| ZIC1 | NM_003412 | zinc finger protein of the cerebellum 1 |
| ZIC4 | NM_032153 | zinc finger protein of the cerebellum 4 |
| ZIM3 | NM_052882 | zinc finger, imprinted 3 |
| ZKSCAN1 | NM_003439 | zinc finger protein 36 |
| ZMYM6 | NM_007167 | zinc finger protein 258 |
| ZNF114 | NM_153608 | zinc finger protein 114 |
| ZNF134 | NM_003435 | zinc finger protein 134 |
| ZNF136 | NM_003437 | zinc finger protein 136 (clone pHZ-20) |
| ZNF137 | NM_003438 | zinc finger protein 137 (clone pHZ-30) |
| ZNF14 | NM_021030 | zinc finger protein 14 |
| ZNF140 | NM_003440 | zinc finger protein 140 (clone pHZ-39) |
| ZNF148 | NM_021964 | zinc finger protein 148 (pHZ-52) |
| ZNF155 | NM_003445 | zinc finger protein 155 |
| ZNF160 | NM_033288 | zinc finger protein 160 |
| ZNF161 | NM_007146 | zinc finger protein 161 |
| ZNF177 | NM_003451 | zinc finger protein 177 |
| ZNF180 | NM_013256 | zinc finger protein 180 (HHZ168) |
| ZNF187 | NM_001023560 | zinc finger protein 187 |
| ZNF192 | NM_006298 | zinc finger protein 192 |
| ZNF195 | NM_007152 | zinc finger protein 195 |
| ZNF197 | NM_006991 | zinc finger protein 197 isoform 1 |
| ZNF2 | NM_001017396 | zinc finger protein 2 isoform b |
| ZNF202 | NM_003455 | zinc finger protein 202 |
| ZNF213 | NM_004220 | zinc finger protein 213 |
| ZNF217 | NM_006526 | zinc finger protein 217 |
| ZNF23 | NM_145911 | zinc finger protein 23 |
| ZNF236 | NM_007345 | zinc finger protein 236 |
| ZNF238 | NM_006352 | zinc finger protein 238 isoform 2 |
| ZNF239 | NM_005674 | zinc finger protein 239 |
| ZNF25 | NM_145011 | zinc finger protein 25 |
| ZNF264 | NM_003417 | zinc finger protein 264 |
| ZNF271 | NM_006629 | zinc finger protein 271 |
| ZNF28 | NM_006969 | zinc finger protein 28 (KOX 24) |
| ZNF282 | NM_003575 | zinc finger protein 282 |
| ZNF295 | NM_020727 | zinc finger protein 295 |
| ZNF304 | NM_020657 | zinc finger protein 304 |
| ZNF307 | NM_019110 | zinc finger protein 307 |
| ZNF31 | NM_145238 | zinc finger protein 31 |
| ZNF320 | NM_207333 | zinc finger protein 320 |
| ZNF329 | NM_024620 | zinc finger protein 329 |
| ZNF331 | NM_018555 | zinc finger protein 331 |
| ZNF333 | NM_032433 | zinc finger protein 333 |
| ZNF336 | NM_022482 | zinc finger protein 336 |
| ZNF337 | NM_015655 | zinc finger protein 337 |
| ZNF33A | NM_006974 | zinc finger protein 33a |
| ZNF346 | NM_012279 | zinc finger protein 346 |
| ZNF347 | NM_032584 | zinc finger protein 347 |
| ZNF367 | NM_153695 | zinc finger protein 367 |
| ZNF385 | NM_015481 | zinc finger protein 385 |
| ZNF394 | NM_032164 | zinc finger protein 99 |
| ZNF398 | NM_020781 | zinc finger 398 isoform b |
| ZNF417 | NM_152475 | zinc finger protein 417 |
| ZNF43 | NM_003423 | zinc finger protein 43 (HTF6) |
| ZNF430 | NM_025189 | zinc finger protein 430 |
| ZNF431 | NM_133473 | zinc finger protein 431 |
| ZNF440 | NM_152357 | zinc finger protein 440 |
| ZNF445 | NM_181489 | zinc finger protein 445 |
| ZNF452 | NM_052923 | zinc finger protein 452 |
| ZNF454 | NM_182594 | zinc finger protein 454 |
| ZNF468 | NM_001008801 | zinc finger protein ZNF468 isoform 2 |
| ZNF473 | NM_001006656 | zinc finger protein 473 |
| ZNF482 | NM_006626 | zinc finger protein 482 |
| ZNF483 | NM_001007169 | zinc finger protein 483 isoform b |
| ZNF490 | NM_020714 | zinc finger protein 490 |
| ZNF498 | NM_145115 | zinc finger protein 498 |
| ZNF500 | NM_021646 | zinc finger protein 500 |
| ZNF502 | NM_033210 | zinc finger protein 502 |
| ZNF510 | NM_014930 | zinc finger protein 510 |
| ZNF512 | NM_032434 | zinc finger protein 512 |
| ZNF514 | NM_032788 | zinc finger protein 514 |
| ZNF518 | NM_014803 | zinc finger protein 518 |
| ZNF526 | NM_133444 | zinc finger protein 526 |
| ZNF528 | NM_032423 | zinc finger protein 528 |
| ZNF532 | NM_018181 | zinc finger protein 532 |
| ZNF536 | NM_014717 | zinc finger protein 536 |
| ZNF542 | NM_194319 | zinc finger protein 542 |
| ZNF546 | NM_178544 | zinc finger protein 546 |
| ZNF549 | NM_153263 | zinc finger protein 549 |
| ZNF551 | NM_138347 | zinc finger protein 551 |
| ZNF554 | NM_152303 | zinc finger protein 554 |
| ZNF556 | NM_024967 | zinc finger protein 556 |
| ZNF561 | NM_152289 | zinc finger protein 561 |
| ZNF562 | NM_017656 | zinc finger protein 562 |
| ZNF565 | NM_152477 | zinc finger protein 565 |
| ZNF566 | NM_032838 | zinc finger protein 566 |
| ZNF577 | NM_032679 | zinc finger protein 577 |
| ZNF585A | NM_152655 | zinc finger protein 585A |
| ZNF587 | NM_032828 | zinc finger protein 587 |
| ZNF588 | NM_001013746 | zinc finger protein 588 |
| ZNF595 | NM_182524 | zinc finger protein 595 |
| ZNF597 | NM_152457 | zinc finger protein 597 |
| ZNF599 | NM_001007247 | zinc finger protein 599 isoform b |
| ZNF600 | NM_198457 | zinc finger protein 600 |
| ZNF620 | NM_175888 | zinc finger protein 620 |
| ZNF621 | NM_198484 | zinc finger protein 621 |
| ZNF623 | NM_014789 | zinc finger protein 623 |
| ZNF627 | NM_145295 | zinc finger protein 627 |
| ZNF651 | NM_145166 | zinc finger protein 651 |
| ZNF652 | NM_014897 | zinc finger protein 652 |
| ZNF655 | NM_001009956 | zinc finger protein 655 isoform e |
| ZNF662 | NM_207404 | zinc finger protein 662 |
| ZNF665 | NM_024733 | zinc finger protein 665 |
| ZNF667 | NM_022103 | zinc finger protein 667 |
| ZNF669 | NM_024804 | zinc finger protein 669 |
| ZNF671 | NM_024833 | zinc finger protein 671 |
| ZNF680 | NM_178558 | zinc finger protein 680 |
| ZNF684 | NM_152373 | zinc finger protein 684 |
| ZNF69 | NM_021915 | zinc finger protein 69 (Cos5) |
| ZNF696 | NM_030895 | zinc finger protein 696 |
| ZNF70 | NM_021916 | zinc finger protein 70 |
| ZNF701 | NM_018260 | zinc finger protein 701 |
| ZNF702 | NM_024924 | zinc finger protein 702 |
| ZNF704 | NM_001033723 | zinc finger protein 704 |
| ZNF708 | NM_021269 | zinc finger protein 15-like 1 (KOX 8) |
| ZNF71 | NM_021216 | zinc finger protein 71 |
| ZNF721 | NM_133474 | zinc finger protein 721 |
| ZNF81 | NM_007137 | zinc finger protein 81 (HFZ20) |
| ZNFN1A4 | NM_022465 | zinc finger protein, subfamily 1A, 4 |
| ZNFX1 | NM_021035 | zinc finger, NFX1-type containing 1 |
| ZSWIM3 | NM_080752 | zinc finger, SWIM domain containing 3 |
| ZSWIM4 | NM_023072 | zinc finger, SWIM domain containing 4 |
| ZXDB | NM_007157 | zinc finger, X-linked, duplicated B |
| ZYG11A | NM_001004339 | hypothetical protein LOC440590 |
| ZYG11B | NM_024646 | hypothetical protein LOC79699 |
| ZZEF1 | NM_015113 | zinc finger, ZZ type with EF hand domain 1 |
| ZZZ3 | NM_015534 | zinc finger, ZZ domain containing 3 |

TABLE 4
hsa-miR-20a targets that exhibited altered mRNA expression levels in human
cancer cells after transfection with pre-miR hsa-miR-20a.

| | RefSeq | |
|---|---|---|
| Gene | Transcript ID | |
| Symbol | (Pruitt et al., 2005) | Description |
| ABCA1 | NM_005502 | ATP-binding cassette, sub-family A member 1 |
| ANTXR1 | NM_018153 | tumor endothelial marker 8 isoform 3 precursor |
| ARTS-1 | NM_016442 | type 1 tumor necrosis factor receptor shedding |
| ATP6V0E | NM_003945 | ATPase, H+ transporting, lysosomal, V0 subunit |
| ATP9A | NM_006045 | ATPase, Class II, type 9A |
| BICD2 | NM_001003800 | bicaudal D homolog 2 isoform 1 |
| BTG3 | NM_006806 | B-cell translocation gene 3 |
| BTN3A2 | NM_007047 | butyrophilin, subfamily 3, member A2 precursor |
| C19orf2 | NM_003796 | RPB5-mediating protein isoform a |
| C21orf25 | NM_199050 | hypothetical protein LOC25966 |
| C6orf120 | NM_001029863 | hypothetical protein LOC387263 |
| CCND1 | NM_053056 | cyclin D1 |
| CDC37L1 | NM_017913 | cell division cycle 37 homolog (S. |
| CLIC4 | NM_013943 | chloride intracellular channel 4 |
| COL4A1 | NM_001845 | alpha 1 type IV collagen preproprotein |
| COL4A2 | NM_001846 | alpha 2 type IV collagen preproprotein |
| CPM | NM_001005502 | carboxypeptidase M precursor |
| CRIPT | NM_014171 | postsynaptic protein CRIPT |
| CXCL5 | NM_002994 | chemokine (C—X—C motif) ligand 5 precursor |
| DAZAP2 | NM_014764 | DAZ associated protein 2 |
| DCBLD2 | NM_080927 | discoidin, CUB and LCCL domain containing 2 |
| DDAH1 | NM_012137 | dimethylarginine dimethylaminohydrolase 1 |
| DNAJB6 | NM_005494 | DnaJ (Hsp40) homolog, subfamily B, member 6 |
| DNAJC15 | NM_013238 | DNAJ domain-containing |
| EIF2C1 | NM_012199 | eukaryotic translation initiation factor 2C, 1 |
| EIF2S1 | NM_004094 | eukaryotic translation initiation factor 2, |
| EREG | NM_001432 | epiregulin precursor |
| F2RL1 | NM_005242 | coagulation factor II (thrombin) receptor-like 1 |
| FAM18B | NM_016078 | hypothetical protein LOC51030 |
| FJX1 | NM_014344 | four jointed box 1 |
| FLJ31568 | NM_152509 | hypothetical protein LOC150244 |
| FTS | NM_001012398 | fused toes homolog |
| FYCO1 | NM_024513 | FYVE and coiled-coil domain containing 1 |
| FZD7 | NM_003507 | frizzled 7 |
| GATA6 | NM_005257 | GATA binding protein 6 |
| GNS | NM_002076 | glucosamine (N-acetyl)-6-sulfatase precursor |
| GOLPH2 | NM_016548 | golgi phosphoprotein 2 |
| HCCS | NM_005333 | holocytochrome c synthase (cytochrome c |
| HIC2 | NM_015094 | hypermethylated in cancer 2 |
| HMGA2 | NM_001015886 | high mobility group AT-hook 2 isoform c |
| HN1 | NM_001002032 | hematological and neurological expressed 1 |
| IL11 | NM_000641 | interleukin 11 precursor |
| IL8 | NM_000584 | interleukin 8 precursor |
| KCNMA1 | NM_001014797 | large conductance calcium-activated potassium |
| KIAA0494 | NM_014774 | hypothetical protein LOC9813 |
| KLF10 | NM_001032282 | Kruppel-like factor 10 isoform b |
| LEPROT | NM_017526 | leptin receptor gene-related protein |
| LHFP | NM_005780 | lipoma HMGIC fusion partner |
| LIMK1 | NM_002314 | LIM domain kinase 1 isoform 1 |
| LRRC54 | NM_015516 | Tsukushi |
| M6PR | NM_002355 | cation-dependent mannose-6-phosphate receptor |
| MAP3K2 | NM_006609 | mitogen-activated protein kinase kinase kinase |
| MGC11332 | NM_032718 | hypothetical protein LOC84804 |
| MICA | NM_000247 | MHC class I chain-related gene A protein |
| NAGK | NM_017567 | N-Acetylglucosamine kinase |
| NPAS2 | NM_002518 | neuronal PAS domain protein 2 |
| NPTX1 | NM_002522 | neuronal pentraxin I precursor |
| NRP2 | NM_018534 | neuropilin 2 isoform 4 precursor |
| NUPL1 | NM_001008564 | nucleoporin like 1 isoform b |
| OSTM1 | NM_014028 | osteopetrosis associated transmembrane protein |
| PDCD4 | NM_014456 | programmed cell death 4 isoform 1 |
| PELI2 | NM_021255 | pellino 2 |
| PFKP | NM_002627 | phosphofructokinase, platelet |
| PLAU | NM_002658 | urokinase plasminogen activator preproprotein |
| PLCB1 | NM_015192 | phosphoinositide-specific phospholipase C beta 1 |
| PON2 | NM_000305 | paraoxonase 2 isoform 1 |
| PTHLH | NM_198965 | parathyroid hormone-like hormone isoform 1 |
| QKI | NM_206853 | quaking homolog, KH domain RNA binding isoform |
| RAB22A | NM_020673 | RAS-related protein RAB-22A |
| RHOC | NM_175744 | ras homolog gene family, member C |
| RNH1 | NM_002939 | ribonuclease/angiogenin inhibitor |
| RRM2 | NM_001034 | ribonucleotide reductase M2 polypeptide |
| SERPINE1 | NM_000602 | plasminogen activator inhibitor-1 |
| SESN1 | NM_014454 | sestrin 1 |
| SGPL1 | NM_003901 | sphingosine-1-phosphate lyase 1 |
| SLC1A4 | NM_003038 | solute carrier family 1, member 4 |
| SLC2A3 | NM_006931 | solute carrier family 2 (facilitated glucose |
| SNAP23 | NM_003825 | synaptosomal-associated protein 23 isoform |
| SPARC | NM_003118 | secreted protein, acidic, cysteine-rich |
| SPFH2 | NM_007175 | SPFH domain family, member 2 isoform 1 |
| STC1 | NM_003155 | stanniocalcin 1 precursor |
| SYNE1 | NM_015293 | nesprin 1 isoform beta |
| TBC1D2 | NM_018421 | TBC1 domain family, member 2 |
| TGFBR2 | NM_001024847 | TGF-beta type II receptor isoform A precursor |
| TNFRSF10B | NM_003842 | tumor necrosis factor receptor superfamily, |
| TXLNA | NM_175852 | Taxilin |
| UEV3 | NM_018314 | ubiquitin-conjugating enzyme E2-like |
| USP46 | NM_022832 | ubiquitin specific protease 46 |
| VANGL1 | NM_138959 | vang-like 1 |
| VLDLR | NM_001018056 | very low density lipoprotein receptor isoform b |
| WNT5A | NM_003392 | wingless-type MMTV integration site family, |
| ZNF331 | NM_018555 | zinc finger protein 331 |

Certain embodiments of the invention include determining expression of one or more marker, gene, or nucleic acid segment representative of one or more genes, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Proteins are typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on an miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, 4, and/or 5.

TABLE 5
Tumor associated mRNAs altered by hsa-miR-20a having prognostic or therapeutic value
for the treatment of various malignancies.

| Gene | | Cellular | | |
|---|---|---|---|---|
| Symbol | Gene Title | Process | Cancer Type | Reference |
| ANG | angiogenin | angiogenesis | BC, OC, M, PaC, UC, CeC | (Barton et al., 1997; Montero et al., 1998; Hartmann et al., |
| | | | | 1999; Miyake et al., 1999; Shimoyama et al., 1999; Bodner-Adler |
| | | | | et al., 2001) |
| CCND1 | cyclin D1 | Cell cycle | MCL, BC, SCCHN, OepC, | (Donnellan and Chetty, 1998) |
| | | | HCC, CRC, BldC, EC, OC, | |
| | | | M, AC, GB, GC, PaC | |
| CCNG1 | cyclin G1 | Cell cycle | OS, BC, PC | (Skotzko et al., 1995; Reimer et al., 1999) |
| CEBPD | C/EBP delta | transcription | PC | (Yang et al., 2001) |
| EPHB2 | EPH receptor | Signal | PC, GC, CRC, OC, G, BC | (Huusko et al., 2004; Nakada et al., 2004; Wu et al., 2004a; Jubb et al., |
| | B2 | transduction | | 2005; Davalos et al., 2007; Guo et al., 2006; Kokko et al., 2006; |
| | | | | Wu et al., 2006b) |
| EREG | epiregulin | Signal | BldC, CRC, PaC, PC | (Baba et al., 2000; Torring et al., 2000; Zhu et al., 2000; Thogersen |
| | | | | et al., transduction 2001) |
| ETS2 | ETS-2 | transcription | CeC, PC, TC, CRC, ESCC | (Simpson et al., 1997; Sementchenko et al., 1998; de Nigris et al., |
| | | | | 2001; Ito et al., 2002; Li et al., 2003) |
| FGFR3 | FGF-R3 | Signal | BldC, CRC, CeC, MM | (L'Hote and Knowles, 2005) |
| | | transduction | | |
| FGFR4 | FGF-R4 | Signal | TC, BC, OC, PaC | (Jaakkola et al., 1993; Shah et al., 2002; Ezzat et al., 2005) |
| | | transduction | | |
| FZD7 | Frizzled-7 | Signal | OepC, GC, HCC | (Tanaka et al., 1998; Kirikoshi et al., 2001; Merle et al., 2004) |
| | | transduction | | |
| ID4 | inhibitor of | transcription | BC, GC, L | (Chan et al., 2003; Yu et al., 2005; de Candia et al., 2006) |
| | DNA binding 4 | | | |
| IGFBP1 | IGFBP-1 | Signal | BC, CRC | (Firth and Baxter, 2002) |
| | | transduction | | |
| IL8 | IL-8 | Signal | BC, CRC, PaC, NSCLC, PC, | (Akiba et al., 2001; Sparmann and Bar-Sagi, 2004) |
| | | transduction | HCC | |
| JAK1 | Janus kinase 1 | Signal | PC | (Rossi et al., 2005) |
| | | transduction | | |
| JUN | c-Jun | transcription | HL, HCC | (Eferl et al., 2003; Weiss and Bohmann, 2004) |
| LHFP | lipoma | transcription | Li | (Petit et al., 1999) |
| | HMGIC fusion | | | |
| | partner | | | |
| LIMK1 | LIM kinase 1 | cell motility, | BC, PC | (Yoshioka et al., 2003) |
| | | invasion | | |
| P8 | P8 | transcription | BC, TC, PaC | (Ree et al., 1999; Su et al., 2001; Ito et al., 2005) |
| PDGFRL | PDGFR-like | Signal | CRC, NSCLC, HCC, PC | (Fujiwara et al., 1995; Komiya et al., 1997) |
| | | transduction | | |
| PLCB1 | PLC-beta1 | Signal | AML | (Lo Vasco et al., 2004) |
| | | transduction | | |
| RARRES1 | RAR | migration, | CRC, PC | (Zhang et al., 2004; Wu et al., 2006a) |
| | responder 1 | invasion | | |
| RHOC | RhoC | cell motility, | SCCHN, OepC, CRC, M, PC | (Bellovin et al., 2006; Faried et al., 2006; Kleer et al., 2006; Ruth et al., |
| | | invasion | | 2006; Yao et al., 2006) |
| SKP2 | SKP-2 | proteasomal | PaC, OC, BC, MFS, GB, EC, | (Kamata et al., 2005; Saigusa et al., 2005; Shibahara et al., 2005; |
| | | degradation | NSCLC, PC | Takanami, 2005; Einama et al., 2006; Huang et al., 2006; Sui et al., |
| | | | | 2006; Traub et al., 2006) |
| TGFBR2 | TGF beta | Signal | BC, CRC | (Markowitz, 2000; Lucke et al., 2001; Biswas et al., 2004) |
| | receptor type | transduction | | |
| | II | | | |
| TNFRSF10B | TRAIL-R2 | Apoptosis | NSCLC, SCCHN, GC, BC, | (Adams et al., 2005) |
| | | | NHL | |
| VTN | vitronectin | Cell adhesion | CRC, G, OC, M, BC | (Tomasini-Johansson et al., 1994; Carreiras et al., 1996; Lee et al., |
| | | | | 1998; Carreiras et al., 1999; Uhm et al., 1999; Aaboe |
| | | | | et al., 2003) |
| WNT5A | Wnt-5a | Signal | NSCLC, BC, M, GC, NB | (Saitoh et al., 2002; Blanc et al., 2005; Huang et al., 2005; Leris |
| | | transduction | | et al., 2005) |

Abbreviations:
AC, astrocytoma;
AML, acute myelogenous leukemia;
BC, breast carcinoma;
BldC, bladder carcinoma;
CeC, cervical carcinoma;
CRC, colorectal carcinoma;
EC, endometrial carcinoma;
ESCC, esophageal squamous cell carcinoma;
G, glioma;
GB, glioblastoma;
GC, gastric carcinoma;
HCC, hepatocellular carcinoma;
HL, Hodgkin lymphoma;
L, leukemia;
Li, lipoma;
M, melanoma;
MCL, mantle cell lymphoma;
MFS, myxofibrosarcoma;
MM, multiple myeloma;
NB, neuroblastoma;
NHL, non-Hodgkin lymphoma;
NSCLC, non-small cell lung carcinoma;
OC, ovarian carcinoma;
OepC, oesophageal carcinoma;
OS, osteosarcoma;
PaC, pancreatic carcinoma;
PC, prostate carcinoma;
SCCHN, squamous cell carcinoma of the head and neck;
TC, thyroid carcinoma;
UC, urothelial carcinoma.

It is contemplated that any method or composition described herein can be implemented with respect to any other method or composition described herein and that different embodiments may be combined. It is specifically contemplated that any methods and compositions discussed herein with respect to miRNA molecules, miRNA, genes, and nucleic acids representative of genes may be implemented with respect to synthetic nucleic acids. In some embodiments the synthetic nucleic acid is exposed to the proper conditions to allow it to become a processed or mature nucleic acid, such as a miRNA under physiological circumstances. The claims originally filed are contemplated to cover claims that are multiply dependent on any filed claim or combination of filed claims.

Also, any embodiment of the invention involving specific genes (including representative fragments there of), mRNA, or miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA.

It will be further understood that shorthand notations are employed such that a generic description of a gene or marker thereof, or of an miRNA refers to any of its gene family members (distinguished by a number) or representative fragments thereof, unless otherwise indicated. It is understood by those of skill in the art that a “gene family” refers to a group of genes having the same coding sequence or miRNA coding sequence. Typically, miRNA members of a gene family are identified by a number following the initial designation. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Exceptions to this shorthand notations will be otherwise identified.

Other embodiments of the invention are discussed throughout this application. Any embodiment discussed with respect to one aspect of the invention applies to other aspects of the invention as well and vice versa. The embodiments in the Example and Detailed Description section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION OF THE INVENTION

The present invention is directed to compositions and methods relating to the identification and characterization of genes and biological pathways related to these genes as represented by the expression of the identified genes, as well as use of miRNAs related to such, for therapeutic, prognostic, and diagnostic applications, particularly those methods and compositions related to assessing and/or identifying pathological conditions directly or indirectly related to miR-20a expression or the aberrant expression thereof.

In certain aspects, the invention is directed to methods for the assessment, analysis, and/or therapy of a cell or subject where certain genes have a reduced or increased expression (relative to normal) as a result of an increased or decreased expression of any one or a combination of miR-20 family members. In certain instances the expression profile and/or response to miR-20 expression or inhibition may be indicative of a disease or pathological condition, e.g., cancer.

Prognostic assays featuring any one or combination of the miRNAs listed or the markers listed (including nucleic acids representative thereof) could be used to assess an patient to determine what if any treatment regimen is justified. As with the diagnostic assays mentioned above, the absolute values that define low expression will depend on the platform used to measure the miRNA(s). The same methods described for the diagnostic assays could be used for prognostic assays.

### I. Therapeutic Methods

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to a nucleic acid molecule that is not produced naturally in a cell. In certain aspects the chemical structure deviates from a naturally-occurring nucleic acid molecule, such as an endogenous precursor miRNA or miRNA molecule. While in some embodiments, nucleic acids of the invention do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In some embodiments, there is a miRNA or a synthetic miRNA having a length of between 17 and 130 residues. The present invention concerns miRNA or synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain embodiments, synthetic miRNA have (a) an “miRNA region” whose sequence or binding region from 5′ to 3′ is identical to all or a segment of a mature miRNA sequence, and (b) a “complementary region” whose sequence from 5′ to 3′ is between 60% and 100% complementary to the miRNA sequence. In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA.

The term “complementary region” refers to a region of a synthetic miRNA that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. An miRNA inhibitor is between about 17 to 25 nucleotides in length and comprises a 5′ to 3′ sequence that is at least 90% complementary to the 5′ to 3′ sequence of a mature miRNA. In certain embodiments, an miRNA inhibitor molecule is 17, 18, 19, 20, 21, 22, 23, 24, or 25 nucleotides in length, or any range derivable therein. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for an miRNA inhibitor. Moreover, that portion of the nucleic acid sequence can be altered so that it is still comprises the appropriate percentage of complementarity to the sequence of a mature miRNA.

In some embodiments, of the invention, a synthetic miRNA contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an aminohexyl phosphate group, an acetyl group, 2′O-Me (2′oxygen-methyl), DMTO (4,4′-dimethoxytrityl with oxygen), fluoroscein, a thiol, or acridine, though other replacement groups are well known to those of skill in the art and can be used as well. This design element can also be used with an miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me 2° F. modification, a 2′H modification, a 2′amino modification, a 4′thioribose modification, or a phosphorothioate modification on the carboxy group linked to the carbon at position 6′. In further embodiments, there is one or more sugar modifications in the first or last 2 to 4 residues of the complementary region or the first or last 4 to 6 residues of the complementary region. This design element can also be used with an miRNA inhibitor. Thus, an miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having an miRNA region and a complementary region, there may be flanking sequences as well at either the 5′ or 3′ end of the region. In some embodiments, there is is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell an miRNA inhibitor; or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or an miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA or miRNA function. It is contemplated, however, that the miRNA molecule introduced into a cell is not a mature miRNA but is capable of becoming a mature miRNA under the appropriate physiological conditions. In cases in which a particular corresponding miRNA is being inhibited by a miRNA inhibitor, the particular miRNA will be referred to as the targeted miRNA. It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell. The inventors contemplate that a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s). In certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, that the methods may or may not be limited to providing only one or more synthetic miRNA molecules or only one or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA. Furthermore, any method articulated using a list of miRNAs using Markush group language may be articulated without the Markush group language and a disjunctive article (i.e., or) instead, and vice versa.

In some embodiments, there is a method for reducing or inhibiting cell proliferation in a cell comprising introducing into or providing to the cell an effective amount of (i) an miRNA inhibitor molecule or (ii) a synthetic or nonsynthetic miRNA molecule that corresponds to a miRNA sequence. In certain embodiments the methods involves introducing into the cell an effective amount of (i) a miRNA inhibitor molecule having a 5′ to 3′ sequence that is at least 90% complementary to the 5′ to 3′ sequence of one or more mature miRNA.

Certain embodiments of the invention include methods of treating a pathologic condition, in particular cancer, e.g., lung or liver cancer. In one aspect, the method comprises contacting a target cell with one or more nucleic acid, synthetic miRNA, or miRNA comprising at least one nucleic acid segment having all or a portion of a miRNA sequence. The segment may be 5, 6, 7, 8, 9, 10, 11, 12, 13, 14, 15, 16, 17, 18, 19, 20, 21, 22, 23, 24, 25, 30 or more nucleotides or nucleotide analog, including all integers there between. An aspect of the invention includes the modulation of gene expression, miRNA expression or function or mRNA expression or function within a target cell, such as a cancer cell.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA or gene sequence. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may affect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided an miRNA or miRNA molecule corresponding to a particular miRNA by administering to the cell or organism a nucleic acid molecule that functions as the corresponding miRNA once inside the cell. The form of the molecule provided to the cell may not be the form that acts an miRNA once inside the cell. Thus, it is contemplated that in some embodiments, a synthetic miRNA or a nonsynthetic miRNA is provided such that it becomes processed into a mature and active miRNA once it has access to the cell's miRNA processing machinery. In certain embodiments, it is specifically contemplated that the miRNA molecule provided is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting an miRNA to modulate in a cell or organism. The term “targeting an miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with an miRNA inhibitor, which effectively inhibits the targeted miRNA in the cell or organism (negative modulation).

In some embodiments, the miRNA targeted to be modulated is an miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA or its targets.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of an miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied preventatively, particularly in a patient identified to be potentially in need of the therapy or at risk of the condition or disease for which a therapy is needed.

In addition, methods of the invention concern employing one or more nucleic acids corresponding to an miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, 5-fluorouracil, alemtuzumab, amrubicin, bevacizumab, bleomycin, bortezomib, busulfan, camptothecin, capecitabine, carboplatin, cetuximab, chlorambucil, cisplatin (CDDP), COX-2 inhibitors (e.g., celecoxib), cyclophosphamide, cytarabine, dactinomycin, dasatinib, daunorubicin, dexamethasone, docetaxel, doxorubicin (adriamycin), EGFR inhibitors (gefitinib and cetuximab), erlotinib, estrogen receptor binding agents, etoposide (VP16), everolimus, farnesyl-protein transferase inhibitors, gefitinib, gemcitabine, gemtuzumab, ibritumomab, ifosfamide, imatinib mesylate, larotaxel, lapatinib, lonafarnib, mechlorethamine, melphalan, methotrexate, mitomycin, navelbine, nitrosurea, nocodazole, oxaliplatin, paclitaxel, plicomycin, procarbazine, raloxifene, rituximab, sirolimus, sorafenib, sunitinib, tamoxifen, taxol, taxotere, temsirolimus, tipifarnib, tositumomab, transplatinum, trastuzumab, vinblastin, vincristin, or vinorelbine or any analog or derivative variant of the foregoing.

Generally, inhibitors of miRNAs can be given to decrease the activity of an endogenous miRNA. For example, inhibitors of miRNA molecules that increase cell proliferation can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating or inhibiting a kinase (e.g., Erk), activating/inducing or inhibiting hTert, inhibit stimulation of growth promoting pathway (e.g., Stat 3 signaling), reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

### II. Pharmaceutical Formulations and Delivery

Methods of the present invention include the delivery of an effective amount of a miRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a miRNA or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the tumor or affected area being treated may not, at least initially, be resectable. Treatments with compositions of the invention may increase the resectability of the tumor due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of μg or mg of miRNA or miRNA mimetic. Alternatively, the amount specified may be the amount administered as the average daily, average weekly, or average monthly dose.

miRNA can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a miRNA or an expression construct encoding such or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

Various methods for nucleic acid delivery are described, for example in Sambrook et al., 1989, Molecular Cloning: A Laboratory Manual, Cold Spring Harbor Laboratory, New York; and Ausubel et al., 1994, Current Protocols in Molecular Biology, John Wiley & Sons, New York. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. By way of example, and not by limitation, the nucleic acid delivery system can be provided to the cell by endocytosis; receptor targeting; coupling with native or synthetic cell membrane fragments; physical means such as electroporation; combining the nucleic acid delivery system with a polymeric carrier, such as a controlled release film or nanoparticle or microparticle or biocompatible molecules or biodegradable molecules; with vector. The nucleic acid delivery system can be injected into a tissue or fluid surrounding the cell, or administered by diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

C. Combination Treatments

In certain embodiments, the compositions and methods of the present invention involve a miRNA, or expression construct encoding such. These miRNA composition can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the miRNA or second therapy at the same or different time. This may be achieved by contacting the cell with one or more compositions or pharmacological formulation that includes or more of the agents, or by contacting the cell with two or more distinct compositions or formulations, wherein one composition provides (1) miRNA; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

b. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

e. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 6 is a non-limiting list of several known anti-cancer immunotherapeutic agents and their targets. It is contemplated that one or more of these therapies may be employed with the miRNA therapies described herein.

TABLE 6

| Generic Name | Target |
|---|---|
| Cetuximab | EGFR |
| Panitumumab | EGFR |
| Trastuzumab | erbB2 receptor |
| Bevacizumab | VEGF |
| Alemtuzumab | CD52 |
| gemtuzumab ozogamicin | CD33 |
| Rituximab | CD20 |
| Tositumomab | CD20 |
| Matuzumab | EGFR |
| ibritumomab tiuxetan | CD20 |
| Tositumomab | CD20 |
| HuPAM4 | MUC1 |
| MORAb-009 | Mesothelin |
| G250 | carbonic anhydrase IX |
| mAb 8H9 | 8H9 antigen |
| M195 | CD33 |
| Ipilimumab | CTLA4 |
| HuLuc63 | CS1 |
| Alemtuzumab | CD53 |
| Epratuzumab | CD22 |
| BC8 | CD45 |
| HuJ591 | Prostate specific membrane antigen |
| hA20 | CD20 |
| Lexatumumab | TRAIL receptor-2 |
| Pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| Volociximab | anti-α5β1 integrin |
| GC1008 | TGFβ |
| HCD122 | CD40 |
| Siplizumab | CD2 |
| MORAb-003 | Folate receptor alpha |
| CNTO 328 | IL-6 |
| MDX-060 | CD30 |
| Ofatumumab | CD20 |
| SGN-33 | CD33 |

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p16INK4 has been biochemically characterized as a protein that specifically binds to and inhibits CDK4, and thus may regulate Rb phosphorylation (Serrano et al., 1993; Serrano et al., 1995). Since the p16INK4 protein is a CDK4 inhibitor (Serrano, 1993), deletion of this gene may increase the activity of CDK4, resulting in hyperphosphorylation of the Rb protein. p16 also is known to regulate the function of CDK6.

p16INK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p16INK4 gene are frequent in human tumor cell lines. This evidence suggests that the p16INK4 gene is a tumor suppressor gene. This interpretation has been challenged, however, by the observation that the frequency of the p16INK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16INK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### III. miRNA Molecules

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene or its gene product. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

A. Array Preparation

Certain embodiments of the present invention concerns the preparation and use of mRNA or nucleic acid arrays, miRNA or nucleic acid arrays, and/or miRNA or nucleic acid probe arrays, which are macroarrays or microarrays of nucleic acid molecules (probes) that are fully or nearly complementary (over the length of the prove) or identical (over the length of the prove) to a plurality of nucleic acid, mRNA or miRNA molecules, precursor miRNA molecules, or nucleic acids derived from the various genes and gene pathways modulated by miR-20 miRNAs and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of marker RNA and/or miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect miRNA, or genes or nucleic acid representative of genes; consequently, methods and compositions may be used with a variety of different types of nucleic acid arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to mRNA and/or miRNA targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, 20 to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm².

The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the RNA and/or miRNA of a wide variety of samples can be analyzed using the arrays, index of probes, or array technology of the invention. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells, particularly cancer or hyperproliferative cells. In certain embodiments, samples may be, but are not limited to, biopsy, or cells purified or enriched to some extent from a biopsy or other bodily fluids or tissues. Alternatively, the sample may not be a biological sample, but be a chemical mixture, such as a cell-free reaction mixture (which may contain one or more biological enzymes).

C. Hybridization

After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

D. Differential Expression Analyses

Arrays of the invention can be used to detect differences between two samples. Specifically contemplated applications include identifying and/or quantifying differences between miRNA or gene expression from a sample that is normal and from a sample that is not normal, between a disease or condition and a cell not exhibiting such a disease or condition, or between two differently treated samples. Also, miRNA or gene expression may be compared between a sample believed to be susceptible to a particular disease or condition and one believed to be not susceptible or resistant to that disease or condition. A sample that is not normal is one exhibiting phenotypic or genotypic trait(s) of a disease or condition, or one believed to be not normal with respect to that disease or condition. It may be compared to a cell that is normal with respect to that disease or condition. Phenotypic traits include symptoms of, or susceptibility to, a disease or condition of which a component is or may or may not be genetic, or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Particularly, arrays can be used to evaluate samples with respect to pathological condition such as cancer and related conditions. It is specifically contemplated that the invention can be used to evaluate differences between stages or sub-classifications of disease, such as between benign, cancerous, and metastatic tissues or tumors.

Phenotypic traits to be assessed include characteristics such as longevity, morbidity, expected survival, susceptibility or receptivity to particular drugs or therapeutic treatments (drug efficacy), and risk of drug toxicity. Samples that differ in these phenotypic traits may also be evaluated using the compositions and methods described.

In certain embodiments, miRNA and/or expression profiles may be generated to evaluate and correlate those profiles with pharmacokinetics or therapies. For example, these profiles may be created and evaluated for patient tumor and blood samples prior to the patient's being treated or during treatment to determine if there are miRNA or genes whose expression correlates with the outcome of the patient's treatment. Identification of differential miRNAs or genes can lead to a diagnostic assay for evaluation of tumor and/or blood samples to determine what drug regimen the patient should be provided. In addition, it can be used to identify or select patients suitable for a particular clinical trial. If an expression profile is determined to be correlated with drug efficacy or drug toxicity that profile is relevant to whether that patient is an appropriate patient for receiving a drug, for receiving a combination of drugs, or for a particular dosage of the drug.

In addition to the above prognostic assay, samples from patients with a variety of diseases can be evaluated to determine if different diseases can be identified based on miRNA and/or related gene expression levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, which is hereby incorporated by reference in its entirety.

E. Other Assays

In addition to the use of arrays and microarrays, it is contemplated that a number of different assays could be employed to analyze miRNAs or related genes, their activities, and their effects. Such assays include, but are not limited to, nucleic acid amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### IV. Nucleic Acids

The present invention concerns nucleic acids, modified or mimetic nucleic acids, miRNAs, mRNAs, genes, and representative fragments thereof that can be labeled, used in array analysis, or employed in diagnostic, therapeutic, or prognostic applications, particularly those related to pathological conditions such as cancer. The molecules may have been endogenously produced by a cell, or been synthesized or produced chemically or recombinantly. They may be isolated and/or purified. Each of the miRNAs described herein and include the corresponding SEQ ID NO and accession numbers for these miRNA sequences. The name of a miRNA is often abbreviated and referred to without a “hsa-” prefix and will be understood as such, depending on the context. Unless otherwise indicated, miRNAs referred to in the application are human sequences identified as miR-X or let-X, where X is a number and/or letter.

In certain aspects, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the world wide web at sanger.ac.uk. Moreover, in some embodiments, a miRNA probe is used that does not correspond to a known human miRNA. It is contemplated that these non-human miRNA probes may be used in embodiments of the invention or that there may exist a human miRNA that is homologous to the non-human miRNA. In other embodiments, any mammalian cell, biological sample, or preparation thereof may be employed.

In some embodiments of the invention, methods and compositions involving miRNA may concern miRNA, markers (mRNAs), and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, mRNA, mRNA probes, control nucleic acids, and other probes and primers.

In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or other nucleic acid or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of SEQ ID NO: 1 through SEQ ID NO:269, accession number, or any other sequence disclosed herein. Typically, the commonly used name of the miRNA is given (with its identifying source in the prefix, for example, “hsa” for human sequences) and the processed miRNA sequence. Unless otherwise indicated, a miRNA without a prefix will be understood to refer to a human miRNA. Moreover, a lowercase letter in a miRNA name may or may not be lowercase; for example, hsa-mir-130b can also be referred to as miR-130B. The term “miRNA probe” refers to a nucleic acid probe that can identify a particular miRNA or structurally related miRNAs.

It is understood that some nucleic acids are derived from genomic sequences or a gene. In this respect, the term “gene” is used for simplicity to refer to the genomic sequence encoding the precursor nucleic acid or miRNA for a given miRNA or gene. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, miRNA may encompass a molecule that comprises one or more complementary or self-complementary strand(s) or “complement(s)” of a particular sequence. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

It is understood that a “synthetic nucleic acid” of the invention means that the nucleic acid does not have all or part of a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

While embodiments of the invention may involve synthetic miRNAs or synthetic nucleic acids, in some embodiments of the invention, the nucleic acid molecule(s) need not be “synthetic.” In certain embodiments, a non-synthetic nucleic acid or miRNA employed in methods and compositions of the invention may have the entire sequence and structure of a naturally occurring mRNA or miRNA precursor or the mature mRNA or miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not an miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA.” Corresponding miRNA sequences that can be used in the context of the invention include, but are not limited to, all or a portion of those sequences in SEQ ID NOs: 1-269, as well as any other miRNA sequence, miRNA precursor sequence, or any sequence complementary thereof. In some embodiments, the sequence is or is derived from or contains all or part of a sequence identified herein to target a particular miRNA (or set of miRNAs) that can be used with that sequence. Any 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130 140, 150, 160, 170, 180, 190, 200, 210, 220, 230, 240, 250, 260 or any number or range of sequences there between may be selected to the exclusion of all non-selected sequences.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

A. Nucleobase, Nucleoside, Nucleotide, and Modified Nucleotides

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring. Different types of covalent attachment(s) of a nucleobase to a nucleobase linker moiety are known in the art (Kornberg and Baker, 1992).

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids include those in: U.S. Pat. Nos. 5,681,947, 5,652,099 and 5,763,167, 5,614,617, 5,670,663, 5,872,232, 5,859,221, 5,446,137, 5,886,165, 5,714,606, 5,672,697, 5,466,786, 5,792,847, 5,223,618, 5,470,967, 5,378,825, 5,777,092, 5,623,070, 5,610,289, 5,602,240, 5,858,988, 5,214,136, 5,700,922, 5,708,154, 5,728,525, 5,637,683, 6,251,666, 5,480,980, and 5,728,525, each of which is incorporated herein by reference in its entirety.

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, Biosearch Technologies and NEN. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Preparation of Nucleic Acids

A nucleic acid may be made by any technique known to one of ordinary skill in the art, such as for example, chemical synthesis, enzymatic production, or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

Alternatively, nucleic acid synthesis is performed according to standard methods. See, for example, Itakura and Riggs (1980) and U.S. Pat. Nos. 4,704,362, 5,221,619, and 5,583,013, each of which is incorporated herein by reference. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. See also Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

C. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried and resuspended in a liquid and volume appropriate for subsequent manipulation.

### V. Labels and Labeling Techniques

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed. Terminal transferase catalyzes the addition of nucleotides to the 3′ terminus of a nucleic acid. Polynucleotide phosphorylase can polymerize nucleotide diphosphates without the need for a primer.

B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phicoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

C. Visualization Techniques

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### VI. Kits

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (acute myeloid leukemia), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ ID NOS: 1-267. In certain embodiments, a kit or array of the invention can contain one or more probes for the miRNAs identified by SEQ ID NOS:1-267. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Gene Expression Analysis Following Transfection with HSA-MiR-20a

miRNAs are believed to regulate gene expression by binding to target mRNA transcripts and (1) initiating transcript degradation or (2) altering protein translation from the transcript. Translational regulation leading to an up or down change in protein expression may lead to changes in activity and expression of downstream gene products and genes that are in turn regulated by those proteins. These numerous regulatory effects may be revealed as changes in the global mRNA expression profile. Microarray gene expression analyses were performed to identify genes that are mis-regulated by hsa-miR-20a expression.

Synthetic Pre-miR-20a (Ambion) was reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAmp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1 supra.

Manipulation of the expression levels of the genes listed in Table 1 represents a potentially useful therapy for cancer and other diseases in which increased or reduced expression of hsa-miR-20a has a role in the disease.

### Example 2

### Cellular pathways affected by HSA-MiR-20a

The mis-regulation of gene expression by hsa-miR-20a (Table 1) affects many cellular pathways that represent potential therapeutic targets for the control of cancer and other diseases and disorders. The inventors determined the identity and nature of the cellular genetic pathways affected by the regulatory cascade induced by hsa-miR-20a expression. Cellular pathway analyses were performed using Ingenuity Pathways Analysis (Ingenuity® Systems, Redwood City, Calif.). The most significantly affected pathways following over-expression of hsa-miR-20a in A549 cells are shown in Table 2 supra.

These data demonstrate that hsa-miR-20a directly or indirectly affects the expression of numerous cellular growth-, cellular proliferation-, cell signaling-, and cell development-related genes and thus primarily affects functional pathways related to, cellular growth, cellular development, and cell proliferation. Those cellular processes all have integral roles in the development and progression of various cancers. Manipulation of the expression levels of genes in the cellular pathways shown in Table 2 represents a potentially useful therapy for cancer and other diseases in which increased or reduced expression of hsa-miR-20a has a role in the disease.

### Example 3

### Predicted Gene Targets of Hsa-MiR-20a

Gene targets for binding of and regulation by hsa-miR-20a were predicted using the proprietary algorithm miRNA Target™ (Asuragen) and are shown in Table 3 supra.

The predicted gene targets that exhibited altered mRNA expression levels in human cancer cells, following transfection with pre-miR hsa-miR-20a, are shown in Table 4 supra.

The predicted gene targets of hsa-miR-20a whose mRNA expression levels are affected by hsa-miR-20a represent particularly useful candidates for cancer therapy and therapy of other diseases through manipulation of their expression levels.

### Example 4

### Cancer Related Gene Expression Altered by HSA-MiR-20a

Cell proliferation and survival pathways are commonly altered in tumors (Hanahan and Weinberg, 2000). The inventors have shown that hsa-miR-20a directly or indirectly regulates the transcripts of proteins that are critical in the regulation of these pathways. Many of these targets have inherent oncogenic or tumor suppressor activity. Hsa-miR-20a targets that are associated with various cancer types are shown in Table 5.

Hsa-miR-20a targets of particular interest are genes and their products that function in the regulation of intracellular signal transduction. When deregulated, many of these proteins contribute to the malignant phenotype in vitro and in vivo. Hsa-miR-20a affects intracellular signaling at various layers and controls the expression of secretory growth factors, transmembrane growth factor receptors, and cytoplasmic signaling molecules. Examples of secreted proteins regulated by hsa-miR-20a are Eregulin (EREG), Wnt5a and the inflammatory chemokine IL-8. Eregulin (EREG) belongs to the epidermal growth factor (EGF) family and binds to EGF receptors such as ErbB (Shelly et al., 1998). Eregulin expression is rare in adult tissues but is elevated in various cancer types (Toyoda et al., 1997). Eregulin may also play a direct role in tumorigenesis, as it contributes to tumor formation of colon cancer cells (Baba et al., 2000). Since transfection of hsa-miR-20a decreases levels of EREG transcripts, hsa-miR-20a might intervene with the oncogenic activity of Eregulin. Wnt family members are cysteine-rich proteins that function as growth factors. Wnt5a plays a role in patterning decisions in the embryonic nervous system during development and is linked to the progression of melanoma and the invasion of ductal breast carcinomas (Jonsson et al., 2002; Weeraratna et al., 2002). Transmembrane receptors targeted by hsa-miR-20a include platelet-derived growth factor receptor-like (PDGFR-L, also known as PDGF-receptor beta-like tumor suppressor, PRLTS), transforming growth factor beta (TGF-β) receptor 2 (TGFBR2), tumor necrosis factor-related apoptosis inducer ligand (TRAIL) receptor 2 (TRAIL-R2; also known as tumor necrosis factor receptor superfamily member B10; TNFSFB10), retinoic acid receptor responder 1 (RARRES1), ephrin B2 receptor (EphB2) and fibroblast growth factor receptors (FGFR) 3 and 4. FGFR-3 and FGFR-4 are commonly overexpressed in multiple cancer types and appear to have angiogenic activity (Chandler et al., 1999). In contrast, PDGFR-L, TRAIL-R2, RARRES1 and TGFBR-2 are putative tumor suppressors. PDGFR-L shows loss of function in a broad variety of cancers either by loss of heterozygosity (LOH) or missense and frame-shift mutation (Fujiwara et al., 1995; Komiya et al., 1997). TRAIL-R2 interacts with TRAIL and stimulates pro-apoptotic pathways in various cell types (Fesik, 2005). The corresponding gene is located at a chromosomal region (8p22-23) that is a frequent site of LOH in numerous human neoplasias (Adams et al., 2005). Therefore, loss of TRAIL-R2 may contribute to the malignant phenotype of these cancers. RARRES1 is a transmembrane protein that is lost or shows decreased expression levels in several types of cancer (Wu et al., 2006a and references therein). TGFBR-2 forms a functional complex with TGFBR-1 and is the primary receptor for TGF-β (Massague et al., 2000). Central role of TGF-β is inhibition of cellular growth of numerous cell types, such as epithelial, endothelial, hematopoietic neural and mesenchymal cells. Many mammary and colorectal carcinomas with microsatellite instability harbor inactivating mutations of TGFBR-2, and therefore escape the growth-inhibitory function of TGF-β (Markowitz et al., 1995; Lucke et al., 2001). Ephrin B2 receptor may have a suppressor role in prostate and colorectal carcinomas, as inactivation of EphB2 accelerates tumorigenesis (Guo et al., 2006). Cytoplasmic signaling molecules regulated by hsa-miR-20a include RhoC and phospholipase C beta-1 (PLC beta-1). RhoC is a small GTPase that regulates cell motility in normal cells and promotes metastasis during tumorigenesis (Wheeler and Ridley, 2004; Wu et al., 2004b). Accordingly, RhoC levels are progressively increased as tumors become more aggressively metastatic. PLC beta-1 catalyzes the generation of inositol-1,4,5-trisphosphate (IP3) and diacylglycerol (DAG) from phosphatidylinositol-bis-phosphate (PIP2), regulating proliferative signals and checkpoints of the cell cycle (Lo Vasco et al., 2004).

Another class of genes regulated by hsa-miR-20a encodes transcription factors. Among these are the basic region/leucine zipper proteins (bZIP) Jun and CCAAT/enhancer-binding protein delta (C/EBP delta), the former of which is the cellular homolog of the avian oncoprotein v-Jun (Maki et al., 1987). Hsa-miR-20a also regulates the transcription factor ETS2 which is the mammalian homolog of the v-Ets oncoprotein originally isolated from the transforming erythroblastosis virus E26 (Leprince et al., 1983). The corresponding ETS2 gene is frequently subject to chromosomal translocation in acute myeloid leukemia (AML) and may be critical in the development of the disease (Sacchi et al., 1986). Exogenous introduction of hsa-miR-20a induces elevated expression of ID4 (inhibitor of DNA-binding 4), a potential tumor suppressor that is selectively silenced by methylation in leukemia (Yu et al., 2005). ID4 carries a helix-loop-helix domain but lacks an intact DNA-binding domain. Thus, ID4 functions as a dominant negative to other HLH transcription factors, e.g. c-Myc which is deregulated in the vast majority of human cancers (Grandori et al., 2000; Nesbit et al., 1999).

Further growth-related genes regulated by hsa-miR-20a are the cyclins D1 and G1, as well as S-phase kinase-associated protein 2 (Skp2). Cyclins are co-factors of cyclin-dependent kinases (CDKs) and function in the progression of the cell cycle. Cyclin D1 is required for the transition from G1 into S phase and is overexpressed in numerous cancer types (Donnellan and Chetty, 1998). Hsa-miR-20a negatively regulates cyclin D1 expression and therefore might interfere with abnormal cell growth that depends on high levels of cyclin D1. In contrast, cyclin G1 has growth inhibitory activity and is upregulated by hsa-miR-20a (Zhao et al., 2003). Skp2 is a component of the multi-subunit E3 ubiquitin ligase complex that ear-marks proteins for proteasomal degradation. A well characterized target is the CDK inhibitor p27 which offers an explanation for the cell cycle promoting activity of Skp2 (Carrano et al., 1999). Skp2 is inherently oncogenic and shows elevated levels in various cancer types (Gstaiger et al., 2001; Kamata et al., 2005; Saigusa et al., 2005; Einama et al., 2006).

In summary, hsa-miR-20a governs the activity of proteins that are critical regulators of cell proliferation and survival. These targets are frequently deregulated in human cancer. Based on this review of the genes and related pathways that are regulated by miR-20a, introduction of hsa-miR-20a or an anti-hsa-miR-20a into a variety of cancer cell types would likely result in a therapeutic response.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,870,287
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,384,261
U.S. Pat. No. 5,399,363
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,445,934
U.S. Pat. No. 5,446,137
U.S. Pat. No. 5,466,468
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,543,158
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,641,515
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,739,169
U.S. Pat. No. 5,744,305
U.S. Pat. No. 5,760,395
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,846,225
U.S. Pat. No. 5,846,945
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent Ser. No. 09/545,207,
U.S. patent Ser. No. 10/667,126
U.S. patent Ser. No. 11/141,707
U.S. patent Ser. No. 11/273,640
U.S. patent Ser. No. 11/349,727
U.S. Patent Ser. No. 60/575,743
U.S. Patent Ser. No. 60/649,584
U.S. Patent Ser. No. 60/650,807
Aaboe et al., Biochim Biophys Acta, 1638(1):72-82, 2003.
Adams et al., Cancer Lett, 220(2):137-144, 2005.
Akiba et al., Int J Oncol, 18(2):257-264, 2001.
Ambros, Cell, 107(7):823-826, 2001.
Arap et al., Cancer Res., 55(6):1351-1354, 1995.
Austin-Ward and Villaseca, Revista Medica de Chile, 126(7):838-845, 1998.
Baba et al., Cancer Res, 60(24):6886-6889, 2000.
Bagga et al., Cell, 122(4):553-563, 2005.
Barton et al., Clin Cancer Res, 3(9):1579-1586, 1997.
Bellovin et al., Oncogene, 25(52):6959-6967, 2006.
Biswas et al., Cancer Res, 64(14):4687-4692, 2004.
Blanc et al., Cancer Lett, 228(1-2):117-123, 2005.
Bodner-Adler et al., Anticancer Res, 21(1B):809-812, 2001.
Brennecke et al., Cell, 113(1):25-36, 2003.
Bukowski et al., Clinical Cancer Res., 4(10):2337-2347, 1998.
Caldas et al., Cancer Res., 54:3568-3573, 1994.
Caldas et al., Nat. Genet., 8(1):27-32, 1994.
Calin et al., Proc. Natl. Acad. Sci. USA, 99(24):15524-15529, 2002.
Carrano et al., Nat Cell Biol, 1(4):193-199, 1999.
Carreiras et al., Gynecol Oncol, 62(2):260-267, 1996.
Carreiras et al., Gynecol Oncol, 72(3):312-322, 1999.
Carrington and Ambros, Science, 301(5631):336-338, 2003.
Chan et al., Oncogene, 22(44):6946-6953, 2003.
Chandler et al., Int J Cancer, 81(3):451-458, 1999.
Cheng et al., Cancer Res., 54(21):5547-5551, 1994.
Christodoulides et al., Microbiology, 144 (Pt 11):3027-3037, 1998.
Cummins et al., In: IRT: Nucleosides and nucleosides, La Jolla Calif., 72, 1996.
Davalos et al., Oncogene, 26(2):308-311, 2006.
Davidson et al., J. Immunother., 21(5):389-398, 1998.
de Candia et al., Hum Pathol, 37(8):1032-1041, 2006.
de Nigris et al., Cancer Res, 61(5):2267-2275, 2001.
Denli et al., Trends Biochem. Sci., 28:196, 2003.
Didenko, Biotechniques, 31(5):1106-1116, 1118, 1120-1121, 2001.
Dillman, Cancer Biother. Radiopharm., 14(1):5-10, 1999.
Donnellan and Chetty, Mol Pathol, 51(1):1-7, 1998.
Eferl et al., Cell, 112(2):181-192, 2003.
Einama et al., Pancreas, 32(4):376-381, 2006.
Emptage et al., Neuron, 29(1):197-208, 2001.
EP 266,032
EP 373 203
EP 785 280
EP 799 897
Esquela-Kerscher and Slack, Nat Rev Cancer, 6(4):259-269, 2006.
Ezzat et al., Clin Cancer Res, 11(3):1336-1341, 2005.
Faried et al., Eur J Cancer, 42(10):1455-1465, 2006.
Fesik, Nat Rev Cancer, 5(11):876-885, 2005.
Firth and Baxter, Endocr Rev, 23(6):824-854, 2002.
Fodor et al., Science, 251:767-777, 1991.
Frochler et al., Nucleic Acids Res., 14(13):5399-5407, 1986.
Fujiwara et al., Oncogene, 10(5):891-895, 1995.
Grandori et al., Annu Rev Cell Dev Biol, 16:653-699, 2000.
Griffey et al., J. Mass Spectrom, 32(3):305-13, 1997.
Gstaiger et al., Proc Natl Acad Sci USA, 98(9):5043-5048, 2001.
Guo et al., Carcinogenesis, 27(3):454-464, 2006.
Hanahan and Weinberg, Cell, 100(1):57-70, 2000.
Hanibuchi et al., Int. J. Cancer, 78(4):480-485, 1998.
Hartmann et al., Cancer Res, 59(7):1578-1583, 1999.
He et al., Nature, 435(7043):828-833, 2005a.
He et al., Proc. Natl. Acad. Sci. USA, 102(52):19075-19080, 2005b.
Hellstrand et al., Acta Oncologica, 37(4):347-353, 1998.
Huang et al., Clin Cancer Res, 12(2):487-498, 2006.
Huang et al., J Clin Oncol, 23(34):8765-8773, 2005.
Hui and Hashimoto, Infection Immun., 66(11):5329-5336, 1998.
Hussussian et al., Nat. Genet., 8(1):15-21, 1994.
Huusko et al., Nat Genet, 36(9):979-983, 2004.
Itakura and Riggs, Science, 209:1401-1405, 1980.
Ito et al., Anticancer Res, 22(3): 1581-1584, 2002.
Ito et al., Anticancer Res, 25(5):3419-3423, 2005.
Jaakkola et al., Int J Cancer, 54(3):378-382, 1993.
Jonsson et al., Cancer Res, 62(2):409-416, 2002.
Ju et al., Gene Ther., 7(19):1672-1679, 2000.
Jubb et al., Clin Cancer Res, 11(14):5181-5187, 2005.
Kamata et al., J Cancer Res Clin Oncol, 131(9):591-596, 2005.
Kamb et al., Nat. Genet., 8(1):23-26, 1994.
Kamb et al., Science, 2674:436-440, 1994.
Kirikoshi et al., Int J Oncol, 19(1):111-115, 2001.
Kleer et al., Clin Cancer Res, 12(15):4485-4490, 2006.
Klostermeier and Millar, Biopolymers, 61(3):159-79, 2002.
Kokko et al., BMC Cancer, 6:145, 2006.
Komiya et al., Jpn J Cancer Res, 88(4):389-393, 1997.
Kornberg and Baker, DNA Replication, 2nd Ed., Freeman, San Francisco, 1992.
Lagos-Quintana et al., Science, 294(5543):853-858, 2001.
Lau et al., Science, 294(5543):858-862, 2001.
Lee and Ambros, Science, 294(5543):862-864, 2001.
Lee et al., Cell Struct Funct, 23(4):193-199, 1998.
Leprince et al., Nature, 306(5941):395-397, 1983.
Leris et al., Anticancer Res, 25(2A):731-734, 2005.
L'Hote and Knowles, Exp Cell Res, 304(2):417-431, 2005.
Li et al., World J Gastroenterol, 9(2):205-208, 2003.
Lim et al., Nature, 433(7027):769-773, 2005
Lo Vasco et al., Leukemia, 18(6):1122-1126, 2004.
Lu et al., Nature, 435(7043):834-838, 2005.
Lucke et al., Cancer Res, 61(2):482-485, 2001.
Maki et al., Proc Natl Acad Sci USA, 84(9):2848-2852, 1987.
Markowitz et al., Science, 268(5215):1336-1338, 1995.
Markowitz, Biochim Biophys Acta, 1470(1):M13-20, 2000.
Marsters et al., Recent Prog. Horm. Res., 54:225-234, 1999.
Massague et al., Cell, 103(2):295-309, 2000.
Merle et al., Gastroenterology, 127(4):1110-1122, 2004.
Miyake et al., Cancer, 86(2):316-324, 1999.
Montero et al., Clin Cancer Res, 4(9):2161-2168, 1998.
Mori et al., Cancer Res., 54(13):3396-3397, 1994.
Nakada et al., Cancer Res, 64(9):3179-3185, 2004.
Nesbit et al., Oncogene, 18(19):3004-3016, 1999.
Nobri et al, Nature (London), 368:753-756, 1995.
O'Donnell et al., Nature, 435:839-843, 2005.
Okamoto et al., Proc. Natl. Acad. Sci. USA, 91(23): 11045-11049, 1994.
Olsen et al., Dev. Biol., 216:671, 1999.
Orlow et al., Cancer Res, 54(11):2848-2851, 1994.
Orlow et al., Int. J. Oncol., 15(1):17-24, 1994.
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/21944.
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
PCT Appln. WO0138580
Petit et al., Genomics, 57(3):438-441, 1999.
Pietras et al., Oncogene, 17(17):2235-2249, 1998.
Pruitt et al., Nucleic Acids Res, 33(1):D501-D504, 2005.
Qin et al., Proc. Natl. Acad. Sci. USA, 95(24):14411-14416, 1998.
Ree et al., Cancer Res, 59(18):4675-4680, 1999.
Reimer et al., J Biol Chem, 274(16):11022-11029, 1999.
Reinhart et al., Nature, 403(6772):901-906, 2000.
Remington's Pharmaceutical Sciences, 15^{th} ed., pages 1035-1038 and 1570-1580, Mack Publishing Company, Easton, Pa., 1980.
Rossi et al., Cancer Genet Cytogenet, 161(2):97-103, 2005.
Ruth et al., J Invest Dermatol, 126(4):862-868, 2006.
Sacchi et al., Science, 231(4736):379-382, 1986.
Saigusa et al., Cancer Sci, 96(10):676-683, 2005.
Saitoh et al., Int J Mol Med, 9(5):515-519, 2002.
Sambrook and Russell, Molecular Cloning: A Laboratory Manual 3^{rd} Ed., Cold Spring Harbor Laboratory Press, 2001.
Sambrook et al., In: DNA microarrays: a molecular cloning manual, Cold Spring Harbor Laboratory Press, Cold Spring Harbor, N.Y., 2003.
Sambrook et al., In: Molecular cloning: a laboratory manual, 2^{nd} Ed., Cold Spring Harbor Laboratory Press, Cold Spring Harbor, N.Y., 1989.
Seggerson et al., Dev. Biol., 243:215, 2002.
Sementchenko et al., Oncogene, 17(22):2883-2888, 1998.
Serrano et al., Nature, 366:704-707, 1993.
Serrano et al., Science, 267(5195):249-252, 1995.
Shah et al., Oncogene, 21(54):8251-8261, 2002.
Shelly et al., J Biol Chem, 273(17):10496-10505, 1998.
Shibahara et al., Anticancer Res, 25(3B):1881-1888, 2005.
Shimoyama et al., Clin Cancer Res, 5(5):1125-1130, 1999.
Simpson et al., Oncogene, 14(18):2149-2157, 1997.
Skotzko et al., Cancer Res, 55(23):5493-5498, 1995.
Sparmann and Bar-Sagi, Cancer Cell, 6(5):447-458, 2004.
Su et al., Clin Cancer Res, 7(5):1320-1324, 2001.
Sui et al., Oncol Rep, 15(4):765-771, 2006.
Takanami, Oncol Rep, 13(4):727-731, 2005.
Tanaka et al., Proc Natl Acad Sci USA, 95(17):10164-10169, 1998.
Thogersen et al., Cancer Res, 61(16):6227-6233, 2001.
Tomasini-Johansson et al., Exp Cell Res, 214(1):303-312, 1994.
Torring et al., Anticancer Res, 20(1A):91-95, 2000.
Toyoda et al., Biochem J, 326 (Pt 1):69-75, 1997.
Traub et al., Breast Cancer Res Treat, 99(2):185-191, 2006.
Uhm et al., Clin Cancer Res, 5(6):1587-1594, 1999.
UK 1,529,202
UK 8 803 000
Volinia et al., Proc Natl Acad Sci USA, 103(7):2257-2261, 2006.
Weeraratna et al., Cancer Cell, 1(3):279-288, 2002.
Weiss and Bohmann, Cell Cycle, 3(2):111-113, 2004.
Wheeler and Ridley, Exp Cell Res, 301(1):43-49, 2004.
Wu et al., Breast Cancer Res Treat, 84(1):3-12, 2004b.
Wu et al., Eur J Cancer, 42(4):557-565, 2006a.
Wu et al., Gynecol Oncol, 102(1):15-21, 2006b.
Wu et al., Pathol Oncol Res, 10(1):26-33, 2004a.
Xu et al., Curr. Biol., 13(9):790-795, 2003.
Yang et al., J Androl, 22(3):471-480, 2001.
Yao et al., Oncogene, 25(16):2285-2296, 2006.
Yoshioka et al., Proc Natl Acad Sci USA, 100(12):7247-7252, 2003.
Yu et al., Nat Genet, 37(3):265-274, 2005.
Zhang et al., Oncogene, 23(12):2241-2249, 2004.
Zhao et al., Mol Cancer Res, 1(3):195-206, 2003.
Zhu et al., Biochem Biophys Res Commun, 273(3):1019-1024, 2000.

1. A method of modulating gene expression in a cell comprising administering to the cell an amount of an isolated nucleic acid comprising a miR-20 nucleic acid sequence in an amount sufficient to modulate the expression of one or more genes identified in Table 1, 3, 4, or 5.

2. The method of claim 1, wherein the cell is in a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

3. The method of claim 2, wherein the infectious disease or condition is a parasitic, bacterial, viral, or fungal infection.

4. The method of claim 2, wherein the cancerous condition is astrocytoma, acute myelogenous leukemia, breast carcinoma, bladder carcinoma, cervical carcinoma, colorectal carcinoma, endometrial carcinoma, esophageal squamous cell carcinoma, glioma, glioblastoma, gastric carcinoma, hepatocellular carcinoma, Hodgkin lymphoma, leukemia, lipoma, melanoma, mantle cell lymphoma, myxofibrosarcoma, multiple myeloma, neuroblastoma, non-Hodgkin lymphoma, lung carcinoma, non-small cell lung carcinoma, ovarian carcinoma, esophageal carcinoma, osteosarcoma, pancreatic carcinoma, prostate carcinoma, squamous cell carcinoma of the head and neck, thyroid carcinoma, urothelial carcinoma wherein the modulation of one or more gene is sufficient for a therapeutic response.

5. The method of claim 1, wherein the expression of a gene is down-regulated.

6. The method of claim 1, wherein the expression of a gene is up-regulated.

7. The method of claim 1, wherein the cell is an epithelial, an endothelial, a mesothelial, a stromal, or a mucosal cell.

8. The method of claim 1, wherein the cell is a brain, a neuronal, a blood, an esophageal, a lung, a cardiovascular, a liver, a breast, a bone, a thyroid, a glandular, an adrenal, a pancreatic, a stomach, a intestinal, a kidney, a bladder, a prostate, a uterus, an ovarian, a testicular, a splenic, a skin, a smooth muscle, a cardiac muscle, a striated muscle cell.

9. The method of claim 1, wherein the cell is a cancer cell.

10. The method of claim 9, wherein the cancer cell is a neuronal, glial, lung, liver, brain, breast, bladder, blood, leukemic, colon, endometrial, stomach, skin, ovarian, fat, bone, cervical, esophageal, pancreatic, prostate, kidney, or thyroid cell.

11. The method of claim 1, wherein the isolated miR-20 nucleic acid is a recombinant nucleic acid.

12-16. (canceled)

17. The method of claim 1, wherein the miR-20 nucleic acid is a synthetic nucleic acid.

18. The method of claim 17, wherein the nucleic acid is administered at a dose of 0.01 mg/kg of body weight to 10 mg/kg of body weight.

19. The method of claim 1, wherein the miR-20 is a hsa-miR-20.

20. (canceled)

21. The method of claim 1, wherein the nucleic acid is administered enterally or parenterally.

22-23. (canceled)

24. The method of claim 1, wherein the nucleic acid is comprised in a pharmaceutical formulation.

25. The method of claim 24, wherein the pharmaceutical formulation is a lipid composition or a nanoparticle composition.

26. (canceled)

27. The method of claim 24, wherein the pharmaceutical formulation consists of biocompatible and biodegradable molecules.

28.-43. (canceled)

44. A method of treating a patient diagnosed with or suspected of having or suspected of developing a pathological condition or disease related to a gene modulated by a miRNA comprising the steps of:
(a) administering to the patient an amount of an isolated nucleic acid comprising a miR-20 nucleic acid sequence in an amount sufficient to modulate a cellular pathway or a physiologic pathway; and
(b) administering a second therapy, wherein the modulation of the cellular pathway or physiologic pathway sensitizes the patient to the second therapy.

45-49. (canceled)

50. A method of assessing a cell, tissue, or subject comprising assessing expression of miR-20 in combination with assessing expression of one or more gene from Table 1, 3, 4, or 5 in at least one sample.

51. (canceled)


SEQUENCE LISTING (269 sequences)

SEQ ID NO: 1 - RNA, 23 nt, Homo sapiens
uaaagugcuuauagugcagguag

SEQ ID NO: 2 - RNA, 23 nt, Homo sapiens
caaagugcucauagugcagguag

SEQ ID NO: 3 - RNA, 22 nt, Ateles geoffroyi
uaaagugcuuauagugcaggua

SEQ ID NO: 4 - RNA, 23 nt, Bos taurus
uaaagugcuuauagugcagguag

SEQ ID NO: 5 - RNA, 22 nt, Bos taurus
caaagugcucacagugcaggua

SEQ ID NO: 6 - RNA, 23 nt, Danio rerio
uaaagugcuuauagugcagguag

SEQ ID NO: 7 - RNA, 22 nt, Danio rerio
acugcagugugagcacuugaag

SEQ ID NO: 8 - RNA, 23 nt, Danio rerio
caaagugcucacagugcagguag

SEQ ID NO: 9 - RNA, 23 nt, Fugu rubripes
uaaagugcuuauagugcagguag

SEQ ID NO: 10 - RNA, 23 nt, Gallus gallus
uaaagugcuuauagugcagguag

SEQ ID NO: 11 - RNA, 23 nt, Gallus gallus
caaagugcucauagugcagguag

SEQ ID NO: 12 - RNA, 22 nt, Gorilla gorilla
uaaagugcuuauagugcaggua

SEQ ID NO: 13 - RNA, 22 nt, Lemur catta
uaaagugcuuauagugcaggua

SEQ ID NO: 14 - RNA, 22 nt, Lagothrix lagotricha
uaaagugcuuauagugcaggua

SEQ ID NO: 15 - RNA, 23 nt, Monodelphis domestica
uaaagugcuuauagugcagguag

SEQ ID NO: 16 - RNA, 22 nt, Macaca mulatta
uaaagugcuuauagugcaggua

SEQ ID NO: 17 - RNA, 23 nt, Mus musculus
uaaagugcuuauagugcagguag

SEQ ID NO: 18 - RNA, 22 nt, Mus musculus
caaagugcucauagugcaggua

SEQ ID NO: 19 - RNA, 22 nt, Macaca nemestrina
uaaagugcuuauagugcaggua

SEQ ID NO: 20 - RNA, 22 nt, Pan paniscus
uaaagugcuuauagugcaggua

SEQ ID NO: 21 - RNA, 22 nt, Pongo pygmaeus
uaaagugcuuauagugcaggua

SEQ ID NO: 22 - RNA, 22 nt, Pan troglodytes
uaaagugcuuauagugcaggua

SEQ ID NO: 23 - RNA, 23 nt, Rattus norvegicus
uaaagugcuuauagugcagguag

SEQ ID NO: 24 - RNA, 21 nt, Rattus norvegicus
acugcauuacgagcacuuaca

SEQ ID NO: 25 - RNA, 21 nt, Rattus norvegicus
caaagugcucauagugcaggu

SEQ ID NO: 26 - RNA, 21 nt, Rattus norvegicus
acugcagugugagcacuucug

SEQ ID NO: 27 - RNA, 22 nt, Saguinus labiatus
uaaagugcuuauagugcaggua

SEQ ID NO: 28 - RNA, 22 nt, Sus scrofa
uaaagugcuuauagugcaggua

SEQ ID NO: 29 - RNA, 23 nt, Tetraodon nigrovirdis
uaaagugcuuauagugcagguag

SEQ ID NO: 30 - RNA, 23 nt, Xenopus laevis
caaagugcucauagugcagguag

SEQ ID NO: 31 - RNA, 23 nt, Xenopus tropicalis
uaaagugcuuauagugcagguag

SEQ ID NO: 32 - RNA, 21 nt, Xenopus tropicalis
acugcauaaugagcacuuaaa

SEQ ID NO: 33 - RNA, 23 nt, Xenopus tropicalis
caaagugcucauagugcagguag

SEQ ID NO: 34 - RNA, 71 nt, Homo sapiens
guagcacuaaagugcuuauagugcagguaguguuuaguuaucuacugcauuaugagcacuuaaaguacugc

SEQ ID NO: 35 - RNA, 69 nt, Homo sapiens
aguaccaaagugcucauagugcagguaguuuuggcaugacucuacuguaguaugggcacuuccaguacu

SEQ ID NO: 36 - RNA, 71 nt, Ateles geoffroyi; identical to SEQ ID NO: 34

SEQ ID NO: 37 - RNA, 71 nt, Bos taurus; identical to SEQ ID NO: 34

SEQ ID NO: 38 - RNA, 69 nt, Bos taurus
aguaccaaagugcucacagugcagguaguuuuggcagcgcucuacuguagugugggcacuuccaguacu

SEQ ID NO: 39 - RNA, 153 nt, Danio rerio
ucuuaaauaaaucuccuaauguugcaguuguguuagaguuucagcagugcuaaagugcuuauagugcagguaguauuucugucaucuacugcagugugagcacuugaaguacuucuagcuuaaugcgucugggauccgaagauuucugcuaga

SEQ ID NO: 40 - RNA, 94 nt, Danio rerio
gaguuuguccuggcaguuccaaagugcucacagugcagguagugccaguggaucuacugcaaugucugcacuucaaguauugccggacgccuuc

SEQ ID NO: 41 - RNA, 80 nt, Fugu rubripes
agcaguauuaaagugcuuauagugcagguaguuacugauggucuacugcagugugagcacuucuaguacuucuagaugca

SEQ ID NO: 42 - RNA, 98 nt, Gallus gallus
ugacagcucuuguagcacuaaagugcuuauagugcagguaguguucacuaaucuacugcauuauaagcacuuaaaguacugcuagcuguagaacuaca

SEQ ID NO: 43 - RNA, 85 nt, Gallus gallus
cuagcaguaucaaagugcucauagugcagguagcuuggcauuggaccuacuguaaugugggcacuuacaguacuguuagauaaag

SEQ ID NO: 44 - RNA, 71 nt, Gorilla gorilla; identical to SEQ ID NO: 34

SEQ ID NO: 45 - RNA, 71 nt, Lemur catta
guagcacuaaagugcuuauagugcagguaguguuuaguuaucuacugcaucaugagcacuuaaaguacugc

SEQ ID NO: 46 - RNA, 71 nt, Lagothrix lagotricha; identical to SEQ ID NO: 34

SEQ ID NO: 47 - RNA, 102 nt, Monodelphis domestica
ugaaaggacagcuuuuguagcacuaaagugcuuauagugcagguaguguuuaguuaucuacugcauuaugagcacuugaaguacugcuagcuguagaacuac

SEQ ID NO: 48 - RNA, 71 nt, Macaca mulatta; identical to SEQ ID NO: 34

SEQ ID NO: 49 - RNA, 107 nt, Mus musculus
gugugaugugacagcuucuguagcacuaaagugcuuauagugcagguaguguguagccaucuacugcauuacgagcacuuaaaguacugccagcuguagaacuccag

SEQ ID NO: 50 - RNA, 80 nt, Mus musculus
ccuaguagugccaaagugcucauagugcagguaguuuuuauaccacucuacugcagugugagcacuucuaguacuccugg

SEQ ID NO: 51 - RNA, 142 nt, Macaca nemestrina
guagcacuaaagugcuuauagugcagguaguguuuaguuaucuacugcauuaugagcacuuaaaguacugcuguucuaaggugcaucuagugcagauagugaaguagauuagcaucuacugcccuaagugcuccuucuggca

SEQ ID NO: 52 - RNA, 71 nt, Pan paniscus; identical to SEQ ID NO: 34

SEQ ID NO: 53 - RNA, 71 nt, Pongo pygmaeus; identical to SEQ ID NO: 34

SEQ ID NO: 54 - RNA, 71 nt, Pan troglodytes; identical to SEQ ID NO: 34

SEQ ID NO: 55 - RNA, 85 nt, Rattus norvegicus
cagcuucuguagcacuaaagugcuuauagugcagguagugugucgucaucuacugcauuacgagcacuuacaguacugccagcug

SEQ ID NO: 56 - RNA, 72 nt, Rattus norvegicus
guagugccaaagugcucauagugcagguagguuuugcugcacucuacugcagugugagcacuucugguacuc

SEQ ID NO: 57 - RNA, 71 nt, Saguinus labiatus; identical to SEQ ID NO: 34

SEQ ID NO: 58 - RNA, 71 nt, Sus scrofa; identical to SEQ ID NO: 34

SEQ ID NO: 59 - RNA, 73 nt, Tetraodon nigrovirdis
gcaguauuaaagugcuuauagugcagguaguuuucucagggucuacugcaguaugagcacuucuaguacuucu

SEQ ID NO: 60 - RNA, 74 nt, Xenopus laevis
gcaguuccaaagugcucauagugcagguaguuguauucauguucuacuguaauaugggcacuuacaguacugcu

SEQ ID NO: 61 - RNA, 84 nt, Xenopus tropicalis
gcuaaaguggugcuaaagugcuuauagugcagguaguuuuucuguauucuacugcauaaugagcacuuaaaguacuccuagcug

SEQ ID NO: 62 - RNA, 74 nt, Xenopus tropicalis
gcaguuccaaagugcucauagugcagguaguuguauugauguucuacuguaauaugggcacuuacaguacugcu

SEQ ID NO: 63 - RNA, 24 nt, Ateles geoffroyi
aaaagugcuuacagugcagguagc

SEQ ID NO: 64 - RNA, 21 nt, Ateles geoffroyi
uaaagugcugacagugcagau

SEQ ID NO: 65 - RNA, 20 nt, Ateles geoffroyi
acugcagugaaggcacuugu

SEQ ID NO: 66 - RNA, 24 nt, Ateles geoffroyi
caaagugcuuacagugcagguagu

SEQ ID NO: 67 - RNA, 22 nt, Ateles geoffroyi
uaaggugcaucuagugcagaua

SEQ ID NO: 68 - RNA, 22 nt, Ateles geoffroyi
aaagugcuguucgugcagguag

SEQ ID NO: 69 - RNA, 22 nt, Bos taurus
aaaagugcuuacagugcaggua

SEQ ID NO: 70 - RNA, 20 nt, Bos taurus
acugcagugaaggcacuugu

SEQ ID NO: 71 - RNA, 24 nt, Bos taurus
caaagugcuuacagugcagguagu

SEQ ID NO: 72 - RNA, 22 nt, Bos taurus
uaaggugcaucuagugcagaua

SEQ ID NO: 73 - RNA, 22 nt, Bos taurus
uaaggugcaucuagugcaguua

SEQ ID NO: 74 - RNA, 22 nt, Bos taurus
caaagugcuguucgugcaggua

SEQ ID NO: 75 - RNA, 22 nt, Danio rerio
caaagugcuuacagugcaggua

SEQ ID NO: 76 - RNA, 22 nt, Danio rerio
acugcaguggaggcacuucuag

SEQ ID NO: 77 - RNA, 22 nt, Danio rerio
uaaggugcaucuagugcagaua

SEQ ID NO: 78 - RNA, 22 nt, Danio rerio
uaaggugcauuuagugcagaua

SEQ ID NO: 79 - RNA, 22 nt, Danio rerio
cugcccuaagugccccuucugg

SEQ ID NO: 80 - RNA, 22 nt, Danio rerio
uaaggugcaucuuguguaguua

SEQ ID NO: 81 - RNA, 22 nt, Danio rerio
aaaagugcuguuugugcaggua

SEQ ID NO: 82 - RNA, 22 nt, Fugu rubripes
caaagugcuuacagugcaggua

SEQ ID NO: 83 - RNA, 22 nt, Fugu rubripes
uaaggugcaucuagugcagaua

SEQ ID NO: 84 - RNA, 22 nt, Gallus gallus
aaaagugcuuacagugcaggua

SEQ ID NO: 85 - RNA, 20 nt, Gallus gallus
acugcagugaaggcacuugu

SEQ ID NO: 86 - RNA, 24 nt, Gallus gallus
caaagugcuuacagugcagguagu

SEQ ID NO: 87 - RNA, 22 nt, Gallus gallus
uaaggugcaucuagugcagaua

SEQ ID NO: 88 - RNA, 22 nt, Gallus gallus
uaaggugcaucuagugcaguua

SEQ ID NO: 89 - RNA, 24 nt, Gorilla gorilla
aaaagugcuuacagugcagguagc

SEQ ID NO: 90 - RNA, 21 nt, Gorilla gorilla
uaaagugcugacagugcagau

SEQ ID NO: 91 - RNA, 20 nt, Gorilla gorilla
acugcagugaaggcacuugu

SEQ ID NO: 92 - RNA, 24 nt, Gorilla gorilla
caaagugcuuacagugcagguagu

SEQ ID NO: 93 - RNA, 22 nt, Gorilla gorilla
uaaggugcaucuagugcagaua

SEQ ID NO: 94 - RNA, 22 nt, Gorilla gorilla
aaagugcuguucgugcagguag

SEQ ID NO: 95 - RNA, 24 nt, Homo sapiens
aaaagugcuuacagugcagguagc

SEQ ID NO: 96 - RNA, 21 nt, Homo sapiens
uaaagugcugacagugcagau

SEQ ID NO: 97 - RNA, 20 nt, Homo sapiens
acugcagugaaggcacuugu

SEQ ID NO: 98 - RNA, 24 nt, Homo sapiens
caaagugcuuacagugcagguagu

SEQ ID NO: 99 - RNA, 22 nt, Homo sapiens
uaaggugcaucuagugcagaua

SEQ ID NO: 100 - RNA, 21 nt, Homo sapiens
acugcccuaagugcuccuucu

SEQ ID NO: 101 - RNA, 22 nt, Homo sapiens
uaaggugcaucuagugcaguua

SEQ ID NO: 102 - RNA, 22 nt, Homo sapiens
aaagugcuguucgugcagguag

SEQ ID NO: 103 - RNA, 20 nt, Homo sapiens
acugcagugaaggcacuugu

SEQ ID NO: 104 - RNA, 24 nt, Lemur catta
caaagugcuuacagugcagguagu

SEQ ID NO: 105 - RNA, 22 nt, Lemur catta
uaaggugcaucuagugcagaua

SEQ ID NO: 106 - RNA, 21 nt, Lagothrix lagotricha
uaaagugcugacagugcagau

SEQ ID NO: 107 - RNA, 20 nt, Lagothrix lagotricha
acugcagugaaggcacuugu

SEQ ID NO: 108 - RNA, 24 nt, Lagothrix lagotricha
caaagugcuuacagugcagguagu

SEQ ID NO: 109 - RNA, 22 nt, Lagothrix lagotricha
uaaggugcaucuagugcagaua

SEQ ID NO: 110 - RNA, 22 nt, Lagothrix lagotricha
aaagugcuguucgugcagguag

SEQ ID NO: 111 - RNA, 21 nt, Monodelphis domestica
acugcagugaaggcacuugua

SEQ ID NO: 112 - RNA, 24 nt, Monodelphis domestica
caaagugcuuacagugcagguagu

SEQ ID NO: 113 - RNA, 22 nt, Monodelphis domestica
uaaggugcaucuagugcagaua

SEQ ID NO: 114 - RNA, 22 nt, Monodelphis domestica
aaagugcuguucgugcagguag

SEQ ID NO: 115 - RNA, 24 nt, Macaca mulatta
aaaagugcuuacagugcagguagc

SEQ ID NO: 116 - RNA, 21 nt, Macaca mulatta
uaaagugcugacagugcagau

SEQ ID NO: 117 - RNA, 20 nt, Macaca mulatta
acugcagugaaggcacuugu

SEQ ID NO: 118 - RNA, 24 nt, Macaca mulatta
caaagugcuuacagugcagguagu

SEQ ID NO: 119 - RNA, 22 nt, Macaca mulatta
uaaggugcaucuagugcagaua

SEQ ID NO: 120 - RNA, 22 nt, Macaca mulatta
aaagugcuguucgugcagguag

SEQ ID NO: 121 - RNA, 22 nt, Mus musculus
caaagugcuaacagugcaggua

SEQ ID NO: 122 - RNA, 21 nt, Mus musculus
uaaagugcugacagugcagau

SEQ ID NO: 123 - RNA, 21 nt, Mus musculus
acugcagugagggcacuugua

SEQ ID NO: 124 - RNA, 24 nt, Mus musculus
caaagugcuuacagugcagguagu

SEQ ID NO: 125 - RNA, 22 nt, Mus musculus
uaaggugcaucuagugcagaua

SEQ ID NO: 126 - RNA, 23 nt, Mus musculus
caaagugcuguucgugcagguag

SEQ ID NO: 127 - RNA, 24 nt, Macaca nemestrina
aaaagugcuuacagugcagguagc

SEQ ID NO: 128 - RNA, 21 nt, Macaca nemestrina
uaaagugcugacagugcagau

SEQ ID NO: 129 - RNA, 20 nt, Macaca nemestrina
acugcagugaaggcacuugu

SEQ ID NO: 130 - RNA, 24 nt, Macaca nemestrina
caaagugcuuacagugcagguagu

SEQ ID NO: 131 - RNA, 22 nt, Macaca nemestrina
uaaggugcaucuagugcagaua

SEQ ID NO: 132 - RNA, 22 nt, Macaca nemestrina
aaagugcuguucgugcagguag

SEQ ID NO: 133 - RNA, 24 nt, Pan paniscus
aaaagugcuuacagugcagguagc

SEQ ID NO: 134 - RNA, 21 nt, Pan paniscus
uaaagugcugacagugcagau

SEQ ID NO: 135 - RNA, 20 nt, Pan paniscus
acugcagugaaggcacuugu

SEQ ID NO: 136 - RNA, 24 nt, Pan paniscus
caaagugcuuacagugcagguagu

SEQ ID NO: 137 - RNA, 22 nt, Pan paniscus
uaaggugcaucuagugcagaua

SEQ ID NO: 138 - RNA, 22 nt, Pan paniscus
aaagugcuguucgugcagguag

SEQ ID NO: 139 - RNA, 24 nt, Pongo pygmaeus
aaaagugcuuacagugcagguagc

SEQ ID NO: 140 - RNA, 21 nt, Pongo pygmaeus
uaaagugcugacagugcagau

SEQ ID NO: 141 - RNA, 20 nt, Pongo pygmaeus
acugcagugaaggcacuugu

SEQ ID NO: 142 - RNA, 24 nt, Pongo pygmaeus
caaagugcuuacagugcagguagu

SEQ ID NO: 143 - RNA, 22 nt, Pongo pygmaeus
uaaggugcaucuagugcagaua

SEQ ID NO: 144 - RNA, 22 nt, Pongo pygmaeus
aaagugcuguucgugcagguag

SEQ ID NO: 145 - RNA, 24 nt, Pan troglodytes
aaaagugcuuacagugcagguagc

SEQ ID NO: 146 - RNA, 21 nt, Pan troglodytes
uaaagugcugacagugcagau

SEQ ID NO: 147 - RNA, 20 nt, Pan troglodytes
acugcagugaaggcacuugu

SEQ ID NO: 148 - RNA, 24 nt, Pan troglodytes
caaagugcuuacagugcagguagu

SEQ ID NO: 149 - RNA, 22 nt, Pan troglodytes
uaaggugcaucuagugcagaua

SEQ ID NO: 150 - RNA, 22 nt, Pan troglodytes
aaagugcuguucgugcagguag

SEQ ID NO: 151 - RNA, 21 nt, Rattus norvegicus
uaaagugcugacagugcagau

SEQ ID NO: 152 - RNA, 24 nt, Rattus norvegicus
caaagugcuuacagugcagguagu

SEQ ID NO: 153 - RNA, 22 nt, Rattus norvegicus
uaaggugcaucuagugcagaua

SEQ ID NO: 154 - RNA, 23 nt, Rattus norvegicus
caaagugcuguucgugcagguag

SEQ ID NO: 155 - RNA, 24 nt, Saguinus labiatus
aaaagugcuuacagugcagguagc

SEQ ID NO: 156 - RNA, 21 nt, Saguinus labiatus
uaaagugcugacagugcagau

SEQ ID NO: 157 - RNA, 20 nt, Saguinus labiatus
acugcagugaaggcacuugu

SEQ ID NO: 158 - RNA, 24 nt, Saguinus labiatus
caaagugcuuacagugcagguagu

SEQ ID NO: 159 - RNA, 22 nt, Saguinus labiatus
uaaggugcaucuagugcagaua

SEQ ID NO: 160 - RNA, 22 nt, Saguinus labiatus
aaagugcuguucgugcagguag

SEQ ID NO: 161 - RNA, 24 nt, Sus scrofa
aaaagugcuuacagugcagguagc

SEQ ID NO: 162 - RNA, 22 nt, Sus scrofa
uaaggugcaucuagugcagaua

SEQ ID NO: 163 - RNA, 22 nt, Tetraodon nigrovirdis
caaagugcuuacagugcaggua

SEQ ID NO: 164 - RNA, 22 nt, Tetraodon nigrovirdis
uaaggugcaucuagugcagaua

SEQ ID NO: 165 - RNA, 23 nt, Xenopus laevis
uaaggugcaucuagugcaguuag

SEQ ID NO: 166 - RNA, 23 nt, Xenopus laevis
caaagugcucauagugcagguag

SEQ ID NO: 167 - RNA, 24 nt, Xenopus tropicalis
aaaagugcuuauagugcagguaga

SEQ ID NO: 168 - RNA, 20 nt, Xenopus tropicalis
acugcagugaaggcacuugu

SEQ ID NO: 169 - RNA, 24 nt, Xenopus tropicalis
caaagugcuuacagugcagguagu

SEQ ID NO: 170 - RNA, 22 nt, Xenopus tropicalis
uaaggugcaucuagugcagaua

SEQ ID NO: 171 - RNA, 23 nt, Xenopus tropicalis
uaaggugcaucuagugcaguuag

SEQ ID NO: 172 - RNA, 23 nt, Xenopus tropicalis
caaagugcuguucgugcagguag

SEQ ID NO: 173 - RNA, 23 nt, Xenopus tropicalis
ucaagugcuguucgugcagguag

SEQ ID NO: 174 - RNA, 81 nt, Ateles geoffroyi
ccuuggccauguaaaagugcuuacagugcagguagcuuuuugagaucuacugcaaugcaagcacuucuuacauuaccaugg

SEQ ID NO: 175 - RNA, 82 nt, Ateles geoffroyi
ccugccggggcuaaagugcugacagugcagauagugguccucuccgugcuaccgcacuguggguacuugcugcuccagcagg

SEQ ID NO: 176 - RNA, 84 nt, Ateles geoffroyi
gucagaauaaugucaaagugcuuacagugcagguagugauaugugcaucuacugcagugaaggcacuuguagcauuauggugac

SEQ ID NO: 177 - RNA, 71 nt, Ateles geoffroyi
uguucuaaggugcaucuagugcagauagugaaguagauuagcaucuacugcccuaagugcuccuucuggca

SEQ ID NO: 178 - RNA, 80 nt, Ateles geoffroyi
cugggggcuccaaagugcuguucgugcagguagugugauuaccugaccuacugcugagcuagcacuucccgagcccccgg

SEQ ID NO: 179 - RNA, 81 nt, Bos taurus; identical to SEQ ID NO: 174

SEQ ID NO: 180 - RNA, 84 nt, Bos taurus; identical to SEQ ID NO: 176

SEQ ID NO: 181 - RNA, 71 nt, Bos taurus; identical to SEQ ID NO: 177

SEQ ID NO: 182 - RNA, 43 nt, Bos taurus
cuuguguuaaggugcaucuagugcaguuagugaagcagcucag

SEQ ID NO: 183 - RNA, 77 nt, Bos taurus
cugggggcuccaaagugcuguucgugcagguagugugaucaccugaccuacugcugagccagcacuucccgagcccc

SEQ ID NO: 184 - RNA, 138 nt, Danio rerio
ggacuuucuugaguggacuugguuggugucaauguauugucaaagugcuuacagugcagguaguauuauggaauaucuacugcaguggaggcacuucuagcaauacacuugaccauuuuaaccuuccuccaggcaucc

SEQ ID NO: 185 - RNA, 83 nt, Danio rerio
gucacuguagugucaaagugcuuacagugcagguaguucaauauaaucuacugcaguggaggcacuucaagcuuuaccgugac

SEQ ID NO: 186 - RNA, 83 nt, Danio rerio
ggcuuugugcuaaggugcaucuagugcagauagugaaguagacuagcaccuacugcccuaagugcuccuucuggcacgagggu

SEQ ID NO: 187 - RNA, 87 nt, Danio rerio
uacugcuuaugcuaaggugcauuuagugcagauagugaaauagacuagcaccuacugcccuaagugccccuucuggcauaaggggcu

SEQ ID NO: 188 - RNA, 85 nt, Danio rerio
gccuuccugcuaaggugcaucuuguguaguuagugaaguagucuaguaucuacugcgcuagauguuccuuuuggcaggaguagcu

SEQ ID NO: 189 - RNA, 74 nt, Danio rerio
guguguguuaaaagugcuguuugugcagguaguguguuuccucuacuguaggagcagcacuucacaacacacac

SEQ ID NO: 190 - RNA, 86 nt, Fugu rubripes
gucuguguauugccaaagugcuuacagugcagguaguucuaugugacaccuacugcaauggaggcacuuacagcaguacucuugac

SEQ ID NO: 191 - RNA, 82 nt, Fugu rubripes
gucaaaguagugucaaagugcuuacagugcagguaguuugaugacaucuacugcagugaaggcacuuucagcgcuauucuga

SEQ ID NO: 192 - RNA, 76 nt, Fugu rubripes
uugugcuaaggugcaucuagugcagauagugaaauagacuagcaccuacugcccuaagugcuccuucuggcauaag

SEQ ID NO: 193 - RNA, 81 nt, Gallus gallus
ccuugaguugugcaaaagugcuuacagugcagguagagcucagcaccuacugcaguauaagcacuucuggcaugaccgugg

SEQ ID NO: 194 - RNA, 85 nt, Gallus gallus
gucagaguaaugucaaagugcuuacagugcagguagugauauauagaaccuacugcagugaaggcacuuguagcauuauguugac

SEQ ID NO: 195 - RNA, 93 nt, Gallus gallus
gugcuuuuuguacuaaggugcaucuagugcagauagugaaguagauuagcaucuacugcccuaagugcuccuucuggcauaagaaguuauguc

SEQ ID NO: 196 - RNA, 84 nt, Gallus gallus
cgugguccuuguguuaaggugcaucuagugcaguuagugacguagcguagaaucuacugcccuaaaugcuccuucuggcacaag

SEQ ID NO: 197 - RNA, 81 nt, Gorilla gorilla; identical to SEQ ID NO: 174

SEQ ID NO: 198 - RNA, 82 nt, Gorilla gorilla; identical to SEQ ID NO: 175

SEQ ID NO: 199 - RNA, 84 nt, Gorilla gorilla; identical to SEQ ID NO: 176

SEQ ID NO: 200 - RNA, 71 nt, Gorilla gorilla; identical to SEQ ID NO: 177

SEQ ID NO: 201 - RNA, 80 nt, Gorilla gorilla
cugggggcuccaaagugcuguucgugcagguagugugauuacccgaccuacugcugagcuagcacuucccgagcccccgg

SEQ ID NO: 202 - RNA, 81 nt, Homo sapiens
ccuuggccauguaaaagugcuuacagugcagguagcuuuuugagaucuacugcaauguaagcacuucuuacauuaccaugg

SEQ ID NO: 203 - RNA, 82 nt, Homo sapiens; identical to SEQ ID NO: 175

SEQ ID NO: 204 - RNA, 84 nt, Homo sapiens; identical to SEQ ID NO: 176

SEQ ID NO: 205 - RNA, 71 nt, Homo sapiens; identical to SEQ ID NO: 177

SEQ ID NO: 206 - RNA, 71 nt, Homo sapiens
uguguuaaggugcaucuagugcaguuagugaagcagcuuagaaucuacugcccuaaaugccccuucuggca

SEQ ID NO: 207 - RNA, 80 nt, Homo sapiens
cugggggcuccaaagugcuguucgugcagguagugugauuacccaaccuacugcugagcuagcacuucccgagcccccgg

SEQ ID NO: 208 - RNA, 84 nt, Lemur catta; identical to SEQ ID NO: 176

SEQ ID NO: 209 - RNA, 71 nt, Lemur catta; identical to SEQ ID NO: 177

SEQ ID NO: 210 - RNA, 81 nt, Lagothrix lagotricha
ccugccggggcuaaagugcugacagugcagauaguguccucuccgugcuaccgcacuguggguacuugcugcuccagcagg

SEQ ID NO: 211 - RNA, 84 nt, Lagothrix lagotricha; identical to SEQ ID NO: 176

SEQ ID NO: 212 - RNA, 71 nt, Lagothrix lagotricha; identical to SEQ ID NO: 177

SEQ ID NO: 213 - RNA, 80 nt, Lagothrix lagotricha
cucggggcuccaaagugcuguucgugcagguagugugauuaccugaccuacugcugagcuagcacuucccgagcccccgg

SEQ ID NO: 214 - RNA, 85 nt, Monodelphis domestica
gucagaauaaugucaaagugcuuacagugcagguagugauauguagaaucuacugcagugaaggcacuuguagcauuauagugac

SEQ ID NO: 215 - RNA, 72 nt, Monodelphis domestica
uuguucuaaggugcaucuagugcagauagugaaguagauuagcaucuacugcccuaagugcuccuucuggca

SEQ ID NO: 216 - RNA, 89 nt, Monodelphis domestica
agucuuggggggcuccaaagugcuguucgugcagguagugugauaaccugaccuacugcugagcuagcacuuccagagccccugggaca

SEQ ID NO: 217 - RNA, 81 nt, Macaca mulatta; identical to SEQ ID NO: 174

SEQ ID NO: 218 - RNA, 82 nt, Macaca mulatta
ccugcuggggcuaaagugcugacagugcagauagugguccucuccgugcuaccgcacuguggguacuugcugcuccagcagg

SEQ ID NO: 219 - RNA, 84 nt, Macaca mulatta; identical to SEQ ID NO: 176

SEQ ID NO: 220 - RNA, 71 nt, Macaca mulatta; identical to SEQ ID NO: 177

SEQ ID NO: 221 - RNA, 80 nt, Macaca mulatta
cugggggcuccaaagugcuguucgugcagguagugugauuaaccgaccuacugcugagcuagcacuucccgagcccccag

SEQ ID NO: 222 - RNA, 65 nt, Mus musculus
augucaaagugcuaacagugcagguagcuuuuugaguucuacugcagugccagcacuucuuacau

SEQ ID NO: 223 - RNA, 82 nt, Mus musculus
ccugcugggacuaaagugcugacagugcagauagugguccucucugugcuaccgcacuguggguacuugcugcuccagcagg

SEQ ID NO: 224 - RNA, 84 nt, Mus musculus
gucagaauaaugucaaagugcuuacagugcagguagugaugugugcaucuacugcagugagggcacuuguagcauuaugcugac

SEQ ID NO: 225 - RNA, 96 nt, Mus musculus
ugcgugcuuuuuguucuaaggugcaucuagugcagauagugaaguagacuagcaucuacugcccuaagugcuccuucuggcauaagaaguuauguc

SEQ ID NO: 226 - RNA, 88 nt, Mus musculus
agucaugggggcuccaaagugcuguucgugcagguaguguaauuaccugaccuacugcugagcuagcacuucccgagcccccaggaca

SEQ ID NO: 227 - RNA, 81 nt, Macaca nemestrina; identical to SEQ ID NO: 174

SEQ ID NO: 228 - RNA, 82 nt, Macaca nemestrina; identical to SEQ ID NO: 218

SEQ ID NO: 229 - RNA, 84 nt, Macaca nemestrina; identical to SEQ ID NO: 176

SEQ ID NO: 230 - RNA, 71 nt, Macaca nemestrina; identical to SEQ ID NO: 177

SEQ ID NO: 231 - RNA, 80 nt, Macaca nemestrina; identical to SEQ ID NO: 221

SEQ ID NO: 232 - RNA, 81 nt, Pan paniscus; identical to SEQ ID NO: 174

SEQ ID NO: 233 - RNA, 82 nt, Pan paniscus; identical to SEQ ID NO: 175

SEQ ID NO: 234 - RNA, 84 nt, Pan paniscus; identical to SEQ ID NO: 176

SEQ ID NO: 235 - RNA, 71 nt, Pan paniscus; identical to SEQ ID NO: 177

SEQ ID NO: 236 - RNA, 80 nt, Pan paniscus; identical to SEQ ID NO: 201

SEQ ID NO: 237 - RNA, 81 nt, Pongo pygmaeus; identical to SEQ ID NO: 174

SEQ ID NO: 238 - RNA, 82 nt, Pongo pygmaeus; identical to SEQ ID NO: 175

SEQ ID NO: 239 - RNA, 84 nt, Pongo pygmaeus
gucagaauaaugucaaagugcuuacagugcagguagugauacgugcaucuacugcagugaaggcacuuguagcauuauggugac

SEQ ID NO: 240 - RNA, 71 nt, Pongo pygmaeus; identical to SEQ ID NO: 177

SEQ ID NO: 241 - RNA, 80 nt, Pongo pygmaeus; identical to SEQ ID NO: 201

SEQ ID NO: 242 - RNA, 81 nt, Pan troglodytes; identical to SEQ ID NO: 174

SEQ ID NO: 243 - RNA, 82 nt, Pan troglodytes; identical to SEQ ID NO: 175

SEQ ID NO: 244 - RNA, 84 nt, Pan troglodytes; identical to SEQ ID NO: 176

SEQ ID NO: 245 - RNA, 71 nt, Pan troglodytes; identical to SEQ ID NO: 177

SEQ ID NO: 246 - RNA, 80 nt, Pan troglodytes; identical to SEQ ID NO: 201

SEQ ID NO: 247 - RNA, 82 nt, Rattus norvegicus; identical to SEQ ID NO: 223

SEQ ID NO: 248 - RNA, 84 nt, Rattus norvegicus
gucaggauaaugucaaagugcuuacagugcagguaguggugugugcaucuacugcagugaaggcacuuguggcauugugcugac

SEQ ID NO: 249 - RNA, 96 nt, Rattus norvegicus; identical to SEQ ID NO: 225

SEQ ID NO: 250 - RNA, 87 nt, Rattus norvegicus
agucaugggggcuccaaagugcuguucgugcagguagugcauugccugaccuacugcugagcuagcacuucccgagcccccaggaca

SEQ ID NO: 251 - RNA, 81 nt, Saguinus labiatus; identical to SEQ ID NO: 174

SEQ ID NO: 252 - RNA, 82 nt, Saguinus labiatus; identical to SEQ ID NO: 175

SEQ ID NO: 253 - RNA, 84 nt, Saguinus labiatus; identical to SEQ ID NO: 176

SEQ ID NO: 254 - RNA, 71 nt, Saguinus labiatus; identical to SEQ ID NO: 177

SEQ ID NO: 255 - RNA, 80 nt, Saguinus labiatus; identical to SEQ ID NO: 207

SEQ ID NO: 256 - RNA, 81 nt, Sus scrofa
ccuuggccguguaaaagugcuuacagugcagguagcuuuuugagaucuacugcaaugcaagcacuucuuacauuaccaugg

SEQ ID NO: 257 - RNA, 92 nt, Sus scrofa
gugcuuuuuguucuaaggugcaucuagugcagauagugaaguagauuagcaucuacugcccuaagugcuccuucuggcauaagaaguuaugu

SEQ ID NO: 258 - RNA, 86 nt, Tetraodon nigrovirdis
gucuguguauugccaaagugcuuacagugcagguaguacuauguaacaccuacugcaauggaggcacuuacagcaguacucuugac

SEQ ID NO: 259 - RNA, 82 nt, Tetraodon nigrovirdis
gucaccguagugucaaagugcuuacagugcagguaguuaaaagaaaucuacugcagugaaggcacuuucagcacuauucuga

SEQ ID NO: 260 - RNA, 76 nt, Tetraodon nigrovirdis; identical to SEQ ID NO: 192

SEQ ID NO: 261 - RNA, 79 nt, Xenopus laevis
ccuuguguuaaggugcaucuagugcaguuagugacauagcguagcaucuacugcccuaaaugcuccuuuuggcacaggg

SEQ ID NO: 262 - RNA, 63 nt, Xenopus tropicalis
ugcaaaagugcuuauagugcagguagaauuuaaaccuacugcaccauaagcacuuccugcauc

SEQ ID NO: 263 - RNA, 87 nt, Xenopus tropicalis
gucaagaguaaugucaaagugcuuacagugcagguagugauuuaacagaaccuacugcagugaaggcacuuguagcauuauauugac

SEQ ID NO: 264 - RNA, 87 nt, Xenopus tropicalis
gugcuuuuuguccuaaggugcaucuagugcagauagugaaguagauuagcaucuacugcccuaagugcuccuucuggcauaaaaagu

SEQ ID NO: 265 - RNA, 78 nt, Xenopus tropicalis
ccuuguguuaaggugcaucuagugcaguuagugacauaguguagcaucuacugcccuaaaugcuccuuuuggcacagg

SEQ ID NO: 266 - RNA, 73 nt, Xenopus tropicalis
augggcuucaaagugcuguucgugcagguagcuuaauaacagaccuacugcaugggcggcacuucccaagccc

SEQ ID NO: 267 - RNA, 64 nt, Xenopus tropicalis
cuucaagugcuguucgugcagguagcuuaauaacagaccuacugcaugggcggcacuucccaag

SEQ ID NO: 268 - RNA, 17 nt, Artificial
  Artificial primer
sugcwnhnnrkgyasnu

SEQ ID NO: 269 - RNA, 21 nt, Artificial
  Artificial primer
yaaagugcuyayagugcaggu